(19) United States
(12) Patent Application Publication
(10) Pub. No.: US 2025/0269046 A1
(43) Pub. Date: Aug. 28, 2025

(54) CELLULAR UPTAKE OF LARGE BIOMOLECULES ENABLED BY CELL-SURFACE-REACTIVE CELL-PENETRATING PEPTIDE ADDITIVES

(71) Applicant: FORSCHUNGSVERBUND BERLIN E.V., Berlin (DE)

(72) Inventors: Christian HACKENBERGER, Berlin (DE); Anselm SCHNEIDER, Berlin (DE); Martin LEHMANN, Blankenfelde (DE); Jan Vincent ARAFILES, Berlin (DE)

(21) Appl. No.: 18/547,393

(22) PCT Filed: Feb. 25, 2022
(86) PCT No.: PCT/EP2022/054869

§ 371 Date: Aug. 22, 2023

(30) Foreign Application Priority Data
Feb. 26, 2021 (EP) 21159630.9

(51) Int. Cl. A61K 47/64; C07K 7/08

(52) U.S. Cl. CPC A61K 47/645; C07K 7/08

(57) ABSTRACT

The present invention is directed to a method for delivering a cargo into a cell, the method comprising incubating a compound comprising a moiety capable to bind to the cell surface and a guanidine moiety together with a cargo and a cell, wherein the cargo is connected with a group comprising guanidine moiety, thereby allowing delivering of the peptide or protein into the cell. The invention is further directed to a compound comprising a moiety capable to bind to the cell surface and guanidine moiety for use in delivering a cargo into a cell, distinct compounds, distinct compounds for use in delivering a cargo into a cell, a kit for use in delivering a cargo into a cell comprising a compound comprising a moiety capable to bind to the cell surface and a guanidine moiety.

### CROSS-REFERENCE TO RELATED APPLICATIONS

The present application claims the benefit of priority of European Patent Application No. 21159630.9 filed 26 Feb. 2021, the content of which is hereby incorporated by reference in its entirety for all purposes.

### TECHNICAL FIELD OF THE INVENTION

The present invention is directed to a method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the peptide or protein into the cell. The invention is further directed to a compound comprising a moiety capable to bind to the cell surface and guanidine moiety for use in delivering a cargo into a cell, distinct compounds, distinct compounds for use in delivering a cargo into a cell, a kit for use in delivering a cargo into a cell comprising a compound comprising a moiety capable to bind to the cell surface and a guanidine moiety.

### BACKGROUND OF THE INVENTION

Enabling the cellular delivery and cytosolic bioavailability of functional proteins constitutes a major challenge for the life sciences. The conjugation with cell-penetrating peptides (CPPs) is frequently used to mediate the cellular uptake of various protein cargoes. At low μM-concentrations, protein-CPP conjugates can undergo endosomal uptake, which necessitates endosomal escape to avoid entrapment in endosomes, whereas at higher concentrations they can cross cell membranes directly and reach the cytosol via a non-endosomal delivery pathway.

Proteins offer a tremendous structural and functional diversity, which makes them indispensable tools for biological and pharmacological applications. However, proteins are large and hydrophilic, and thus usually not cell-permeable, which severely limits their potential in both research and therapy. Consequently, the intracellular delivery of functional proteins remains one of the biggest challenges in the molecular life sciences, although considerable progress has been made recently (Fu, A., Tang, R., Hardie, J., Farkas, M. E. & Rotello, V. M. Promises and pitfalls of intracellular delivery of proteins. Bioconjug Chem 25, 1602-1608, doi:10.1021/bc500320j (2014). Du, S., Liew, S. S., Li, L. & Yao, S. Q. Bypassing Endocytosis: Direct Cytosolic Delivery of Proteins. J Am Chem Soc, doi:10.1021/jacs.8b06584 (2018)). Amongst other methods, cell-penetrating peptides (CPPs) have established themselves as potent tools in the delivery of a variety of cargoes (Wang, F. et al. Recent progress of cell-penetrating peptides as new carriers for intracellular cargo delivery. J Control Release 174, 126-136, doi:10.1016/j.jconrel.2013.11.020 (2014)).

The first cell-penetrating peptides or “protein transduction domains” (PTDs) have been discovered about 30 years ago originating from the transactivator of transcription (TAT) protein of the human immunodeficiency virus (HIV) (Viscidi, R. P., Mayur, K., Lederman, H. M. & Frankel, A. D. Inhibition of antigen-induced lymphocyte proliferation by Tat protein from HIV-1. Science 246, 1606-1608, doi:10.1126/science.2556795 (1989)) and the Drosophila antennapedia homeodomain (penetratin) (Joliot, A. H., Triller, A., Volovitch, M., Pernelle, C. & Prochiantz, A. alpha-2,8-Polysialic acid is the neuronal surface receptor of antennapedia homeobox peptide. New Biol 3, 1121-1134 (1991). Derossi, D., Joliot, A. H., Chassaing, G. & Prochiantz, A. The third helix of the Antennapedia homeodomain translocates through biological membranes. J Biol Chem 269, 10444-10450 (1994)). Since then, many CPPs have been described, both from natural and synthetic origin. Several studies have investigated their mechanism of action and used them in various applications in biology (Borrelli, A., Tornesello, A. L., Tornesello, M. L. & Buonaguro, F. M. Cell Penetrating Peptides as Molecular Carriers for Anti-Cancer Agents. Molecules 23, doi:10.3390/molecules23020295 (2018). Guidotti, G., Brambilla, L. & Rossi, D. Cell-Penetrating Peptides: From Basic Research to Clinics. Trends Pharmacol Sci 38, 406-424, doi:10.1016/j.tips.2017.01.003 (2017)); nevertheless, how exactly CPPs enter cells is still often controversially discussed. Much effort has been devoted to cationic CPPs, which can bind to cell membranes through ionic interactions before being endocytosed (Richard, J. P. et al. Cellular uptake of unconjugated TAT peptide involves clathrin-dependent endocytosis and heparan sulfate receptors. J Biol Chem 280, 15300-15306, doi:10.1074/jbc.M401604200 (2005). Fittipaldi, A. et al. Cell membrane lipid rafts mediate caveolar endocytosis of HIV-1 Tat fusion proteins. J Biol Chem 278, 34141-34149, doi:10.1074/jbc.M303045200 (2003)). Equally, many reports describe CPP-mediated uptake at cold temperatures, where active uptake processes should not occur, or in the presence of endocytosis inhibitors (Ben-Dov, N. & Korenstein, R. The uptake of HIV Tat peptide proceeds via two pathways which differ from macropinocytosis. Biochim Biophys Acta 1848, 869-877, doi:10.1016/j.bbamem.2014.12.015 (2015). Herce, H. D., Garcia, A. E. & Cardoso, M. C. Fundamental molecular mechanism for the cellular uptake of guanidinium-rich molecules. J. Am. Chem. Soc. 136, 17459-17467, doi:10.1021/ja507790z (2014)). The latter process, commonly referred to as “transduction”, is reported to be dependent on the concentration (Duchardt, F., Fotin-Mleczek, M., Schwarz, H., Fischer, R. & Brock, R. A comprehensive model for the cellular uptake of cationic cell-penetrating peptides. Traffic 8, 848-866, doi:10.1111/j.1600-0854.2007.00572.x (2007). Futaki, S. & Nakase, I. Cell-Surface Interactions on Arginine-Rich Cell-Penetrating Peptides Allow for Multiplex Modes of Internalization. Acc Chem Res 50, 2449-2456, doi:10.1021/acs.accounts.7b00221 (2017)) and also on the cargo attached to the peptide (He, L., Sayers, E. J., Watson, P. & Jones, A. T. Contrasting roles for actin in the cellular uptake of cell penetrating peptide conjugates. Sci Rep 8, 7318, doi:10.1038/s41598-018-25600-8 (2018)). This can be seen when using linear CPPs to transport small cargoes such as fluorophores and peptides, which typically leads to localization in the cytosol, whereas larger protein cargoes are often trapped in endosomes (Patel, S. G. et al. Cell-penetrating peptide sequence and modification dependent uptake and subcellular distribution of green florescent protein in different cell lines. Sci Rep 9, 6298, doi:10.1038/s41598-019-42456-8 (2019). Tunnemann, G. et al. Cargo-dependent mode of uptake and bioavailability of TAT-containing proteins and peptides in living cells. FASEB J. 20, 1775-1784, doi:10.1096/fj.05-5523com (2006)).

To further improve the delivery of CPP-linked cargoes researchers have implemented additional modules into uptake protocols. For instance, the addition of the enzyme sphingomyelinase resulted in an increased uptake of cationic CPPs by generating ceramide on the cell surface (Verdurmen, W. P., Thanos, M., Ruttekolk, I. R., Gulbins, E. & Brock, R. Cationic cell-penetrating peptides induce ceramide formation via acid sphingomyelinase: implications for uptake. J Control Release 147, 171-179, doi:10.1016/j.jconrel.2010.06.030 (2010)). Alternatively, the addition of pyrenebutyrate as a hydrophobic counterion to cells before adding the CPP-conjugate led to an improved uptake (Takeuchi, T. et al. Direct and rapid cytosolic delivery using cell-penetrating peptides mediated by pyrenebutyrate. ACS Chem Biol 1, 299-303, doi:10.1021/cb600127m (2006)). Finally, the addition of peptides or small molecules has been pursued to mediate endosomal leakage for the release of cargoes into the cytosol (Wadia, J. S., Stan, R. V. & Dowdy, S. F. Transducible TAT-HA fusogenic peptide enhances escape of TAT-fusion proteins after lipid raft macropinocytosis. Nat Med 10, 310-315, doi:10.1038/nm996 (2004). Erazo-Oliveras, A. et al. Protein delivery into live cells by incubation with an endosomolytic agent. Nat Methods 11, 861-867, doi:10.1038/nmeth.2998 (2014). Allen, J. et al. Cytosolic Delivery of Macromolecules in Live Human Cells Using the Combined Endosomal Escape Activities of a Small Molecule and Cell Penetrating Peptides. ACS Chem Biol, doi:10.1021/acschembio.9b00585 (2019). Akishiba, M. et al. Cytosolic antibody delivery by lipid-sensitive endosomolytic peptide. Nature Chemistry, doi:10.1038/nchem.2779 (2017). Morris, M. C., Depollier, J., Mery, J., Heitz, F. & Divita, G. A peptide carrier for the delivery of biologically active proteins into mammalian cells. Nat Biotechnol 19, 1173-1176, doi:10.1038/nbt1201-1173 (2001).

An important element in the design of effective CPPs for the transport of cargo to the cytosol is cyclization (Reichart, F., Horn, M. & Neundorf, I. Cyclization of a cell-penetrating peptide via click-chemistry increases proteolytic resistance and improves drug delivery. J Pept Sci 22, 421-426, doi:10.1002/psc.2885 (2016). Dougherty, P. G., Sahni, A. & Pei, D. Understanding Cell Penetration of Cyclic Peptides. Chem Rev 119, 10241-10287, doi:10.1021/acs.chemrev.9b00008 (2019)). It was demonstrated that cyclization leads to a remarkable increase in both efficiency and speed of membrane transduction (Lattig-Tunnemann, G. et al. Backbone rigidity and static presentation of guanidinium groups increases cellular uptake of arginine-rich cell-penetrating peptides. Nat Commun 2, 453, doi:10.1038/ncomms1459 (2011)), which likely stems from the presentation of the positive charges on the peptide. We have previously shown that cyclization and subsequent conjugation of a single TAT-peptide can be used to transport GFP into the cytosol of cells, which was impossible with the linear variant (Nischan, N. et al. Covalent attachment of cyclic TAT peptides to GFP results in protein delivery into live cells with immediate bioavailability. Angewandte Chemie 54, 1950-1953, doi:10.1002/anie.201410006 (2015)). Since then, we have been able to apply this methodology to the cytosolic transport of nanobodies as well as the intracellular targeting of fluorescent proteins (Schneider, A. F. L., Wallabregue, A. L. D., Franz, L. & Hackenberger, C. P. R. Targeted Subcellular Protein Delivery Using Cleavable Cyclic Cell-Penetrating Peptides. Bioconjug Chem 30, 400-404, doi:10.1021/acs.bioconjchem.8b00855 (2019). Herce, H. D. et al. Cell-permeable nanobodies for targeted immunolabelling and antigen manipulation in living cells. Nat Chem 9, 762-771, doi:10.1038/nchem.2811 (2017)). Still, to achieve transduction of proteins into the cytosol, rather high concentrations of the cargo protein must be applied, ranging from 10 μM of a small antibody fragment (nanobody) to up to over 100 μM of EGFP. These concentrations are much higher than required for the transduction of small cargoes (Lattig-Tunnemann, G. et al. Backbone rigidity and static presentation of guanidinium groups increases cellular uptake of arginine-rich cell-penetrating peptides. Nat Commun 2, 453, doi:10.1038/ncomms1459 (2011)) and suggest that there is a size-dependence of the cargo on transduction. Consequently, it might not be possible at all to deliver even larger molecules in an energy-independent manner.

Therefore, advancing the delivery of CPP-protein and CPP-antibody conjugates with even better uptake properties remains challenging and requires often laborious chemical protein engineering.

In view of this, the present invention underlies the technical problem to provide further methods and compounds which allow an efficient delivery of cargoes such as proteins or antibodies into a cell.

Encouraged by previous findings, which report the cooperative interaction of arginine-rich CPPs with physiological membranes in a concentration-dependent manner (Medina, S. H. et al. An Intrinsically Disordered Peptide Facilitates Non-Endosomal Cell Entry. Angewandte Chemie 55, 3369-3372, doi:10.1002/anie.201510518 (2016). Jones, A. T. & Sayers, E. J. Cell entry of cell penetrating peptides: tales of tails wagging dogs. J Control Release 161, 582-591, doi:10.1016/j.jconrel.2012.04.003 (2012). Robison, A. D. et al. Polyarginine Interacts More Strongly and Cooperatively than Polylysine with Phospholipid Bilayers. J Phys Chem B 120, 9287-9296, doi:10.1021/acs.jpcb.6b05604 (2016). Shi, J. & Schneider, J. P. De novo Design of Selective Membrane-Active Peptides by Enzymatic Control of Their Conformational Bias on the Cell Surface. Angewandte Chemie 58, 13706-13710, doi:10.1002/anie.201902470 (2019)), the impact of CPPs has been probed that were added to cells in addition to protein-CPP cargoes. Starting with simple cysteine-containing, unbound CPPs as additives, it has been found that thiol-reactivity plays an important role in this additive-approach, which is consistent with previous reports using thiol-reactive cargoes (Aubry, S. et al. Cell-surface thiols affect cell entry of disulfide-conjugated peptides. FASEB J 23, 2956-2967, doi:10.1096/fj.08-127563 (2009). Gasparini, G., Sargsyan, G., Bang, E. K., Sakai, N. & Matile, S. Ring Tension Applied to Thiol-Mediated Cellular Uptake. Angewandte Chemie 54, 7328-7331, doi:10.1002/anie.201502358 (2015)).

### SUMMARY

The present invention is directed to a method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell.

The invention is also directed to a compound comprising a moiety capable to bind to a cell surface and a guanidine moiety for use in delivering a cargo into a cell.

Further, the invention is directed to a compound of formula (1):

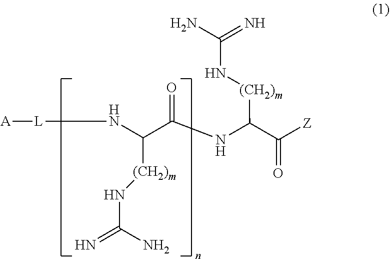

wherein:
A is a moiety capable to bind to a cell surface;
L is a linker or a bond;
m is each independently an integer ranging from 0 to 10;
n is an integer ranging from 1 to 20;
Z is selected from the group consisting of NR¹R², OR³, an amino acid, a peptide comprising 2 to 10 amino acids, and a hydrophobic moiety;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring;
R³ is hydrogen or (C₁-C₆)alkyl;
or a pharmaceutically acceptable salt thereof.

The invention is also directed to a compound according to the invention for a use according to the invention.

The invention is also directed to a compound comprising a moiety capable to bind to a cell surface and a guanidine moiety for use in delivering a cargo into a cell, wherein the compound is a compound according to the invention.

The invention is also directed to a method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, and wherein the compound is a compound according to the invention.

The invention is also directed to a method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, wherein
the moiety of the compound capable to bind to the cell surface is a thiol-reactive moiety, or wherein the moiety is capable to bind to the cell surface via an enzymatic reaction, preferably wherein the moiety is capable to bind to a tag, such as a Halotag; and/or
the compound comprising a moiety capable to bind to the cell surface and a guanidine moiety further comprises a hydrophobic moiety, and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours, preferably for 5 min to 60 minutes, and/or at a temperature of 4° C. to 37° C.

A method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, the method comprising:
(a) incubating the compound comprising a moiety capable to bind to the cell surface and a guanidine moiety together with the cargo connected with the group comprising a guanidine moiety to obtain a solution comprising the compound and the cargo connected with the group,
(b) incubating the solution of step (a) with the cell, thereby allowing delivering of the cargo into the cell, preferably wherein in (b) the incubating the solution of (a) with the cell is carried out for a time of 1 minute to 24 hours, preferably for 5 min to 60 minutes, and/or at a temperature of 4° C. to 37° C., and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours, preferably for 5 min to 60 minutes, and/or at a temperature of 4° C. to 37° C.

Further, the invention is directed to a kit for use in delivering a cargo into a cell, the kit comprising a compound comprising a moiety capable to bind to a cell surface and a guanidine moiety.

### BRIEF DESCRIPTION OF THE FIGURES

FIG. 1 shows the concentration dependent delivery of CPP-bearing red fluorescent cargoes into HeLa Kyoto cells at 37 and 4° C. a, Different modes of uptake at 37 and 4° C. b, Cellular uptake of TAMRA-cR10 at 37 and 4° C. and 1 μM concentration. c, Cellular uptake of fluorescently-labelled GBP1 nanobody (Structure: 3K1K) with cR10 peptide at 37 and 4° C. and 1 and 10 μM concentration, and at 1 μM concentration with 5 μM additional cR10 1. Here, the nuclear GFP fluorescence of the GFP-PCNA fusion protein is shown instead of the Hoechst staining. d, Cellular uptake of cR10-modified NLS-mCherry I (Structure: 2H5Q) at 37 and 4° C. and 10, 30 and 50 μM concentration, and at 5 μM concentration with 5 μM additional cR10. e, Cellular uptake of linear R10-modified NLS-mCherry II with added 5 μM linear R10 2. Scale bars 20 μm. Uppercase R is L-arginine while lower case R is D-arginine. Split channel images and additional concentrations in FIG. 9.

FIG. 2 shows TNB-R10 and its performance in delivering CPP-bearing cargoes into cells. a, Quantitative microscopy data showing the mean fluorescence intensity in the nucleus of mCherry-R10 conjugates I and II with R10-peptides 1-5 with representative microscopy images (pictures of at least 150 cells were taken in triplicates for each condition). Shown are individual values and mean±standard deviation. Unpaired t-test, ***=P<0.0005, **=P<0.005, n.s.=not significant. b, Time-lapse experiments showing the cellular uptake of fluorescent R10 peptides with different head groups. The arrowheads indicate nucleation zones where fluorescence is enriched before uptake into the cell. Scale bars 20 μm.

FIG. 3 shows Protein transduction into cells through CPP-labelled cell membranes. a, Time-lapse experiment of the simultaneous uptake of the TNB-R10-TAMRA 8 and Maleimide-R10-Cy5 13 peptides into cells in cell medium. The arrowheads indicate nucleation zones stained by both peptides. b, Time-lapse of the co-delivery of NLS-mCherry-R10 II together with the Maleimide-R10-Cy5 peptide 13 on HeLa Kyoto cells in cell medium. The arrowheads indicate nucleation zones and blue arrowheads the appearance of nucleolar staining of the mCherry. c, Cellular uptake of NLS-mCherry-cR10 I in presence of chloroalkane-modified “Halo-R10” 17 on HeLa Kyoto cells transfected with EGFP-transmembrane-Halotag plasmid, 1 hour at 37° C. in cell medium. SP: Signal peptide, TM: transmembrane sequence. The arrowheads show mCherry in the nucleoli of EGFP-positive cells. Uptake experiments with peptides 16 and 18-20 in FIG. 26. Scale bars 20 μm.

FIG. 4 shows co-delivery with cysteine-reactive R10 peptides in different cell lines and with different cargoes. a, Confocal microscopy images of four cancer cell lines treated with mCherry-R10 II and TNB-R10 5, for 1 hour at 37° C. b, Quantification of cells showing nucleolar fluorescence. At least 150 cells were counted over three biological replicates. Unpaired t-test, ****=P<0.0001, ***=P<0.0005. c, Cellular uptake of K10-modified mCherry with or without TNB-R10 5 at 37° C. leads to endosomal staining and no nucleolar fluorescence. d, Cellular uptake of mCherry with recombinant R10 (exR10) shows endosomal or nucleolar staining in absence and presence of TNB-R10 5, respectively. e, Scheme showing the cre stoplight reporter plasmid and flow cytometry data of HeLa CCL-2 cells transfected with it and subsequently treated with Cre-exR8 with or without Cys-R10 (2). f, Disulfide CPP modification of mCherry and in situ cellular uptake. Scale bars 20 μm.

FIG. 5 shows the application of TNB-R10 in IgG antibody delivery. a, Method of delivering antibodies into cells using TNB-R10. b, Cellular uptake of 500 nM Atto488-labelled Brentuximab into HeLa CCL-2 cells in presence and absence of TNB-R10 5 and at 37 and 4° C. Cells counterstained with Hoechst 33342 to demonstrate exclusion of the antibody from the nucleus. c, Cellular uptake of 500 nM Alexa594-labelled anti-GFP antibody into HeLa CCL-2 cells transfected with Lifeact-mVenus. d, Cellular uptake of 500 nM Atto488-labelled anti-TOMM20 antibody into HeLa CCL-2 cells, simultaneously treated with MitoTracker Red CMXROS. Scale bars 20 μm.

FIG. 6 shows the structures and UV-purity of peptides used in this study. a, TAMRA-cR10, HRMS: Calc.: 372.7257 [M+5H]. exp.: 372.7282. b, Cys-TAMRA, HRMS: Calc.: 504.7284 [M+2H], exp.: 504.7448. c, Cys-cR10 1, HRMS: Calc.: 553.8385 [M+4H], exp.: 553.9105. d, Maleimide-cR10, HRMS: Calc.: 449.8720 [M+5H], exp.: 449.8819. e, Cys-R10 2, HRMS: Calc.: 494.0568[M+4H], exp.: 494.0788. f, Maleimide-R10 (14), HRMS: Calc.: 402.2473 [M+5H], exp.: 402.2525. g, AA-R10 3, HRMS: Calc.: 508.3121 [M+4H], exp.: 508.3181. h, di-R10 4, HRMS: Calc.: 395.2453 [M+10H], exp.: 395.2491. i, TNB-R10 5, HRMS: Calc.: 543.3013 [M+4H], exp.: 543.3025. j, Maleimide-K10, HRMS: Calc.: 346.0343 [M+5H], exp.: 346.0502. k, Cys-R10-TAMRA 6, HRMS: Calc.: 419.7465 [M+5H], exp.: 419.7466. I, AA-R10-TAMRA 7, HRMS: Calc.: 367.9292 [M+6H], exp.: 367.9271. m, TNB-R10-TAMRA 8, HRMS: Calc.: 452.5769 [M+6H], exp.: 452.5703. n, TAMRA-R5-Cys-R5 9, HRMS: Calc.: 419.8458 [M+5H], exp.: 419.8387. o, TAMRA-R5-AA-R5 10, HRMS: Calc.: 431.2501 [M+5H], exp.: 431.2468. p, TAMRA-R10-Cys 11, HRMS: Calc.: 419.6451 [M+5H], exp.: 419.6376. q, TAMRA-R10-AA 12, HRMS: Calc.: 431.2501 [M+5H], exp.: 431.2383. r, Maleimide-R10-Cy5 13, HRMS: Calc.: 434.1036 [M+6H], exp.: 434.0886. s, Maleimide-R10-Biotin 15, HRMS: Calc.: 472.8811 [M+5H], exp.: 472.8772. t, Halo-R10 16, HRMS: Calc.: 428.5252 [M+4H], exp.: 428.5278. u, Halo-R10 17, HRMS: Calc.: 334.5444 [M+6H], exp.: 334.5357. v, Halo-R10 18, HRMS: Calc.: 573.8500 [M+4H], exp.: 573.8497. w, Halo-R10 19, HRMS: Calc.: 646.3869 [M+4H], exp.: 646.3752. x, Halo-R10 20, HRMS: Calc.: 718.9239 [M+4H], exp.: 718.9248. y, Maleimide-R5, HRMS: Calc.: 613.8546 [M+2H], exp.: 613.8481. z, Maleimide-R8, HRMS: Calc.: 424.2559 [M+4H], exp.: 424.2505.

FIG. 7 shows characterization of anti-GFP-nanobody GBP1 and its CPP conjugate. a, Strategy for the semi-synthesis of thiol- and fluorophore modified GBP1 nanobody via expressed protein ligation. b, SDS-PAGE gel, stained with Coomassie and fluorescence imaging of TAMRA on the bottom, of GBP1-TAMRA after expressed protein ligation (1) and after conjugation to the Maleimide-cR10 (2). Synthetic details in supplementary methods. c, High resolution mass spectrum of GBP1-TAMRA after EPL. Calc.: 13977 [M+H]; Exp.: 13975. d, High resolution mass spectrum of GBP1-TAMRA-cR10, Calc.: 16222 [M+H], 16240 [M+H2O+H](Maleimide ring opening hydrolysis); Exp.: 16240.

FIG. 8 shows the characterization of NLS-mCherry and its CPP conjugates. For synthetic details, see Protein-CPP conjugation section in the supplementary methods. a, SDS-PAGE gel showing the purity and conversion of NLS-mCherry (lane 1) and the linear R10, cyclic R10 and K10 peptide conjugates (lanes 2-4). The protein shows two lower molecular weight bands, which are an artefact of the sample preparation (boiling) for SDS-PAGE (Gross, L. A., Baird, G. S., Hoffman, R. C., Baldridge, K. K. & Tsien, R. Y. The structure of the chromophore within DsRed, a red fluorescent protein from coral. Proc Natl Acad Sci USA 97, 11990-11995, doi:10.1073/pnas.97.22.11990 (2000)). b, High resolution mass spectrum of NLS-mCherry, Calc.: 28339 [M+H]; Exp.: 28338. c, High resolution mass spectrum of NLS-mCherry-R10 II, Calc.: 30344 [M+H], 30362 [M+H2O+H]; Exp.: 30362. d, High resolution mass spectrum of NLS-mCherry-cR10 I, Calc.: 30583 [M+H], 30601 [M+H2O+H]; Exp.: 30602. e, High resolution mass spectrum of NLS-mCherry-K10 III, Calc.: 30064 [M+H], 30082 [M+H2O+H]; Exp.: 30064, 30082.

FIG. 9 shows the full set of confocal microscopy pictures after cellular uptake of R10-bearing cargoes into HeLa cells at 37 and 4° C. a, Uptake of TAMRA-cR10 at 1 μM, at 37 and 4° C. b, Uptake of 1-10 μM GBP1-TAMRA-cR10 (with additional Cys-cR10 1) at 37 and 4° C. c, Uptake of 1-50 μM NLS-mCherry-cR10 I and NLS-mCherry-R10 Ib (with additional Cys-cR10 1 or Cys-R10 2) at 37 and 4° C. Scale bars 20 μm.

FIG. 10 shows the additional experiments in the uptake of TAMRA-cR10 and NLS-mCherry. a, Uptake of 1 μM TAMRA-cR10 at 37° C., followed by washing with 25 μg/mL heparin to remove residual CPP bound to the cell membrane. b, Counted cells with nuclear or nucleolar fluorescence after uptake of 5 μM NLS-mCherry-R10 II with added Cys-R10 CPP 2. Over 150 cells were counted manually in three independent replicates. Shown are individual values with mean and standard deviation. c, Uptake of 5 μM NLS-mCherry-R10 II with added endosomolytic peptide ppTG21 at 37° C. for 1 hour. At the higher concentration, large aggregates are formed that persist after washing and are highly fluorescent. Scale bars 20 μm.

FIG. 11 shows the uptake of NLS-mCherry-R10 and Alexa647-Transferrin with endocytosis inhibitors. NLS-mCherry-R10 II at a 5 μM concentration was added to cells in combination with 25 μg/mL Alexa647-Transferrin (Invitrogen) as endocytosis control. Where indicated, 5 μM of the Cys-R10 peptide 2 were added to induce nucleolar delivery of the mCherry. For the incubation with sodium azide and Dynasore, the cells were pre-incubated with the inhibitors for 30 minutes, for pitstop 2 for 15 minutes, the inhibitors were then also added to the solution of mCherry and Transferrin. Nucleolar mCherry was seen anytime the Cys-R10 was present. In contrast, uptake of transferrin was strongly reduced in presence of azide and almost completely blocked in presence of pitstop 2, indicating that the inhibitors block endocytic uptake. Scale bars 20 μm.

FIG. 12 shows the representative images used in quantitative microscopy experiments. NLS-mCherry derivatives were added in the indicated concentration and with the indicated CPPs to HeLa CCL-2 cells for 1 hour at 37° C., and they were counterstained with Hoechst 33342. The cells were fixed after thorough washing to prevent an effect of the long microscopy time required. Confocal microscopy pictures were then taken, of at least 100 cells in independent triplicates at a 60× magnification to allow proper spatial separation of the nuclei and the endosomes. Scale bars 20 μm.

FIG. 13 shows the full graphs of relative and absolute quantification of cellular uptake. a, Absolute quantification of red fluorescence originating from the mCherry protein within the nucleus of HeLa cells. Images of at least 100 cells were taken in independent triplicates. Shown are individual values and mean±sd. b, Ratio of red fluorescence in the nucleus of cells divided by the total measured fluorescence in each frame. This is a measure of the efficiency of delivery to the nucleus and nucleolus relative to mCherry outside of the nucleus, predominantly a result of endosomal entrapment. Individual values and mean±sd. n.s.=not significant, *=P<0.05, **=P<0.005, ***=P<0.0005, ****=P<0.0001.

FIG. 14 shows the titration of NLS-mCherry-R10 II into cells with constant concentration of additive CPP. a, Microscopy pictures of the uptake of different concentrations of NLS-mCherry-R10 II in presence of constant 10 μM TNB-R10 (5). b, Quantification of the fluorescence intensity of a 20×20 pixel ROI in the nucleoli of 10 different cells per condition. Shown is the mean±SD for each concentration. A linear fit shows the linear relationship between applied concentration and resulting fluorescence (R²=0.92.)

FIG. 15 shows the montage of timelapse experiments of the cellular uptake of TAMRA-labelled R10 peptides with different N-terminal head groups. a, Uptake at 20 μM concentration. b, Uptake at 10 μM concentration. Insets show the appearance of nucleation zones (bright spots, immediately followed by uptake). c, Uptake at 5 μM concentration. The arrowheads show nucleation zones. Scale bars 20 μm.

FIG. 16 shows the montage of timelapse experiments of the cellular uptake of TNB-R10-TAMRA 8 in cells pre-treated with a small-molecule maleimide. a, Control uptake of the peptide without pre-treatment at 10 μM concentration. b, Uptake into cells that were treated first for 10 minutes with 1 mM of N,N-maleoyl glycine, followed by removal of the maleimide solution and addition of the peptide. c, Uptake into cells in presence of the anionic polysaccharide heparin. Scale bars 20 μm.

FIG. 17 shows the montage of timelapse experiments of the cellular uptake of AA-R10-TAMRA in cells co-incubated with cysteine. a, Control uptake of the peptide without cysteine at 10 μM concentration. b, Uptake into cells in presence of 10 μM L-cysteine. Scale bars 20 μm.

FIG. 18 shows the montage of timelapse experiments of the cellular uptake of Cys-R10-TAMRA with competition with free cysteine. a, b, Cellular uptake of the peptide with 10 (a) or 100 (b) μM L-cysteine. Scale bars 20 μm.

FIG. 19 shows the montage of timelapse experiments of the cellular uptake of R10-TAMRA peptides with cysteine at different positions, in comparison with acetylated variants. a, Cellular uptake of 10 μM cysteine-containing TAMRA-R5-Cys-R5 peptide. b, Uptake of the acetylated variant of a. c, Uptake of the cysteine containing TAMRA-R10-Cys peptide. d, Uptake of the acetylated variant of c. Scale bars 20 μm.

FIG. 20 shows the fluorescent labelling of accessible cell-surface thiols using cell-impermeable fluorophore. a, Labelling of accessible cell surface thiols with 10 μM of the membrane impermeant (sulfated) fluorophore atto 488 (Zhang, M., Li, M., Zhang, W., Han, Y. & Zhang, Y. H. Simple and efficient delivery of cell-impermeable organic fluorescent probes into live cells for live-cell superresolution imaging. Light Sci Appl 8, 73, doi:10.1038/s41377-019-0188-0 (2019)) functionalized with a maleimide. b, To confirm that Ellman's reagent and the fluorophore label the same thiols, cells were first treated with 50 μM Ellman's reagent for 10 minutes, then washed once and then treated with 10 μM of the fluorophore. Labelling is dramatically reduced. Scale bars 20 μm.

FIG. 21 shows the montage of timelapse experiments of the cellular uptake of the Maleimide-R10-Cy5 peptide 13 alone or in combination with TNB-R10-TAMRA 8 and NLS-mCherry-R10 II. a, Full dataset of uptake of 5 μM TNB-R10-TAMRA 8 with 5 μM Maleimide-R10-Cy5 13. b, Uptake of 10 μM Maleimide-R10-Cy5 13. c, Uptake of 5 μM NLS-mCherry-R10 II into cells in presence of 10 μM Maleimide-R10-Cy5 13. Scale bars 20 μm.

FIG. 22 shows the treatment of cells with maleimide-R10-Cy5 peptide 13 followed by washing reveals membrane bound peptide. a, Washing with cell medium. b, Washing with 50 μM Triton X-100 in PBS (van de Ven, A. L., Adler-Storthz, K. & Richards-Kortum, R. Delivery of optical contrast agents using Triton-X100, part 1: reversible permeabilization of live cells for intracellular labeling. J Biomed Opt 14, 021012, doi:10.1117/1.3090448 (2009)). In both cases, cells also show mitochondrial staining of the Cy5. Cy5 has an affinity for mitochondria (Lorenz, S., Tomcin, S. & Mailander, V. Staining of mitochondria with Cy5-labeled oligonucleotides for long-term microscopy studies. Microsc Microanal 17, 440-445, doi:10.1017/S1431927611000249 (2011)), and the labelling of mitochondria may indicate partial degradation of the peptide. Proteolytic degradation of CPPs can occur within minutes (Palm, C., Jayamanne, M., Kjellander, M. & Hallbrink, M. Peptide degradation is a critical determinant for cell-penetrating peptide uptake. Biochim Biophys Acta 1768, 1769-1776, doi:10.1016/j.bbamem.2007.03.029 (2007)). Enlarged are areas where two cells are in contact. The membrane staining is more apparent at these interfaces due to the nature of the confocal images. Scale bars 20 μm.

FIG. 23 shows the treatment of cells with maleimide-R10-Cy5 peptide 13 followed by washing and subsequent delivery of mCherry. a, Washing with cell medium. b, Washing with 25 μg/mL heparin in PBS. In both cases, cells show nucleolar mCherry fluorescence. As shown in SI FIG. 19a, an unreactive CPP additive does not deliver mCherry into nucleoli even without washing with heparin. Scale bars 20 μm.

FIG. 24 shows “Pre-loading” of CPPs on cells followed by cellular uptake of NLS-mCherry-R10 II. a, 5 μM NLS-mCherry-R10 II together with 10 μM AA-R10 (3). b, 5 μM NLS-mCherry-R10 II together with 10 μM Cys-R10 (3). c, 5 μM NLS-mCherry-R10 II together with 10 μM TNB-R10 (2). d, 5 μM NLS-mCherry-R10 together with 10 μM Maleimide-R10. Scale bars 20 μm.

FIG. 25 shows Volcano plots of label-free quantification after protein identification of streptavidin pulldown samples by mass spectrometry. a, Cells were either untreated or treated with 20 μM of the Maleimide-R10-Biotin peptide. b, Cells were treated either with 20 μM of commercially available Biotin-Maleimide or with the Maleimide-R10-Biotin peptide. In both cases, several membrane bound proteins were highly enriched by the cell-penetrating peptide. Amongst those are two membrane-bound metalloproteases (NRD1 and MMP15), an amino acid transporter (SLC7A5) and a caveolae-associated protein (PTRF). See Methods section for experimental details.

FIG. 26 shows the cellular uptake of thiol-reactive CPPs in presence of Annexin V. a, HeLa Kyoto cells were treated with 10 μM TNB-R10-TAMRA 8 in presence of Annexin V—Atto 488 conjugate (1:50) in annexin V buffer (10 mM Hepes (pH 7.4), 140 mM NaCl, 2.5 mM CaCl₂). b, HeLa Kyoto cells were treated with 10 μM Maleimide-R10-Cy5 13 in presence of Annexin V—Atto 488 conjugate (1:50) in annexin V buffer (10 mM Hepes (pH 7.4), 140 mM NaCl, 2.5 mM CaCl₂). c, As a positive control for binding of annexin V to apoptotic cells under the same conditions, HeLa Kyoto cells were incubated for 5 minutes at 37° C. with Annexin V—Atto 488 in annexin V buffer with 50 μM Triton-X 100. Scale bars 20 μm.

FIG. 27 shows the cellular uptake of Maleimide-R10-Cy5 peptide 13 in presence of Flipper-TR membrane tension probe. a, HeLa Kyoto cells were pre-incubated in DMEM with 2 μM Flipper-TR (Spirochrome). Afterwards, DMEM or 10 μM Maleimide-R10-Cy5 13 in DMEM were added to the cells. Fluorescence lifetime images were acquired every 15 seconds for 60 seconds. Shown are the Cy5 photon count and the FastFLIM images. The arrows indicate a site where the CPP is enriched (Cy5 channel) and where the lifetime of the Flipper-TR probe decreases. c, Four ROls in membrane regions for each time-lapse were chosen. A double exponential fit was applied to the fluorescence decay, and the weighted average lifetime was calculated for each ROI. This value was then plotted over time. Shown are individual values and the mean as a line. At nucleation zones, the membrane tension decreases throughout the experiment. Scale bars 20 μm.

FIG. 28 shows Halotag-tethering of CPP and delivery of NLS-mCherry-cR10 I into Halotag-expressing cells. a, Cells transfected with the Halotag-EGFP reporter plasmid express EGFP inside the cell and Halotag on the cell surface. Transfected cells were treated with 1 μM JF646-Halotag-Iigand (Promega). The fluorophore shows staining of the cell membrane (and secretory pathway) in EGFP-expressing cells only. b, Delivery of 5 μM NLS-mCherry-cR10 I on cells transfected with the reporter plasmid. c, Delivery of 5 μM NLS-mCherry-cR10 I in presence of 20 μM “Halo-R10” variants on cells transfected with the reporter plasmid. The arrowheads show nucleoli with mCherry fluorescence. See also main text FIG. 3. Scale bars 20 μm.

FIG. 29 shows confocal microscopy images from all channels from the screen of different cell lines in the co-delivery of NLS-mCherry-R10 II with TNB-R10 5. Scale bars 20 μm.

FIG. 30 shows cellular uptake of NLS-mCherry-R10 II with or without added TNB-R10 5 at 4° C. in various cell lines. Scale bars 20 μm.

FIG. 31 shows confocal microscopy images of cellular uptake of NLS-mCherry-K10 III with or without TNB-R10 5. Scale bars 20 μm.

FIG. 32 shows cell viability assays of cells treated with TNB-R10 5. a, WST-1 assay of HeLa CLL-2 cells. Absorbance at 440 nm is indicative of cellular metabolic activity in the processing of WST-1 to the absorbing Formazan. TNB-R10 had no effect on metabolism up to 50 μM peptide, while the positive control staurosporine had a significant, detrimental effect at a 10 μM concentration under the same treatment conditions. n.s.=not significant, ***=P<0.0005. b, Calcein AM cell viability assay of HeLa Kyoto cells. The cells were treated with either 5 μM NLS-mCherry-R10 II in DMEM alone (lower panel) or with 10 μM TNB-R10 5 in DMEM (upper panel). After one-hour incubation, cells were washed again in DMEM and treated with 5 μM Calcein AM in DMEM. The morphology of the cells as shown in the differential interference contrast (DIC) images is also unaffected. Scale bars 50 μm. c, Co-delivery of NLS-mCherry-R10 II in presence of Sytox Blue dead cell stain with or without added TNB-R10 5. Scale bars 20 μm.

FIG. 33 shows uptake of NLS-mCherry-R10 II in presence of additional Cys-R10 2 or AA-R10 3 and serum. a, Microscopy pictures showing the fluorescence of NLS-mCherry-R10 II after uptake with additional CPP in varying amounts of serum, at 37° C. Scale bars 20 μm. b, Quantification of relative nuclear fluorescence intensities from microscopy pictures. n.s.=not significant, ***=P<0.0005.

FIG. 34 shows characterization of NLS-mCherry-exR10 IV. a, SDS-PAGE gel showing the purity of mCherry-exR10 IV. b, High resolution mass spectrum of NLS-mCherry-exR10 IV, Calc.: 31883 [M+H], 32060 [M+Gluconoylation+H](Geoghegan, K. F. et al. Spontaneous alpha-N-6-phosphogluconoylation of a “His tag” in Escherichia coli: the cause of extra mass of 258 or 178 Da in fusion proteins. Anal Biochem 267, 169-184, doi:10.1006/abio.1998.2990 (1999)); Exp.: 31883, 32060.

FIG. 35 show confocal microscopy images of cellular uptake of NLS-mCherry-exR10 IV with or without TNB-R10 5. Scale bars 20 μm.

FIG. 36 shows characterization of NLS-mCherry-R5 and -R8. a, SDS-PAGE gel showing the purity and conversion of NLS-mCherry (lane 1) to the R5 and R8 conjugates (lanes 2-3). b, High resolution mass spectrum of NLS-mCherry-5, Calc.: 29564 [M+H]; Exp.: 29563. c, High resolution mass spectrum of NLS-mCherry-R8, Calc.: 30033 [M+H]; Exp.: 30033.

FIG. 37 shows confocal microscopy images of cellular uptake of NLS-mCherry-R5 and -R8. a, Cellular uptake of NLS-mCherry-R5 with or without additive TNB-R10 5. b, Cellular uptake of NLS-mCherry-R8 with or without additive TNB-R10 5. Scale bars 20 μm.

FIG. 38 shows in situ uptake of TAMRA-labelled GBP1 nanobody after 30-minute incubation with TNB-R10. The GBP1 nanobody with a free cysteine (after expressed protein ligation and size-exclusion chromatography (see supplementary methods and scheme in SI FIG. 2) was incubated with TNB-R10 (5) for 30 minutes at room temperature. The mixture was then added to HeLa Kyoto cells expressing GFP-PCNA. After 1 hour at 37° C., the cells were washed, counterstained with Hoechst 33342 and imaged. Scale bar 20 μm.

FIG. 39 shows characterization of Cre-exR8. a, SDS-PAGE gel showing the purity of Cre-exR8. b, High resolution mass spectrum of Cre-exR8, Calc.: 42876 [M+H], 43054 [M+Gluconoylation+H]; Exp.: 42877, 43055.

FIG. 40 shows epifluorescence microscopy pictures of HeLa CCL-2 cells transiently transfected with Cre-Stoplight 2.4 and treatment with Cre-exR8. Cells treated with Cre-exR8 in presence of additional Cys-R10 show higher incidence of red fluorescence.

FIG. 41 shows the gating strategy for flow cytometry data.

FIG. 42 is a schematic drawing of the concept to loosen the membrane lipid packing using a CPP-additive with a hydrophobic moiety, and cellular uptake of the CPP-cargo with NLS-mCherry-R10 as example.

FIG. 43 shows spinning disk microscopy images of cells treated with 5 μM of CPP additive with a hydrophobic amino acid moiety ILFF, followed by 5 μM NLS-mCherry-R10.

FIG. 44 shows spinning disk microscopy images of cells treated with 1 μM of CPP additive with a hydrophobic amino acid moiety ILFF, followed by 2.5 μM NLS-mCherry-R10.

FIG. 45 shows spinning disk microscopy images of cells treated with Mal-PEG₂-R10 or Mal-PEG₂-R10-fluorous tag, followed by 5 μM NLS-mCherry-R10.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as one, two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary.

The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

Method for Delivering a Cargo into a Cell

The present invention is directed to a method for delivering a cargo into a cell, the method comprising incubating a compound comprising a moiety capable to bind to the cell surface and a guanidine moiety together with cargo and a cell, wherein the cargo is connected with a group comprising a guanidine moiety, thereby allowing delivering of the cargo into the cell.

In a preferred embodiment of the invention, the cargo is connected such that the group is conjugated with or fused to a group comprising a guanidine moiety.

Accordingly, it is foreseen that the cargo is modified with a group comprising a guanidine moiety. In a preferred embodiment of the invention, the connection of the group to the cargo may be in form of a conjugation of the group to the cargo. In particular, the cargo may be connected to the group comprising a guanidine moiety via a covalent bond. In a further preferred embodiment of the invention, the connection of the group to the cargo may be such that the cargo and the group are fused to each other. Such a fusion may be for example a fusion protein.

The group comprising a guanidine moiety, which is connected with the cargo, can be any chemical moiety which is suitable for comprising a guanidine moiety. In a preferred embodiment of the present invention, the group comprising a guanidine moiety may be a cell-penetrating peptide (CPP). Preferably, the CPP may be conjugated to the cargo. In a further preferred embodiment, the CPP is fused to the cargo.

In a preferred embodiment of the invention, the cargo is selected from peptide, protein, enzyme, nanobody, oligonucleotide, nanoparticle and antibody.

Accordingly, a cargo is any kind of load, in particular a biological load, which is suitable to be transported into a cell. In particular, it is foreseen that the cargo is preferably a peptide or protein. Further preferred, the cargo may be an oligonucleotide or a nanoparticle.

Also preferred, the cargo is any kind of antibody, such as an IgG, IgM, IgA, IgE, or IgD antibody. The antibody is in particular preferred a full-length antibody.

In accordance with the present invention, the compound comprises a moiety which is capable to bind to the cell surface. Any moiety which can bind to a cell surface, in particular by forming a covalent bond to the cell surface, can be used. Suitable chemical moieties, which are capable to bind to the cell surface, are known to a person skilled in the art. A person skilled in the art knows to select suitable moieties which are capable to bind to the cell surface. In this context, the cell surface has a functional group that can form a bond, in particular a covalent bond, with the moiety of the compound capable to bind to the cell surface. As illustrative, non-limiting examples, the functional group on the cell surface can be a thiol group (—SH), an amino group, a hydroxy group (e.g. a hydroxy group of a carbohydrate, in particular the anomeric hydroxy group of a carbohydrate), and/or a carboxy group. Preferably, the functional group on the cell surface is a thiol group or an amino group. More preferably, the functional group on the cell surface is a thiol group. In particular, the functional group can be part of a target structure which is present on the cell surface; in other words, a target structure on the cell surface can comprise the functional group, which can form a bond, in particular a covalent bond, with the moiety capable to bind to the cell surface. Accordingly, the moiety capable to bind to the cell surface can be also regarded as a moiety capable to bind to a target structure on a cell surface. As illustrative examples, the target structure can be a protein, a peptide, a glycolipid, a glycoprotein, a tag or a bioorthogonal chemical reporter. A thiol group, as illustrative example, is present in a cysteine moiety of the target structure (e.g. a protein), or can be generated by reduction of an intramolecular disulfide bond of the target structure. It is also contemplated to generate a thiol group by reaction of an amino group of a lysine moiety of the target structure using 2-iminothiolane (Traut's reagent) or another thiol generating reagent. The moiety capable to bind to the cell surface is not particularly limited, and any moiety can be used which is capable of forming a bond, in particular a covalent bond, with a functional group on the cell surface. Preferably, when the functional group on the cell surface is a thiol group (—SH), an amino group, a hydroxy group (e.g., a hydroxy group of a carbohydrate, in particular the anomeric hydroxy group of a carbohydrate), and/or a carboxy group, the moiety capable to bind to the cell surface is or comprises an electrophilic moiety. As illustrative examples, the formation of a bond between the functional group on the cell surface and the moiety capable to bind to the cell surface may involve a substitution reaction (e.g., nucleophilic substitution) or addition reaction (e.g., addition to a double bond or triple bond of the moiety capable to bind to the cell surface). As illustrative examples, suitable moieties which are capable to bind to the cell surface are described herein further below as A group within the context of the compounds of the invention. Accordingly, any A group described herein may be the moiety capable to bind to the cell surface.

In a preferred embodiment of the invention, the moiety of the compound capable to bind to the cell surface is a thiol-reactive moiety, or the moiety is capable to bind to the cell surface via an enzymatic reaction, preferably the moiety is capable to bind to a tag, such as a Halotag.

A tag, as used herein, may refer to a peptide sequence which can be attached to the cell surface for various purposes. In general, tags are known to a person skilled in the art and can be suitably selected. Non limiting examples for tags are affinity tags, solubilization tags, chromatography tags epitope tags and reporter enzymes. Preferably, in embodiments of the present invention, the tag is a Halotag.

As used herein, a thiol-reactive moiety may refer to any moiety or functional group which is capable of reacting with a thiol group (SH), such as e.g. with a thiol group present on a cell surface. In general, such reaction of a thiol-reactive moiety with a thiol group leads to formation of a covalent bond. For example, reaction of a thiol-reactive moiety with the thiol group can involve substitution (e.g., nucleophilic substitution) or addition (e.g., addition of the thiol group to a double bond or triple bond of the thiol-reactive moiety). Suitable thiol-reactive moieties are known to a person skilled in the art. A person skilled in the art knows to select suitable thiol-reactive moieties. For example, an A group as described herein within the context of the compounds of the invention can react with a thiol group.

In general, the moiety capable to bind to a cell surface, as described herein with regard to the compound comprising a moiety capable to bind to the cell surface and a guanidine moiety, can be also used to connect the cargo with the group comprising a guanidine moiety. Accordingly, a compound comprising such moiety, e.g. an A group as described herein within the context of the compounds of the invention, and a group comprising a guanidine moiety can be reacted with the cargo to conjugate the group comprising a guanidine moiety with the cargo. In this context, the cargo has a functional group that can form a bond, in particular a covalent bond, with the moiety. As illustrative, non-limiting examples, the functional group of the cargo can be a thiol group (—SH), an amino group, a hydroxy group (e.g. a hydroxy group of a carbohydrate, in particular the anomeric hydroxy group of a carbohydrate), and/or a carboxy group. Preferably, the functional group of the cargo is a thiol group or an amino group. More preferably, the functional group of the cargo is a thiol group. In some embodiments, the compound comprising a moiety capable to bind to the cell surface and a guanidine moiety, and the compound which can be reacted with the cargo to conjugate the group comprising a guanidine moiety with the cargo, are identical. In some embodiments, the compound comprising a moiety capable to bind to the cell surface and a guanidine moiety, and the compound which can be reacted with the cargo to conjugate the group comprising a guanidine moiety with the cargo, are different.

Also, in accordance with the present invention, the compound, which comprises a moiety capable to bind to the cell surface, further comprises a guanidine moiety. A guanidine moiety, as known to a person skilled in the art, has the following structure:

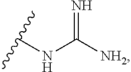 [SMILES: *NC(=N)N]

wherein [symbol] indicates the attachment point to other parts of the compound. The number of guanidine moieties in the compound is not particularly limited. As illustrative example, the compound may comprise one or more guanidine moieties. The compound may comprise 3 or more guanidine moieties. The compound may comprise 5 or more guanidine moieties. The compound may comprise 8 or more guanidine moieties. The compound may comprise 10 or more guanidine moieties. In addition or alternatively, the compound may comprise 25 or less guanidine moieties. The compound may comprise 20 or less guanidine moieties. The compound may comprise 15 or less guanidine moieties. The compound may comprise 12 or less guanidine moieties. The guanidine moiety or the guanidine moieties may be comprised in a group of the compound comprising the moiety capable to bind to the cell surface. Accordingly, the guanidine moiety or the guanidine moieties may be comprised in the group, which group is comprised in the compound comprising the moiety capable to bind to the cell surface. Accordingly, the compound comprising a moiety capable to bind to a cell surface may comprise a group comprising the guanidine moiety, or the guanidine moieties. Accordingly, the group may comprise one or more guanidine moieties. The group may comprise 3 or more guanidine moieties. The group may comprise 5 or more guanidine moieties. The group may comprise 8 or more guanidine moieties. The group may comprise 10 or more guanidine moieties. In addition or alternatively, the group may comprise 25 or less guanidine moieties. The group may comprise 20 or less guanidine moieties. The group may comprise 15 or less guanidine moieties. The group may comprise 12 or less guanidine moieties. The group may be any chemical moiety suitable for comprising a guanidine moiety. With regard to the compound comprising the moiety capable to bind to the cell surface, the group comprising the guanidine moiety or the guanidine moieties may be also denoted as “first group”. In particular, the group (i.e. the first group) may comprise or may be a peptide, which comprises the guanidine moiety or the guanidine moieties; for example, an arginine-rich peptide. Preferably, the group of the compound, which comprises the moiety capable to bind to the cell surface (i.e. the first group), comprises a repeating unit having the following structure:

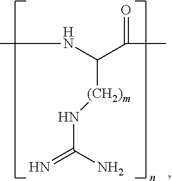

wherein m is each independently an integer ranging from 0 to 10 and n is an integer ranging from 1 to 20. Preferably, m is each independently an integer ranging from 1 to 10. More preferably, m is each independently an integer ranging from 1 to 8. Still more preferably, m is each independently an integer ranging from 1 to 6. Still more preferably, m is each independently an integer ranging from 1 to 5. Still more preferably, m is each independently an integer ranging from 2 to 4. Most preferably, m is each 3. Preferably, n is an integer ranging from 3 to 19. More preferably, n is an integer ranging from 4 to 19. Still more preferably, n is an integer ranging from 4 to 17. Still more preferably, n is an integer ranging from 5 to 15. Still more preferably, n is an integer ranging from 6 to 13. Still more preferably, n is an integer ranging from 7 to 11. Still more preferably, n is an integer ranging from 8 to 10. Most preferably, n is 9 or 10. In some embodiments, n is an integer ranging from 5 to 20. In preferred embodiments, m is each 3. When m is each 3, the group comprises an arginine unit. Preferably, the group is an oligoarginine or polyarginine. Accordingly, in preferred embodiments, m is each 3 so that the group (i.e. the first group) comprises a repeating unit having the following structure:

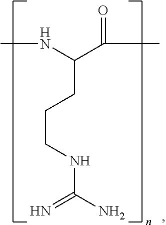

wherein n is as defined herein. More preferably, when arginine has its natural configuration (L or S configuration), the repeating unit has the following structure:

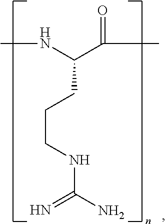

wherein n is as defined herein. The group (i.e. the first group) comprising the repeating unit(s) may be linear or cyclic. Preferably, the group comprising the repeating unit(s) is linear. As illustrative examples, the group (i.e. the first group) may be a linear oligoarginine or polyarginine. In some embodiments, the group (i.e. the first group) comprising the repeating units is cyclic. When the group is cyclic, the integer n may range from 5 to 20; for example, the integer n may range from 8 to 15; in particular, the integer n may be 10. As an illustrative example, a cyclic group may have the following structure, which comprises 10 arginine units and wherein [symbol] indicates the attachment point:

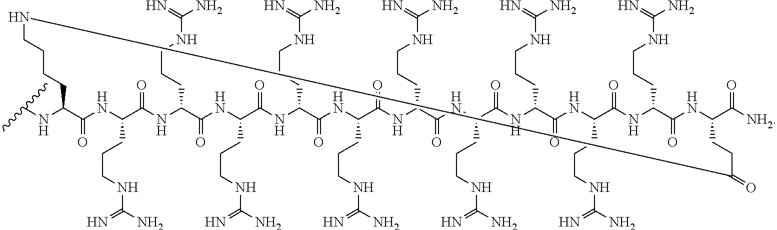 [SMILES: *N[C@H]1CCCCNC(=O)CC[C@@H](C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O]

The compound, which comprises a moiety capable to bind to the cell surface and a guanidine moiety, may further comprise a hydrophobic moiety. In particular, such moiety may be a terminal hydrophobic moiety. As used herein, the term “terminal hydrophobic moiety” refers to a hydrophobic moiety which is arranged at the end of a molecule, such as e.g. at the end of a chain-like molecule. As an illustrative example, when the compound, which comprises a moiety capable to bind to the cell surface and a guanidine moiety, comprises a peptide (for example, as described herein, the first group may comprise or may be a peptide which comprises the guanidine moiety or the guanidine moieties, such as, for example, a linear peptide), the hydrophobic moiety may be bound to the C-terminus of the peptide. As an illustrative example, when the compound, which comprises a moiety capable to bind to the cell surface and a guanidine moiety, comprises an oligoarginine or polyarginine (for example, as described herein, the first group may be an oligoarginine or polyarginine, such as, for example, a linear oligoarginine or polyarginine), the hydrophobic group may be bound to the C-terminus of the oligoarginine or the polyarginine. Hydrophobic moieties are generally known to a person skilled in the art. Any hydrophobic moiety can be used and will be readily selected by the skilled person. As illustrative, non-limiting examples, the hydrophobic moiety may be or may comprise an alkyl group, an alkenyl group, an alkynyl group, a cycloalkyl group, a cycloalkenyl group, a cycloalkynyl group, and/or an aryl group. Optionally, each of the foregoing groups may be substituted with one or more halogen atoms, such as one or more fluorine atoms. The hydrophobic moiety may comprise or may be a perfluorinated moiety. In particular, each of the foregoing groups may be perfluorinated. As used herein, the term “perfluorinated” means that all hydrogen atoms of a moiety or group are replaced by fluorine atoms. In some embodiments, the hydrophobic moiety may be a hydrophobic peptide. In some embodiments, the hydrophobic moiety may be a hydrophobic moiety as described herein for the group Z of a compound according to the invention (such as, for example, a compound of formula (1)); such as, for example, a peptide comprising 2 to 10 amino acids independently selected from the group consisting of glycine, alanine, valine, leucine, isoleucine, proline, phenylalanine, methionine, tryptophan and one or more hydrophobic unnatural amino acid(s); or such as, for example, the hydrophobic moiety comprises or is (C₁-C₂₀)perfluoroalkyl. It has been surprisingly found that compounds (such as, e.g., compounds of formula (1) described herein), when used to bind to the cell surface and in which Z is a hydrophobic moiety, allow for decreasing the application concentration of the compound used to bind to the cell surface and/or the cargo connected with a group comprising a guanidine moiety (see also Examples 6 and 7). Without being bound to any theory, it is assumed that in particular the compound of the present invention including a hydrophobic moiety allows to accumulate at the cell membrane. It is hypothesized that this is achieved by incorporating of the hydrophobic moiety into the phospholipid bilayer of the cell membrane. Thereby access points are generated which are characterized by a loosened membrane structure. Such an improved membrane lipid packing loosening provides a kind of gaps within the cell membrane, which in turn then allows delivering of a cargo into a cell even at lower concentrations.

Also, in accordance with the present invention, the cargo is connected with a group comprising a guanidine moiety. Also in this regard, a guanidine moiety has the following structure:

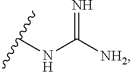 [SMILES: *NC(=N)N]

wherein [symbol] indicates the attachment point to other parts of the compound. The number of guanidine moieties in the group is not particularly limited. As illustrative example, the group may comprise one or more guanidine moieties. The group may comprise 3 or more guanidine moieties. The group may comprise 5 or more guanidine moieties. The group may comprise 8 or more guanidine moieties. The group may comprise 10 or more guanidine moieties. In addition or alternatively, the group may comprise 25 or less guanidine moieties. The group may comprise 20 or less guanidine moieties. The group may comprise 15 or less guanidine moieties. The group may comprise 12 or less guanidine moieties. The group may be any chemical moiety suitable for comprising a guanidine moiety. With regard to the cargo, the group comprising the guanidine moiety or the guanidine moieties may be also denoted as “second group”. In particular, the group (i.e. the second group) may comprise or may be a peptide, which comprises the guanidine moiety or the guanidine moieties; for example, an arginine-rich peptide. Preferably, the group (i.e. the second group) comprises a repeating unit having the following structure:

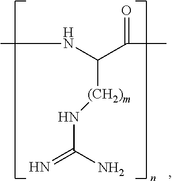

wherein m is each independently an integer ranging from 0 to 10 and n is an integer ranging from 1 to 20. Preferably, m is each independently an integer ranging from 1 to 10. More preferably, m is each independently an integer ranging from 1 to 8. Still more preferably, m is each independently an integer ranging from 1 to 6. Still more preferably, m is each independently an integer ranging from 1 to 5. Still more preferably, m is each independently an integer ranging from 2 to 4. Most preferably, m is each 3. Preferably, n is an integer ranging from 3 to 19. More preferably, n is an integer ranging from 4 to 17. Still more preferably, n is an integer ranging from 5 to 15. Still more preferably, n is an integer ranging from 6 to 13. Still more preferably, n is an integer ranging from 7 to 11. Still more preferably, n is an integer ranging from 8 to 10. Most preferably, n is 9 or 10. In some embodiments, n is an integer ranging from 5 to 20. In preferred embodiments, m is each 3. When m is each 3, the group comprises an arginine unit. Preferably, the group is an oligoarginine or polyarginine. Accordingly, in preferred embodiments, m is each 3 so that the group (i.e. the second group) comprises a repeating unit having the following structure:

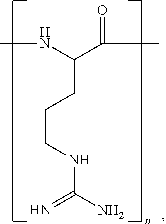

wherein n is as defined herein. More preferably, when arginine has its natural configuration (L or S configuration), the repeating unit has the following structure:

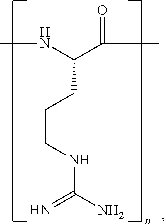

wherein n is as defined herein. The group (i.e. the second group) comprising the repeating unit(s) may be linear or cyclic. Preferably, the group (i.e. the second group) comprising the repeating unit(s) is linear. As illustrative examples, the group may be a linear oligoarginine or polyarginine. In some embodiments, the group (i.e. the second group) comprising the repeating units is cyclic. When the group is cyclic, the integer n may range from 5 to 20; for example, the integer n may range from 8 to 15; in particular, the integer n may be 10. As an illustrative example, a cyclic group may have the following structure, which comprises 10 arginine units:

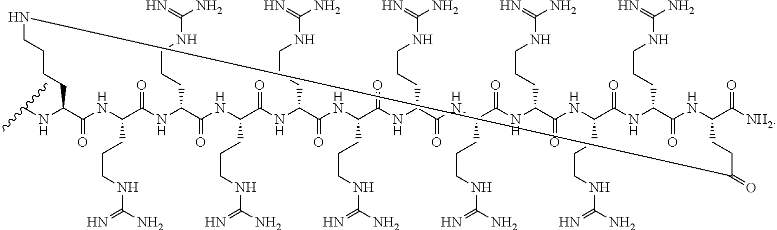

In some embodiments, the group comprising a guanidine moiety which is connected with the cargo (“second group”), and the group comprising a guanidine moiety of the compound comprising a moiety capable to bind to a cell surface (“first group”) are identical. In some embodiments, the group comprising a guanidine moiety which is connected with the cargo (“second group”), and the group comprising a guanidine moiety of the compound comprising a moiety capable to bind to a cell surface (“first group”) are different.

Compounds of the invention are also defined herein further below.

Preferably, the method of the present invention is comprising: (a) incubating the compound comprising a moiety capable to bind to the cell surface and a guanidine moiety together with the cargo connected with the group comprising a guanidine moiety to obtain a solution comprising the compound and the cargo connected with the group, (b) incubating the solution of step (a) with the cell, thereby allowing delivering of the cargo into the cell.

In a preferred embodiment of the invention, the method comprises as a further step before (a), a step (a0) of providing a cell. Accordingly, it may be foreseen that a cell is prepared and incubated in a container suitable for cell culturing, such as a plate, well of a plate, a dish, a flask, or a tube.

Preferably, cells such as primary cells or cell lines are used for the method of the present invention. Preferred cell lines may be HeLa, such as HeLa CCL-2 or HeLa Kyoto, SKBR-3, A549, MDCK-2, SJSA-1 or any other cell lines known for a person skilled in the art. A person skilled in the art is able to choose any kind of cell, such as a primary cell or a distinct cell line which should be used in the desired setting to deliver a cargo in such a cell.

Preferably, the method of the present invention further comprises before (a) a step (a1) of providing the compound comprising a moiety capable to bind to the cell surface and a guanidine moiety.

In a preferred embodiment of the present invention the compound is provided in a concentration of 1 to 50 μM. Advantageously, the delivery of the cargo into the cell can be even achieved at low concentrations of the compound.

In a preferred embodiment of the invention, in (b) the incubating the solution of (a) with the cell is carried out for a time of 1 minute to 24 hours, preferably for 5 min to 60 minutes, and/or at a temperature of 4° C. to 37° C.

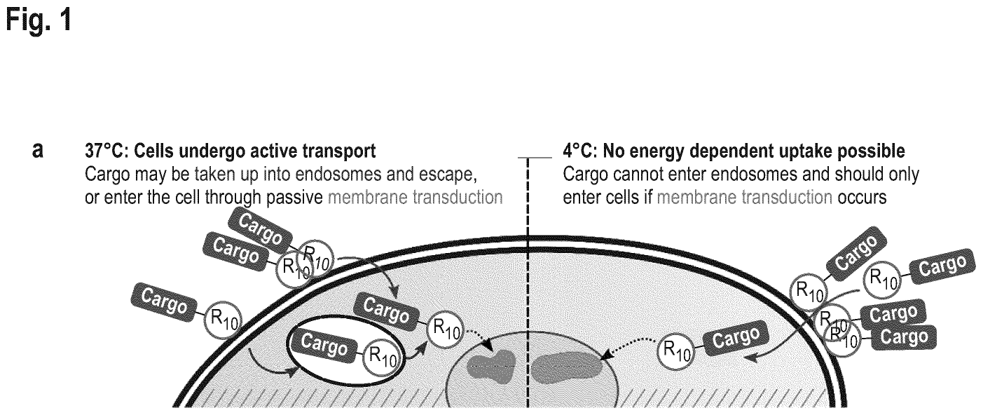

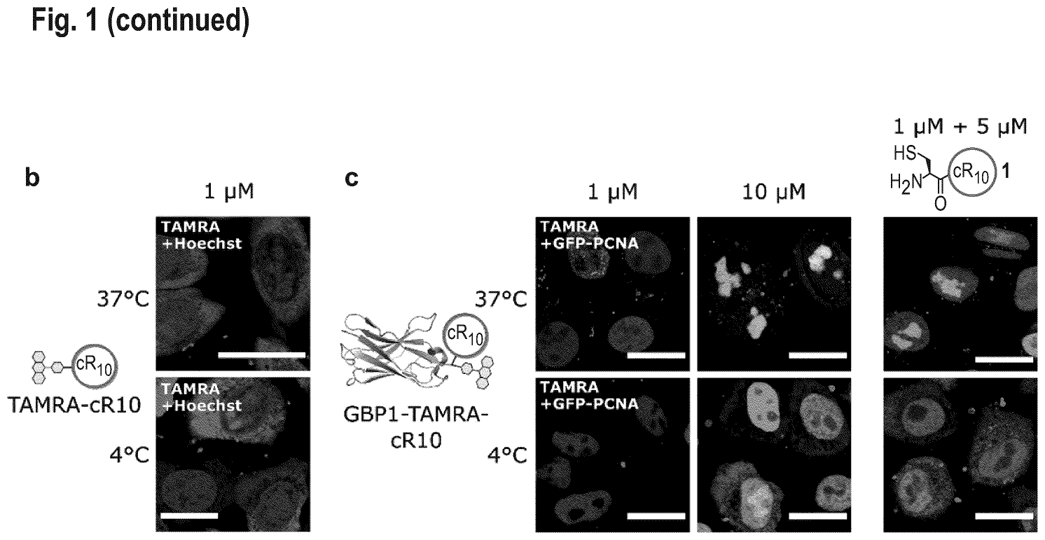

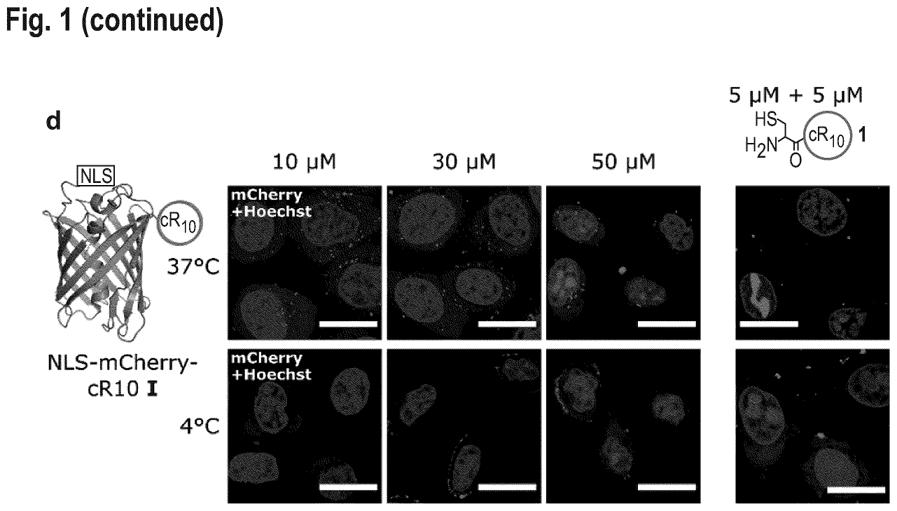

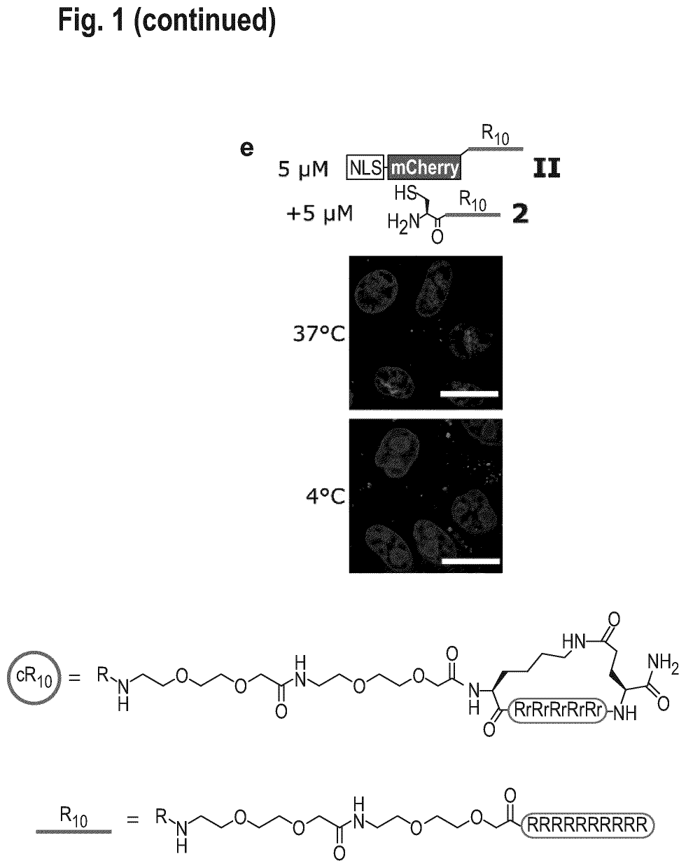

In a preferred embodiment of the invention, the temperature is 4° C. Such a temperature provides the condition that no energy dependent uptake of the cargo is possible. Therefore, the cargo cannot enter endosomes and should only enter cells if membrane transduction occurs (see also FIG. 1a). A non-endosomal pathway allows that the cargo is delivered into the cell in a native form without any structural alterations.

In a further preferred embodiment of the invention, the temperature is 37° C. Such a temperature provides the condition that cell undergo an active transport. Therefore, the cargo may be taken up into endosomes and escape, or enter the cell through passive membrane transduction (see also FIG. 1a). Non-endosomal uptake is also expected at temperature around 37° C. Therefore, the method of the present invention is suitable to be conducted at typically physiological temperatures of the cells, such as 37° C.

Preferably, the method of the present invention foresees that the moiety, which is capable to bind to the cell surface, is capable to bind to a tag or target structure on the cell surface. Accordingly, in preferred embodiments, the method comprises: (a) transfecting a cell with a tag such that the tag is expressed on the cell surface, or modifying the cell surface with a target structure, (b) incubating the compound comprising the moiety capable to bind to the tag or target structure on the cell surface and a guanidine moiety together with the cargo connected with the group comprising a guanidine moiety to obtain a solution comprising the compound and the cargo connected with the group, (c) incubating the solution of step (b) with the cell, thereby allowing delivering of the cargo into the cell.

Any tag known to a person skilled in the art may be used. As illustrative examples, any tag disclosed herein may be used. In a preferred embodiment of the invention, the tag is a Halotag.

Any target structure known to a person skilled in the art which is suitable to bind with the moiety of the compound can be used. As illustrative examples, any target structure described herein may be used. Preferably, the target structure is a bioorthogonal chemical reporter on the cell surface. As it is known, a bioorthogonal chemical reporter is a non-native chemical functionality that is introduced into the naturally occurring biomolecules of a living system, generally through metabolic or protein engineering. These functional groups are subsequently utilized for tagging and visualizing biomolecules. Jennifer Prescher & Carolyn R. Bertozzi, the developers of bioorthogonal chemistry, defined bioorthogonal chemical reporters as “non-native, non-perturbing chemical handles that can be modified in living systems through highly selective reactions with exogenously delivered probes.”

In a preferred embodiment of the invention, the method comprises as a further step before (a), a step (a0) of providing a cell. Accordingly, it may be foreseen that a cell is prepared and incubated in a container suitable for cell culturing, such as a plate, well of a plate, a dish, a flask, or a tube. Transfection of the cells is a standard procedure which is known in the art.

Preferably, the method of the present invention further comprises before (b) a step (b0) of providing the compound comprising a moiety capable to bind to bind to a Halotag and a guanidine moiety.

In a preferred embodiment of the present invention the compound is provided in a concentration of 1 to 50 μM.

In a preferred embodiment of the invention, wherein in (c) the incubating the solution of (b) with the cell is carried out for a time of 1 minute to 24 hours, preferably for 5 min to 60 minutes, and/or at a temperature of 4° C. to 37° C.

In a preferred embodiment of the invention, the temperature is 4° C. Such a temperature provides the condition that no energy dependent uptake of the cargo is possible.

Therefore, the cargo cannot enter endosomes and should only enter cells if membrane transduction occurs (see also FIG. 1a).

In a further preferred embodiment of the invention, the temperature is 37° C. Such a temperature provides the condition that cells undergo an active transport. Therefore, the cargo may be taken up into endosomes and escape, or enter the cell through passive membrane transduction (see also FIG. 1a). Non-endosomal uptake is also expected at temperature around 37° C. Therefore, the method of the present invention is suitable to be conducted at typically physiological temperatures of the cells, such as 37° C.

Preferably, the delivered cargoes are antibodies, preferably full-length antibodies.

The inventors could show that it is possible with the method of the present invention to deliver functional antibodies into a cell. In view of the size of a full-length antibody, it is an advantage of the method of the present invention that the delivery of such large proteins is possible. Preferably, antibodies of IgG class can be delivered. However, any other antibody, such as IgM, IgE, IgA or IgD, may also be delivered with the method of the present invention.

The method of the invention may be carried out in vitro. Accordingly, in some embodiments the method is an in vitro method. Also, the method of the invention may be carried out in vivo. Accordingly, in some embodiments the method is an in vivo method.

Compounds for Use in Delivering a Cargo into a Cell

A further aspect of the invention is directed to a compound comprising a moiety capable to bind to a cell surface and a guanidine moiety for use in delivering a cargo into a cell.

Any compound comprising a moiety capable to bind to a cell surface and a guanidine moiety, which is disclosed herein, can be used. Preferably, the compound for the use according to the invention is characterized such that the moiety capable to bind to the cell surface is a thiol-reactive moiety, or the moiety is capable to bind to the cell surface via an enzymatic reaction. Preferably, the moiety is capable to bind to a tag, such as a Halotag.

In a preferred embodiment, the cargo is connected with a group comprising a guanidine moiety.

Accordingly, it is foreseen that the cargo is modified with a group comprising a guanidine moiety. In a preferred embodiment of the invention, the connection of the group to the cargo may be in form of a conjugation of the group to the cargo. In particular, the cargo may be connected to the group comprising a guanidine moiety via a covalent bond. In a further preferred embodiment of the invention, the connection of the group to the cargo may be such that the cargo and the group are fused to each other. Such a fusion may be for example a fusionprotein.

In a preferred embodiment of the present invention, the group comprising a guanidine moiety may be a cell-penetrating peptide (CPP). Preferably, the CPP may be conjugated to the cargo. In a further preferred embodiment, the CPP is fused to the cargo.

In accordance with the present invention, a cell-penetrating peptide, CPP, can be understood as a distinct example of a group comprising a guanidine moiety according to the present invention.

In a further preferred embodiment of the invention, the cargo is an antibody, preferably a full-length antibody. Preferably, antibodies of IgG class can be delivered. Further, any other antibody, such as IgM, IgE, IgA or IgD, may also be delivered with the compound for use in delivering a cargo into a cell according to the present invention.

In a preferred embodiment of the invention the compound is for use in diagnostic or therapy. Accordingly, the invention is useful for the diagnostic of intracellular structures in form of intracellular immunostaining. Further, the delivery of biopharmaceuticals, such as antibodies or pharmaceutical substances, allow a therapeutic effect. Moreover, the method and compounds of the present invention achieve gene editing, which could be applied to the delivery of cargos, such as functional enzymes.

### Compounds According to the Invention

Compounds of the invention are also described in more detail below. These compounds can be used in any one of the methods and uses of the invention described herein. Preferably, the compounds can be used to bind to the cell surface. It is also possible to use the compounds for conjugating the cargo with a group comprising a guanidine moiety.

Accordingly, the present invention also relates to a compound of formula (1):

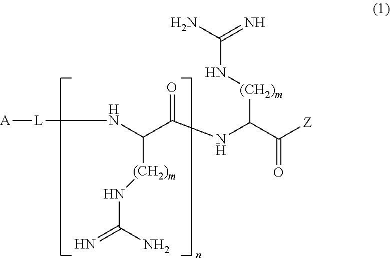

wherein:
A is a moiety capable to bind to a cell surface;
L is a linker or a bond;
m is each independently an integer ranging from 0 to 10;
n is an integer ranging from 1 to 20;
Z is selected from the group consisting of NR¹R², OR³, an amino acid, a peptide comprising 2 to 10 amino acids, and a hydrophobic moiety;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring;
R³ is hydrogen or (C₁-C₆)alkyl;
or a pharmaceutically acceptable salt thereof.

Any A, L, m, n, Z, R¹, R² and R³ as defined herein can be combined with each other.
A is a moiety capable to bind to a cell surface.
L is a linker or a bond. Accordingly, in some embodiments L is a bond. Preferably, L is a linker.
m is each independently an integer ranging from 0 to 10. Preferably, m is each independently an integer ranging from 1 to 10. More preferably, m is each independently an integer ranging from 1 to 8. Still more preferably, m is each independently an integer ranging from 1 to 6. Still more preferably, m is each independently an integer ranging from 1 to 5. Still more preferably, m is each independently an integer ranging from 2 to 4. Most preferably, m is each 3.
n is an integer ranging from 1 to 20. Preferably, n is an integer ranging from 3 to 19. More preferably, n is an integer ranging from 4 to 19. Still more preferably, n is an integer ranging from 4 to 17. Still more preferably, n is an integer ranging from 5 to 15. Still more preferably, n is an integer ranging from 6 to 13. Still more preferably, n is an integer ranging from 7 to 11. Still more preferably, n is an integer ranging from 8 to 10. Most preferably, n is 9. In some embodiments, n is an integer ranging from 5 to 20.

In some embodiments, Z is selected from the group consisting of NR¹R², OR³, an amino acid, a peptide comprising 2 to 10 amino acids, and a hydrophobic moiety. In some embodiments, Z may be selected from the group consisting of NR¹R², OR³, an amino acid, a peptide comprising 2 to 10 amino acids, and a hydrophobic moiety when the compound is used to bind to the cell surface. In some embodiments, Z is selected from the group consisting of NR¹R², OR³, an amino acid, and a peptide comprising 2 to 10 amino acids. In some embodiments, Z may be selected from the group consisting of NR¹R², OR³, an amino acid, and a peptide comprising 2 to 10 amino acids when the compound is used for conjugating the cargo with a group comprising a guanidine moiety.

Accordingly, in some preferred embodiments Z is NR¹R². R¹ and R² are each independently selected from hydrogen and (C₁-C₆)alkyl (preferably (C₁-C₄)alkyl, more preferably methyl, ethyl, propyl or butyl, still more preferably methyl or ethyl); wherein optionally, when R¹ and R² are (C₁-C₆)alkyl, R¹ and R² together with the nitrogen atom to which they are attached form a four- to seven-membered ring, preferably a five- or six-membered ring. In some embodiments, R¹ is hydrogen and R² is (C₁-C₆)alkyl. In some embodiments, R¹ and R² are each (C₁-C₆)alkyl. Preferably, R¹ and R² are each hydrogen; in this case, Z is NH₂.

In some embodiments, Z is OR³. R³ is hydrogen or (C₁-C₆)alkyl. In some embodiments, R³ is (C₁-C₆)alkyl. Preferably, R³ is H; in this case, Z is OH.

“Alkyl” refers to a radical of a straight-chain or branched saturated hydrocarbon group that in one embodiment has from 1 to 6 carbon atoms (“(C₁-C₆)alkyl”). In some embodiments, an alkyl group has 1 to 5 carbon atoms (“(C₁-C₅)alkyl”). In some embodiments, an alkyl group has 1 to 4 carbon atoms (“(C₁-C₄)alkyl”). In some embodiments, an alkyl group has 1 to 3 carbon atoms (“(C₁-C₃)alkyl”). In some embodiments, an alkyl group has 1 to 2 carbon atoms (“(C₁-C₂)alkyl”). In some embodiments, an alkyl group has 1 carbon atom (“C₁alkyl”). In some embodiments, an alkyl group has 2 to 6 carbon atoms (“(C₂-C₆)alkyl”). Examples of (C₁-C₆)alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), amyl (C₅), neopentyl (C₅), 3-methyl-2-butanyl (C₅), tertiary amyl (C₅), and n-hexyl (C₆). An alkyl group, such as a (C₁-C₆)alkyl group, can be unsubstituted or substituted with one or more groups including, but not limited to, —(C₁-C₆)alkyl, —O—(C₁-C₆)alkyl, -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₆) alkyl and aryl.

In some embodiments, Z is an amino acid. For example, Z can be an amino acid when the compound of the invention is produced using biotechnological methods.

The term “amino acid” as used herein refers to an organic compound having a —CH(NH₃)—COOH group. In one embodiment, the term “amino acid” refers to a naturally occurring amino acid. Naturally occurring amino acids may be, as illustrative examples, arginine, lysine, aspartic acid, glutamic acid, glutamine, asparagine, histidine, serine, threonine, tyrosine, cysteine, methionine, tryptophan, alanine, isoleucine, leucine, phenylalanine, valine, proline and glycine. However, the term in its broader meaning also encompasses non-naturally occurring amino acids.

In some embodiments, Z is a peptide comprising 2 to 10 amino acids, preferably 2 to 5 amino acids, more preferably 2 or 3 amino acids. Preferably, the peptide comprises 2 to 5 amino acids. More preferably, the peptide comprises 2 or 3 amino acids. For example, Z can be a peptide when the compound of the invention is produced using biotechnological methods.

The term “peptide” as used herein refers to an organic compound comprising two or more amino acids covalently joined by peptide bonds (amide bond). Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues, e.g. with up to about 30 amino acid residues, are polypeptides.

In some embodiments, Z is a hydrophobic moiety. Hydrophobic moieties are generally known to a person skilled in the art. Any hydrophobic moiety can be used and will be readily selected by the skilled person. As illustrative, non-limiting examples, the hydrophobic moiety may be or may comprise an alkyl group, an alkenyl group, an alkynyl group, a cycloalkyl group, a cycloalkenyl group, a cycloalkynyl group, and/or an aryl group. Optionally, each of the foregoing groups may be substituted with one or more halogen atoms, such as one or more fluorine atoms. The hydrophobic moiety may comprise or may be a perfluorinated moiety. In particular, each of the foregoing groups may be perfluorinated. As used herein, the term “perfluorinated” means that all hydrogen atoms of a moiety or group are replaced by fluorine atoms. In some embodiments, the hydrophobic moiety may be a hydrophobic peptide. It has been surprisingly found that compounds (such as, e.g., compounds of formula (1) described herein), when used to bind to the cell surface and in which Z is a hydrophobic moiety, allow for decreasing the application concentration of the compound used to bind to the cell surface and/or the cargo connected with a group comprising a guanidine moiety (see also Examples 6 and 7). Without being bound to any theory, it is assumed that in particular the compound of the present invention including a hydrophobic moiety allows to accumulate at the cell membrane. It is hypothesized that this is achieved by incorporating of the hydrophobic moiety into the phospholipid bilayer of the cell membrane. Thereby access points are generated which are characterized by a loosened membrane structure. Such an improved membrane lipid packing loosening provides a kind of gaps within the cell membrane, which in turn then allows delivering of a cargo into a cell even at lower concentrations.

In some embodiments, when being a hydrophobic moiety, Z is a peptide comprising 2 to 10, preferably 3 to 9, more preferably 4 to 8, amino acids independently selected from the group consisting of glycine, alanine, valine, leucine, isoleucine, proline, phenylalanine, methionine, tryptophan and one or more hydrophobic unnatural amino acid(s). Each amino acid may be, independently, an L amino acid or a D amino acid. Preferably, each amino acid is an L amino acid. Preferably, the peptide is linear. In the event that a hydrophobic unnatural amino acid is present, the peptide may comprise, independently, one or more hydrophobic unnatural amino acids. The term “hydrophobic unnatural amino acid”, whenever used herein, may refer to any non-naturally occurring amino acid which is hydrophobic. Hydrophobic unnatural amino acids are generally known to a person skilled in the art. Any hydrophobic unnatural amino acid can be used and will be readily selected by the skilled person. Illustrative examples for hydrophobic unnatural amino acids, which can be used herein, include hydrophobic fluorinated amino acids. Illustrative examples for hydrophobic fluorinated amino acids include the aforementioned amino acids (glycine, alanine, valine, leucine, isoleucine, proline, phenylalanine, methionine, tryptophan) which are fluorinated, such as, for example, 3-fluoroalanine, 3-fluoro-valine, 4-fluoro-leucine, 4-fluoroproline, 3-fluorophenylalanine, 4-fluorophenylalanine or 5-fluoro-tryptophan. Further illustrative examples for hydrophobic fluorinated amino acids include difluoroethylglycine (DfeGly), trifluoroethylglycine (TfeGly), m-fluoro-DL-phenylalanine, p-fluoro-DL-phenylalanine, 4-trifluoromethylphenylalanine ((4-CF₃)Phe), 5-fluoro-L-tryptophan, hexafluorovaline (hFVal) and hexafluoroleucine (hFLeu). Further hydrophobic unnatural amino acids, which can be used herein, include unnatural amino acids with hydrophobic aromatic side chains. Illustrative examples for unnatural amino acids with hydrophobic aromatic side chains include 3-(1-naphthyl)-alanine (e.g., 3-(1-naphthyl)-L-alanine), 3-(2-naphthyl)-alanine (e.g., 3-(2-naphthyl)-L-alanine) and 2-anthryl-alanine (e.g., 2-anthryl-L-alanine). In some embodiments, the one or more hydrophobic unnatural amino acid(s) is selected from the group consisting of 3-fluorophenylalanine, 4-fluorophenylalanine, 3-(1-naphthyl)-alanine, 3-(2-naphthyl)-alanine and any combination thereof. In some embodiments, the one or more hydrophobic unnatural amino acid(s) is selected from the group consisting of 3-fluorophenylalanine, 4-fluorophenylalanine and a combination thereof. In some embodiments, the one or more hydrophobic unnatural amino acid(s) is selected from the group consisting of 3-(1-naphthyl)-alanine, 3-(2-naphthyl)-alanine and a combination thereof.

In some embodiments, when being a hydrophobic moiety, Z is a peptide comprising 2 to 10, preferably 3 to 9, more preferably 4 to 8, amino acids independently selected from the group consisting of glycine, alanine, valine, leucine, isoleucine, proline, phenylalanine, methionine and tryptophan. Preferably, when being a hydrophobic moiety, Z is a peptide comprising 2 to 10, preferably 3 to 9, more preferably 4 to 8 amino acids independently selected from the group consisting of glycine, leucine, isoleucine, phenylalanine and tryptophan. More preferably, when being a hydrophobic moiety, Z is a peptide comprising 2 to 10, preferably 3 to 9, more preferably 4 to 8 amino acids independently selected from the group consisting of glycine, leucine, isoleucine and phenylalanine. Each amino acid may be, independently, an L amino acid or a D amino acid. Preferably, each amino acid is an L amino acid. Preferably, the peptide is linear.

Preferably, when being a hydrophobic moiety, Z is:

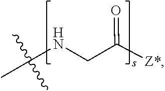

wherein:
Z* is a peptide comprising 2 to 6, preferably 3 to 5 amino acids independently selected from the group consisting of glycine, alanine, valine, leucine, isoleucine, proline, phenylalanine, methionine, tryptophan and one or more hydrophobic unnatural amino acid(s);
preferably wherein Z* is a peptide comprising 2 to 6, preferably 3 to 5 amino acids independently selected from the group consisting of glycine, alanine, valine, leucine, isoleucine, proline, phenylalanine, methionine and tryptophan;
more preferably wherein Z* is a peptide comprising 2 to 6, preferably 3 to 5 amino acids independently selected from the group consisting of glycine, leucine, isoleucine, phenylalanine and tryptophan;
still more preferably wherein Z* is a peptide comprising 2 to 6, preferably 3 to 5 amino acids independently selected from the group consisting of glycine, leucine, isoleucine and phenylalanine;
s is an integer ranging from 1 to 4, preferably s is 2 or 3, more preferably s is 2; and
[symbol] indicates the attachment point to the carbonyl carbon atom. Each amino acid may be, independently, an L amino acid or a D amino acid. Preferably, each amino acid is an L amino acid. Preferably, the peptide is linear.

More preferably, when being a hydrophobic moiety, Z is:

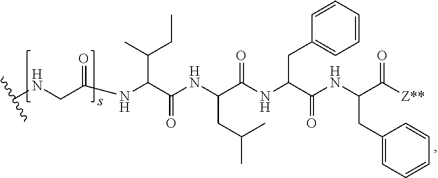

wherein:
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen;
s is an integer ranging from 1 to 4, preferably s is 2 or 3, more preferably s is 2; and
[symbol] indicates the attachment point to the carbonyl carbon atom. Preferably, Z** is NH₂ or OH.

More preferably, Z** is NH₂. Each amino acid may be, independently, an L amino acid or a D amino acid. Preferably, each amino acid is an L amino acid.

Still more preferably, when being a hydrophobic moiety, Z is:

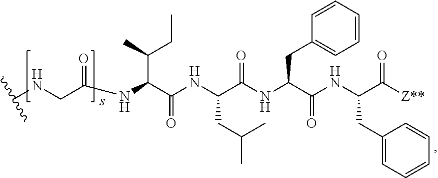

wherein:
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen;
s is an integer ranging from 1 to 4, preferably s is 2 or 3, more preferably s is 2; and
[symbol] indicates the attachment point to the carbonyl carbon atom. Preferably, Z** is NH₂ or OH. More preferably, Z** is NH₂.

In some embodiments, when being a hydrophobic moiety, Z comprises or is (C₁-C₂₀)perfluoroalkyl. As used herein, “perfluoroalkyl” denotes an alkyl group in which all hydrogen atoms are replaced by fluorine atoms. Preferably, Z comprises or is (C₁-C₁₀)perfluoroalkyl. More preferably, Z comprises or is (C₃-C₁₀)perfluoroalkyl. Still more preferably, Z comprises or is (C₄-C₆)perfluoroalkyl.

Preferably, when being a hydrophobic moiety, Z is:

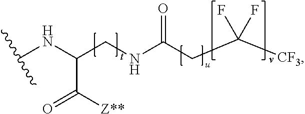

wherein:
t is an integer ranging from 1 to 8, preferably 2 to 6, more preferably t is 4;
u is an integer ranging from 1 to 4, preferably 2 or 3, more preferably u is 2;
v is an integer ranging from 1 to 19 or 1 to 9, preferably 3 to 7, more preferably v is 5; and
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen; and
[symbol] indicates the attachment point to the carbonyl carbon atom. Preferably, Z** is NH₂ or OH. More preferably, Z** is NH₂.

More preferably, when being a hydrophobic moiety, Z is:

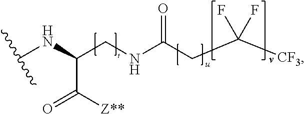

wherein:
t is 4;
u is an integer ranging from 1 to 4, preferably 2 or 3, more preferably u is 2;
v is an integer ranging from 1 to 19 or 1 to 9, preferably 3 to 7, more preferably v is 5; and
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen; and
[symbol] indicates the attachment point to the carbonyl carbon atom. Preferably, Z** is NH₂ or OH. More preferably, Z** is NH₂.

The present invention also relates to a pharmaceutically acceptable salt of the compound. Any pharmaceutically acceptable salt can be used. In particular, “pharmaceutically acceptable salt” refers to a salt of a compound of the invention that is pharmaceutically acceptable and that possesses the desired pharmacological activity of the parent compound. In particular, such salts have low toxicity and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, purely by way of example, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like. A counterion or anionic counterion can be used in a quaternary amine to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, and the like), and carboxylate ions (e.g., acetate, ethanoate, propanoate, benzoate, glycerate, lactate, tartrate, glycolate, and the like).

A is a moiety capable to bind to a cell surface. Any moiety which can bind to a cell surface, in particular by forming a covalent bond to the cell surface, can be used as the A group. In this regard, the cell surface has a functional group that can form a bond, in particular a covalent bond, with the A group. As illustrative, non-limiting examples, the functional group on the cell surface can be a thiol group (—SH), an amino group, a hydroxy group (e.g. a hydroxy group of a carbohydrate, in particular the anomeric hydroxy group of a carbohydrate), and/or a carboxy group. Preferably, the functional group on the cell surface is a thiol group or an amino group. More preferably, the functional group on the cell surface is a thiol group. In particular, the functional group can be part of a target structure which is present on the cell surface; in other words, a target structure on the cell surface can comprise the functional group, which can form a bond, in particular a covalent bond, with the A group. As illustrative examples, the target structure can be a protein, a peptide, a glycolipid, a glycoprotein, a tag or a bioorthogonal chemical reporter. A thiol group, for example, is present in a cysteine moiety of the target structure (e.g. a protein), or can be generated by reduction of an intramolecular disulfide bond of the target structure. It is also contemplated to generate a thiol group by reaction of an amino group of a lysine moiety of the target structure using 2-iminothiolane (Traut's reagent) or another thiol generating reagent. The A group is not particularly limited, and any A group can be used which is capable of forming a bond, in particular a covalent bond, with a functional group on the cell surface. Preferably, when the functional group on the cell surface is a thiol group (—SH), an amino group, a hydroxy group (e.g., a hydroxy group of a carbohydrate, in particular the anomeric hydroxy group of a carbohydrate), and/or a carboxy group, the A group is or comprises an electrophilic moiety. As illustrative examples, the formation of a bond between the functional group on the cell surface and the A group may involve a substitution reaction (e.g., nucleophilic substitution) or addition reaction (e.g., addition to a double bond or triple bond of the A group). Suitable chemical moieties, which can be used as the group A, are known to a person skilled in the art. For example, suitable A groups are described in WO 2004/010957, U.S. Pat. No. 7,659,241, WO 2018/041985 or WO 2019/170710, which are incorporated herein by reference in their entirety. A person skilled in the art knows to select suitable A groups.

In general, a group A which is capable to bind to a cell surface, can be also used to connect the cargo with the group comprising a guanidine moiety. Accordingly, it is possible that a compound of formula (1), which comprises an A group and a group comprising a guanidine moiety, can be also reacted with the cargo to conjugate the group comprising a guanidine moiety with the cargo. In this context, the cargo has a functional group that can form a bond, in particular a covalent bond, with the A group. As illustrative, non-limiting examples, the functional group of the cargo can be a thiol group (—SH), an amino group, a hydroxy group (e.g. a hydroxy group of a carbohydrate, in particular the anomeric hydroxy group of a carbohydrate), and/or a carboxy group. Preferably, the functional group of the cargo is a thiol group or an amino group. More preferably, the functional group of the cargo is a thiol group. In some embodiments, the compound comprising an A group capable to bind to the cell surface and a guanidine moiety, and the compound which can be reacted with the cargo to conjugate the group comprising a guanidine moiety with the cargo, are identical. In some embodiments, the compound comprising an A group capable to bind to the cell surface and a guanidine moiety, and the compound which can be reacted with the cargo to conjugate the group comprising a guanidine moiety with the cargo, are different.

In some embodiments, group A comprises a moiety selected from the group consisting of the following:

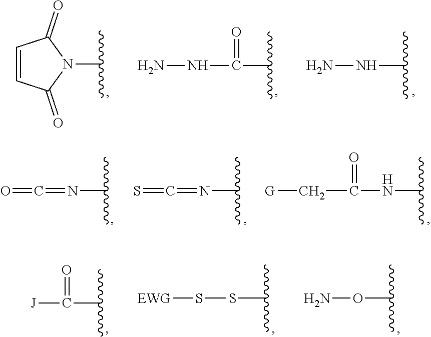

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond; wherein G is selected from —Cl, —Br, —I, —O-mesyl and —O-tosyl; J is selected from —Cl, —Br, —I, —F, —OH, —O—N-succinimide, —O-(4-nitrophenyl), —O-pentafluorophenyl, —O-tetrafluorophenyl and —O—C(O)—OR¹⁸, wherein R¹⁸ is (C₁-C₆)alkyl or aryl; S is sulfur; and EWG is an electron-withdrawing group. EWG can be any suitable electron-withdrawing group. A person skilled in the art knows to select a suitable electron-withdrawing group. As illustrative examples, the electron-withdrawing group may be e.g. an aryl or heteroaryl group, e.g. a pyridine, which is optionally further substituted with one or more electron-withdrawing groups, such as e.g. halo, nitro, cyano, and/or carboxyl. Accordingly, in some embodiments EWG is selected from the group consisting of

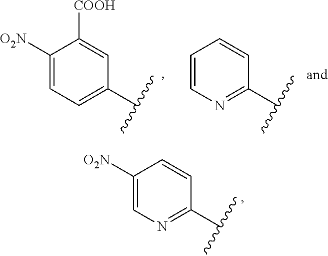 [SMILES: *c1ccc([N+](=O)[O-])c(C(=O)O)c1 ; *c1ccccn1 ; *c1ccc([N+](=O)[O-])cn1]

wherein [symbol] indicates the attachment point to the S. In some embodiments, EWG is

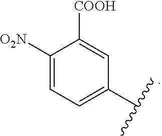 [SMILES: *c1ccc([N+](=O)[O-])c(C(=O)O)c1]

In some embodiments, EWG is

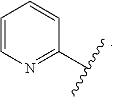 [SMILES: *c1ccccn1]

In some embodiments, EWG is

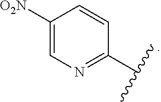 [SMILES: *c1ccc([N+](=O)[O-])cn1]

### Preferably, EWG is

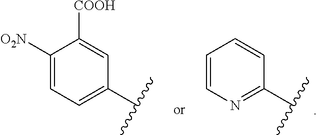 [SMILES: *c1ccc([N+](=O)[O-])c(C(=O)O)c1 ; *c1ccccn1]

More preferably, EWG is

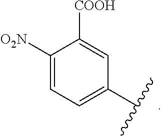 [SMILES: *c1ccc([N+](=O)[O-])c(C(=O)O)c1]

In some embodiments, group A comprises a moiety selected from the group consisting of the following:

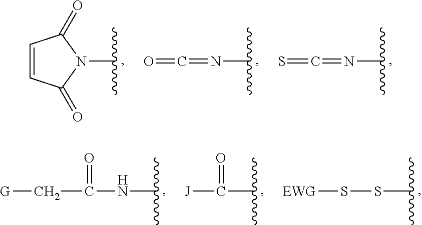

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond; wherein G, J and EWG are as defined herein. In some embodiments, the group A comprises

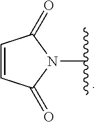 [SMILES: *N1C(=O)C=CC1=O]

In some embodiments, the group A comprises

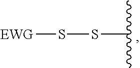

wherein EWG is as defined herein. In some embodiments, the group A comprises a carbon-carbon double bond substituted with an electron-withdrawing group. As illustrative example, the group A may comprise an acrylamide, such as e.g.

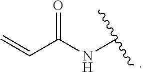 [SMILES: *NC(=O)C=C]

As illustrative example, the group A may comprise an acrylic ester, such as e.g.

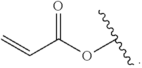 [SMILES: *OC(=O)C=C]

In some embodiments, the group A comprises a carbon-carbon triple bond substituted with an electron-withdrawing group. As illustrative example, the group A may comprise a propargyl amide, such as e.g.

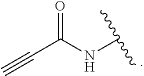 [SMILES: *NC(=O)C#C]

As illustrative example, the group A may comprise a propargylic ester, such as e.g.

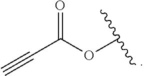 [SMILES: *OC(=O)C#C]

In some embodiments, the group A comprises a phosphorus(V) compound comprising a carbon-carbon double bond, such as, e.g., an alkene phosphonamidate, an alkene phosphonothiolate or an alkene phosphonate. As illustrative example, the group A may comprise

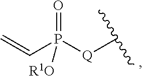

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, the A group may comprise

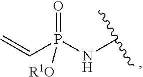

wherein R¹ is as defined herein. In some embodiments, the group A comprises a phosphorus(V) compound comprising a carbon-carbon triple bond, such as, e.g., an alkyne phosphonamidate, an alkyne phosphonothiolate or an alkyne phosphonate. As illustrative example, the group A may comprise

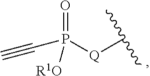

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, the A group may comprise

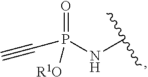

wherein R¹ is as defined herein.

In some embodiments, the group A is a moiety selected from the group consisting of the following:

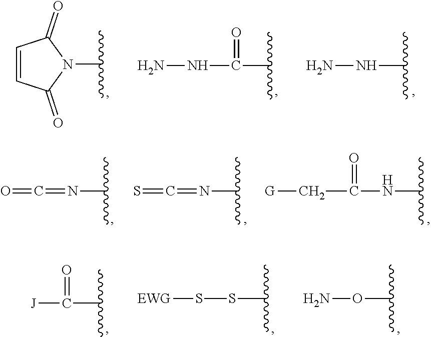

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond; wherein G, J, and EWG are as defined herein. In some embodiments, group A is a moiety selected from the group consisting of the following:

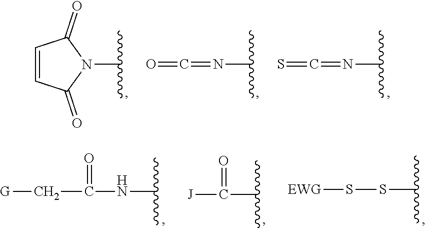

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond; wherein G, J, and EWG are as defined herein. In some embodiments, the group A is

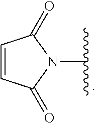 [SMILES: *N1C(=O)C=CC1=O]

In some embodiments, the group A is

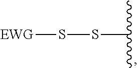

wherein EWG is as defined herein; S is sulfur. In some embodiments, the group A is a carbon-carbon double bond substituted with an electron-withdrawing group. As illustrative example, the group A may be an acrylamide, such as e.g.

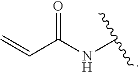 [SMILES: *NC(=O)C=C]

As illustrative example, the group A may be an acrylic ester, such as e.g.

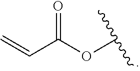 [SMILES: *OC(=O)C=C]

In some embodiments, the group A is a carbon-carbon triple bond substituted with an electron-withdrawing group. As illustrative example, the group A may be a propargyl amide, such as e.g.

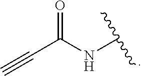 [SMILES: *NC(=O)C#C]

As illustrative example, the group A may be a propargylic ester, such as e.g.

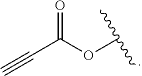 [SMILES: *OC(=O)C#C]

In some embodiments, the group A is a phosphorus(V) compound comprising a carbon-carbon double bond, such as, e.g., an alkene phosphonamidate, an alkene phosphonothiolate or an alkene phosphonate. As illustrative example, the group A may be

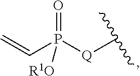

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, the A group may be

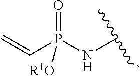

wherein R¹ is as defined herein. In some embodiments, the group A is a phosphorus(V) compound comprising a carbon-carbon triple bond, such as, e.g., an alkyne phosphonamidate, an alkyne phosphonothiolate or an alkyne phosphonate. As illustrative example, the group A may be

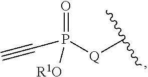

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, the A group may be

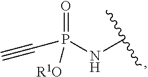

(wherein R¹ is as defined herein.

Preferably, A is a thiol-reactive moiety. As used herein, a “thiol-reactive moiety” is any moiety or functional group which is capable of reacting with a thiol group (SH), such as e.g. a thiol group present on a cell surface. In general, such reaction of a thiol-reactive moiety A with a thiol group leads to formation of a covalent bond. For example, reaction of a thiol-reactive moiety A with the thiol group can involve substitution (e.g., nucleophilic substitution) or addition (e.g., addition of the thiol group to a double bond or triple bond of the A group). Suitable thiol-reactive moieties are known to a person skilled in the art. A person skilled in the art knows to select suitable thiol-reactive moieties. For example, an A group as described above and below can react with a thiol group.

Preferably, A is

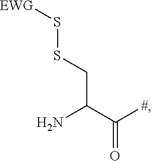

wherein #indicates the attachment point to the L in the compound, and EWG is an electron-withdrawing group. Preferably, the A group may be derived from naturally occurring cysteine, thus having the L configuration (alternatively termed R configuration):

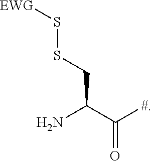

Alternatively, the A group may have the D configuration (alternatively termed S configuration):

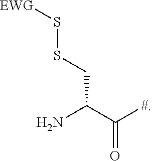

This A group is an example for a thiol-reactive moiety, and it is capable of reacting with a thiol group (SH), e.g. a thiol group on a cell surface, by way of substitution. By reacting with a thiol group, the EWG and the S atom, to which the EWG is attached, are replaced by the sulfur atom of the thiol group, so that a disulfide bond (—S—S—) is formed. EWG can be any suitable electron-withdrawing group. A person skilled in the art knows to select suitable electron-withdrawing groups. As illustrative examples, the electron-withdrawing group may be e.g. an aryl or heteroaryl group, e.g. a pyridine, which is optionally further substituted with one or more electron-withdrawing groups, such as e.g. halo, nitro, cyano, and/or carboxyl. Accordingly, in some embodiments EWG is selected from the group consisting of

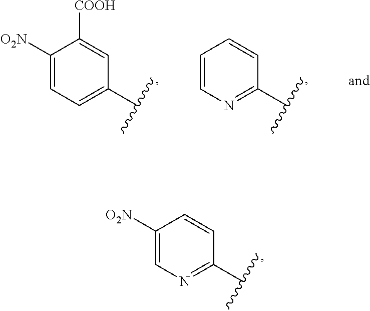 [SMILES: *c1ccc([N+](=O)[O-])c(C(=O)O)c1 ; *c1ccccn1 ; *c1ccc([N+](=O)[O-])cn1]

wherein [symbol] indicates the attachment point to the S. In some embodiments EWG is

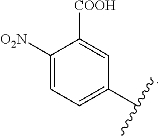 [SMILES: *c1ccc([N+](=O)[O-])c(C(=O)O)c1]

In some embodiments, EWG is

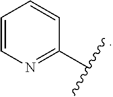 [SMILES: *c1ccccn1]

In some embodiments, EWG is

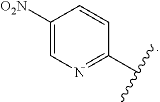 [SMILES: *c1ccc([N+](=O)[O-])cn1]

### Preferably, EWG is

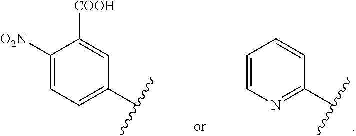 [SMILES: *c1ccc([N+](=O)[O-])c(C(=O)O)c1 ; *c1ccccn1]

More preferably EWG is

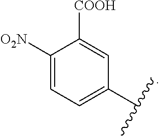 [SMILES: *c1ccc([N+](=O)[O-])c(C(=O)O)c1]

In some embodiments, A is

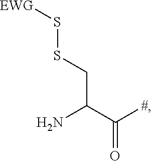

and Z is NR¹R², wherein EWG, #, R¹ and R² are as defined herein; more preferably, EWG is

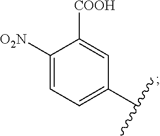 [SMILES: *c1ccc([N+](=O)[O-])c(C(=O)O)c1]

and/or more preferably, R¹ and R² are each hydrogen.

Also preferably, A is

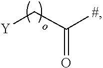

wherein Y is selected from the group consisting of

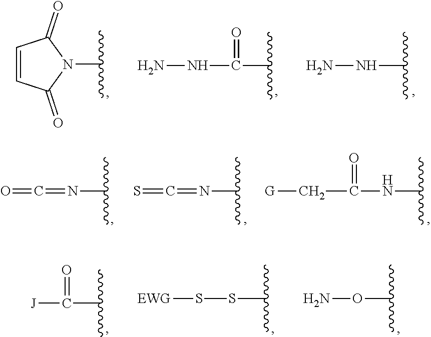

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond; wherein o is an integer ranging from 0 to 10, #indicates the attachment point to the L in the compound, G is selected from —Cl, —Br, —I, —O-mesyl and —O-tosyl; J is selected from —Cl, —Br, —I, —F, —OH, —O—N-succinimide, —O-(4-nitrophenyl), —O-pentafluorophenyl, —O-tetrafluorophenyl and —O—C(O)—OR¹⁸, wherein R¹⁸ is (C₁-C₆)alkyl or aryl; S is sulfur; and EWG is an electron-withdrawing group. These A groups are further examples for thiol-reactive moieties. For example, such A groups may react with a thiol group, e.g. a thiol group on a cell surface, by way of substitution or addition. For example, in A groups which comprise a carbon-carbon double bond or a carbon-carbon triple bond, the carbon-carbon double bond or the carbon-carbon triple bond can be capable of reacting with a thiol group, e.g. a thiol group on a cell surface, by way of addition of the thiol group to the carbon-carbon double bond or to the carbon-carbon triple bond, in order to form a covalent linkage. EWG can be any suitable electron-withdrawing group. A person skilled in the art knows to select a suitable electron-withdrawing group. As illustrative examples, the electron-withdrawing group may be e.g. an aryl or heteroaryl group, e.g. a pyridine, which is optionally further substituted with one or more electron-withdrawing groups, such as e.g. halo, nitro, cyano, and/or carboxyl. Accordingly, in some embodiments the EWG is selected from the group consisting of

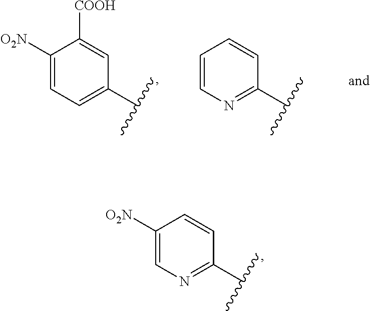 [SMILES: *c1ccc([N+](=O)[O-])c(C(=O)O)c1 ; *c1ccccn1 ; *c1ccc([N+](=O)[O-])cn1]

wherein [symbol] indicates the attachment point to the S. In some embodiments, the EWG is

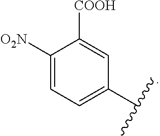 [SMILES: *c1ccc([N+](=O)[O-])c(C(=O)O)c1]

In some embodiments, the EWG is

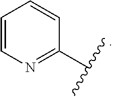 [SMILES: *c1ccccn1]

In some embodiments, the EWG is

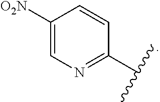 [SMILES: *c1ccc([N+](=O)[O-])cn1]

### Preferably, the EWG is

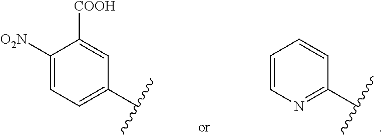 [SMILES: *c1ccc([N+](=O)[O-])c(C(=O)O)c1 ; *c1ccccn1]

More preferably, the EWG is

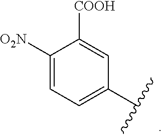 [SMILES: *c1ccc([N+](=O)[O-])c(C(=O)O)c1]

o is an integer ranging from 0 to 10. Preferably, o is an integer ranging from 1 to 10. More preferably, o is an integer ranging from 1 to 8. Still more preferably, o is an integer ranging from 1 to 5. Still more preferably, o is an integer ranging from 1 to 3. Most preferably, o is 1. Preferably, A is

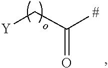

wherein Y is selected from the group consisting of

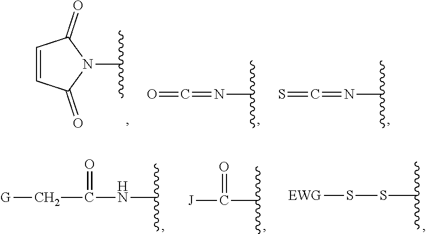

a carbon-carbon double bond substituted with an electron-withdrawing group, a carbon-carbon triple bond substituted with an electron-withdrawing group, a phosphorus(V) compound comprising a carbon-carbon double bond, and a phosphorus(V) compound comprising a carbon-carbon triple bond. More preferably, Y is

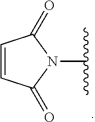 [SMILES: *N1C(=O)C=CC1=O]

Accordingly, in preferred embodiments A is

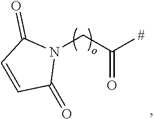

wherein o and # are as defined herein; more preferably, o is 1. In some embodiments, Y is

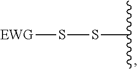

wherein EWG is as defined herein. Accordingly, in some embodiments A is

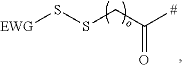

wherein EWG, o and # are as defined herein. In some embodiments, Y is a carbon-carbon double bond substituted with an electron-withdrawing group. As illustrative example, Y may be an acrylamide, such as e.g.

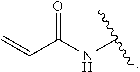 [SMILES: *NC(=O)C=C]

As illustrative example, Y may be an acrylic ester, such as e.g.

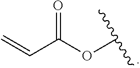 [SMILES: *OC(=O)C=C]

Accordingly, in some embodiments, A is

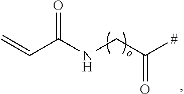

wherein o and # are as defined herein. In some embodiments, A is

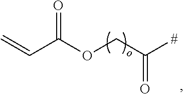

wherein o and # is as defined herein. In some embodiments, Y is a carbon-carbon triple bond substituted with an electron-withdrawing group. As illustrative example, Y may be a propargyl amide, such as e.g.

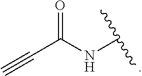 [SMILES: *NC(=O)C#C]

As illustrative example, Y may be a propargylic ester, such as e.g.

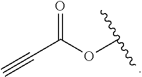 [SMILES: *OC(=O)C#C]

Accordingly, in some embodiments, A is

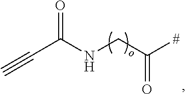

wherein o and # are as defined herein. In some embodiments, A is

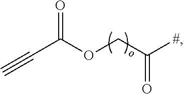

wherein o and # are as defined herein. In some embodiments, Y is a phosphorus(V) compound comprising a carbon-carbon double bond, such as, e.g., an alkene phosphonamidate, an alkene phosphonothiolate or an alkene phosphonate. As illustrative example, Y may be

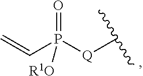

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, Y may be

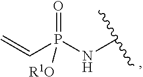

wherein R¹ is as defined herein. Accordingly, in some embodiments, A is

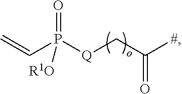

wherein o, #, R¹ and Q are as defined herein; preferably, Q is NH or S, more preferably NH. In some embodiments, Y is a phosphorus(V) compound comprising a carbon-carbon triple bond, such as, e.g., an alkyne phosphonamidate, an alkyne phosphonothiolate or an alkyne phosphonate. As illustrative example, Y may be

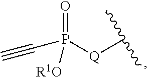

wherein Q is NH, S or O, preferably NH or S, more preferably NH; and R¹ is (C₁-C₆)alkyl, preferably (C₁-C₄)alkyl, more preferably methyl, ethyl, propyl or butyl, still more preferably methyl or ethyl. As illustrative example, Y may be

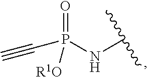

wherein R¹ is as defined herein. Accordingly, in some embodiments A is

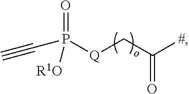

wherein o, #, R¹ and Q are as defined herein; preferably, Q is NH or S, more preferably NH. In some embodiments, A is

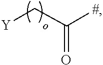

and Z is NR¹R², wherein Y, o, #, R¹ and R² are as defined herein; more preferably, A is

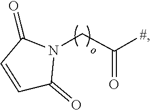

wherein o and # are as defined herein; and/or more preferably, o is 1; and/or more preferably, R¹ and R² are each hydrogen.

Also preferably, A is a moiety which is capable to bind to a cell surface via an enzymatic reaction. Suitable A moieties and target structures, which are located on the cell surface and capable to react via an enzymatic reaction, are known to a person skilled in the art, and can be suitably selected. In general, a covalent bond is formed via the enzymatic reaction between the target structure and A. As an illustrative example, in some embodiments, the target structure may be a tag. Any tag known to a person skilled in the art may be used. As illustrative examples, any tag disclosed herein may be used. Preferably, the tag is a Halotag. Accordingly, in some embodiments, A is capable to bind to a Halotag. Preferably, A is

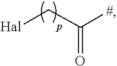

wherein Hal is a halogen (F, Cl, Br, or I, preferably Cl or Br, more preferably Cl), p is an integer ranging from 1 to 10, and #indicates the attachment point to the L in the compound. Preferably, Hal is Cl. Preferably, p is an integer ranging from 2 to 8. More preferably, p is an integer ranging from 3 to 7. Still more preferably, p is an integer ranging from 4 to 6. Most preferably, p is 5. In some embodiments, A is

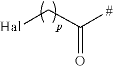

and Z is NR¹R², wherein Hal, p, #, R¹ and R² are as defined herein; more preferably, Hal is Cl; and/or more preferably, p is 5; and/or more preferably, R¹ and R² are each hydrogen.

Preferably, L is a linker. As used herein, a “linker” or “linker moiety” is any chemical moiety that is capable to covalently link the A group to the other parts of the compound. Virtually any linker moiety (linker) can be used. The linker may, for example, be a straight or branched hydrocarbon based moiety. The linker can also comprise cyclic moieties. If the linking moiety is a hydrocarbon-based moiety, the main chain of the linker may comprise only carbon atoms but can also contain heteroatoms such as oxygen (O), nitrogen (N) or sulfur (S) atoms. The linker may for example include a C₁-C₂₀ carbon atom chain or a polyether based chain such as polyethylene glycol based chain with —(O—CH₂—CH₂)-repeating units. In typical embodiments of hydrocarbon based linkers, the linking moiety may comprise between 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18, and 19 main chain atoms. Preferably, the linker is substantially resistant to cleavage (e.g., stable linker or non-cleavable linker) under conditions present in a cell or cell environment, in order to be able to provide a stable covalent bond to the target structure on the cell surface. A person skilled in the art knows to select a suitable linker, in particular a stable linker.

In some embodiments, L is selected from the group consisting of —(C₁-C₁₀)alkylene-C(O)—, —(C₃-C₈)carbocyclo-C(O)—, -arylene-C(O)—, —(C₁-C₁₀)alkylene-arylene-C(O)—, -arylene-(C₁-C₁₀)alkylene-C(O)—, —(C₁-C₁₀)alkylene-(C₃-C₈)carbocyclo-C(O)—, —(C₃-C₈)carbocyclo-(C₁-C₁₀)alkylene-C(O)—, —(C₃-C₈)heterocyclo-C(O)—, —(C₁-C₁₀)alkylene-(C₃-C₈)heterocyclo-C(O)—, —(C₃-C₈)heterocyclo-(C₁-C₁₀)alkylene-C(O)—, —(CH₂CH₂CH₂)ᵣ—C(O)—, —(CH₂CH₂CH₂)ᵣ—CH₂—C(O)—, —(CH₂CH₂NH)ᵣ—C(O)—, —(CH₂CH₂NH)ᵣ—CH₂—C(O)—, —(CH₂CH₂O)ᵣ—C(O)—, and —(CH₂CH₂O)ᵣ—CH₂—C(O)—, wherein r, in each instance, is an integer ranging from 1 to 10, preferably 2 to 8, more preferably 3 to 7, still more preferably 4 to 6, still more preferably 5.

In some embodiments, L is selected from the group consisting of —NR⁴—(C₁-C₁₀)alkylene-C(O)—, —NR⁴—(C₃-C₈)carbocyclo-C(O)—, —NR⁴-arylene-C(O)—, —NR⁴—(C₁-C₁₀)alkylene-arylene-C(O)—, —NR⁴-arylene-(C₁-C₁₀)alkylene-C(O)—, —NR⁴—(C₁-C₁₀)alkylene-(C₃-C₈)carbocyclo-C(O)—, —NR⁴—(C₃-C₈)carbocyclo-(C₁-C₁₀)alkylene-C(O)—, —NR⁴—(C₃-C₈)heterocyclo-C(O)—, —NR⁴—(C₁-C₁₀)alkylene-(C₃-C₈)heterocyclo-C(O)—, —NR⁴—(C₃-C₈)heterocyclo-(C₁-C₁₀)alkylene-C(O)—, —NR⁴—(CH₂CH₂CH₂)ᵣ—C(O)—, —NR⁴—(CH₂CH₂CH₂)ᵣ—CH₂—C(O)—, —NR⁴—(CH₂CH₂NH)ᵣ—C(O)—, —NR⁴—(CH₂CH₂NH)ᵣ—CH₂—C(O)—, —NR⁴—(CH₂CH₂O)ᵣ—C(O)—, and —NR₄—(CH₂CH₂O)ᵣ—CH₂—C(O)—, wherein r, in each instance, is an integer ranging from 1 to 10, preferably 2 to 8, more preferably 3 to 7, still more preferably 4 to 6, still more preferably 5; and R⁴ is each independently selected from the group consisting of hydrogen and (C₁-C₈)alkyl, preferably R⁴ is each hydrogen.

In some embodiments, L is selected from the group consisting of —O—(C₁-C₁₀)alkylene-C(O)—, —O—(C₃-C₈)carbocyclo-C(O)—, —O-arylene-C(O)—, —O—(C₁-C₁₀)alkylene-arylene-C(O)—, —O-arylene-(C₁-C₁₀)alkylene-C(O)—, —O—(C₁-C₁₀)alkylene-(C₃-C₈)carbocyclo-C(O)—, —O—(C₃-C₈)carbocyclo-(C₁-C₁₀)alkylene-C(O)—, —O—(C₃-C₈)heterocyclo-C(O)—, —O—(C₁-C₁₀)alkylene-(C₃-C₈)heterocyclo-C(O)—, —O—(C₃-C₈)heterocyclo-(C₁-C₁₀)alkylene-C(O)—, —O—(CH₂CH₂CH₂)ᵣ—C(O)—, —O—(CH₂CH₂CH₂)ᵣ—CH₂—C(O)—, —O—(CH₂CH₂NH)ᵣ—C(O)—, —O—(CH₂CH₂NH)ᵣ—CH₂—C(O)—, —O—(CH₂CH₂O)ᵣ—C(O)—, and —O—(CH₂CH₂O)ᵣ—CH₂—C(O)—, wherein r, in each instance, is an integer ranging from 1 to 10, preferably 2 to 8, more preferably 3 to 7, still more preferably 4 to 6, still more preferably 5.

A “(C₃-C₈)carbocycle” is a 3-, 4-, 5-, 6-, 7- or 8-membered saturated or unsaturated non-aromatic carbocyclic ring. Representative (C₃-C₈)carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A (C₃-C₈)carbocycle group can be unsubstituted or substituted with one or more groups including, but not limited to, —(C₁—Ce)alkyl, —O—(C₁-C₆)alkyl, -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₈)alkyl and aryl.

A “(C₃-C₈)carbocyclo” refers to a (C₃-C₈)carbocycle group defined above wherein one of the carbocycle groups hydrogen atoms is replaced with a bond.

A “(C₁-C₁₀)alkylene” is a straight chain, saturated hydrocarbon group of the formula —(CH₂)₁₋₁₀—. Examples of a (C₁-C₁₀)alkylene include methylene, ethylene, propylene, butylene, pentylene, hexylene, heptylene, ocytylene, nonylene and decalene.

An “arylene” is an aryl group which has two covalent bonds and can be in the ortho, meta, or para configurations as shown in the following structures:

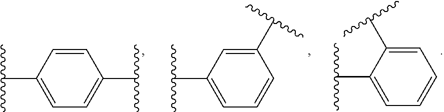

in which the phenyl group can be unsubstituted or substituted with up to four groups including, but not limited to, —(C₁-C₈)alkyl, —O—(C₁-C₈)alkyl, -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₈)alkyl and aryl.

A “(C₃-C₆)heterocycle” refers to an aromatic or non-aromatic (C₃-C₆)carbocycle in which one to four of the ring carbon atoms are independently replaced with a heteroatom from the group consisting of O, S and N. Representative examples of a (C₃—Cs)heterocycle include, but are not limited to, benzofuranyl benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A (C₃-C₆)heterocycle can be unsubstituted or substituted with up to seven groups including, but not limited to, —(C₁-C₆)alkyl, —O—(C₁-C₆)alkyl, -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₆)alkyl and aryl.

“(C₃-C₆)heterocyclo” refers to a (C₃-C₆)heterocycle group defined above wherein one of the heterocycle groups hydrogen atoms is replaced with a bond. A (C₃-C₆)heterocyclo can be unsubstituted or substituted with up to six groups including, but not limited to, —(C₁-C₆)alkyl, —O—(C₁-C₆)alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₆)alkyl and aryl.

“Aryl” refers to a carbocyclic or heterocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —(C₁-C₆)alkyl, —O—(C₁-C₆)alkyl, -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from —(C₁-C₆)alkyl and aryl.

Preferably, L is

—[B¹—(CH₂CH₂B₂)ₕ—(CH₂)ⱼ—C(O)]ₖ—,
wherein B¹ and B² are independently selected from the group consisting of CH₂, NH and O; h is an integer ranging from 1 to 4; j is 1 or 2; and k is an integer ranging from 1 to 10.

Preferably, B¹ is NH and B² is O. Preferably, h is an integer ranging from 2 to 3. More preferably, h is 2. Preferably, j is 1. Preferably, k is an integer ranging from 2 to 8. More preferably, k is an integer ranging from 2 to 7. Still more preferably, k is an integer ranging from 2 to 6. Still more preferably, k is an integer ranging from 2 to 5. Still more preferably, k is an integer ranging from 2 to 4. Still more preferably, k is an integer ranging from 2 to 3. Most preferably, k is 2.

In some preferred embodiments, the compound is a compound of formula (1a):

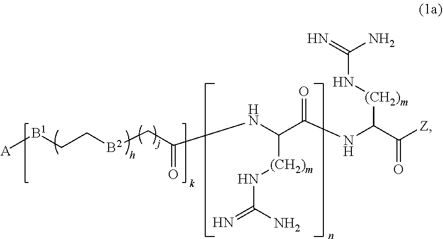

wherein A, B¹, B², h, j, k, m, n and Z are as defined herein. Any A, B¹, B², h, j, k, m, n and Z as defined herein can be combined with each other. More preferably, the compound is a compound of formula (1b):

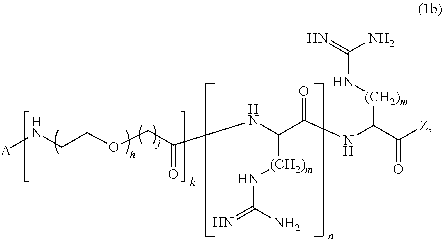

wherein A, h, j, k, m, n and Z are as defined herein. Any A, h, j, k, m, n and Z as defined herein can be combined with each other. Still more preferably, the compound is a compound of formula (1c):

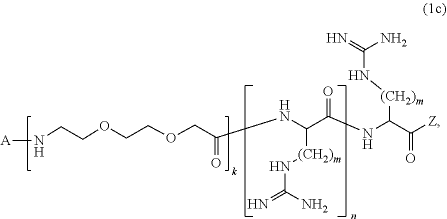

wherein A, k, m, n and Z are as defined herein. Any A, k, m, n and Z as defined herein can be combined with each other.

In preferred embodiments, m is each 3. Accordingly, in some preferred embodiments, the compound is a compound of formula (2):

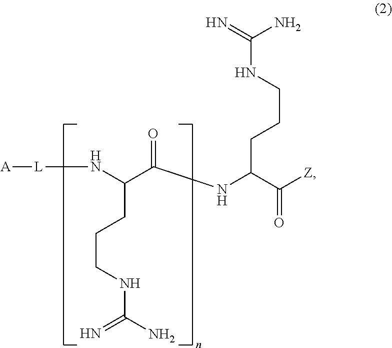

wherein A, L, n and Z are as defined herein. Any A, L, n and Z as defined herein can be combined with each other.

More preferably, the compound is a compound of formula (2a):

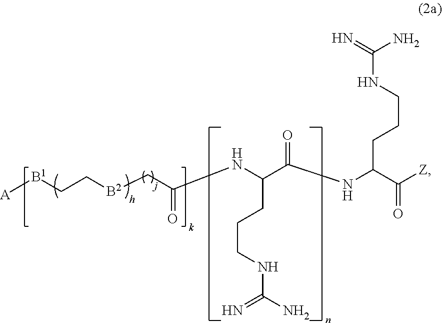

wherein A, B¹, B², h, j, k, n and Z are as defined herein. Any A, B¹, B², h, j, k, n and Z as defined herein can be combined with each other. Still more preferably, the compound is a compound of formula (2b):

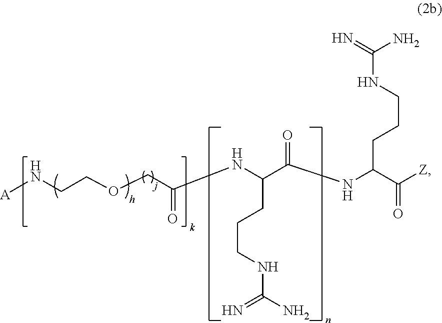

wherein A, h, j, k, n and Z are as defined herein. Any A, h, j, k, n and Z as defined herein can be combined with each other. Still more preferably, the compound is a compound of formula (2c):

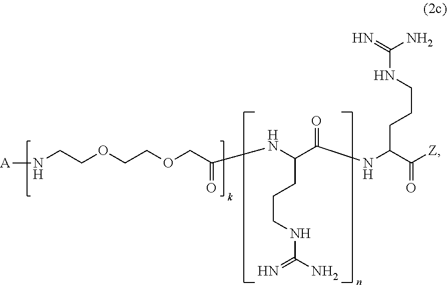

wherein A, k, n and Z are as defined herein. Any A, k, n and Z as defined herein can be combined with each other.

In the compound, each amino acid moiety may independently have the L configuration (or termed S configuration) or the D configuration (or termed R configuration). Preferably, each amino acid moiety has its natural configuration. In some preferred embodiments, each amino acid moiety has the L configuration (or termed S configuration). In some embodiments, each amino acid has the D configuration (or termed R configuration). As an illustrative example, when the integer m is each 3, the amino acid moiety is arginine. Each arginine may have the D configuration (or termed R configuration). Preferably, each arginine moiety has its natural configuration, i.e. the L configuration (or termed S configuration). Accordingly, in some preferred embodiments, the compound is a compound of formula (2*):

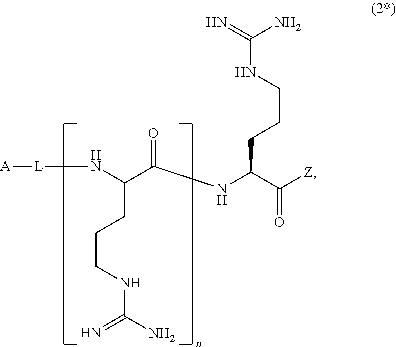

wherein A, L, n and Z are as defined herein. Any A, L, n and Z as defined herein can be combined with each other. More preferably, the compound is a compound of formula (2a*):

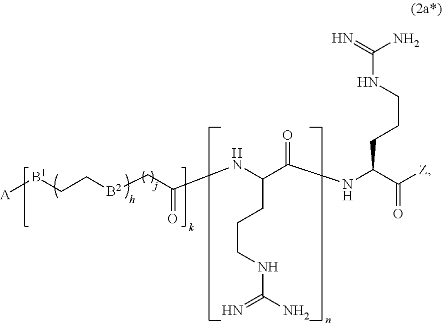

wherein A, B¹, B², h, j, k, n and Z are as defined herein. Any A, B¹, B², h, j, k, n and Z as defined herein can be combined with each other. Still more preferably, the compound is a compound of formula (2b*):

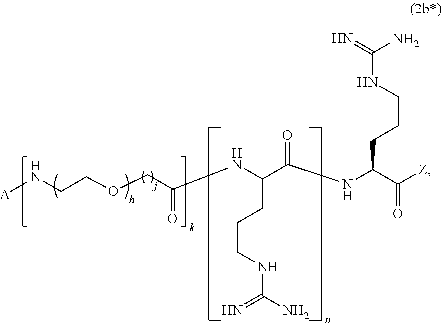

wherein A, h, j, k, n and Z are as defined herein. Any A, h, j, k, n and Z as defined herein can be combined with each other. Still more preferably, the compound is a compound of formula (2c*):

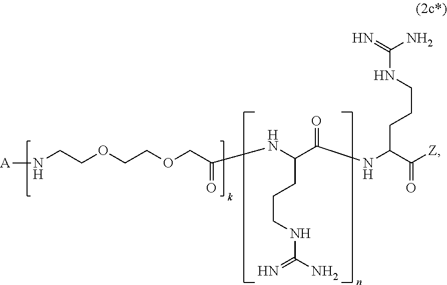

wherein A, k, n and Z are as defined herein. Any A, k, n and Z as defined herein can be combined with each other.

Preferably, the compound is:

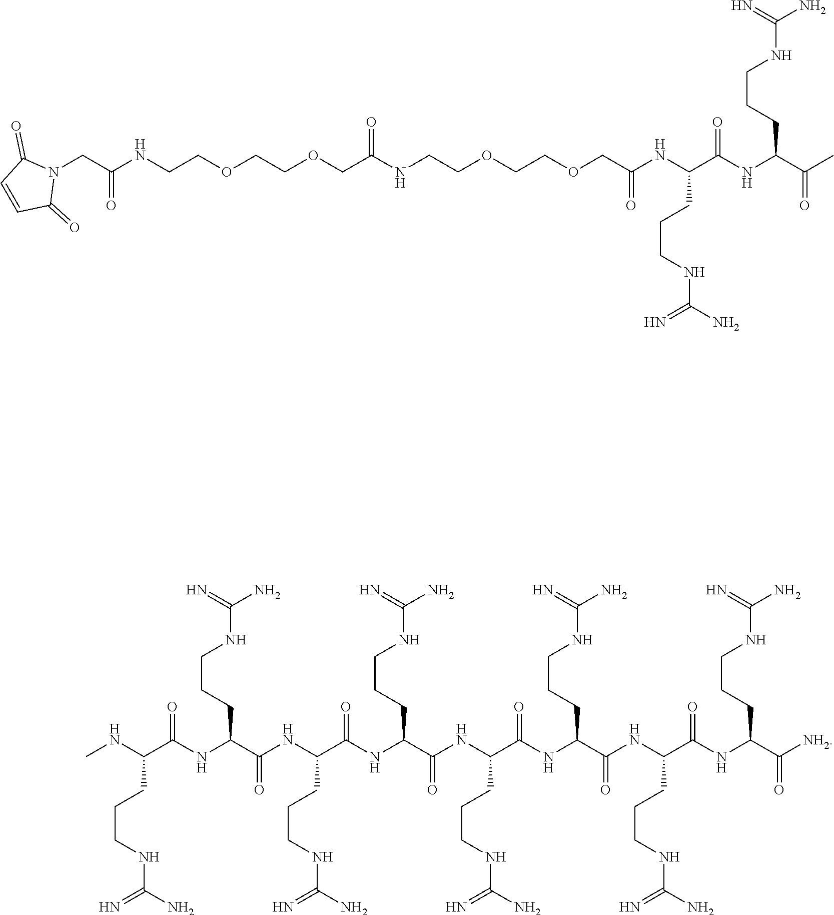

Most preferably, the compound is:

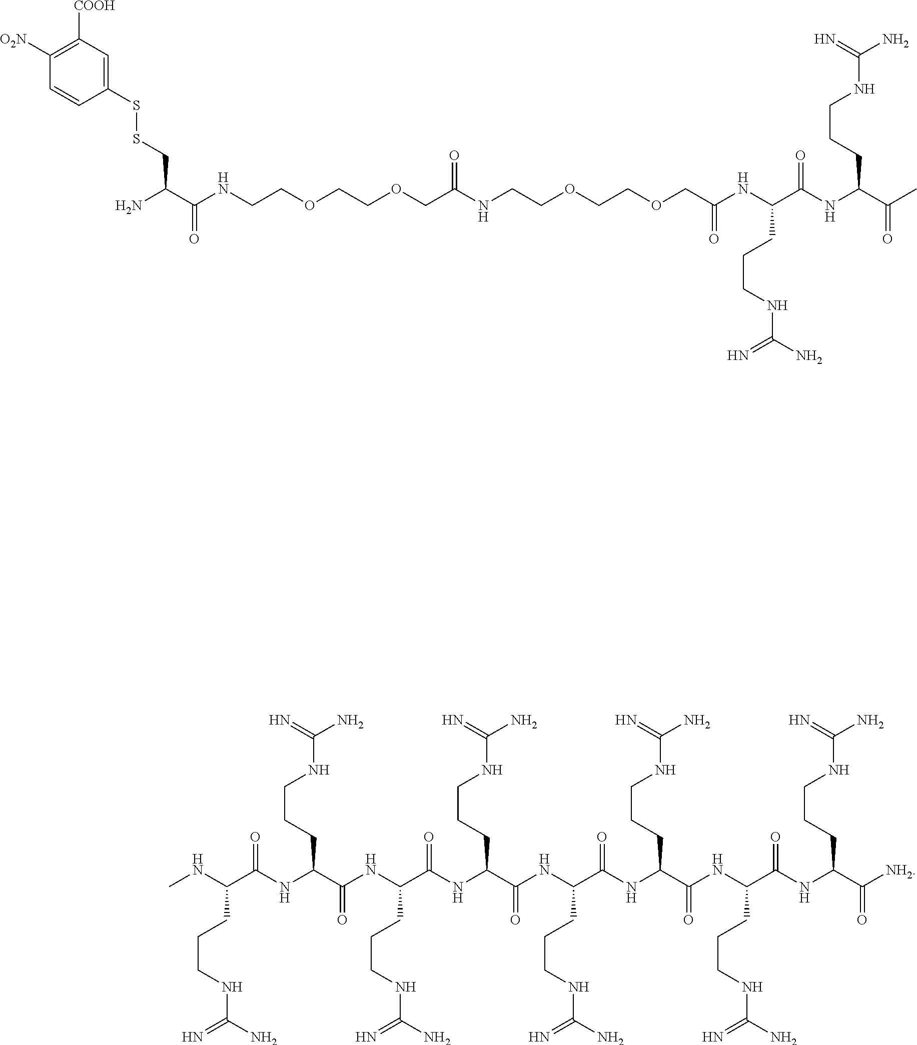

Preferably, the compound is:

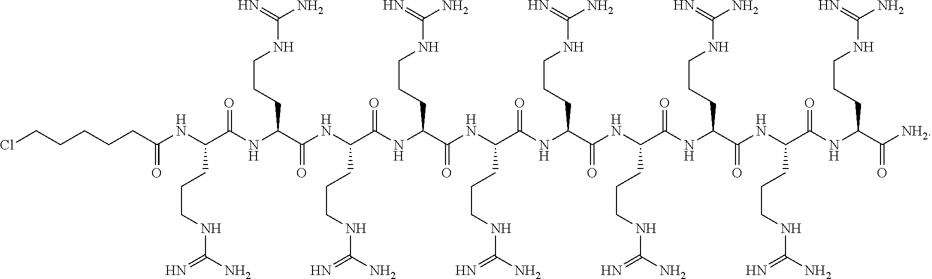 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)CCCCCCl)C(N)=O]

Preferably, the compound is:

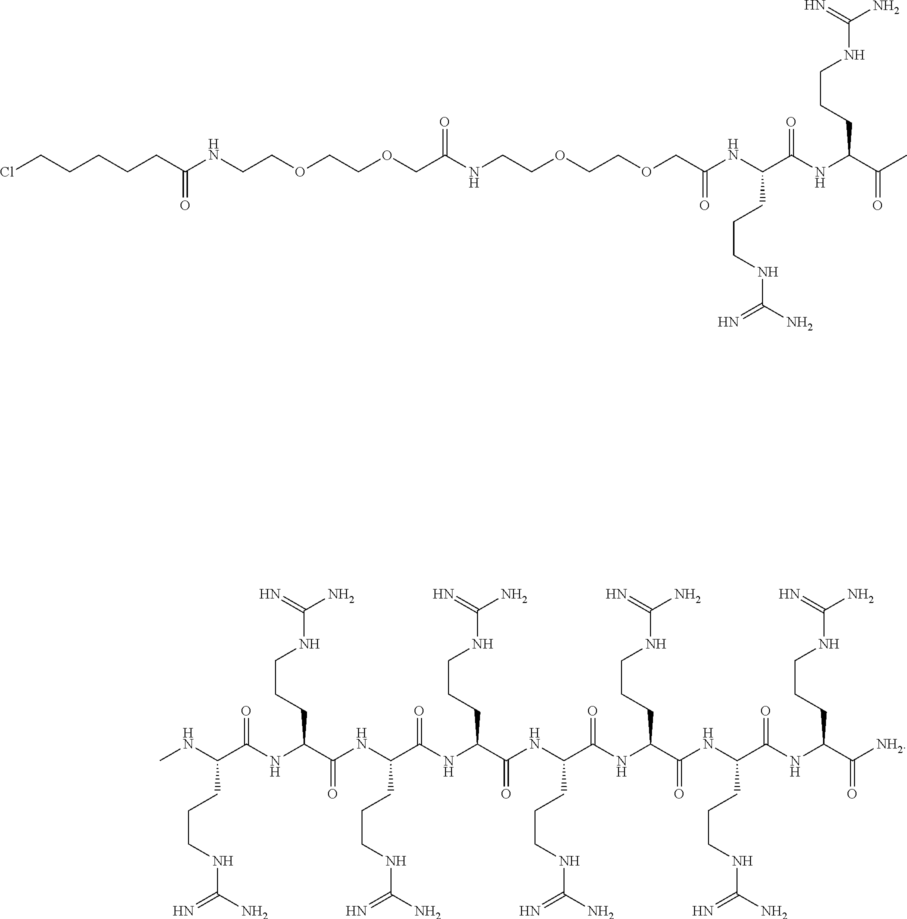

Preferably, the compound is:

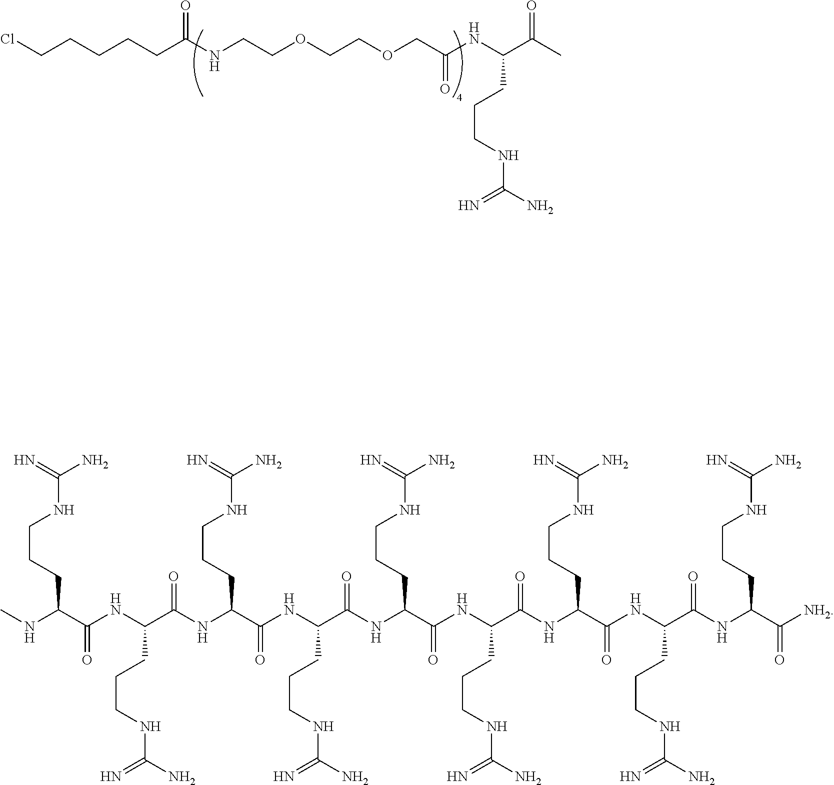

Preferably, the compound is:

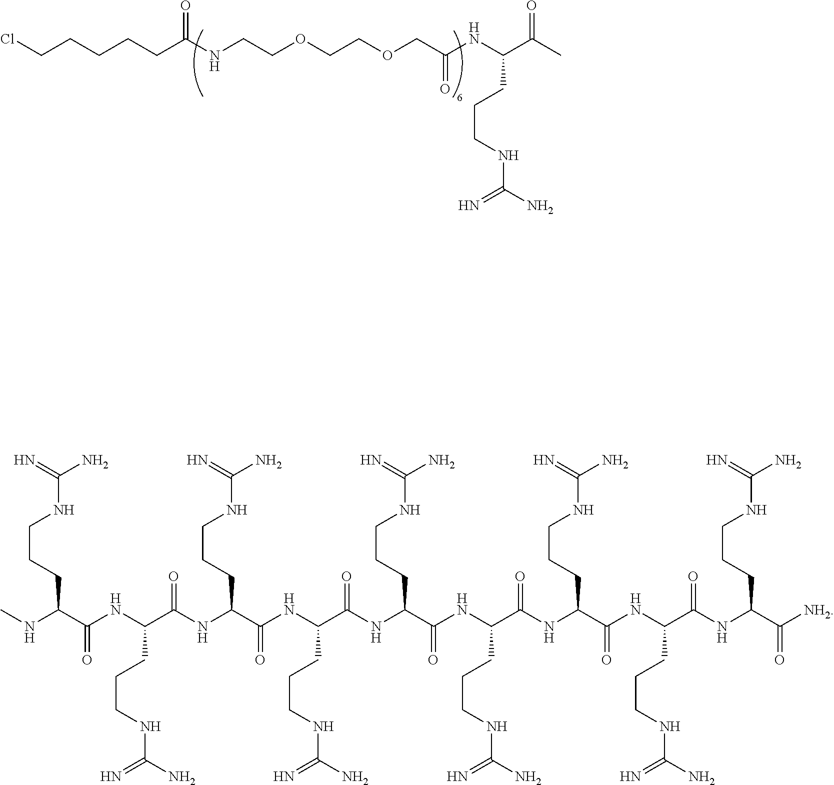

Preferably, the compound is:

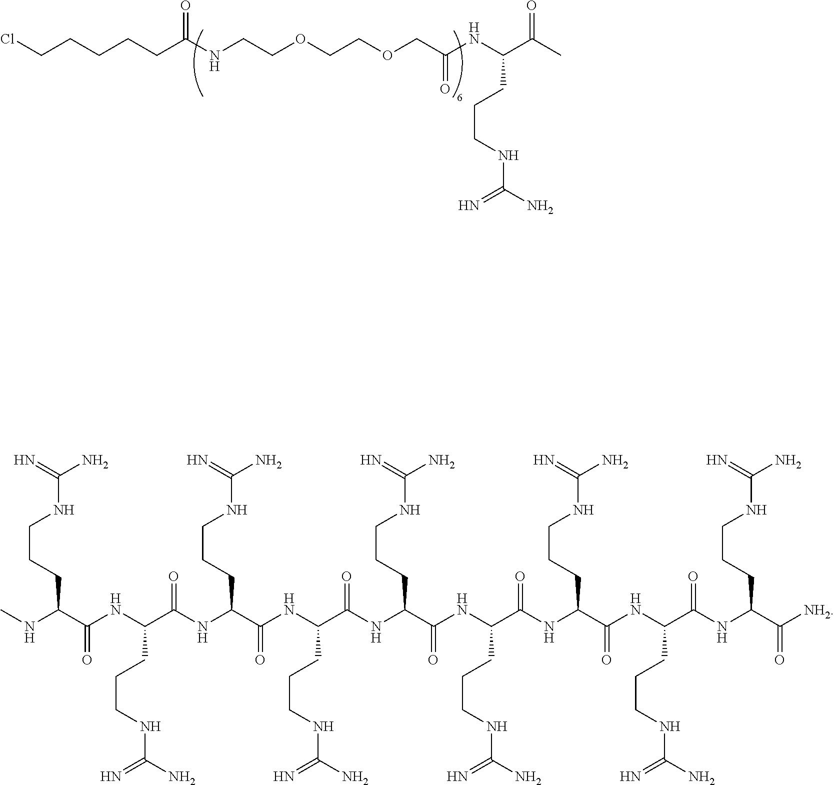

Preferably, the compound is:

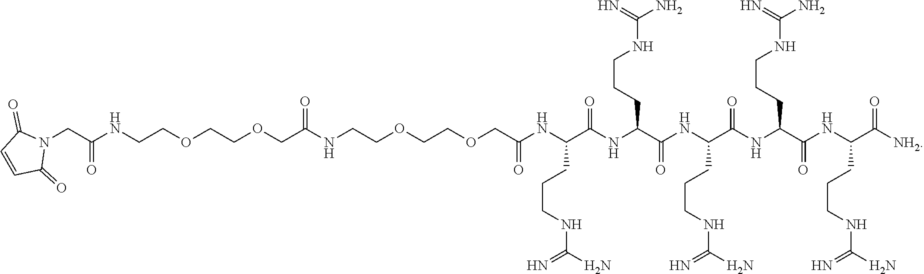 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)COCCOCCNC(=O)COCCOCCNC(=O)CN1C(=O)C=CC1=O)C(N)=O]

Preferably, the compound is:

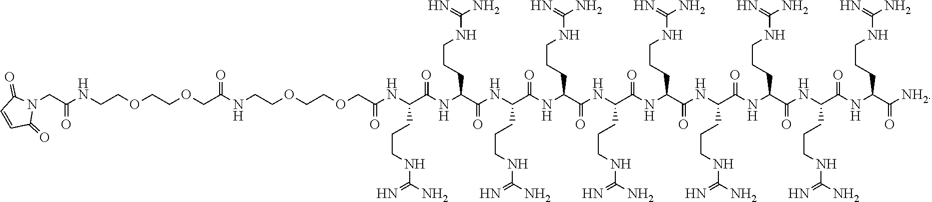 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)COCCOCCNC(=O)COCCOCCNC(=O)CN1C(=O)C=CC1=O)C(N)=O]

Also, a compound may be:

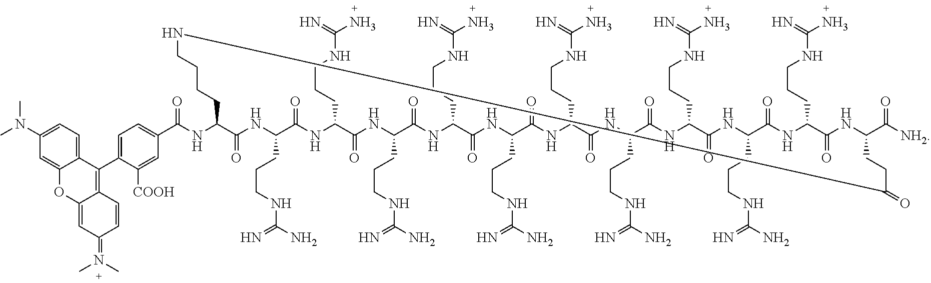

Also, a compound may be:

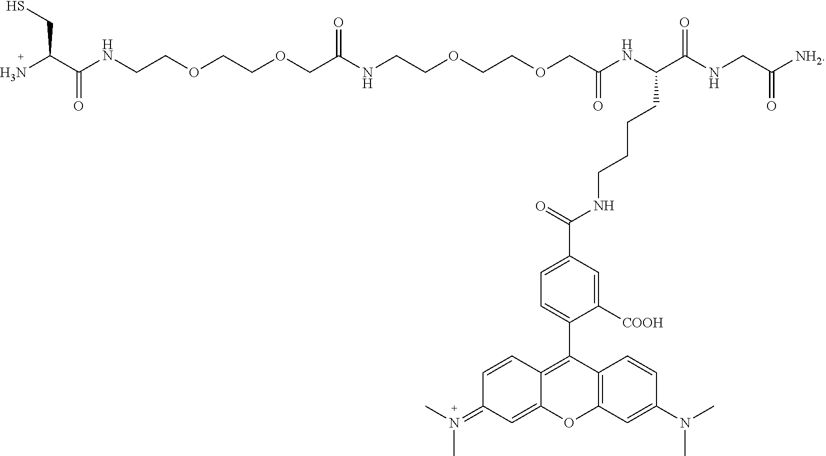

Also, a compound may be:

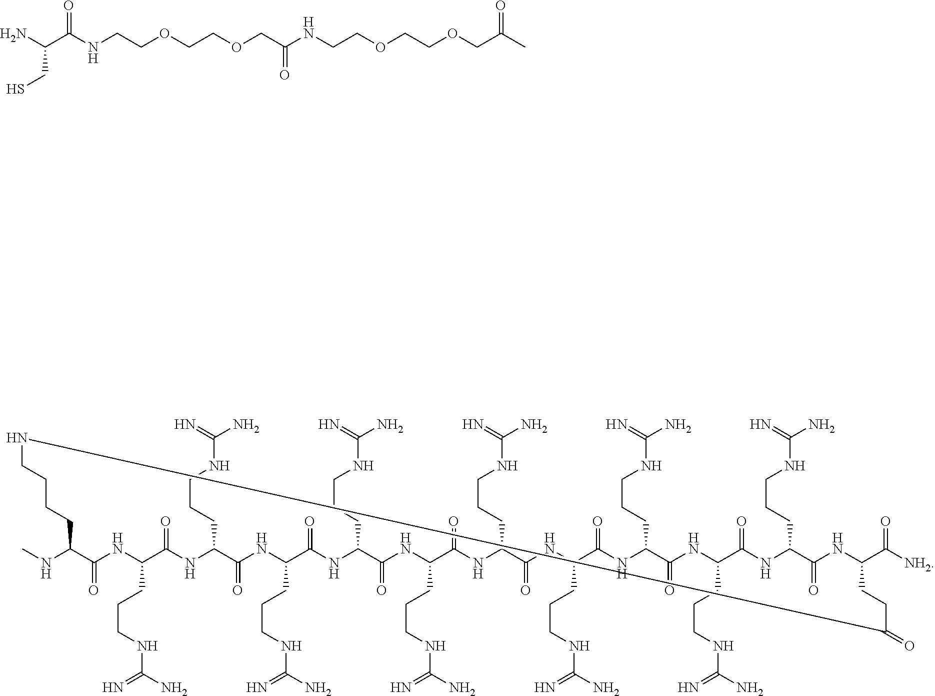 [SMILES: CC(=O)COCCOCCNC(=O)COCCOCCNC(=O)[C@@H](N)CS ; CN[C@H]1CCCCNC(=O)CC[C@@H](C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O]

Also, a compound may be:

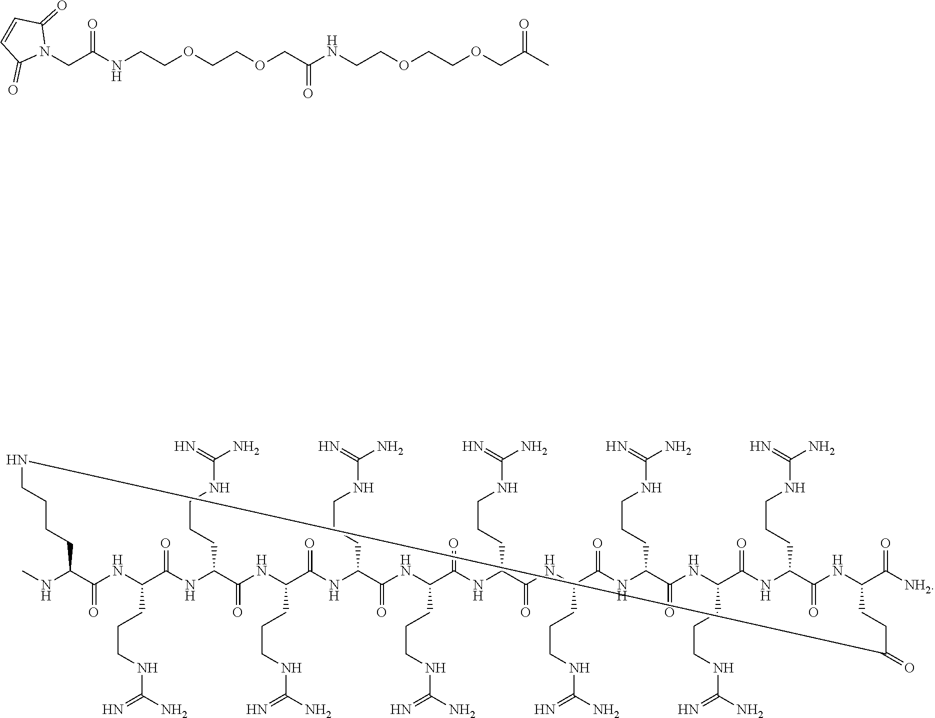

Also, a compound may be:

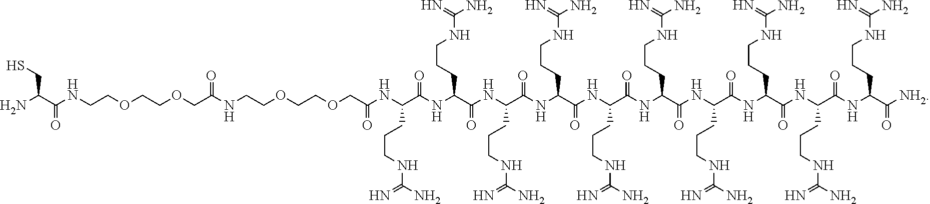 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)COCCOCCNC(=O)COCCOCCNC(=O)[C@@H](N)CS)C(N)=O]

Also, a compound may be:

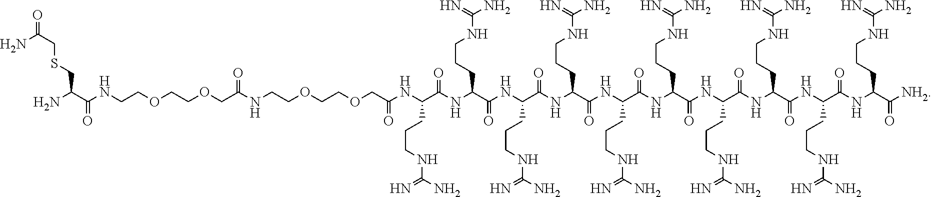 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)COCCOCCNC(=O)COCCOCCNC(=O)[C@@H](N)CSCC(N)=O)C(N)=O]

Also, a compound may be:

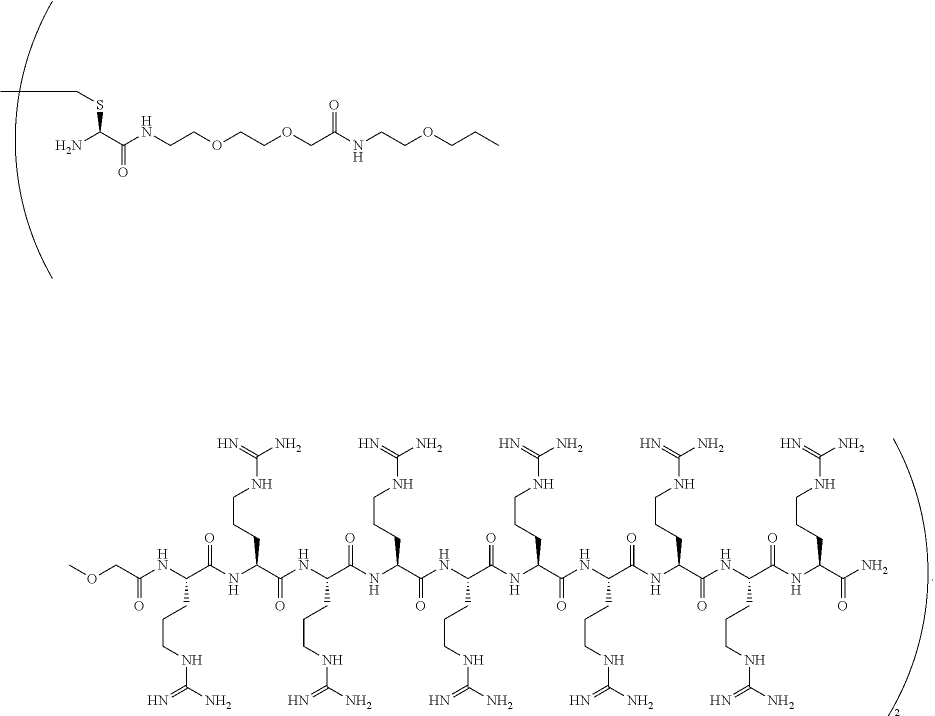 [SMILES: CCCOCCNC(=O)COCCOCCNC(=O)[C@@H](N)SCC ; COCC(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(N)=O]

Also, a compound may be:

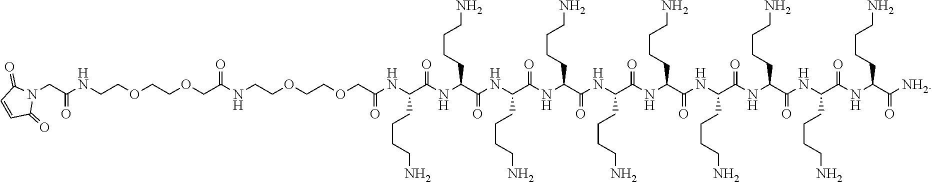 [SMILES: NCCCC[C@H](NC(=O)[C@H](CCCCN)NC(=O)[C@H](CCCCN)NC(=O)[C@H](CCCCN)NC(=O)[C@H](CCCCN)NC(=O)[C@H](CCCCN)NC(=O)[C@H](CCCCN)NC(=O)[C@H](CCCCN)NC(=O)[C@H](CCCCN)NC(=O)[C@H](CCCCN)NC(=O)COCCOCCNC(=O)COCCOCCNC(=O)CN1C(=O)C=CC1=O)C(N)=O]

Also, a compound may be:

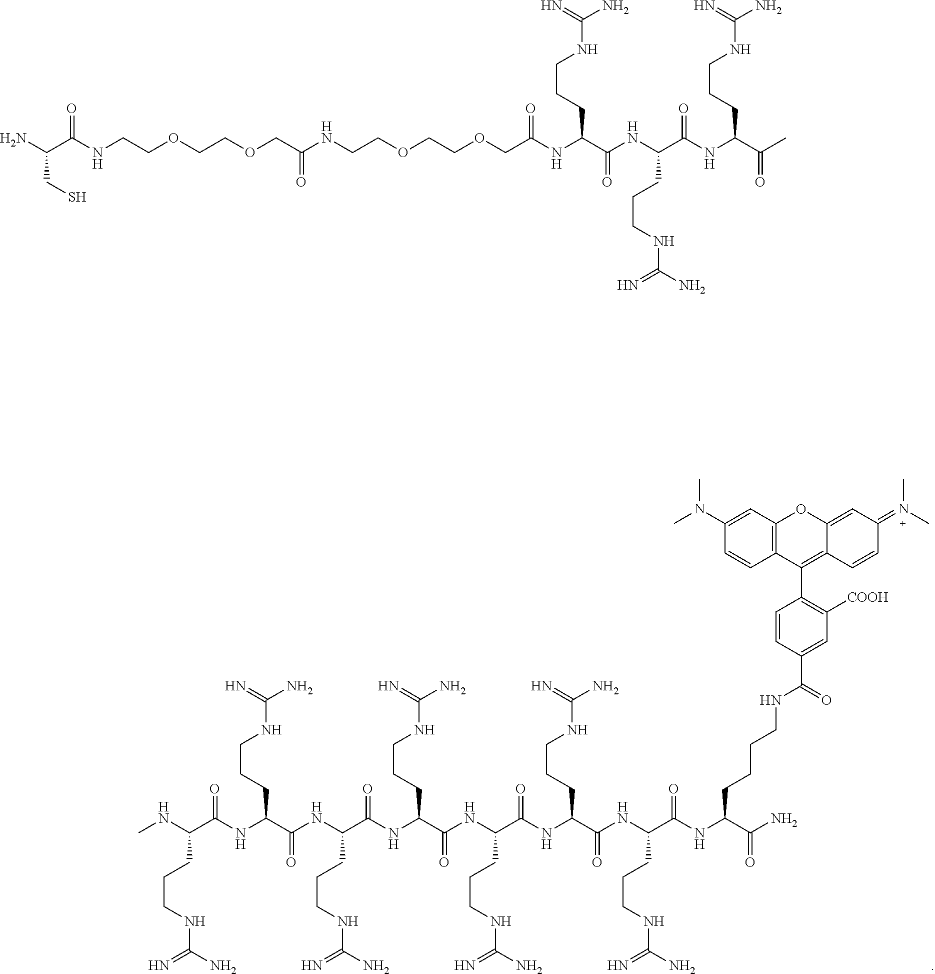

Also, a compound may be:

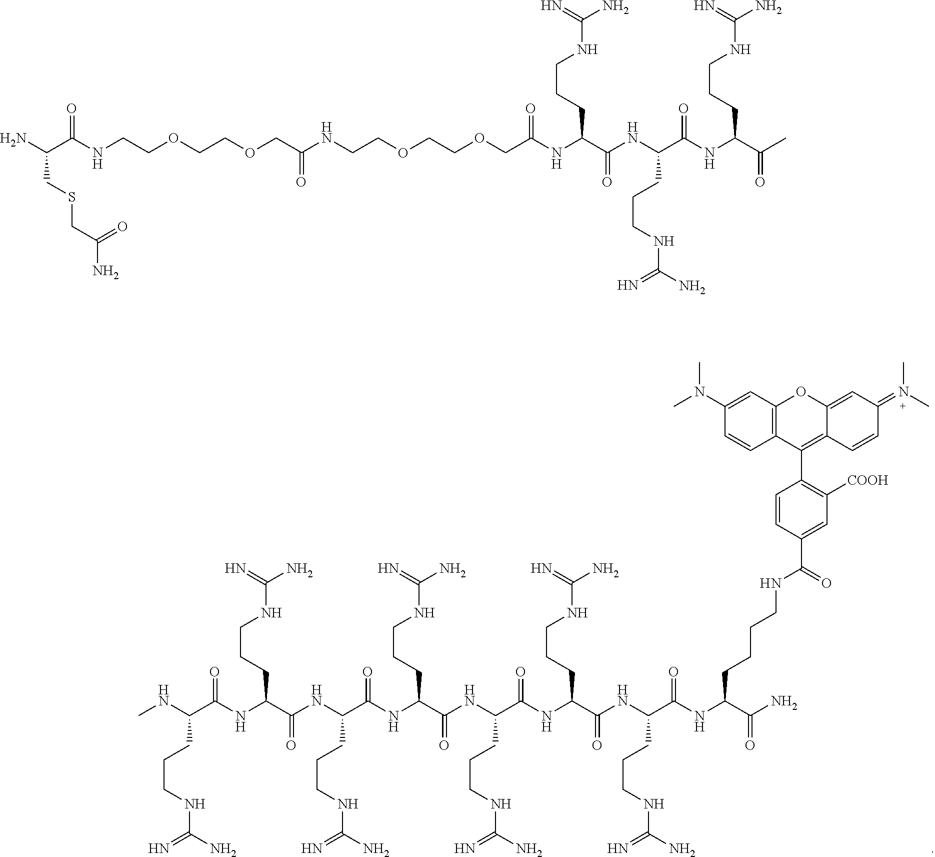

Also, a compound may be:

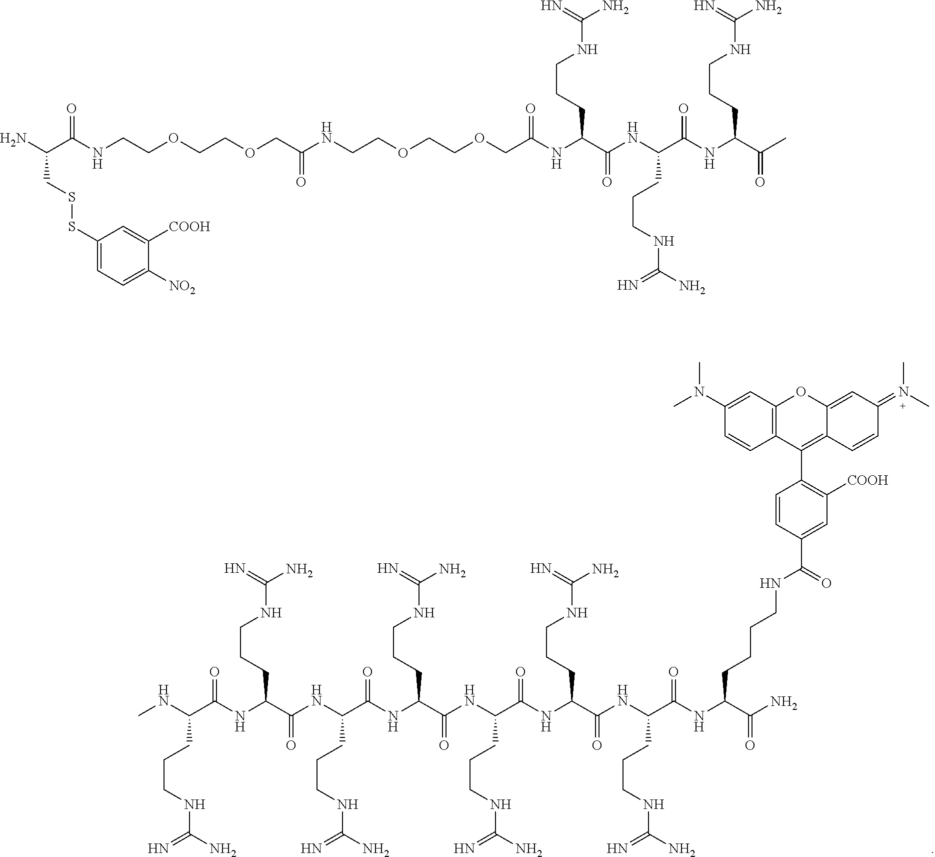

Also, a compound may be:

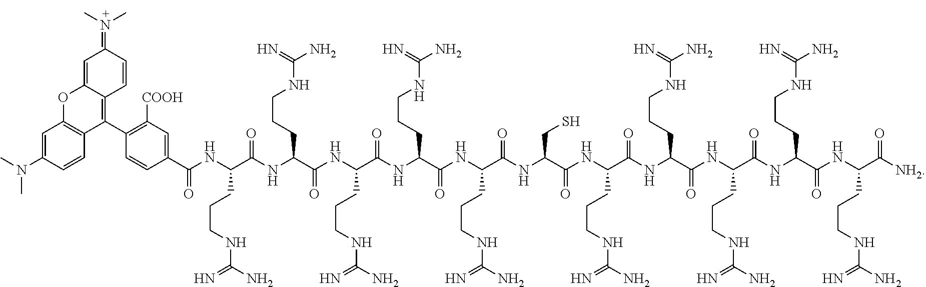

Also, a compound may be:

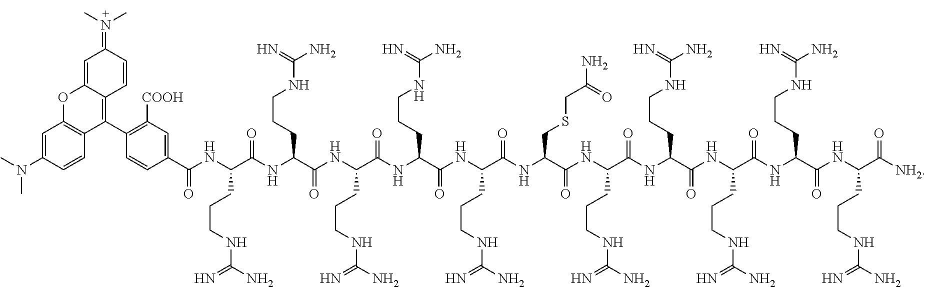

Also, a compound may be:

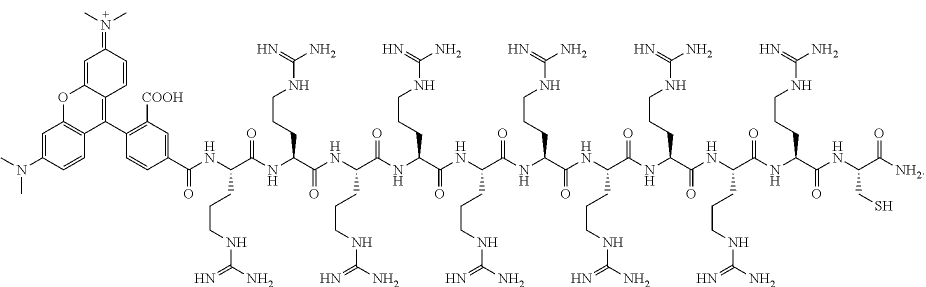

Also, a compound may be:

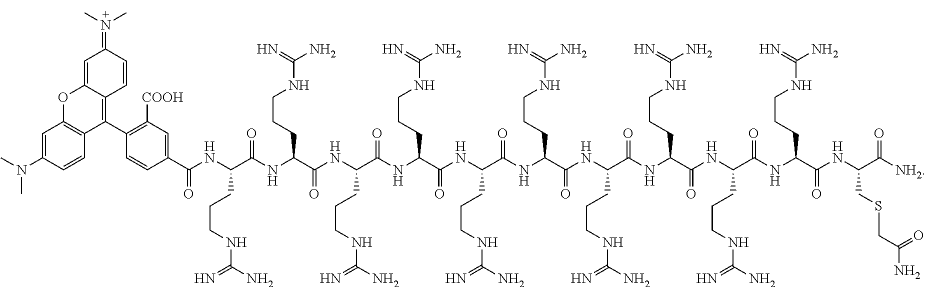

Also, a compound may be:

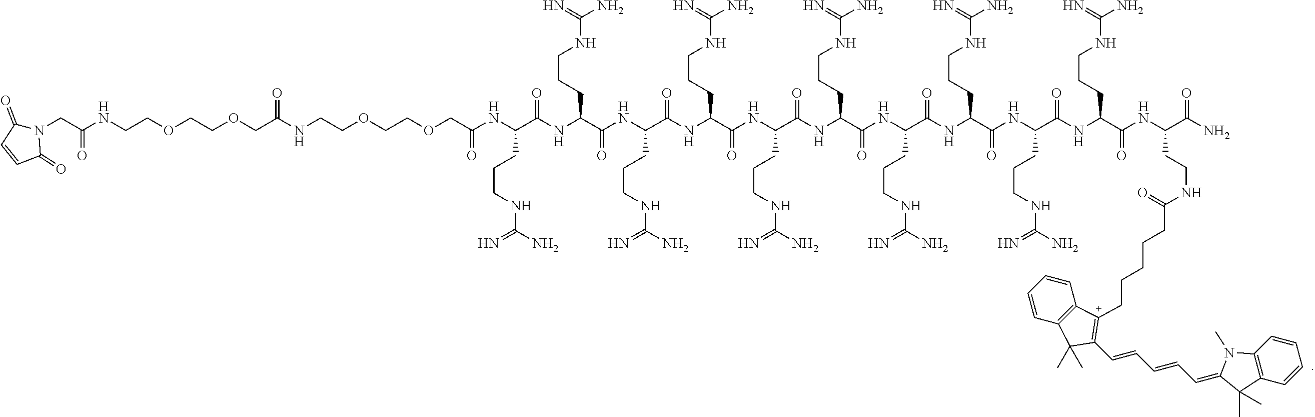

Also, a compound may be:

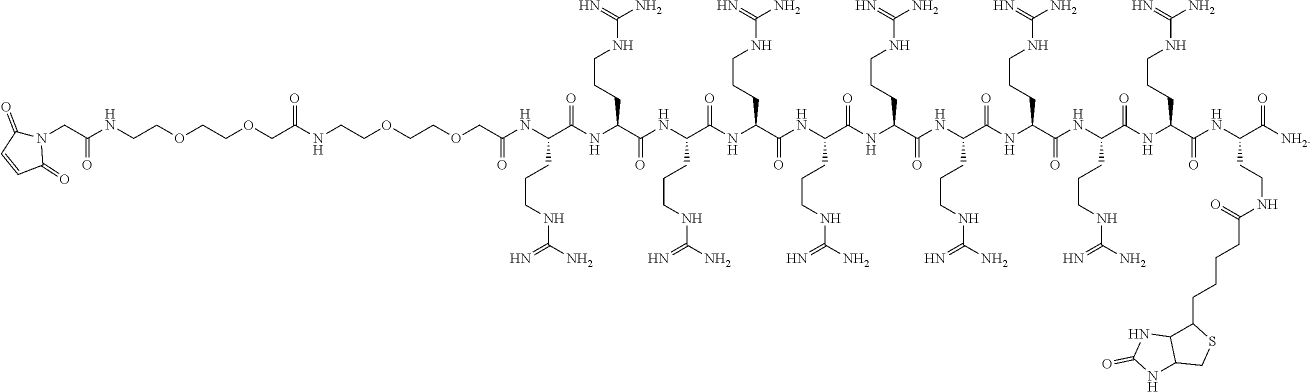 [SMILES: N=C(N)NCCC[C@H](NC(=O)COCCOCCNC(=O)COCCOCCNC(=O)CN1C(=O)C=CC1=O)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCNC(=O)CCCCC1SCC2NC(=O)NC21)C(N)=O]

Preferably, the compound is:

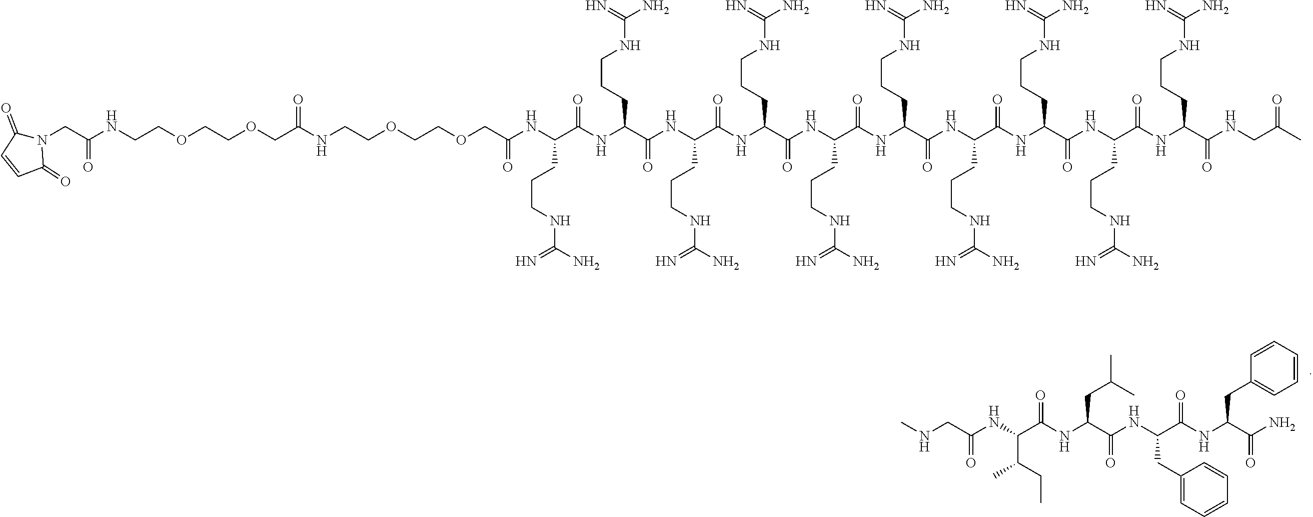

Preferably, the compound is:

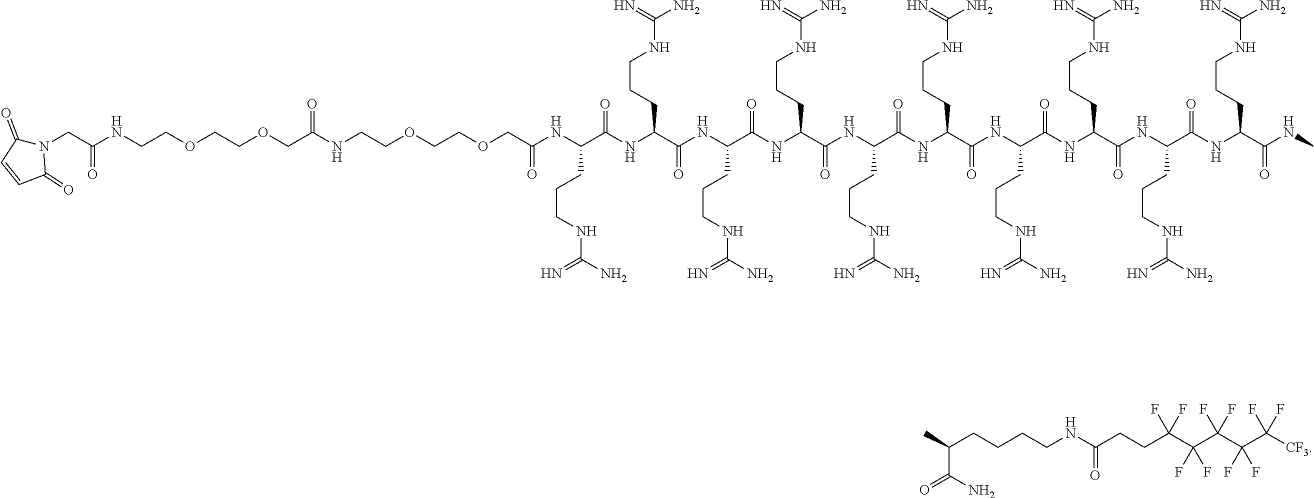

The compounds described herein can be prepared using standard peptide synthesis techniques, as generally known to a person skilled in the art. Accordingly, solid-phase peptide synthesis (SPPS) may be used. The solid phase may be any solid phase known to a person skilled in the art which is suitable for solid phase peptide synthesis. Such solid phases are also known as resins. Illustrative examples for a solid phase suitable for solid phase peptide synthesis include organic and inorganic phases such as a Merrifield polystyrene resin (copolymer from styrene and 1-2% divinylbenzene), polyacrylamide resins, TentaGel (a graft polymer where polyethylene glycol is grafted to polystyrene), Wang resin (typically based on crosslinked polystyrene, such as in a Merrifield resin), or porous glass having defined pore size as an example for an inorganic solid phase. Illustrative examples for commercially available solid supports for solid phase peptide synthesis are Rink amide resins or NovaSyn®TGR resins supplied by Merck Millipore. In particular, a Rink amide resin can be used for synthesizing the compounds described herein. Standard protecting group techniques, which are generally employed in peptide synthesis, can be used. For example, fluorenylmethoxycarbonyl (Fmoc) can be used as protecting group, in particular during the solid-phase peptide synthesis. After completion of the peptide synthesis, the peptide, which is still bound to the solid phase, may be coupled to the linker L, and then the linker L may be coupled with the A group. Alternatively, the linker L may be coupled with the A group, and the resulting conjugate of the linker L and the A group may be coupled with the peptide bound to the solid phase via the linker L. Alternatively, when a linker L is not present, the A group may be coupled directly with the peptide bound to the solid phase. After the coupling reactions, the resulting compound may be cleaved from the solid phase. It is also possible that after completion of the peptide synthesis, the peptide is first cleaved from the solid phase, and then coupled with the linker L, and then the linker L may be coupled with the A group; or, after cleavage of the peptide from the solid phase, the peptide may be coupled with a conjugate of the linker L and the A group; when a linker is not present, after cleavage from the solid support, the peptide may be directly coupled with the A group. Peptides can be also prepared using biotechnological methods known to a person skilled in the art. If needed, peptides or the compounds described herein can be purified using standard techniques known to a person skilled in the art, such as e.g. reverse phase HPLC.

### Kit

A further aspect of the invention is directed to a kit for use in delivering a cargo into a cell, the kit comprising a compound comprising a moiety capable to bind to a cell surface and a guanidine moiety.

In a preferred embodiment of the invention, the compound is selected from any one of the compounds according to the invention.

In accordance with the invention, the kit may comprise any compound according to the invention and preferably a buffer, such as a pharmaceutical acceptable buffer.

The present invention is also characterized by the following items:
1. A method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell.
2. The method according to item 1, wherein the cargo is connected such that the group is conjugated with or fused to a group comprising a guanidine moiety.
3. The method according to item 1 or 2, wherein the cargo is selected from peptide, protein, enzyme, nanobody, oligonucleotide, nanoparticle and antibody.
4. The method according to items 1 to 3, wherein the moiety of the compound capable to bind to the cell surface is a thiol-reactive moiety, or wherein the moiety is capable to bind to the cell surface via an enzymatic reaction, preferably wherein the moiety is capable to bind to a tag, such as a Halotag.
4a. The method according to any one of items 1 to 4, wherein the compound comprising a moiety capable to bind to the cell surface and a guanidine moiety further comprises a hydrophobic moiety.
5. The method according to any one of items 1 to 4a, the method comprising:
(a) incubating the compound comprising a moiety capable to bind to the cell surface and a guanidine moiety together with the cargo connected with the group comprising a guanidine moiety to obtain a solution comprising the compound and the cargo connected with the group,
(b) incubating the solution of step (a) with the cell, thereby allowing delivering of the cargo into the cell.
6. The method according to item 5, wherein in (b) the incubating the solution of (a) with the cell is carried out for a time of 1 minute to 24 hours, preferably for 5 min to 60 minutes, and/or at a temperature of 4° C. to 37° C., in particular preferred at 4° C.
7. The method according to any one of items 4 to 6, wherein the moiety of the compound is capable to bind to a tag or target structure on the cell surface, and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell.
8. The method according to item 7, wherein the tag is preferably a Halotag, or wherein the structure is preferably a bioorthogonal reporter on the cell surface.
9. The method according to any one of items 7 or 8, wherein in (c) the incubating the solution of (b) with the cell is carried out for a time of 1 minute to 24 hours, preferably for 5 min to 60 minutes, and/or at a temperature of 4° C. to 37° C., in particular preferred at 4° C.
10. The method according to any one of the previous items, wherein the delivered cargoes are antibodies, preferably full-length antibodies.
11. A compound comprising a moiety capable to bind to a cell surface and a guanidine moiety for use in delivering a cargo into a cell.
12. The compound for use according to item 11, wherein the cargo is connected with a group comprising a guanidine moiety.
13. The compound for use according to item 11 or 12, wherein the cargo is selected from peptide, protein, enzyme, nanobody, oligonucleotide, nanoparticle and antibody.
14. The compound for use according to any one of items 11 to 13, wherein the moiety of the compound capable to bind to the cell surface is a thiol-reactive moiety, or wherein the moiety is capable to bind on the cell surface via an enzymatic reaction, preferably wherein the moiety is capable to bind to a tag, such as a Halotag.
14a. The compound for use according to any one of items 11 to 14, wherein the compound comprising a moiety capable to bind to the cell surface and a guanidine moiety further comprises a hydrophobic moiety.
15. The compound for use according to item 12 or 13, wherein the cargo is conjugated with or fused to the group comprising a guanidine moiety.
16. The compound for use according to any one of items 11 to 15, wherein the cargo is an antibody, preferably a full-length antibody.
17. The compound for use according to any one of items 11 to 16, wherein the compound is for use in diagnostic or therapy.
18. A compound of formula (1):

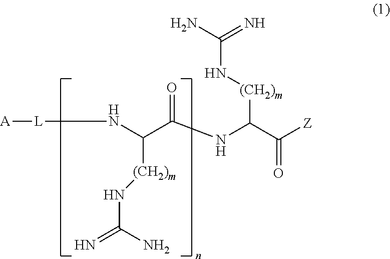

wherein:
A is a moiety capable to bind to a cell surface;
L is a linker or a bond, preferably L is a linker;
m is each independently an integer ranging from 0 to 10, preferably from 1 to 10, more preferably 1 to 8, still more preferably 1 to 6, still more preferably 1 to 5, still more preferably 2 to 4, most preferably 3;
n is an integer ranging from 1 to 20, preferably 3 to 19, more preferably 4 to 19, still more preferably 4 to 17, still more preferably 5 to 15, still more preferably 6 to 13, still more preferably 7 to 11, still more preferably 8 to 10, most preferably 9;
Z is selected from the group consisting of NR¹R², OR³, an amino acid, a peptide comprising 2 to 10 amino acids, and a hydrophobic moiety;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen;
or a pharmaceutically acceptable salt thereof.
19. The compound according to item 18, wherein A is a thiol-reactive moiety.
20. The compound according to item 19, wherein:
A is

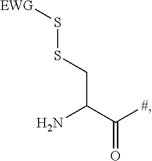

wherein #indicates the attachment point to the L; and EWG is an electron-withdrawing group.
21. The compound according to item 20, wherein EWG is selected from the group consisting of

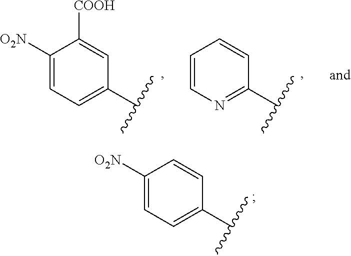 [SMILES: *c1ccc([N+](=O)[O-])c(C(=O)O)c1 ; *c1ccccn1 ; *c1ccc([N+](=O)[O-])cc1]

preferably, EWG is

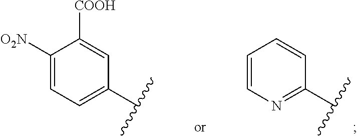 [SMILES: *c1ccc([N+](=O)[O-])c(C(=O)O)c1 ; *c1ccccn1]

more preferably EWG is

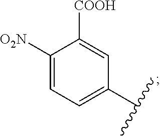 [SMILES: *c1ccc([N+](=O)[O-])c(C(=O)O)c1]

wherein [symbol] indicates the attachment point to the S.
22. The compound according to item 19, wherein:
A is

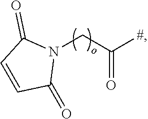

wherein o is an integer ranging from 0 to 10, preferably 1 to 10, more preferably 1 to 8, still more preferably 1 to 5, still more preferably 1 to 3, most preferably 1, and #indicates the attachment point to the L.
23. The compound according to item 18, wherein A is capable to bind to the cell surface via an enzymatic reaction.
24. The compound according to item 23, wherein A is capable to bind to a Halotag.
25. The compound according to item 24, wherein:
A is

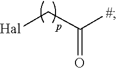

Hal is a halogen, preferably Cl;
p is an integer ranging from 1 to 10, preferably 2 to 8, more preferably 3 to 7, still more preferably 4 to 6, most preferably 5; and
#indicates the attachment point to the L.
25a. The compound according to any one of items 18 to 25, wherein Z is selected from the group consisting of NR¹R², OR³, an amino acid, and a peptide comprising 2 to 10 amino acids.
25b. The compound according to any one of items 18 to 25, wherein Z is a hydrophobic moiety.
25c. The compound according to item 25b, wherein Z is a peptide comprising 2 to 10 amino acids independently selected from the group consisting of glycine, alanine, valine, leucine, isoleucine, proline, phenylalanine, methionine, tryptophan and one or more hydrophobic unnatural amino acid(s);
preferably wherein Z is a peptide comprising 2 to 10 amino acids independently selected from the group consisting of glycine, alanine, valine, leucine, isoleucine, proline, phenylalanine, methionine and tryptophan;
more preferably wherein Z is a peptide comprising 2 to 10 amino acids independently selected from the group consisting of glycine, leucine, isoleucine, phenylalanine and tryptophan.
25d. The compound according to item 25c, wherein Z is:

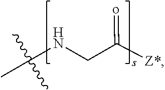

wherein:
Z* is a peptide comprising 2 to 6 amino acids independently selected from the group consisting of glycine, alanine, valine, leucine, isoleucine, proline, phenylalanine, methionine, tryptophan and one or more hydrophobic unnatural amino acid(s),
preferably wherein Z* is a peptide comprising 2 to 6 amino acids independently selected from the group consisting of glycine, alanine, valine, leucine, isoleucine, proline, phenylalanine, methionine and tryptophan;
more preferably wherein Z* is a peptide comprising 2 to 6 amino acids independently selected from the group consisting of glycine, leucine, isoleucine, phenylalanine and tryptophan;
s is an integer ranging from 1 to 4, preferably s is 2 or 3, more preferably s is 2; and
[symbol] indicates the attachment point to the carbonyl carbon atom.
25e. The compound according to item 25d, wherein Z is:

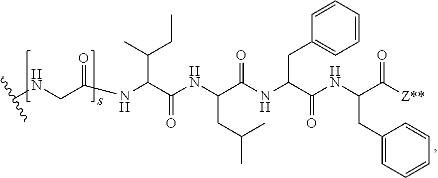

wherein:
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen;
s is an integer ranging from 1 to 4, preferably s is 2 or 3, more preferably s is 2; and
[symbol] indicates the attachment point to the carbonyl carbon atom.
25f. The compound according to item 25e, wherein Z is:

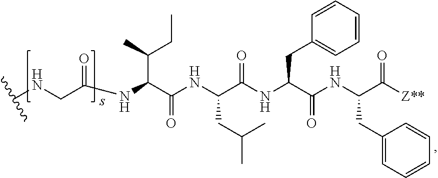

wherein:
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen;
s is an integer ranging from 1 to 4, preferably s is 2 or 3, more preferably s is 2; and
[symbol] indicates the attachment point to the carbonyl carbon atom.
25g. The compound according to item 25b, wherein Z comprises or is (C₁-C₂₀)perfluoroalkyl.
25h. The compound according to item 25g, wherein Z is:

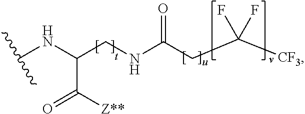

wherein:
t is an integer ranging from 1 to 8, preferably t is 4;
u is an integer ranging from 1 to 4, preferably u is 2;
v is an integer ranging from 1 to 9, preferably 3 to 7, more preferably v is 5; and
Z** is NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen; and
[symbol] indicates the attachment point to the carbonyl carbon atom.
25i. The compound according to item 25h, wherein Z is:

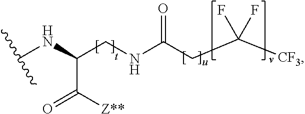

wherein:
t is 4;
u is an integer ranging from 1 to 4, preferably u is 2;
v is an integer ranging from 1 to 9, preferably 3 to 7, more preferably v is 5; and Z** is selected from the group consisting of NR¹R² or OR³;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring, preferably a five- or six-membered ring; preferably, R¹ and R² are each hydrogen;
R³ is hydrogen or (C₁-C₆)alkyl, preferably hydrogen; and
[symbol] indicates the attachment point to the carbonyl carbon atom.
26. The compound according to any one of items 18 to 25i, having formula (2):

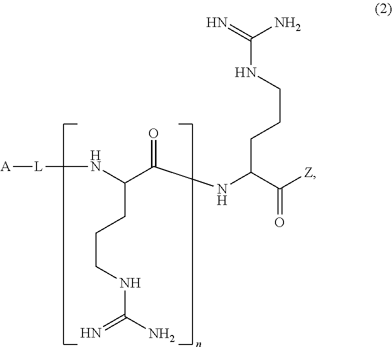

wherein A, L, n and Z are as defined in any one of items 18 to 25i.
27. The compound according to any one of items 18 to 26, wherein L is selected from the group consisting of —(C₁-C₁₀)alkylene-C(O)—, —(C₃-C₈)carbocyclo-C(O)—, -arylene-C(O)—, —(C₁-C₁₀)alkylene-arylene-C(O)—, -arylene-(C₁-C₁₀)alkylene-C(O)—, —(C₁-C₁₀)alkylene-(C₃-C₈)carbocyclo-C(O)—, —(C₃-C₈)carbocyclo-(C₁-C₁₀)alkylene-C(O)—, —(C₃-C₈)heterocyclo-C(O)—, —(C₁-C₁₀)alkylene-(C₃-C₈)heterocyclo-C(O)—, —(C₃-C₈)heterocyclo-(C₁-C₁₀)alkylene-C(O)—, —(CH₂CH₂CH₂)ᵣ—C(O)—, —(CH₂CH₂CH₂)ᵣ—CH₂—C(O)—, —(CH₂CH₂NH)ᵣ—C(O)—, —(CH₂CH₂NH)ᵣ—CH₂—C(O)—, —(CH₂CH₂O)ᵣ—C(O)—, and —(CH₂CH₂O)ᵣ—CH₂—C(O)—, wherein r is an integer ranging from 1 to 10, preferably 2 to 8, more preferably 3 to 7, still more preferably 4 to 6, still more preferably 5.
28. The compound according to any one of items 18 to 26, wherein L is selected from the group consisting of —NR⁴—(C₁-C₁₀)alkylene-C(O)—, —NR⁴—(C₃-C₈)carbocyclo-C(O)—, —NR⁴-arylene-C(O)—, —NR⁴—(C₁-C₁₀)alkylene-arylene-C(O)—, —NR⁴-arylene-(C₁-C₁₀)alkylene-C(O)—, —NR⁴—(C₁-C₁₀)alkylene-(C₃-C₈)carbocyclo-C(O)—, —NR⁴—(C₃-C₈)carbocyclo-(C₁-C₁₀)alkylene-C(O)—, —NR⁴—(C₃-C₈)heterocyclo-C(O)—, —NR⁴—(C₁-C₁₀)alkylene-(C₃-C₈)heterocyclo-C(O)—, —NR⁴—(C₃-C₈)heterocyclo-(C₁-C₁₀)alkylene-C(O)—, —NR⁴—(CH₂CH₂CH₂)ᵣ—C(O)—, —NR⁴—(CH₂CH₂CH₂)ᵣ—CH₂—C(O)—, —NR⁴—(CH₂CH₂NH)ᵣ—C(O)—, —NR⁴—(CH₂CH₂NH)ᵣ—CH₂—C(O)—, —NR⁴—(CH₂CH₂O)ᵣ—C(O)—, and —NR₄—(CH₂CH₂O)ᵣ—CH₂—C(O)—, wherein r is an integer ranging from 1 to 10, preferably 2 to 8, more preferably 3 to 7, still more preferably 4 to 6, still more preferably 5; and R⁴ is each independently selected from the group consisting of hydrogen and (C₁-C₈)alkyl, preferably R⁴ is each hydrogen.
29. The compound according to any one of items 18 to 26, wherein L is selected from the group consisting of —O—(C₁-C₁₀)alkylene-C(O)—, —O—(C₃-C₈)carbocyclo-C(O)—, —O-arylene-C(O)—, —O—(C₁-C₁₀)alkylene-arylene-C(O)—, —O-arylene-(C₁-C₁₀)alkylene-C(O)—, —O—(C₁-C₁₀)alkylene-(C₃-C₈)carbocyclo-C(O)—, —O—(C₃-C₈)carbocyclo-(C₁-C₁₀)alkylene-C(O)—, —O—(C₃-C₈)heterocyclo-C(O)—, —O—(C₁-C₁₀)alkylene-(C₃-C₈)heterocyclo-C(O)—, —O—(C₃-C₈)heterocyclo-(C₁-C₁₀)alkylene-C(O)—, —O—(CH₂CH₂CH₂)ᵣ—C(O)—, —O—(CH₂CH₂CH₂)ᵣ—CH₂—C(O)—, —O—(CH₂CH₂NH)ᵣ—C(O)—, —O—(CH₂CH₂NH)ᵣ—CH₂—C(O)—, —O—(CH₂CH₂O)ᵣ—C(O)—, and —O—(CH₂CH₂O)ᵣ—CH₂—C(O)—, wherein r is an integer ranging from 1 to 10, preferably 2 to 8, more preferably 3 to 7, still more preferably 4 to 6, still more preferably 5.
30. The compound according to any one of items 18 to 26, wherein L is —[B¹—(CH₂CH₂B₂)ₕ—(CH₂); —C(O)]ₖ—, wherein B¹ and B² are independently selected from the group consisting of CH₂, NH and O, preferably B¹ is NH and B² is O; wherein h is an integer ranging from 1 to 4, preferably 2 to 3, more preferably 2; j is 1 or 2, preferably 1; and k is an integer ranging from 1 to 10, preferably 2 to 8, more preferably 2 to 7, still more preferably 2 to 6, still more preferably 2 to 5, still more preferably 2 to 4, still more preferably 2 to 3, most preferably 2.
31. The compound according to item 30, having formula (2b):

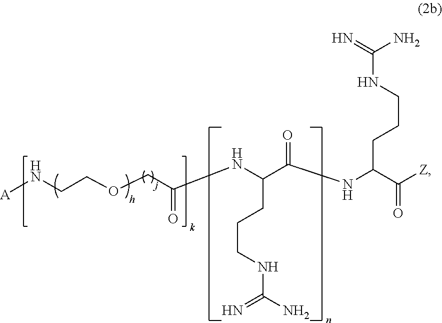

wherein A, h, j, k, n and Z are as defined in any one of items 18 to 30.
32. The compound according to item 31, having formula (2c):

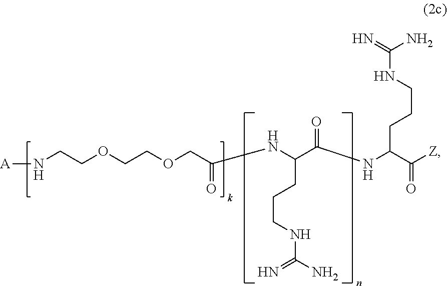

wherein A, k, n and Z are as defined in any one of items 18 to 31.
33. The compound according to any one of items 18 to 32, wherein each amino acid moiety has its natural configuration.
34. The compound according to item 33, wherein the compound is a compound of formula (2*):

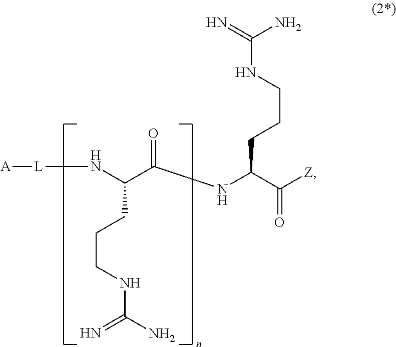

wherein A, L, n and Z are as defined in any one of items 18 to 33.
35. The compound according to item 34, wherein the compound of formula (2*) is a compound of formula (2b*):

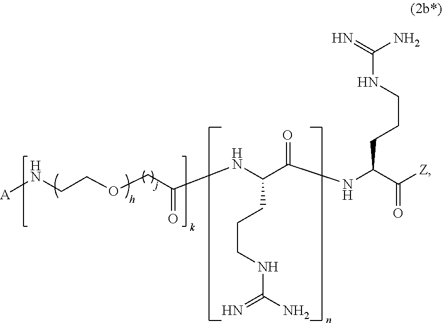

wherein A, h, j, k, n and Z are as defined in any one of items 18 to 34.
36. The compound according to item 35, wherein the compound of formula (2b*) is a compound of formula (2c*):

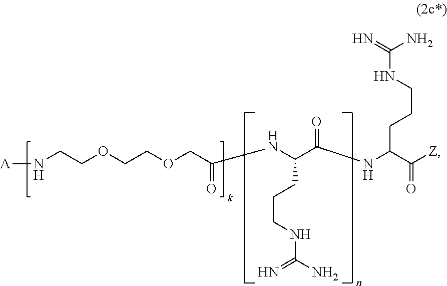

wherein A, k, n and Z are as defined in any one of items 18 to 35.
37. A compound according to any one of items 18 to 36 for a use according to any one of items 11 to 17.
38. The method according to any one of items 1 to 10, wherein the compound comprising a moiety capable to bind to the cell surface and a guanidine moiety is a compound according to any one of items 18 to 36.
39. A compound comprising a moiety capable to bind to a cell surface and a guanidine moiety for use in delivering a cargo into a cell, wherein the compound is a compound according to any one of items 18 to 36.
40. The compound for use according to item 39, wherein
(i) the cargo is connected with a group comprising a guanidine moiety;
(ii) the cargo is conjugated with or fused to the group comprising a guanidine moiety;
(iii) the cargo is selected from peptide, protein, enzyme, nanobody, oligonucleotide, nanoparticle and antibody;
(iv) the cargo is an antibody, preferably a full-length antibody;
(v) the moiety of the compound capable to bind to the cell surface is a thiol-reactive moiety, or wherein the moiety is capable to bind on the cell surface via an enzymatic reaction, preferably wherein the moiety is capable to bind to a tag, such as a Halotag; and/or
(vi) the compound comprising a moiety capable to bind to the cell surface and a guanidine moiety further comprises a hydrophobic moiety.
41. The compound for use according to item 39 or 40, wherein the compound is for use in diagnostic or therapy.
42. A method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, and wherein the compound is a compound according to any one of items 18 to 36.
43. The method according to item 42, wherein
(i) the cargo is connected such that the group is conjugated with or fused to a group comprising a guanidine moiety;
(ii) the cargo is selected from peptide, protein, enzyme, nanobody, oligonucleotide, nanoparticle and antibody;
(iii) the delivered cargoes are antibodies, preferably full-length antibodies;
(iv) the moiety of the compound capable to bind to the cell surface is a thiol-reactive moiety, or wherein the moiety is capable to bind to the cell surface via an enzymatic reaction, preferably wherein the moiety is capable to bind to a tag, such as a Halotag; and/or
(v) the compound comprising a moiety capable to bind to the cell surface and a guanidine moiety further comprises a hydrophobic moiety.
44. The method according to item 42 or 43, the method comprising:
(a) incubating the compound comprising a moiety capable to bind to the cell surface and a guanidine moiety together with the cargo connected with the group comprising a guanidine moiety to obtain a solution comprising the compound and the cargo connected with the group,
(b) incubating the solution of step (a) with the cell, thereby allowing delivering of the cargo into the cell, preferably wherein in (b) the incubating the solution of (a) with the cell is carried out for a time of 1 minute to 24 hours, preferably for 5 min to 60 minutes, and/or at a temperature of 4° C. to 37° C.
45. The method according to item 43 item (iv) or 44, wherein the moiety of the compound is capable to bind to a tag or target structure on the cell surface, and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours, preferably for 5 min to 60 minutes, and/or at a temperature of 4° C. to 37° C.
46. The method according to item 45, wherein the tag is preferably a Halotag, or wherein the structure is preferably a bioorthogonal reporter on the cell surface.
47. A method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, wherein
the moiety of the compound capable to bind to the cell surface is a thiol-reactive moiety, or wherein the moiety is capable to bind to the cell surface via an enzymatic reaction, preferably wherein the moiety is capable to bind to a tag, such as a Halotag; and/or
the compound comprising a moiety capable to bind to the cell surface and a guanidine moiety further comprises a hydrophobic moiety, and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours, preferably for 5 min to 60 minutes, and/or at a temperature of 4° C. to 37° C.
48. A method for delivering a cargo into a cell, the method comprising incubating a compound comprising a moiety capable to bind to the cell surface and a guanidine moiety together with a cargo and a cell, wherein the cargo is connected with a group comprising a guanidine moiety, thereby allowing delivering of the cargo into the cell, the method comprising:
(a) incubating the compound comprising a moiety capable to bind to the cell surface and a guanidine moiety together with the cargo connected with the group comprising a guanidine moiety to obtain a solution comprising the compound and the cargo connected with the group,
(b) incubating the solution of step (a) with the cell, thereby allowing delivering of the cargo into the cell, preferably wherein in (b) the incubating the solution of (a) with the cell is carried out for a time of 1 minute to 24 hours, preferably for 5 min to 60 minutes, and/or at a temperature of 4° C. to 37° C., and wherein the method comprises:
(a) transfecting a cell with a tag such that the tag is expressed on the cell surface, or modifying the cell surface with a target structure,
(b) incubating the compound comprising the moiety capable to bind to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours, preferably for 5 min to 60 minutes, and/or at a temperature of 4° C. to 37° C.
49. The method according to item 47 or 48, wherein the tag is preferably a Halotag, or wherein the structure is preferably a bioorthogonal reporter on the cell surface.
50. A kit for use in delivering a cargo into a cell, the kit comprising a compound comprising a moiety capable to bind to a cell surface and a guanidine moiety.
51. The kit for use according to item 50, wherein the compound is selected from any one of items 18 to 36.

With the present invention, the inventors could demonstrate that a compound comprising a guanidine moiety, in particular thiol-reactive arginine-rich peptide additives, can enhance the cellular uptake of preferably protein-CPP conjugates in a non-endocytic mode even at low μM concentration. They show that preferably thiol- or halotag-reactive compounds can result in covalently-anchored compounds comprising a guanidine moiety, preferably covalently-anchored peptides comprising a guanidine group (such as an arginine-rich peptide, e.g. an oligoarginine or polyarginine) on the cell surface, which are highly effective at co-delivering protein cargoes. Taking advantage of the thiol-reactivity of the most effective CPP-additive, which is one of the preferred compounds according to the invention, it could be shown that Cys-containing proteins can be readily delivered into the cytosol by simple co-addition of a slight excess of this CPP. Furthermore, it could be demonstrated the application of such a so called “CPP-additive technique” in the delivery of functional enzymes, nanobodies and full-length IgG antibodies. This new cellular uptake protocol greatly simplifies both the accessibility and efficiency of protein and antibody delivery with minimal chemical or genetic engineering. These advantages are new and unexpected.

In particular, it could be shown that electrophilic thiol-reactive CPP-additives are highly effective at creating nucleation zones on the cell-surface, which enable efficient transduction of protein-CPP conjugates. The protocol according to the invention proves to be highly effective, simple, and not harmful to the cell. Importantly, it could be shown that the transduction of recombinant CPP-containing proteins as well as a 150 kDa IgG antibody into living cells could be enabled via a non-endosomal uptake mechanism.

Thus, the inventors have shown delivery of various cargoes with both synthetic and recombinant CPPs using thiol-reactive R10 peptides. This uptake seems to be independent of active transport and is not harmful to the cell. In this context, the thiol-reactivity of the compounds according to the present invention appears to be a crucial factor, and the inventors could show that they can covalently label cell-surfaces through which they can then deliver cargoes.

In summary, the inventors provide a method and compounds with the present invention which allow the delivery of a cargo into a cell in a very efficient and easy way without any measurable cellular toxicity. Advantageously, no purification step is necessary. A further advantage is the delivery of the cargo into the cell can be conducted at 4° C. Such a temperature ensures that no active endosomal uptake occurs. This allows the delivery of cargoes that are sensitive to endosomal degradation or toxic for a cell upon prolonged exposure. Nevertheless, non-endosomal uptake is also expected at higher temperatures, such as 37° C. The method comprising the compounds according to the present invention provides a tool for the delivery of a cargo which can be considered as a so-called covalent transfection. A further advantage is that the method and compounds of the present invention provide the ability to employ cargoes, such as proteins from standard recombinant expression, in which the protein cargo is genetically fused to a group according to the invention, such as an oligo-Arg tag, and use them in non-endocytic uptake. According to the present invention, the method for delivery of a cargo into a cell using the compounds according to the invention in combination with the cargo connected with a group according to the present invention can be considered as a co-delivery strategy. Said co-delivery strategy allows for example to deliver active Cre recombinase into cells without any necessary conjugation chemistry. In particular, the inventors achieve efficient gene editing, which could easily be applied to the delivery of other functional enzymes. It is crucial to note that the inventors could demonstrate the cytosolic delivery of three different antibodies using compounds according to the present invention, resulting in expected intracellular localization. Said experimental results demonstrating the advantages according to the present invention are described and shown in detail below.

### EXAMPLES OF THE INVENTION

The present invention is further illustrated by the following examples. Yet, the examples and specific embodiments described therein must not be construed as limiting the invention to such specific embodiments.

### Example 1: Improved Cellular Uptake of Cargoes Mediated by Cell-Penetrating Peptide Additives

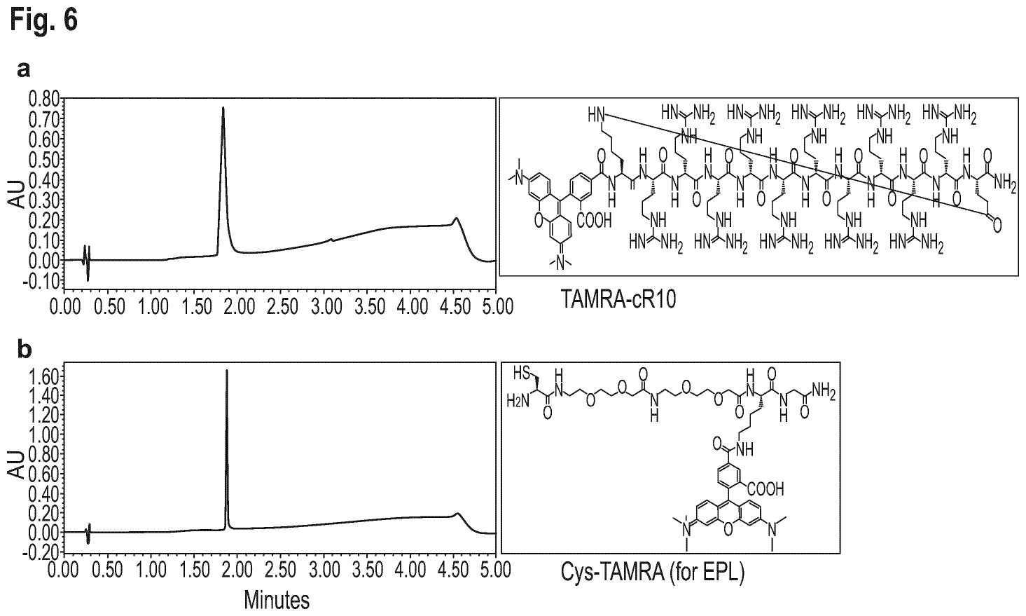

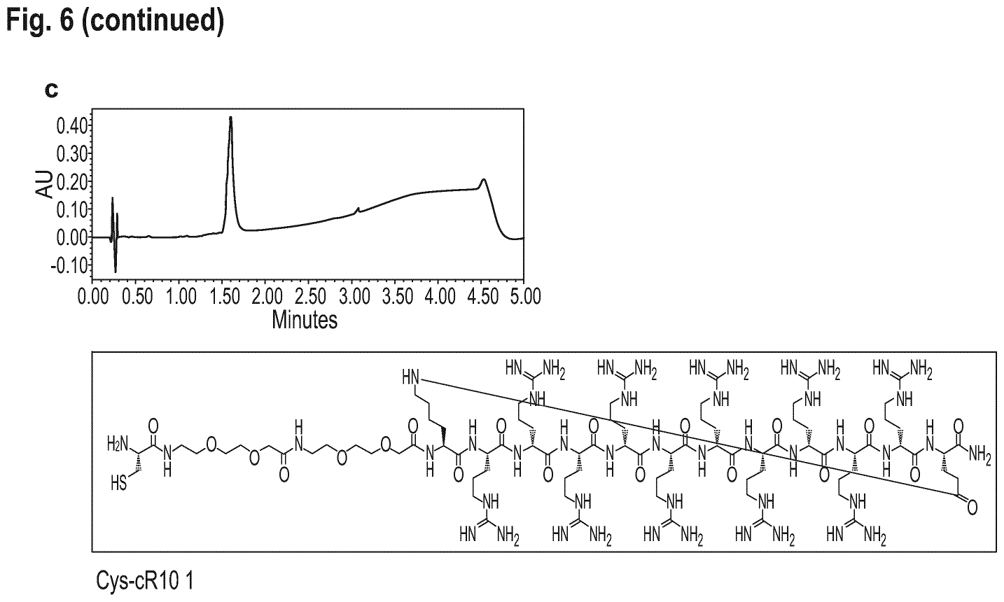

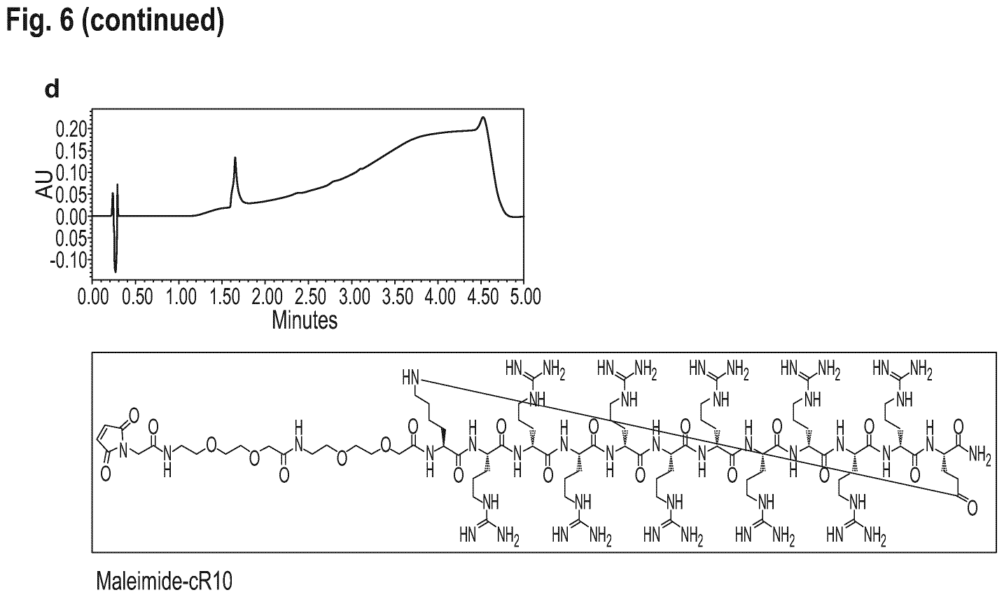

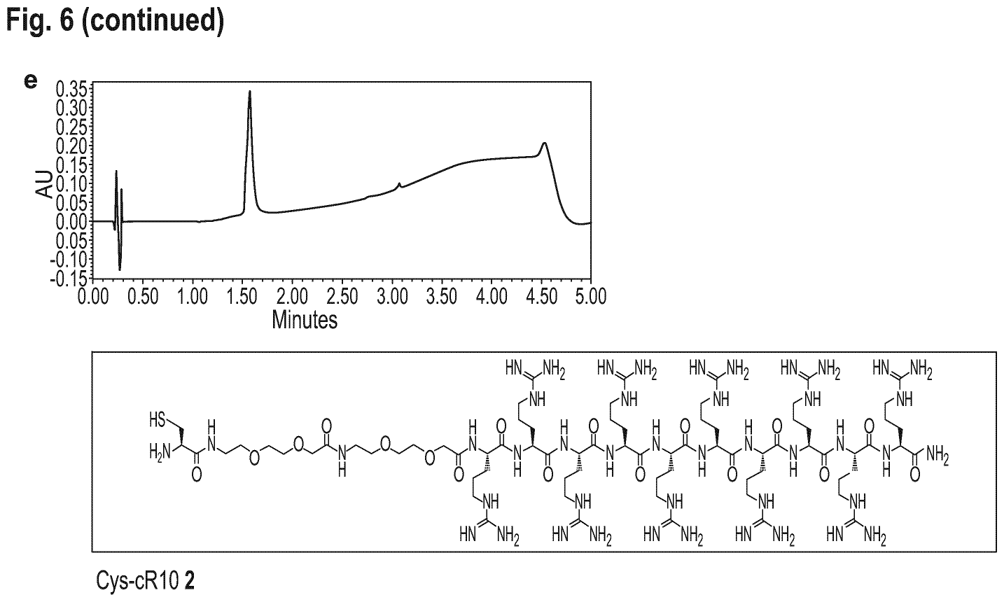

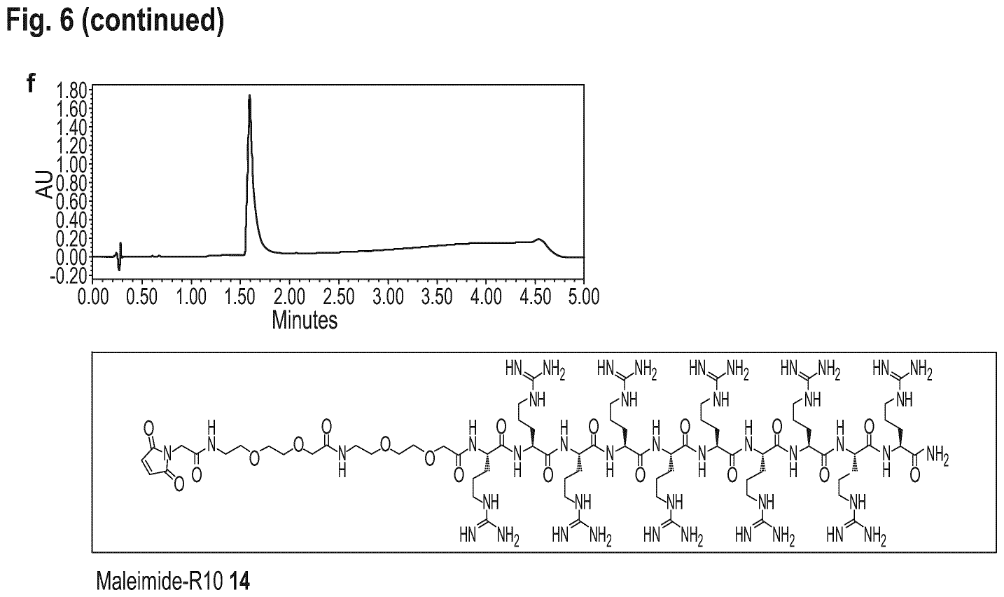

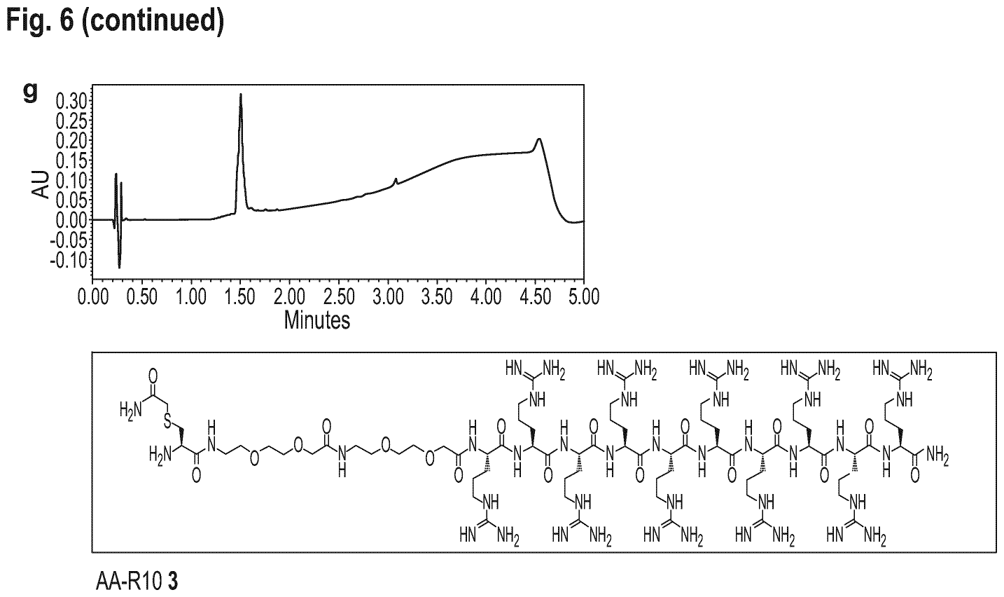

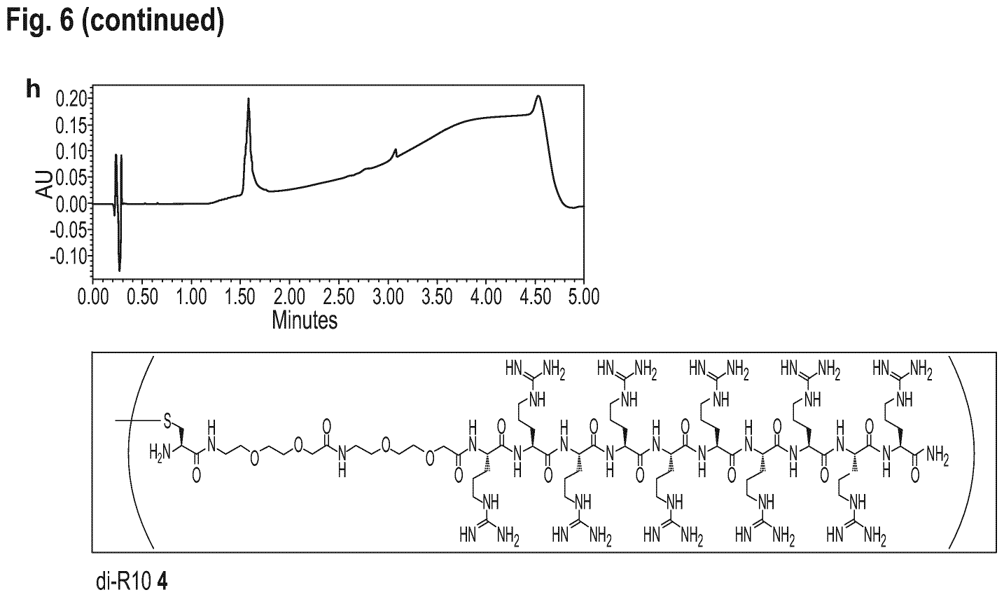

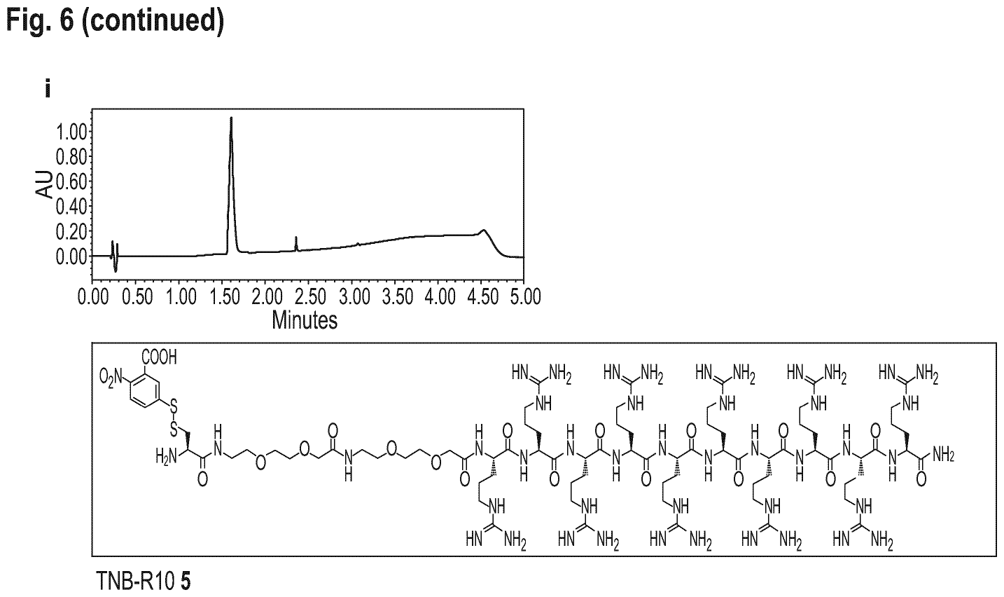

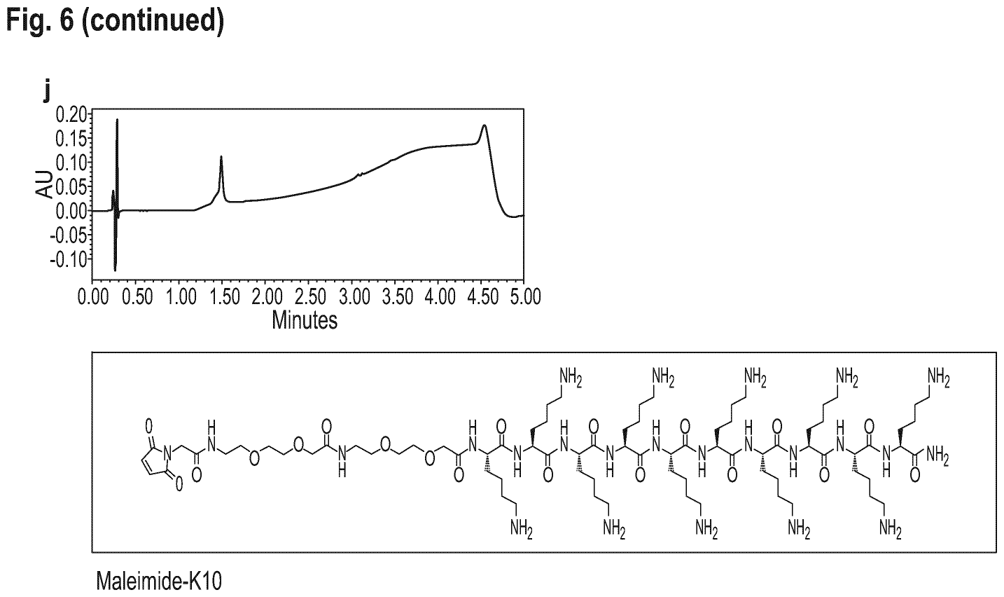

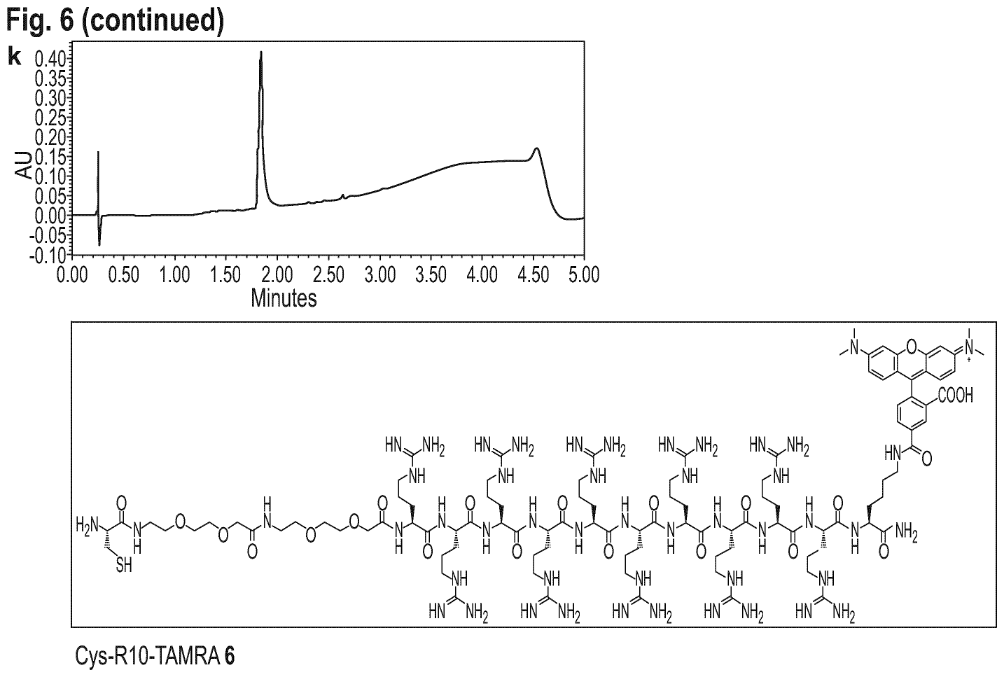

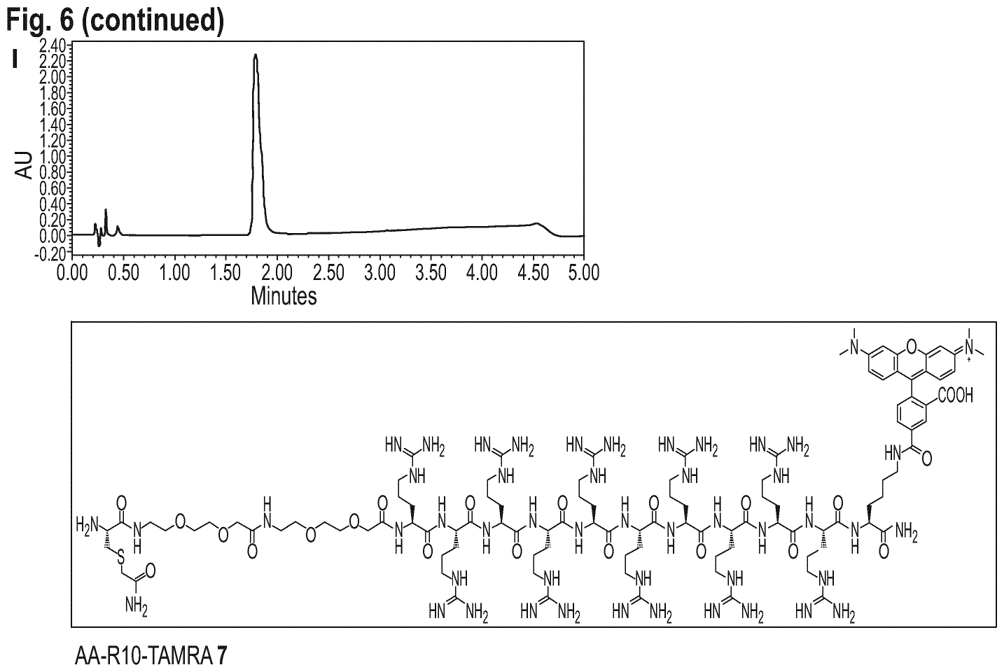

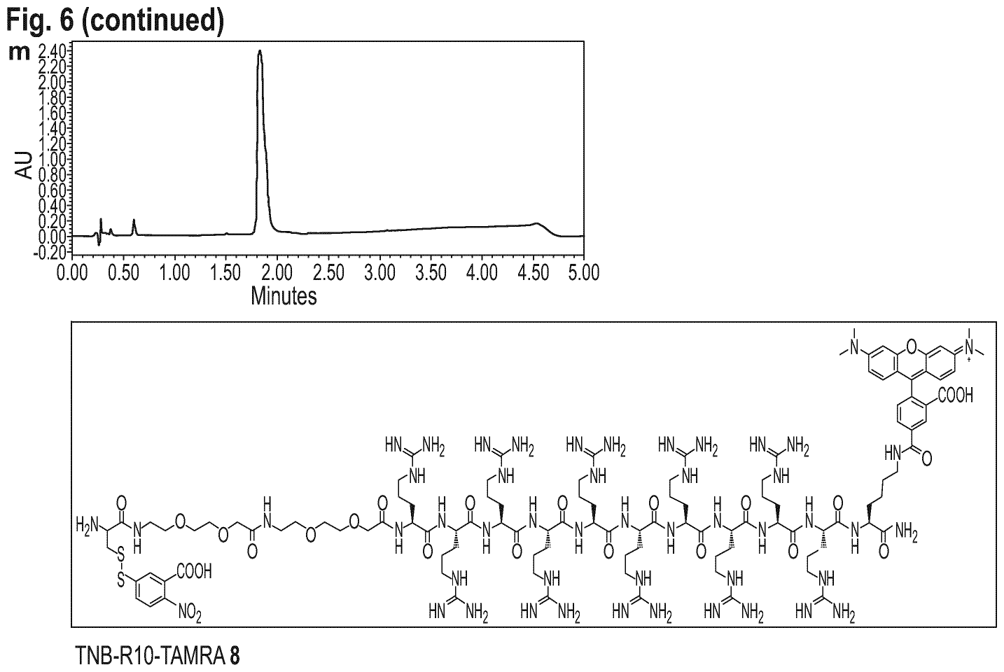

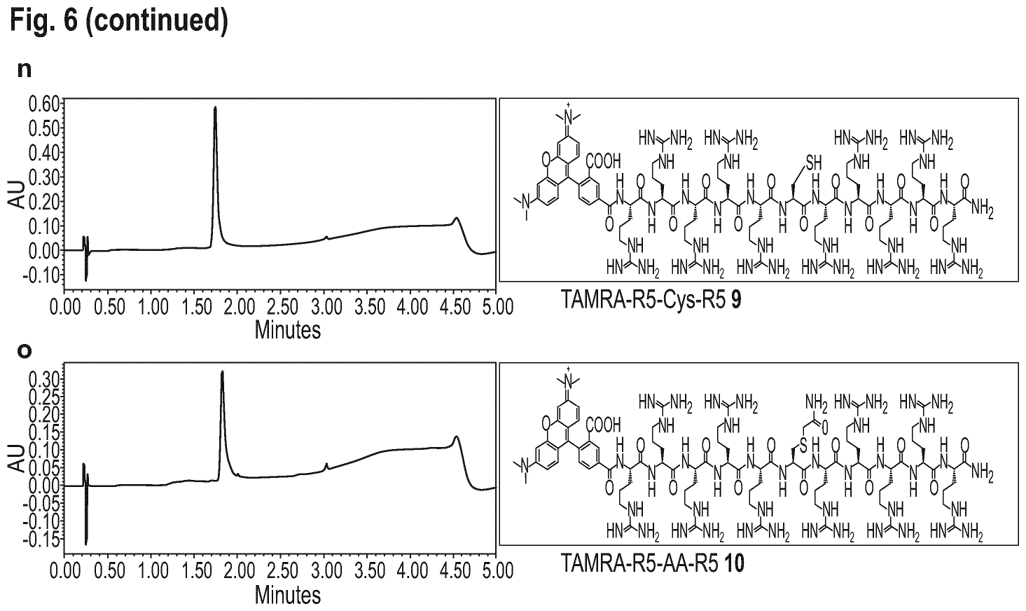

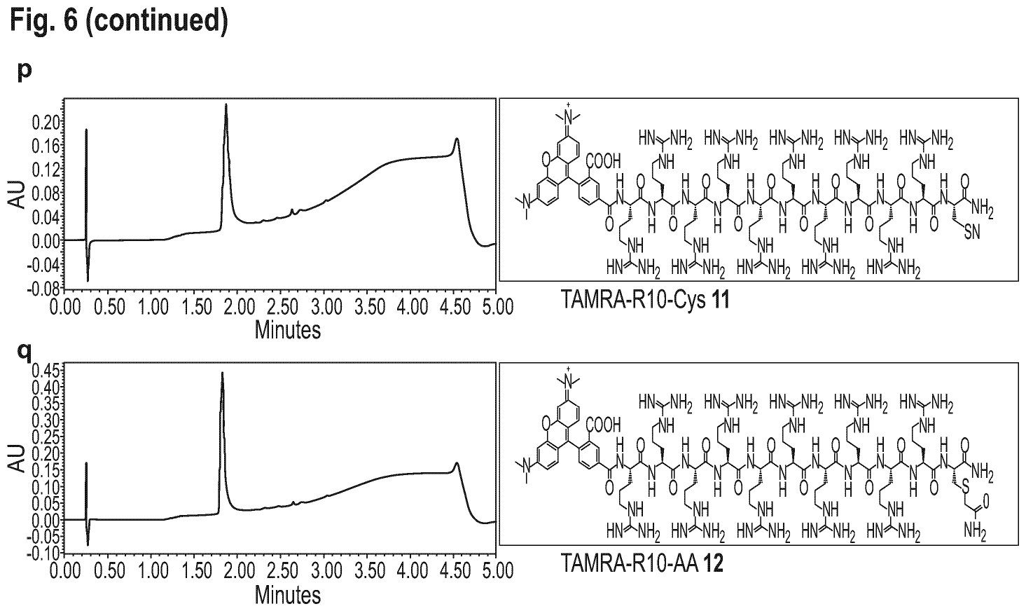

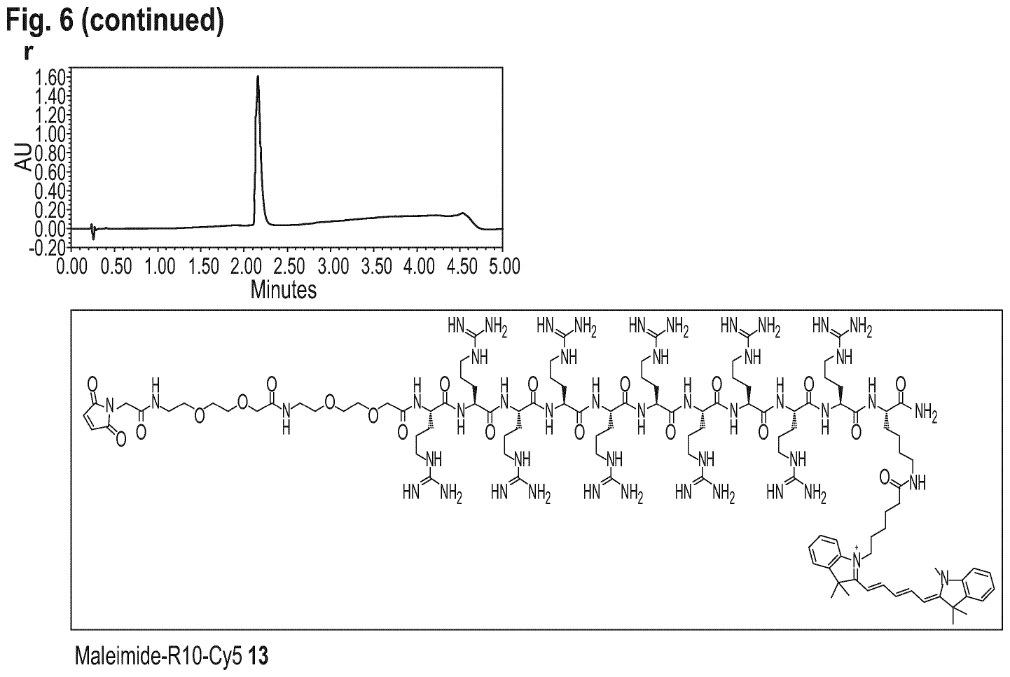

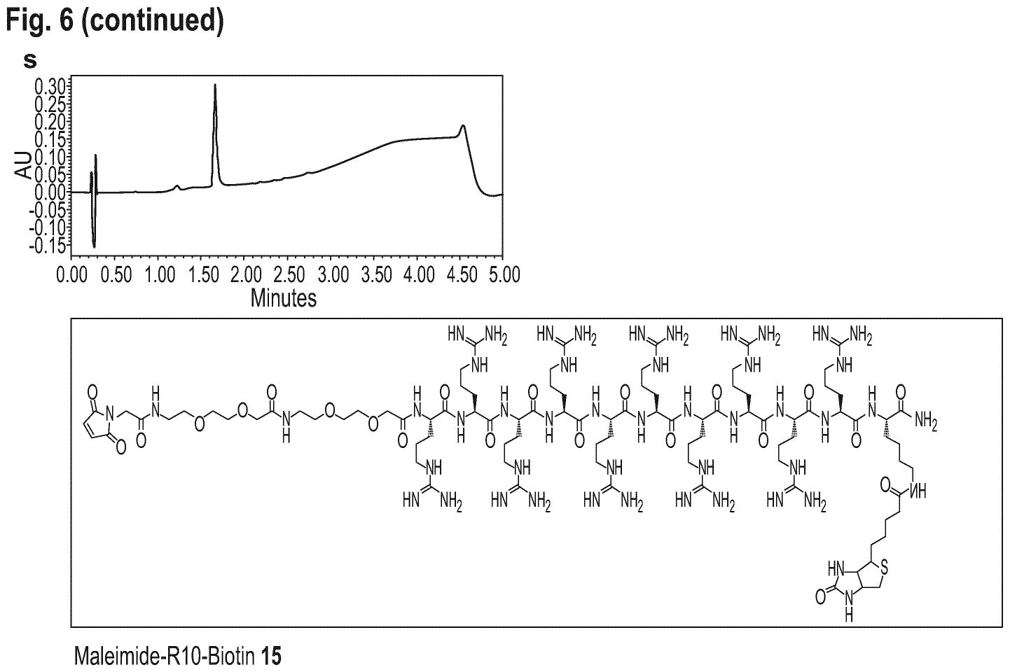

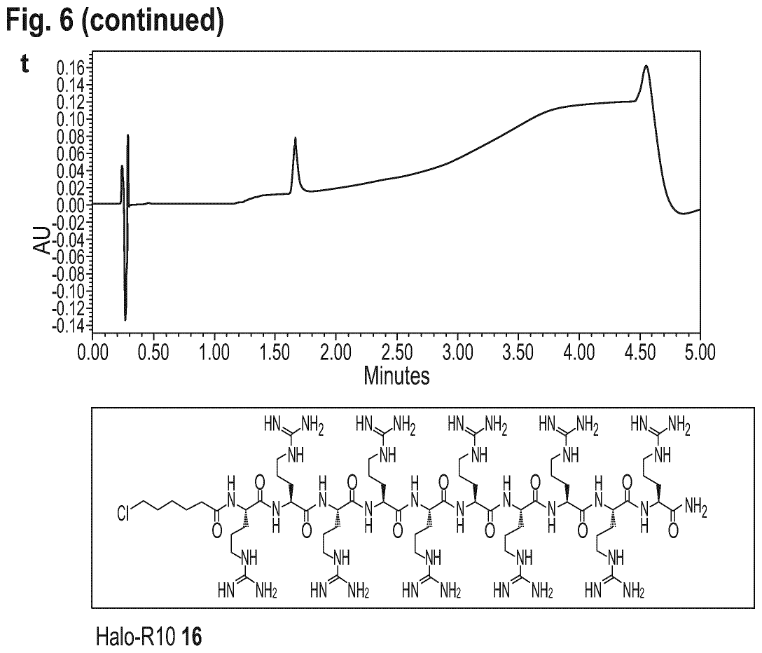

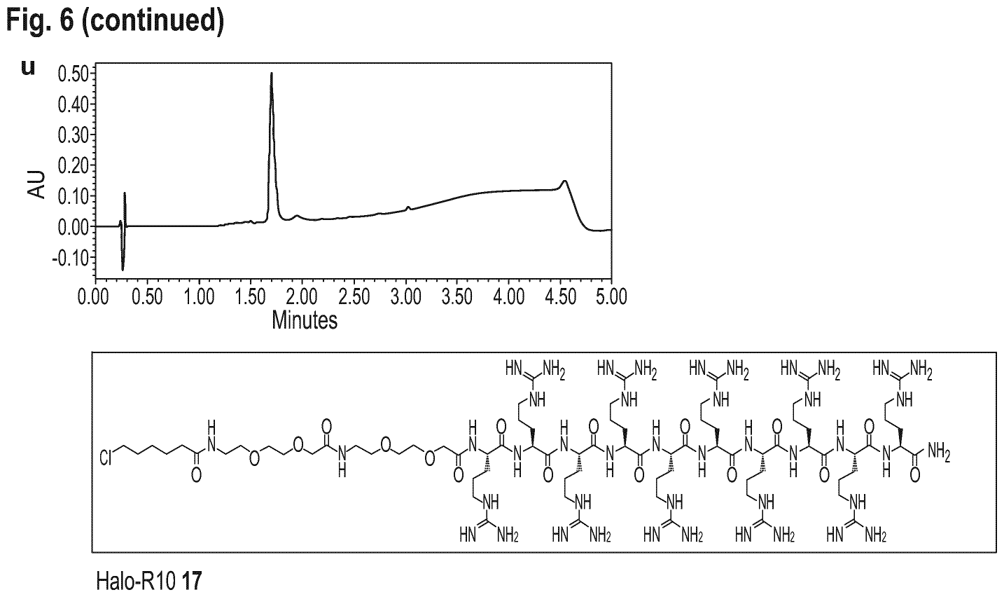

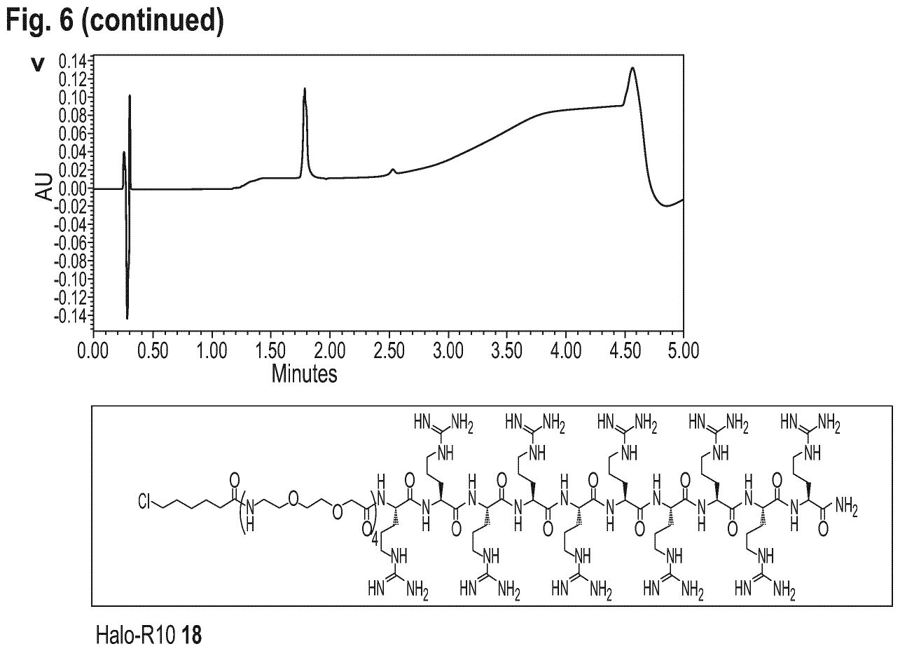

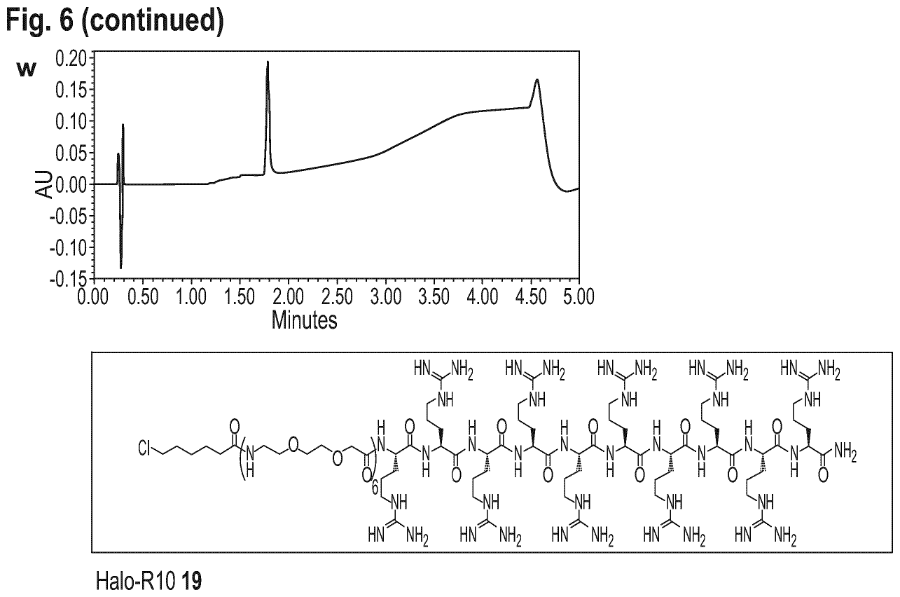

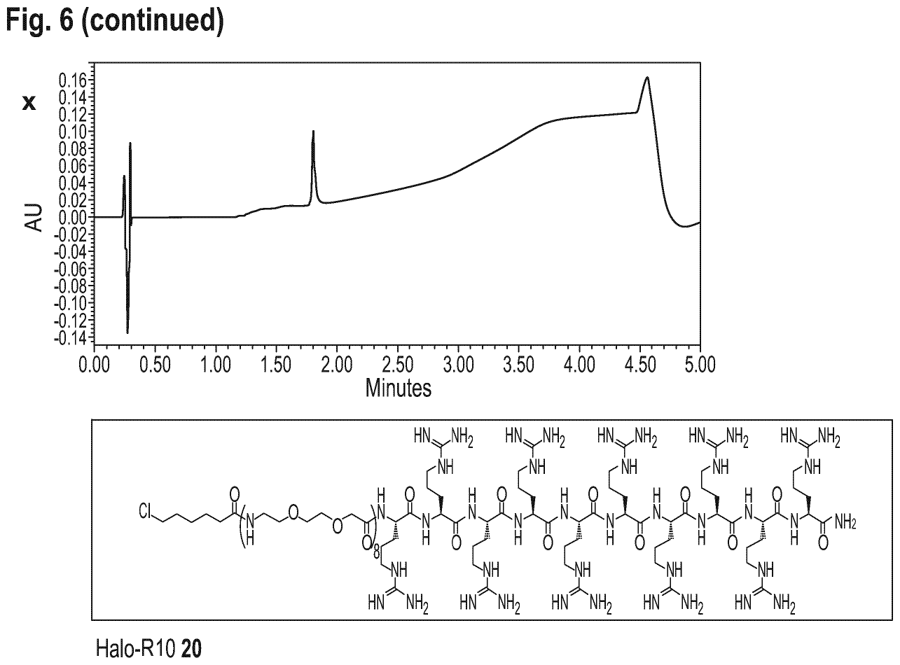

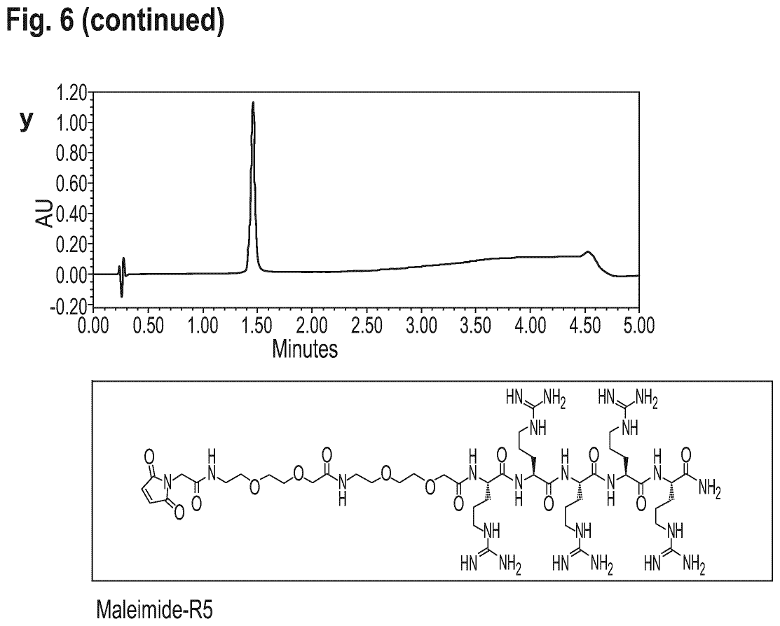

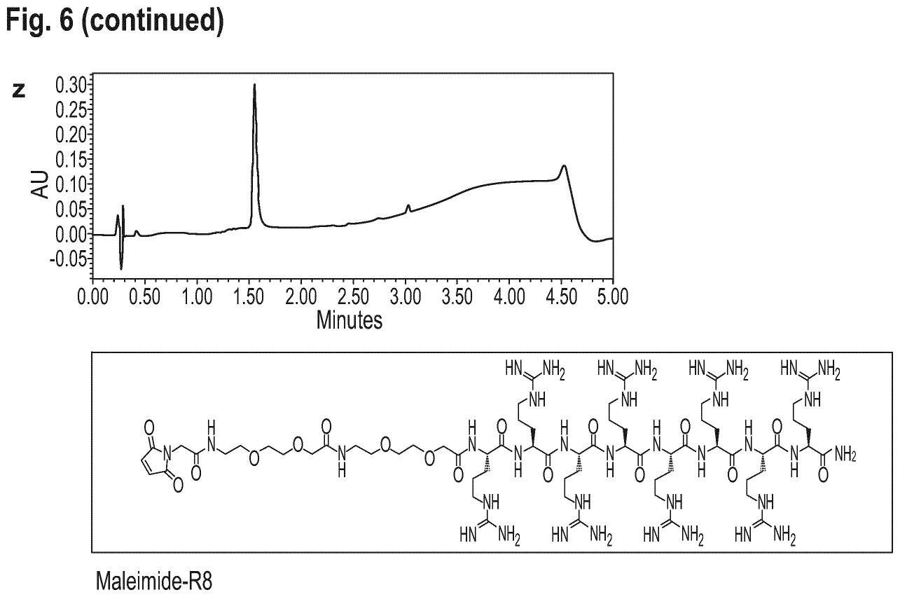

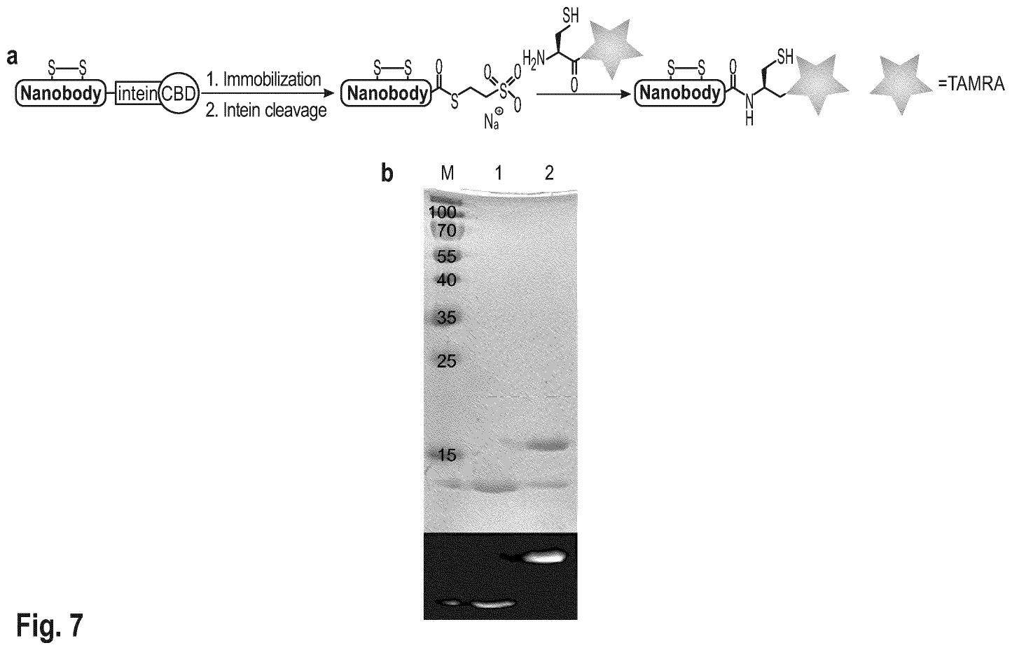

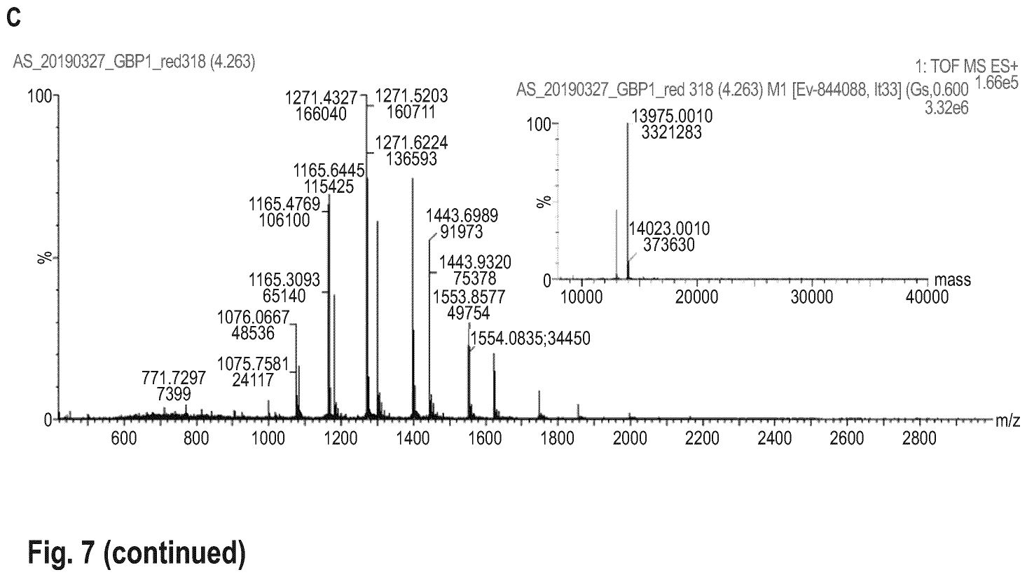

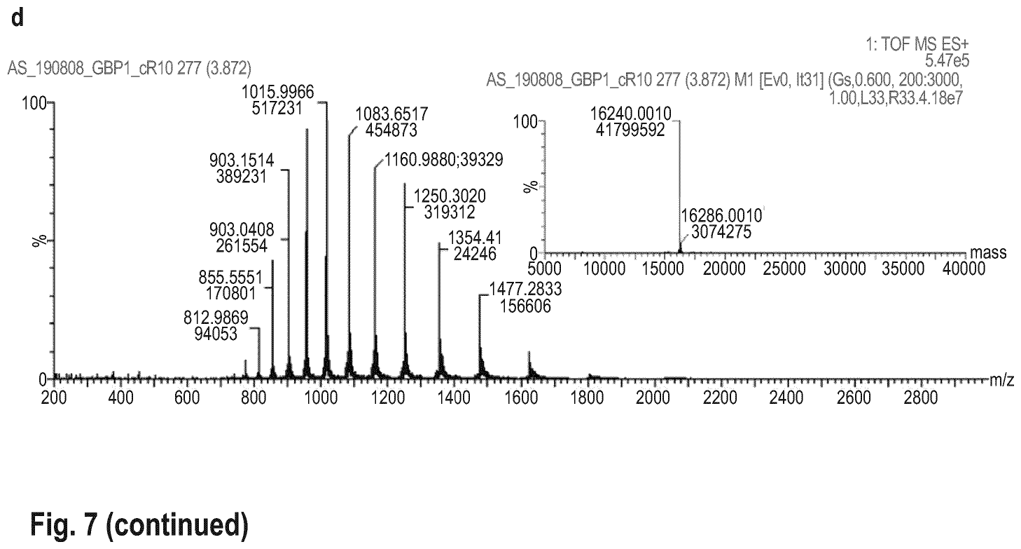

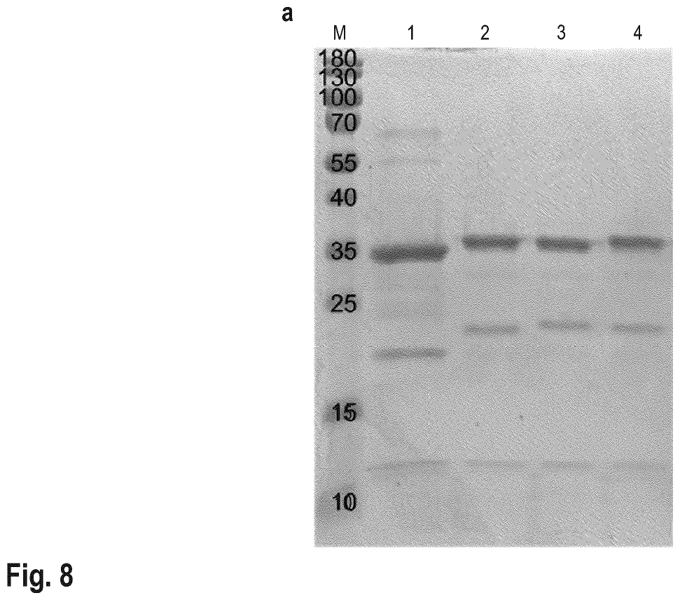

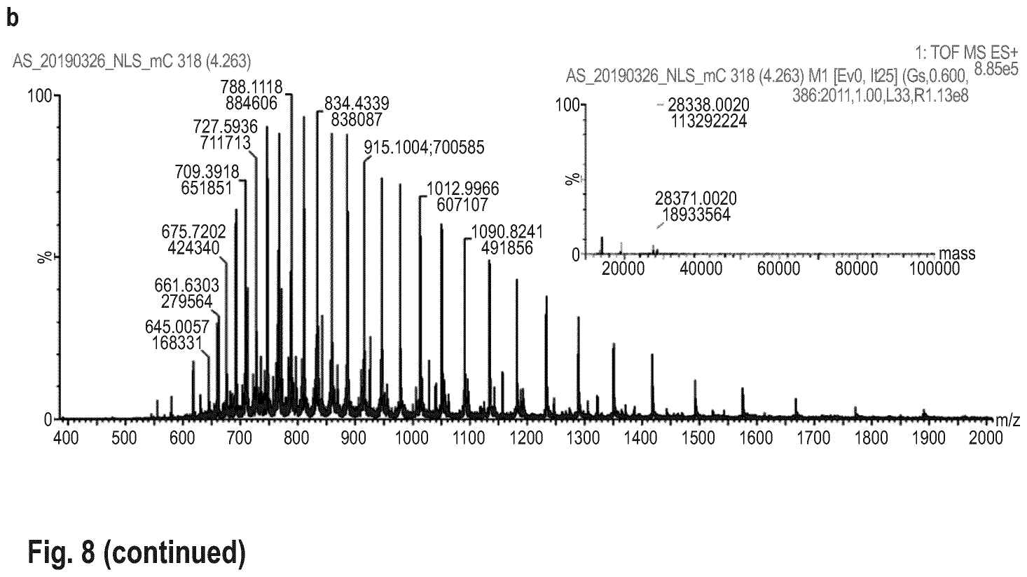

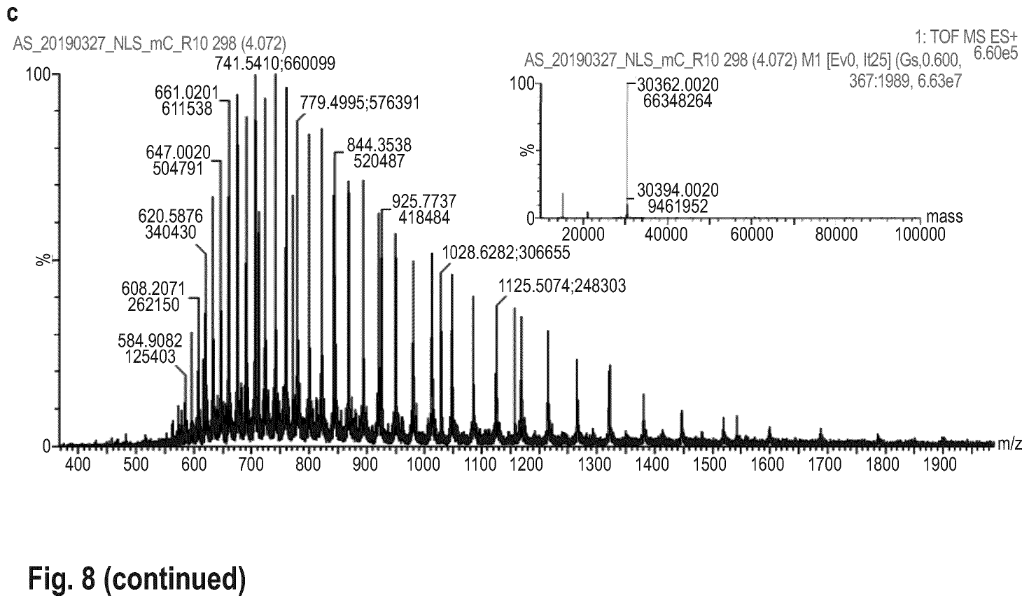

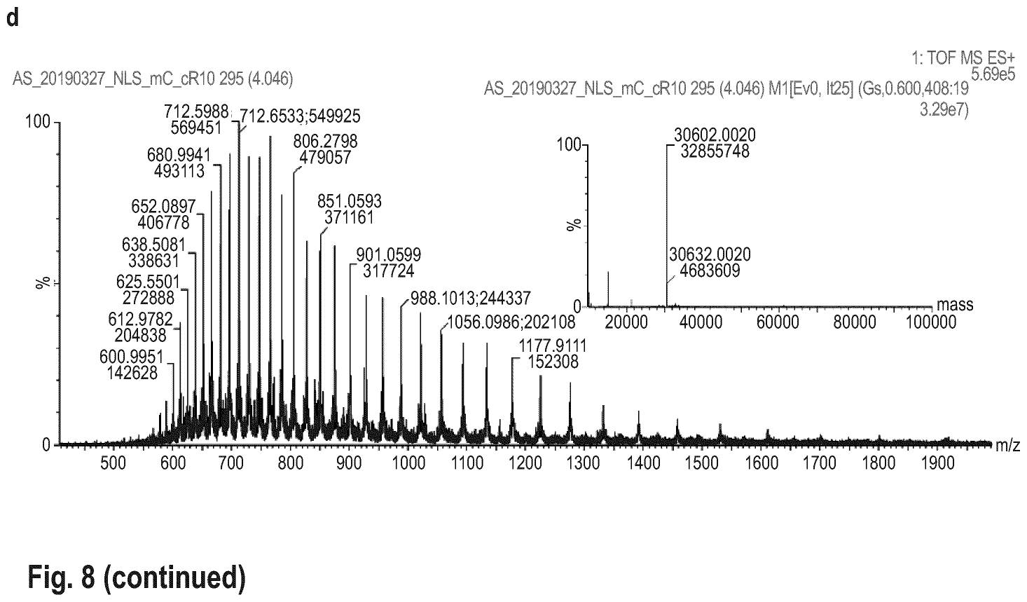

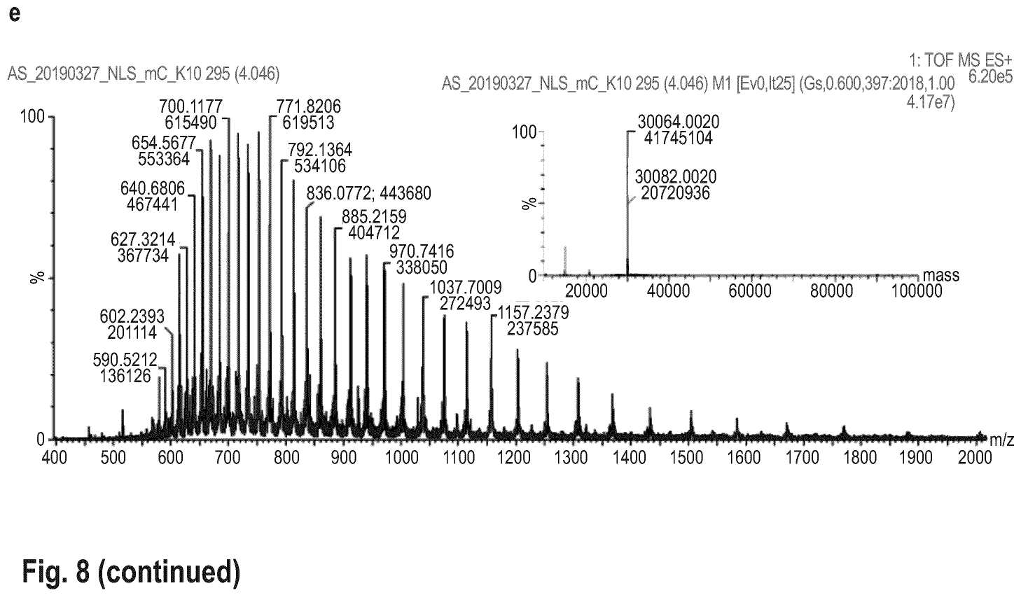

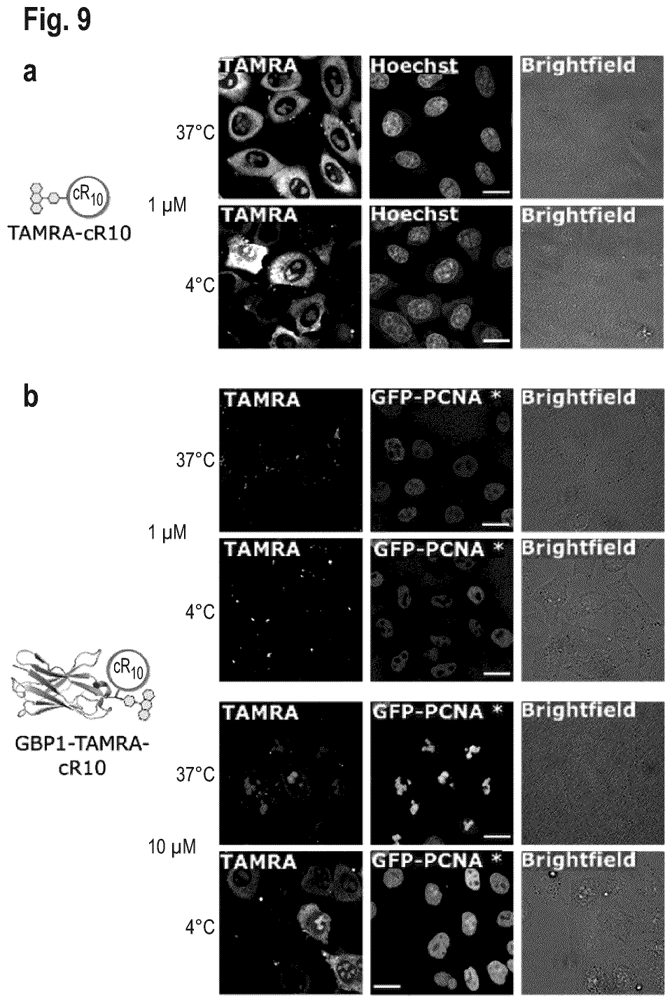

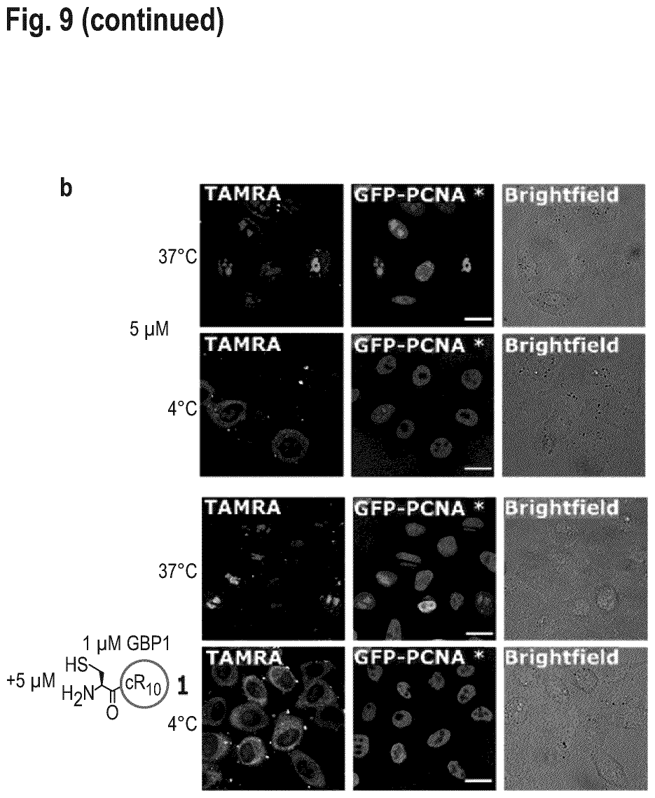

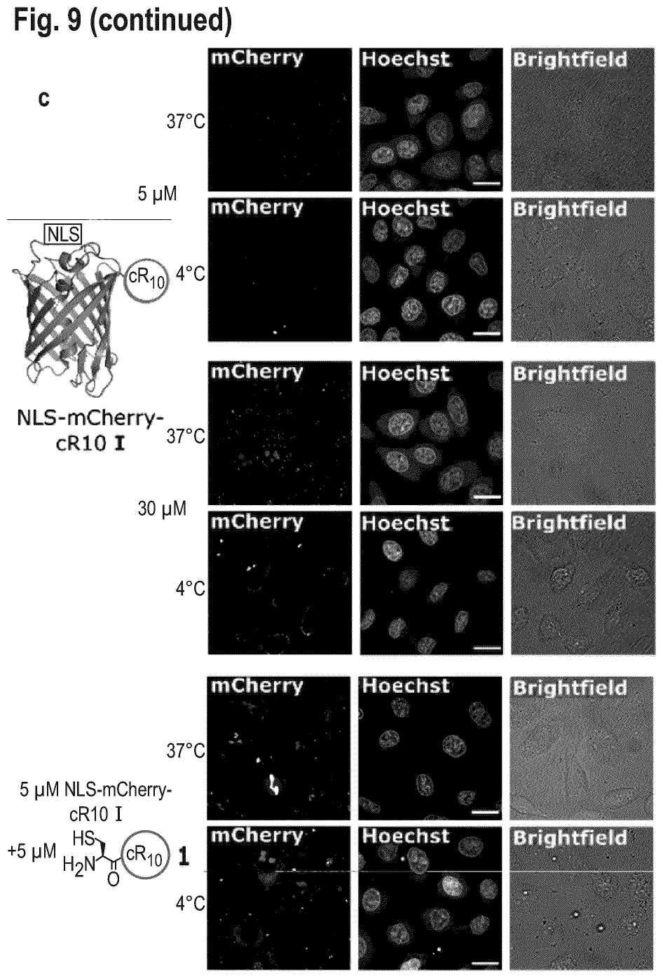

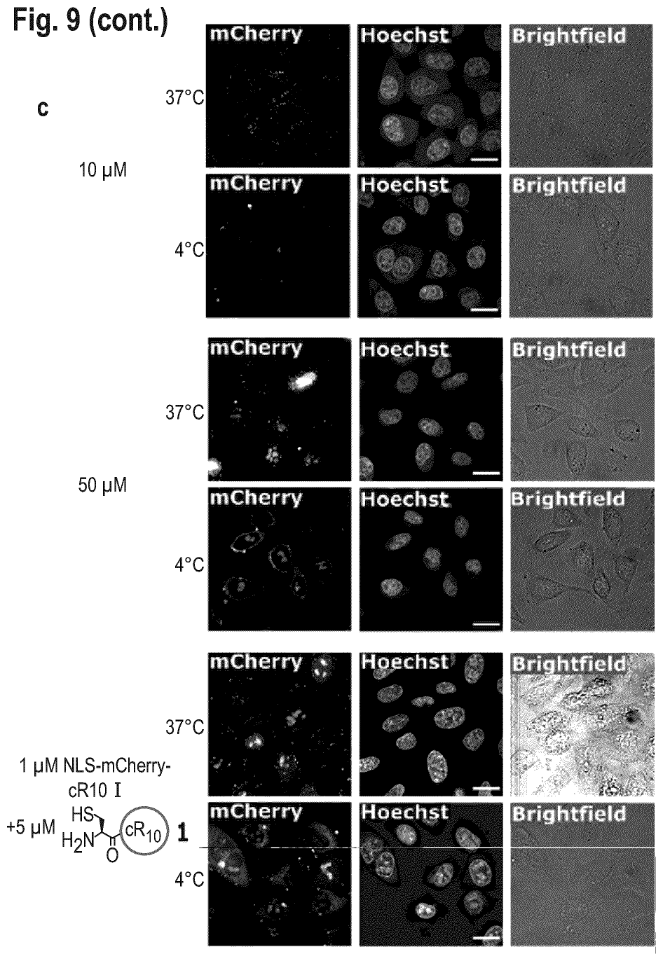

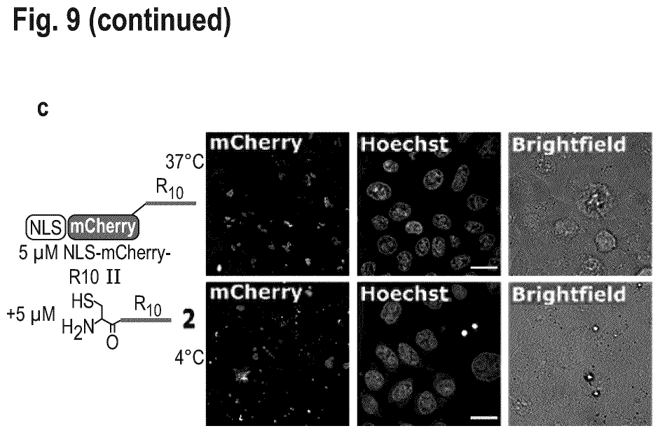

To evaluate the concentration, temperature and cargo-size dependency of added arginine-rich peptides to mediate cytosolic delivery, the inventors chose three distinct cargoes to transport: the organic fluorophore Tetramethylrhodamine (TAMRA, ˜450 Da), the camelid-derived anti-GFP nanobody GBP1 (˜14 kDa) and the fluorescent protein mCherry with a nuclear localization signal (NLS-mCherry, ˜28 kDa). The inventors attached each of the cargoes to a synthetic cyclic R10 (cR10) peptide yielding an intracellularly non-cleavable conjugate, either via an amide bond in the case of TAMRA or using maleimide chemistry for the proteins (analytical data for peptides in FIG. 6, characterization of GBP1 and mCherry and their CPP conjugates in FIGS. 7, 8), following the previous reports (Schneider, A. F. L., Wallabregue, A. L. D., Franz, L. & Hackenberger, C. P. R. Targeted Subcellular Protein Delivery Using Cleavable Cyclic Cell-Penetrating Peptides. Bioconjug Chem 30, 400-404, doi:10.1021/acs.bioconjchem.8b00855 (2019). Herce, H. D. et al. Cell-permeable nanobodies for targeted immunolabelling and antigen manipulation in living cells. Nat Chem 9, 762-771, doi:10.1038/nchem.2811 (2017)). In all cases, successful cytosolic delivery would lead to staining of the cytosol and of the nucleolus, an RNA-rich membrane-less compartment inside the nucleus (brighter area in the nucleus, FIG. 1a), for which cationic CPPs have an affinity (Schneider, A. F. L., Wallabregue, A. L. D., Franz, L. & Hackenberger, C. P. R. Targeted Subcellular Protein Delivery Using Cleavable Cyclic Cell-Penetrating Peptides. Bioconjug Chem 30, 400-404, doi:10.1021/acs.bioconjchem.8b00855 (2019). Martin, R. M., Herce, H. D., Ludwig, A. K. & Cardoso, M. C. Visualization of the Nucleolus in Living Cells with Cell-Penetrating Fluorescent Peptides. Methods Mol Biol 1455, 71-82, doi:10.1007/978-1-4939-3792-9_6 (2016). Martin, R. M., Tunnemann, G., Leonhardt, H. & Cardoso, M. C. Nucleolar marker for living cells. Histochem Cell Biol 127, 243-251, doi:10.1007/s00418-006-0256-4 (2007)). The cR10 peptide, consisting of ten arginines with alternating L- and D-configurations, has previously been shown to be effective in the delivery of functional proteins, albeit only at relatively high concentrations (Schneider, A. F. L., Wallabregue, A. L. D., Franz, L. & Hackenberger, C. P. R. Targeted Subcellular Protein Delivery Using Cleavable Cyclic Cell-Penetrating Peptides. Bioconjug Chem 30, 400-404, doi:10.1021/acs.bioconjchem.8b00855 (2019). Herce, H. D. et al. Cell-permeable nanobodies for targeted immunolabelling and antigen manipulation in living cells. Nat Chem 9, 762-771, doi:10.1038/nchem.2811 (2017)). We then applied all of the synthesized cargoes to HeLa Kyoto cells (expressing nuclear GFP-PCNA (Leonhardt, H. et al. Dynamics of DNA replication factories in living cells. J Cell Biol 149, 271-280, doi:10.1083/jcb.149.2.271 (2000). Chagin, V. O. et al. 4D Visualization of replication foci in mammalian cells corresponding to individual replicons. Nat Commun 7, 11231, doi:10.1038/ncomms11231 (2016)) as antigen for the nanobody), at both 37 and 4° C. (FIGS. 1b-d and individual channels in FIG. 9). At 37° C., the CPP-bearing cargoes can reach the cytosol either via endocytosis and endosomal escape (FIG. 1a) or directly transduce the membrane, circumventing the energy-dependent transport. At 4° C. however, active transport should not occur, and cellular fluorescence would be the result of transduction (Hunt, L. et al. Low-temperature pausing of cultivated mammalian cells. Biotechnol Bioeng 89, 157-163, doi:10.1002/bit.20320 (2005). Goldenthal, K. L., Pastan, I. & Willingham, M. C. Initial steps in receptor-mediated endocytosis. The influence of temperature on the shape and distribution of plasma membrane clathrin-coated pits in cultured mammalian cells. Exp Cell Res 152, 558-564, doi:10.1016/0014-4827(84)90658-x (1984)).

Upon incubation of the cells with TAMRA-cR10 for 1 hour in cell culture medium, the inventors found cytosolic (and nucleolar) localization of the red fluorophore at only 1 μM both when the incubation was done at 4 and 37° C. (FIG. 1b). These experiments show that successful cytosolic delivery for this small fluorophore can be achieved under conditions without endosomal uptake.

For the nanobody-CPP conjugate, 1 μM concentration results in predominantly punctate endosomal fluorescence at 37° C. while at 4° C. the nanobody is excluded from the cell (FIG. 1c). At 5 μM concentration, endosomal uptake is less prominent, and uptake also works to some extent at the cold temperature (FIG. 9b). At 10 μM concentration, all cells show cytosolic (and nucleolar) staining at 37° C. and 4° C. (FIG. 1c) Interestingly, for all proteins we tested the nucleolar staining is more evident at 37° C. whereas cytosolic staining is more pronounced at 4° C., which may be because active nuclear import is also an energy independent process (Melchior, F., Guan, T., Yokoyama, N., Nishimoto, T. & Gerace, L. GTP hydrolysis by Ran occurs at the nuclear pore complex in an early step of protein import. J Cell Biol 131, 571-581, doi:10.1083/jcb.131.3.571 (1995)).

Following the proposal, the inventors thought it may be possible to rescue the cytosolic delivery of the protein at low concentrations by addition of unbound CPP. Indeed, when the inventors co-incubated 5 μM of a cysteine-containing cR10 peptide (Cys-cR10, 1), which the inventors previously used for the semi-synthesis of nanobodies by expressed protein ligation (Herce, H. D. et al. Cell-permeable nanobodies for targeted immunolabelling and antigen manipulation in living cells. Nat Chem 9, 762-771, doi:10.1038/nchem.2811 (2017)), with 1 μM of the nanobody-cR10 conjugate for 1 hour on HeLa cells, the nanobody showed efficient cytosolic and nucleolar staining at both temperatures (FIG. 1c and SI FIG. 9b).

For the NLS-mCherry-cR10 conjugate (1), the required concentration to achieve cytosolic uptake is even more restrictive, with anything below 50 μM leading to dominant endosomal uptake without nucleolar localization at 37° C. and no uptake at all at 4° C. (FIG. 1d). Analogous to the nanobody experiments, the addition of 5 μM peptide 1 allowed energy-independent transduction of mCherry at a low concentration of 5 μM (FIG. 1d). Delivery could even be achieved at 1 μM protein and 5 μM peptide (FIG. 9c), although under these conditions the fluorescence of the mCherry was faint and difficult to detect.

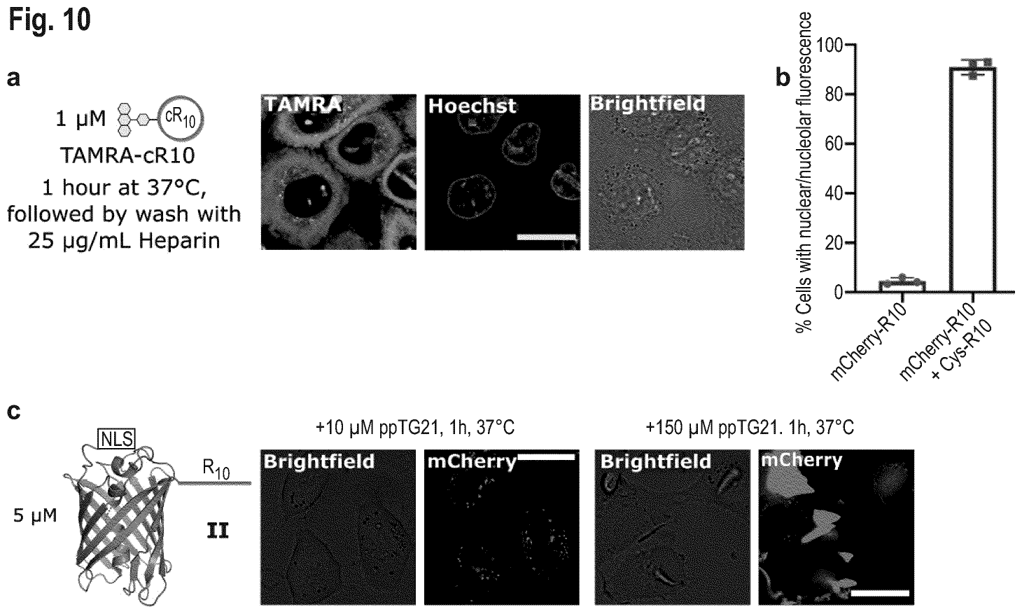

Encouraged by these findings, the inventors subsequently probed the performance of linear CPP sequences in the cargo-conjugates and additives. Although CPP cyclization is known to improve cell permeability, (Lattig-Tunnemann, G. et al. Backbone rigidity and static presentation of guanidinium groups increases cellular uptake of arginine-rich cell-penetrating peptides. Nat Commun 2, 453, doi:10.1038/ncomms1459 (2011). Nischan, N. et al. Covalent attachment of cyclic TAT peptides to GFP results in protein delivery into live cells with immediate bioavailability. Angewandte Chemie 54, 1950-1953, doi:10.1002/anie.201410006 (2015), the inventors now also observed efficient nucleolar delivery of 5 μM NLS-mCherry linked to a linear R10-peptide (conjugate 1l) with 5 μM of a linear CPP-additive (Cys-R10 2), in which both sequences consist of ten L-arginine residues. (FIG. 1e). With the CPP-additive 2, nucleolar red fluorescence could be detected in more than 90% of cells, but in less than 5% without the additive (FIG. 10b). As comparison, the inventors tested the delivery of mCherry-R10 II in presence of 10 or 150 μM of the endosomolytic peptide ppTG21^{44,45} Neither concentration led to efficient endosomal release, but instead to the formation of large, fluorescent aggregates in cells (FIG. 10c).

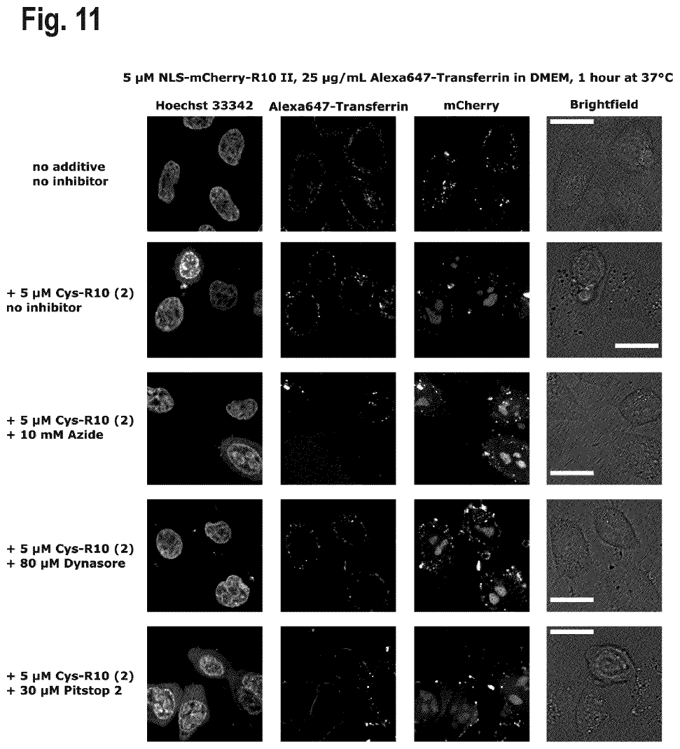

Finally, the inventors performed uptake at 37° C. with NLS-mCherry-R10 II and the CPP-additive 2 in presence of Alexa647-labelled Transferrin (which undergoes receptor-mediated endocytosis (Mayle, K. M., Le, A. M. & Kamei, D. T. The intracellular trafficking pathway of transferrin. Biochim Biophys Acta 1820, 264-281, doi:10.1016/j.bbagen.2011.09.009 (2012). Ter-Avetisyan, G. et al. Cell entry of arginine-rich peptides is independent of endocytosis. J. Biol. Chem. 284, 3370-3378, doi:10.1074/jbc.M805550200 (2009)) and endocytosis inhibitors (sodium azide, dynasore and pitstop 2). While the inventors could see inhibition of the endocytosis of transferrin, the inventors could detect nucleolar mCherry regardless of the inhibitor used (FIG. 11).

### Example 2: A Thiol-Reactive Deca-Arginine is a Highly Effective Additive for Delivering CPP-Conjugated Proteins

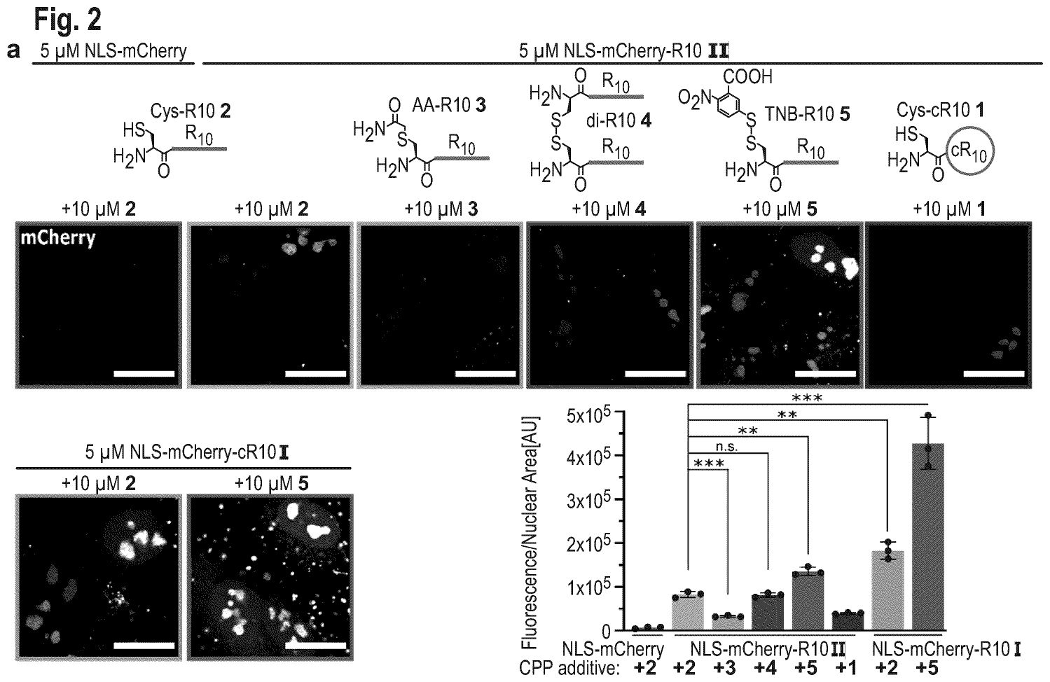

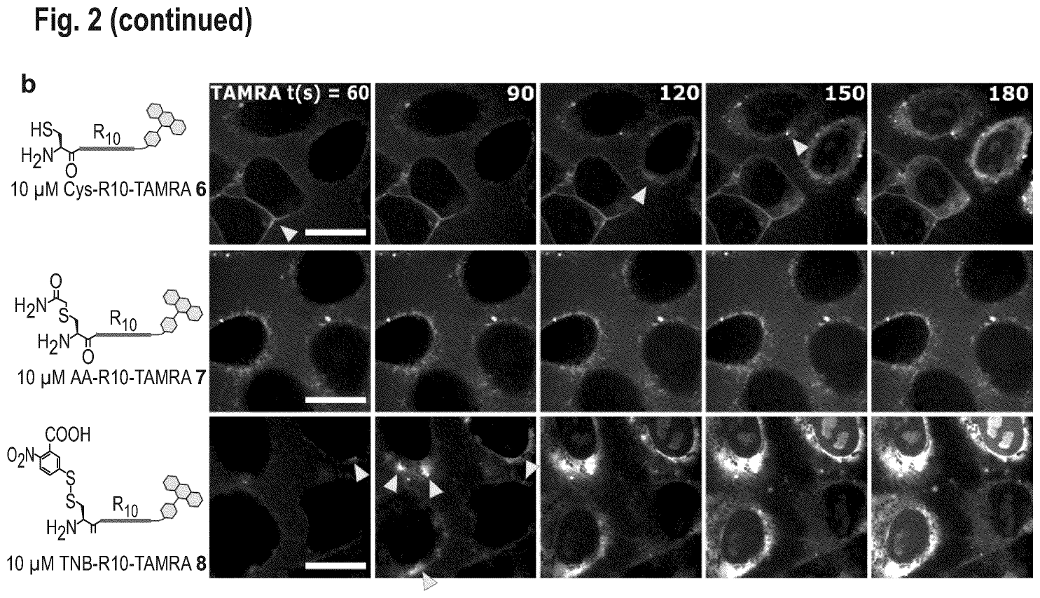

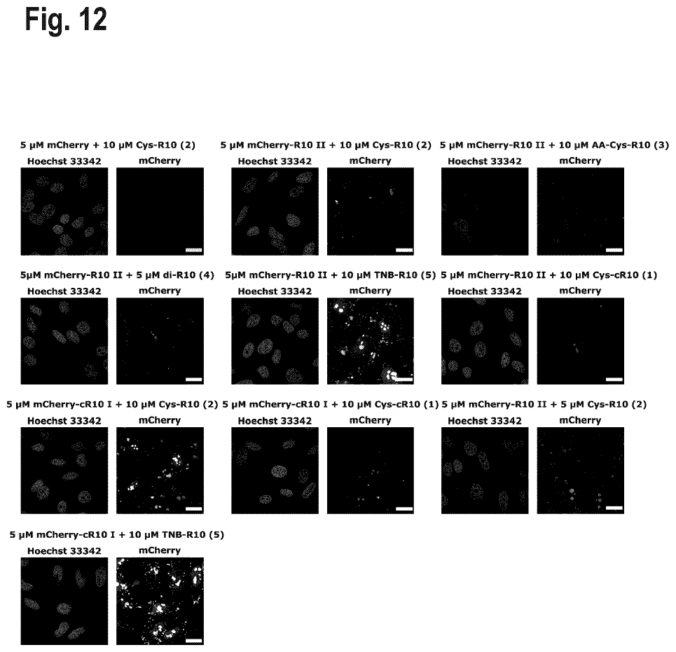

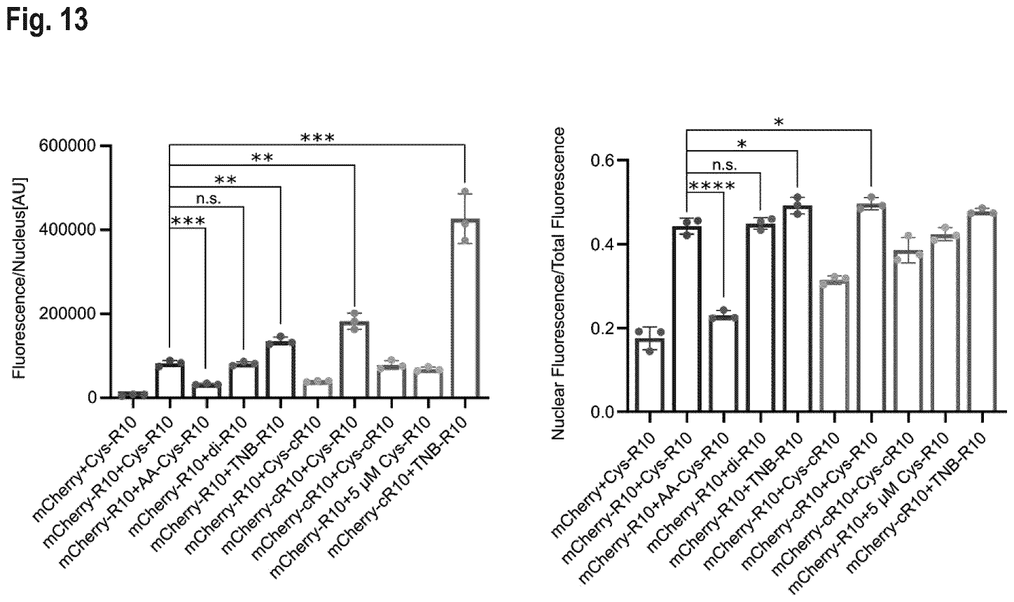

To further evaluate the high efficiency of the CPP-peptide additives 1 and 2 in the additive protocol and to probe the impact of the N-terminal thiol functionality, the inventors synthesized additional linear CPPs 3-5 with different thiol derivatives (FIG. 2a). We then co-delivered 5 μM of CPP-conjugates of mCherry either linked to a linear (11) or cyclic R10 (1) (as above in FIG. 1 d-e) together with the newly synthesized peptides 1-5 into HeLa Kyoto cells and used microscopy to measure both nuclear and total fluorescence (representative pictures for all tested conditions in FIG. 12). Thereby, the inventors quantified the desirable delivery to the nucleus and nucleoli (FIG. 2a), and also the amount of unwanted endosomal entrapment (FIG. 13, see example 6 with supporting methods and general methods for details). Using unconjugated mCherry as a cargo did not result into any detectable intracellular florescence, while using mCherry-R10 conjugate II in combination with additive R10 peptide 2 led to nuclear/nucleolar staining as before (FIG. 2a, first two bars).

To exclude thiol-based interactions of the peptide, the inventors capped the N-terminal Cys-residue with iodoacetamide in CPP 3. Using 3, the inventors observed a sharp decrease in efficiency of the nuclear protein delivery in comparison to additive 2, (FIG. 2a, second and third bars from the left). To probe a potential dimerization of the CPP, following the previous observation of using disulfide-linked TAT dimers (Erazo-Oliveras, A. et al. Protein delivery into live cells by incubation with an endosomolytic agent. Nat Methods 11, 861-867, doi:10.1038/nmeth.2998 (2014)), the inventors employed the dimer of Cys-R10 (4, fourth bar from the left); however, no increase in efficiency over using the monomer 2 was visible in our case.

Based on these observations the inventors hypothesized that the better delivery using peptide-additive 2 is due to the formation of disulfide bridges on the cell-surface (Gasparini, G. et al. Cellular uptake of substrate-initiated cell-penetrating poly(disulfide)s. J Am Chem Soc 136, 6069-6074, doi:10.1021/ja501581b (2014). Fu, J., Yu, C., Li, L. & Yao, S. Q. Intracellular Delivery of Functional Proteins and Native Drugs by Cell-Penetrating Poly(disulfide)s. J Am Chem Soc 137, 12153-12160, doi:10.1021/jacs.5b08130 (2015)). Therefore, the inventors synthesized a thio-nitro-benzoic acid activated R10-peptide (TNB-R10, 5), which carries an electrophilic disulfide to accelerate the disulfide formation.

Indeed, the inventors observed more than 50% increase in nuclear mCherry fluorescence intensity as compared to the Cys-variant 2 (fourth bar from the right, FIG. 2a), and a significant increase in the fraction of nuclear fluorescence (FIG. 13).

Using the cyclic R10-peptide 1 as co-delivery agent decreased the uptake efficiency (FIG. 2a, third bar from the right), but using the mCherry-conjugate with the cyclic-R10 (1) made the delivery with additive 2 even more efficient (FIG. 2a, second bar from the right).

By far the highest nuclear fluorescence was observed with conjugate (1) in combination with the electrophilic disulfide additive 5 (FIG. 2a, first bar from the right), although endosomal fluorescence was also increased under these conditions (FIG. 13).

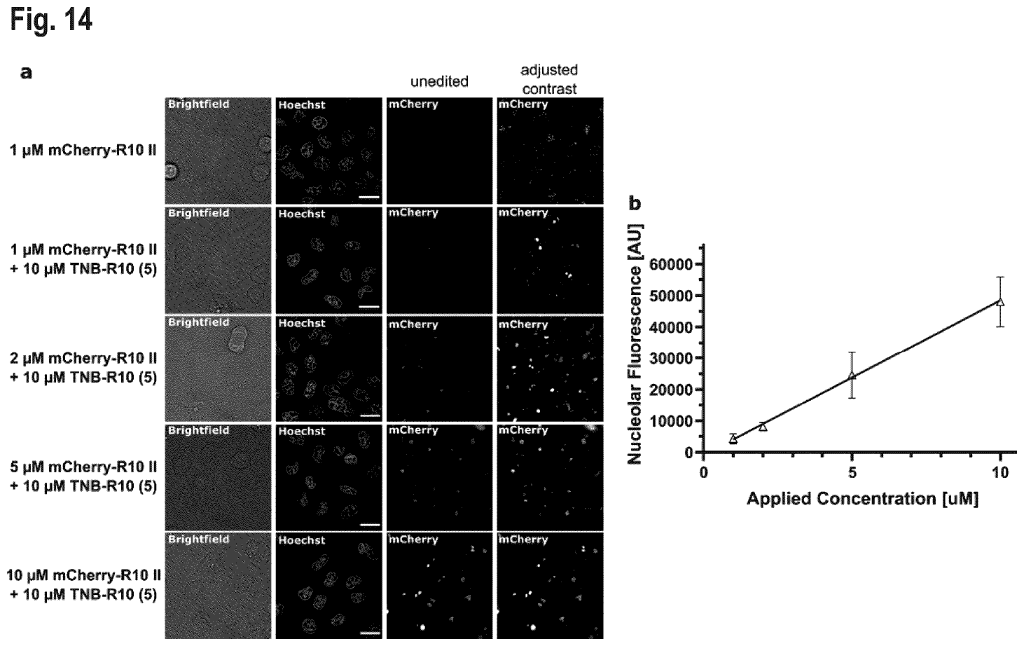

Another clear advantage of using an additive to control the cytosolic delivery of cargoes is that it may allow more control over the delivered cargo concentration. To test this proposal, the inventors added various amounts of NLS-mCherry-R10 to cells together with a constant concentration of peptide additive 5. the inventors could see a linear relationship between the amount of mCherry added to the cells and the resulting nucleolar fluorescence, indicating that it is possible to titrate a cargo into cells precisely (FIG. 14).

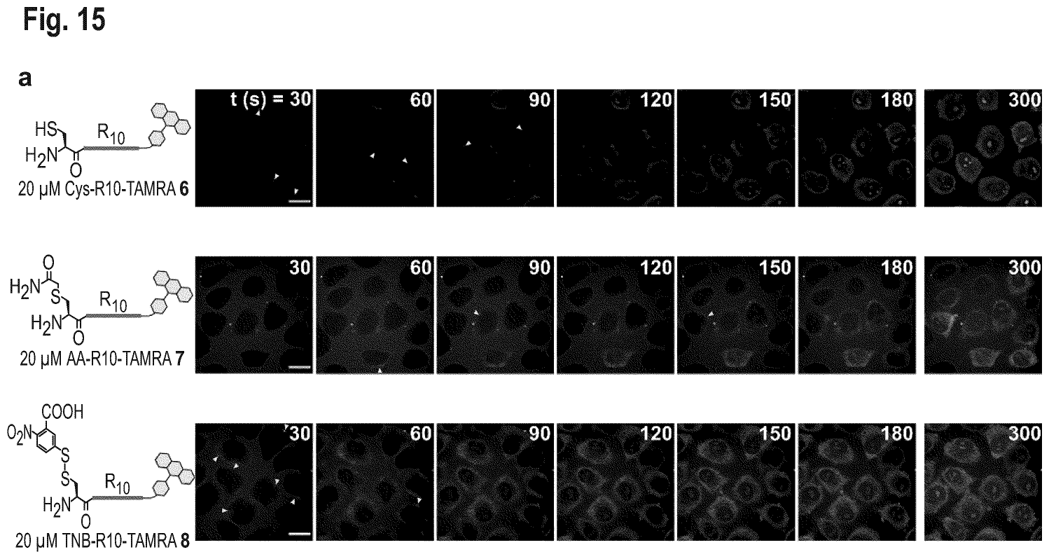

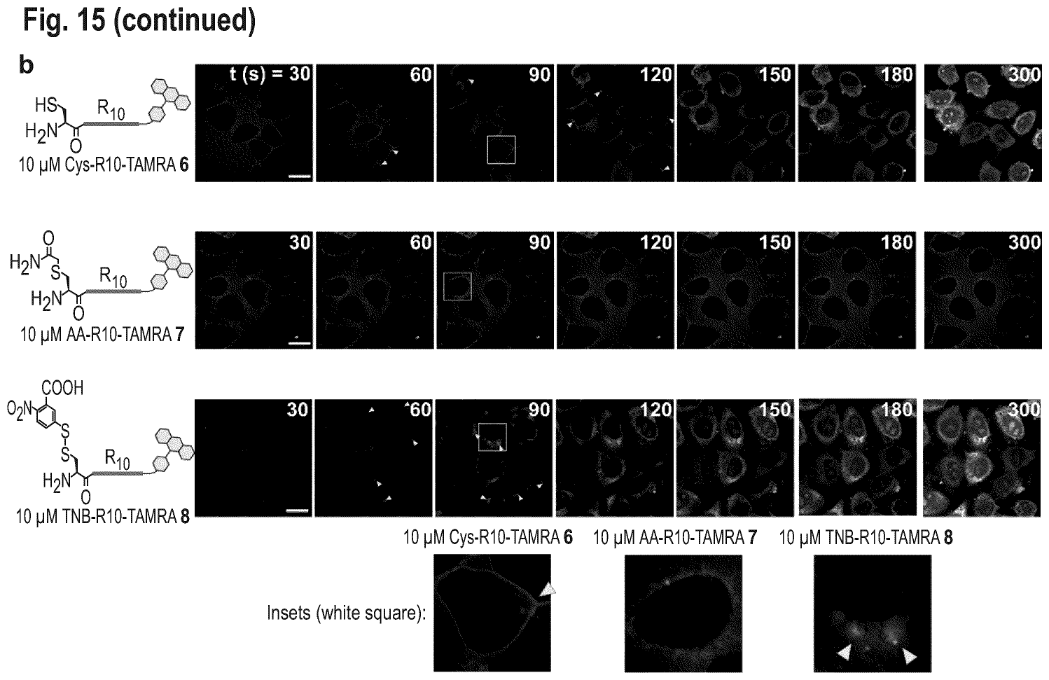

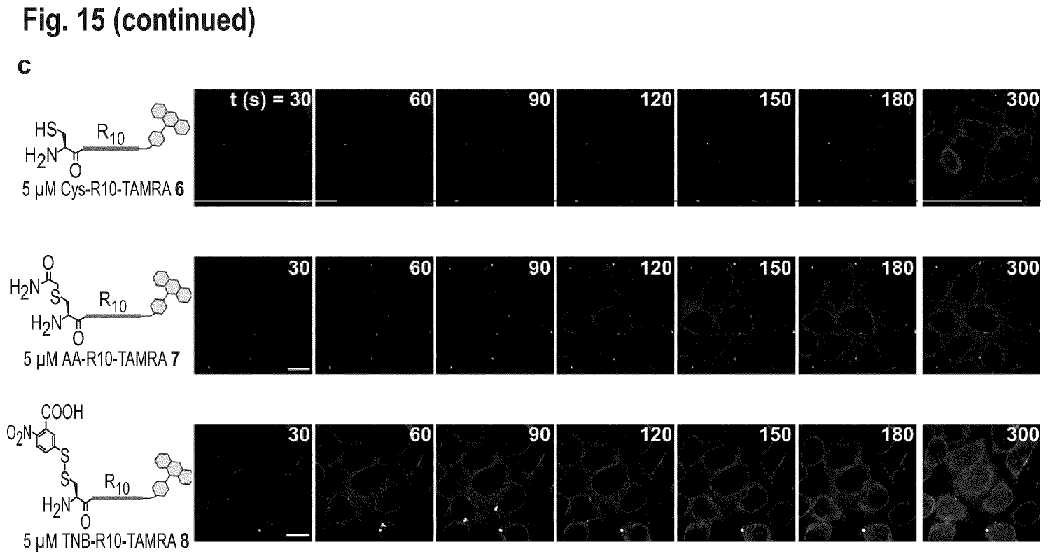

To obtain a better understanding of how the cysteine- and TNB-containing peptide additives perform better in cargo delivery, the inventors synthesized fluorescent variants 6-8 of peptides 2, 3 and 5. The inventors performed time-lapse uptake experiments of the peptides alone at 5, 10 and 20 μM concentration (FIG. 2b and complete data set in FIG. 15). All peptides showed rapid uptake into cells at 20 μM concentration, immediately after the appearance of bright spots on the membrane, which were previously described as “nucleation zones” (Duchardt, F., Fotin-Mleczek, M., Schwarz, H., Fischer, R. & Brock, R. A comprehensive model for the cellular uptake of cationic cell-penetrating peptides. Traffic 8, 848-866, doi:10.1111/j.1600-0854.2007.00572.x (2007). Wallbrecher, R. et al. Membrane permeation of arginine-rich cell-penetrating peptides independent of transmembrane potential as a function of lipid composition and membrane fluidity. J Control Release 256, 68-78, doi:10.1016/j.jconrel.2017.04.013 (2017)). At 10 μM concentration, the acetylated peptide 7 did not show any uptake during the first 3 minutes, whereas the uptake was already complete for 6 and 8 (FIG. 2b). Notably, the TNB-modified peptide 8 (FIG. 2b, bottom row) showed very quick uptake and very frequent formation of nucleation zones (bright arrowheads, enlarged insets in FIG. 15). Similar observations could also be made with 5 μM peptide, although uptake was slower (FIG. 15). These findings suggest that the thiol-reactive head groups assist the peptide in forming these zones and crossing the membrane.

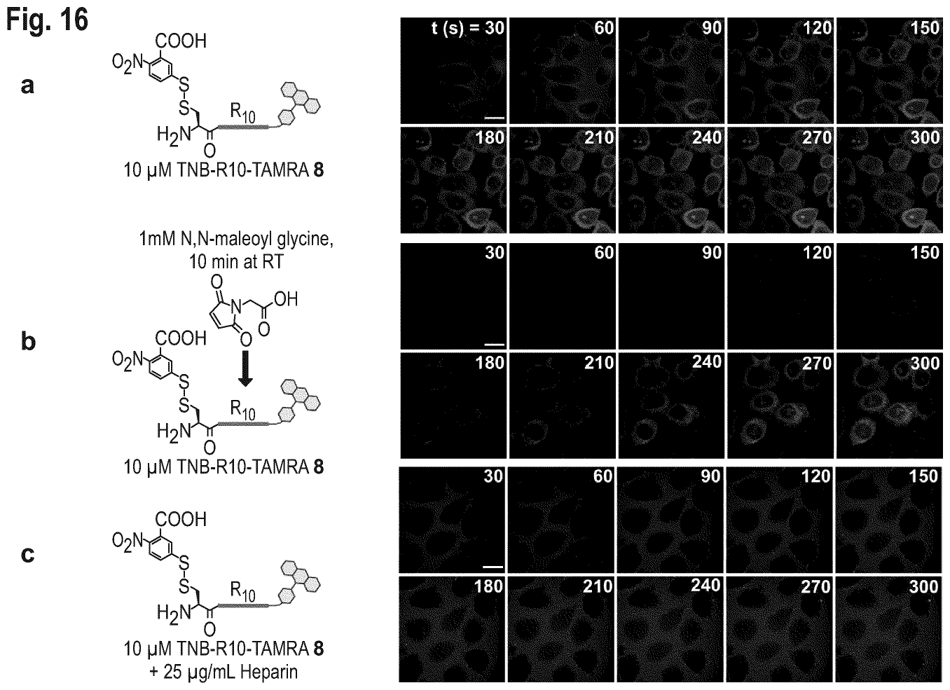

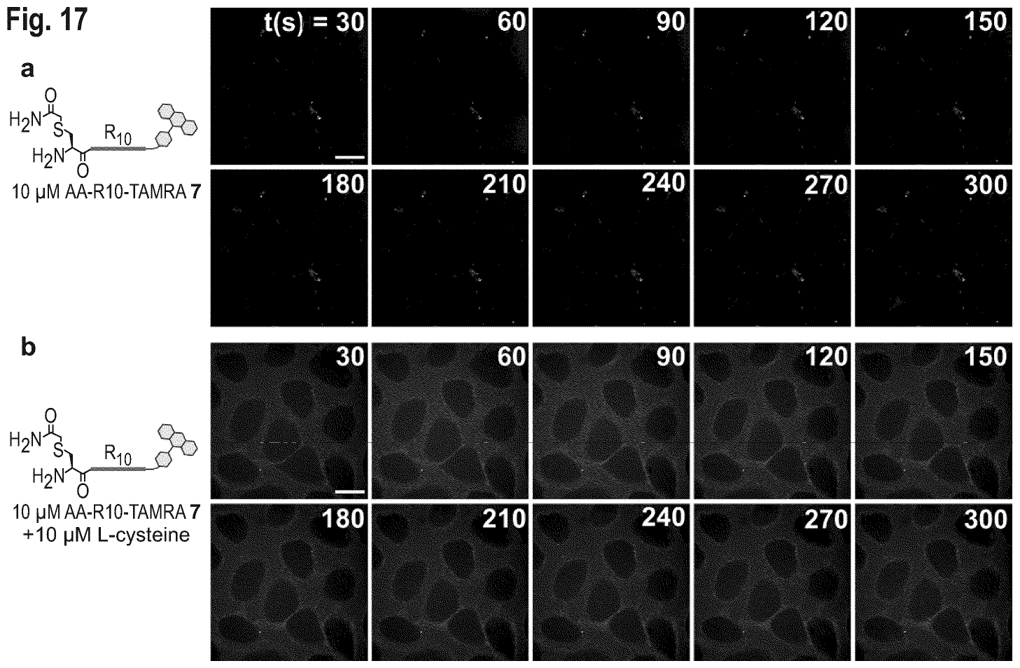

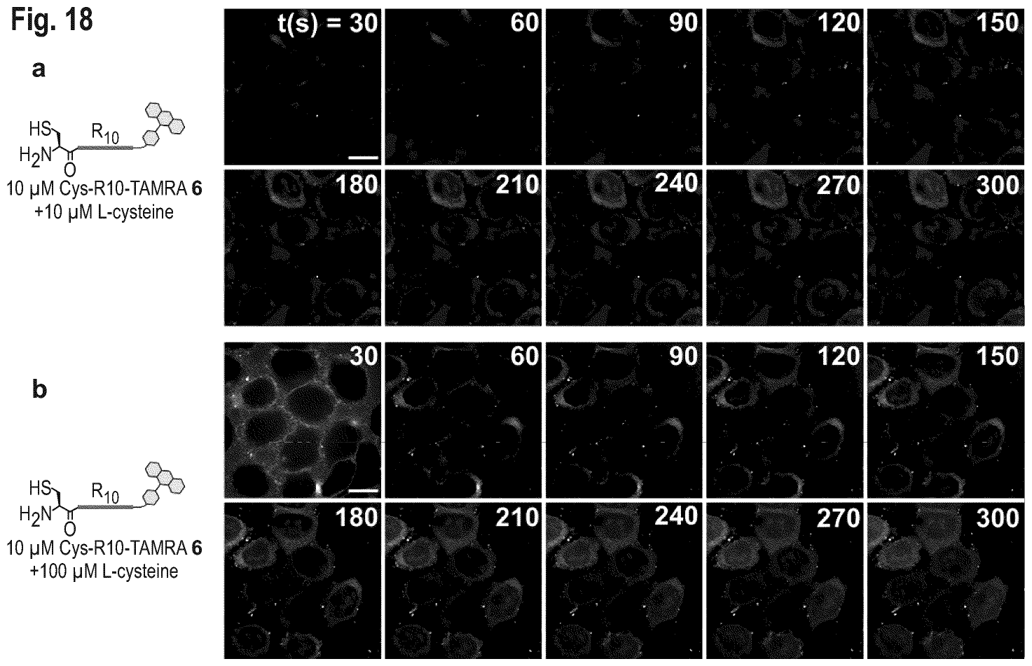

To ensure that this effect is due to the thiol-reactivity of the TNB-group, the inventors pre-treated cells with a thiol-reactive maleimide that should at least partially block accessible cell-surface thiols. After this pre-treatment, the uptake of the TNB-R10 8 was indeed slowed down considerably (FIG. 16b). The peptide was also not taken up at all in presence of the anionic polysaccharide heparin (FIG. 16c), showing that the electrostatic interactions between the polyarginine and cell are also crucial for uptake. The inventors also investigated the addition of free, reduced cysteine into the cell medium during uptake (Wei, Y., Tang, T. & Pang, H. B. Cellular internalization of bystander nanomaterial induced by TAT-nanoparticles and regulated by extracellular cysteine. Nat Commun 10, 3646, doi:10.1038/s41467-019-11631-w (2019)). Addition of cysteine neither sped up the uptake of the acetylated peptide 7 (FIG. 17) nor slowed down the uptake of the cysteine containing peptide 6 (FIG. 18).

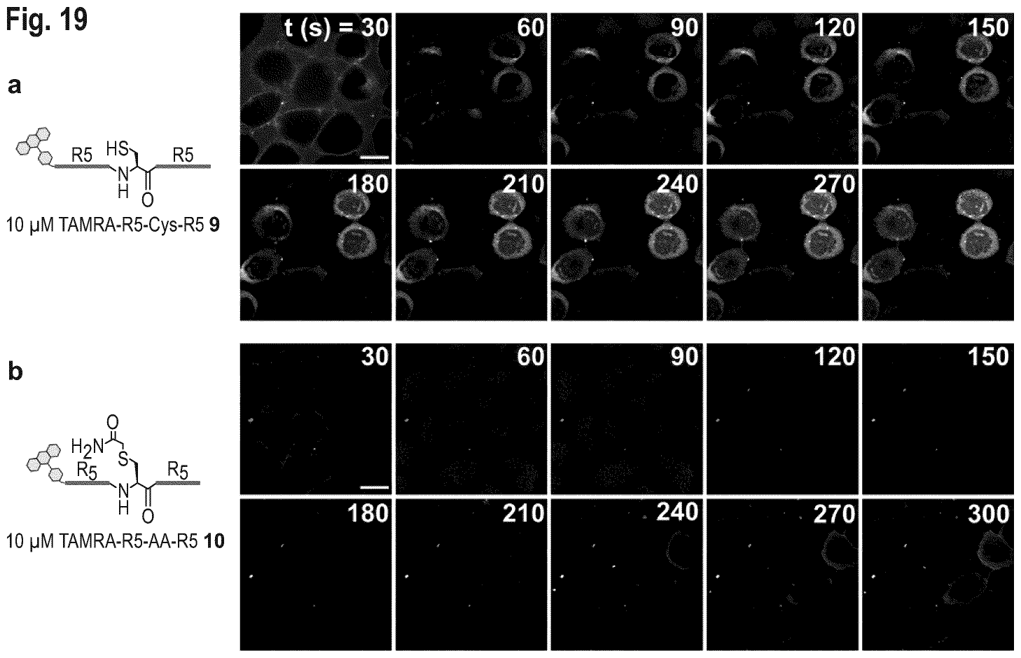

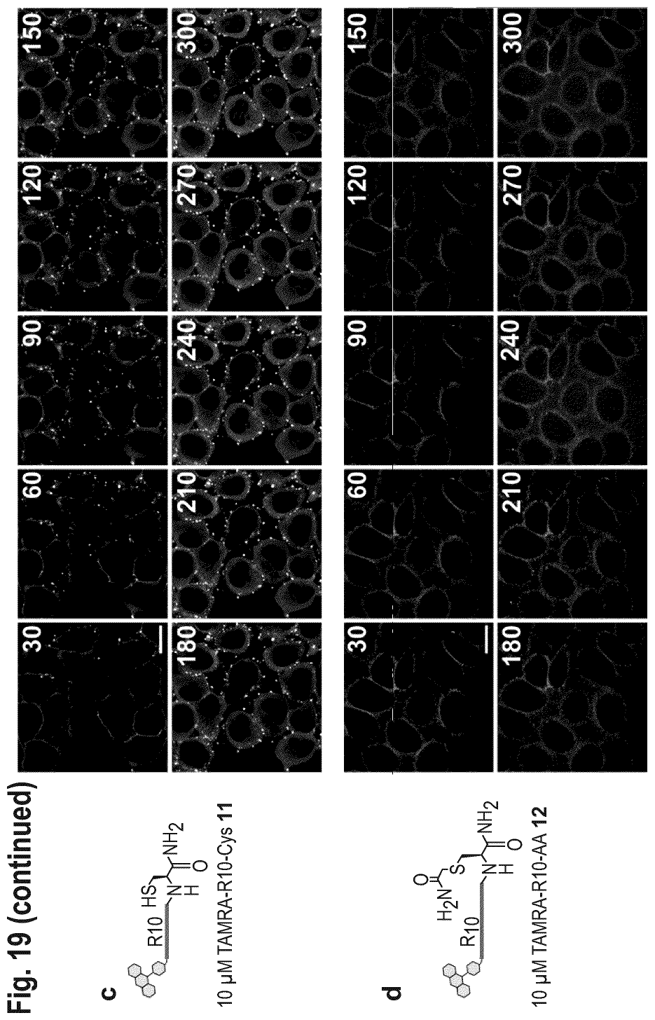

To verify that these effects are independent of the position of the cysteine within the peptide, the inventors also synthesized two additional cysteine-containing, fluorescent R10 peptides in which the cysteine was in a different position (within the polyarginine sequence or at the C-terminus). These peptides showed comparable rates of uptake to the previous peptide and they were also much faster than their acetylated counterparts (peptides 9-12, FIG. 19).

### Example 3: Covalent Immobilization of CPPs on the Cell-Surface Allows Delivery of Large Cargoes Through the Membrane

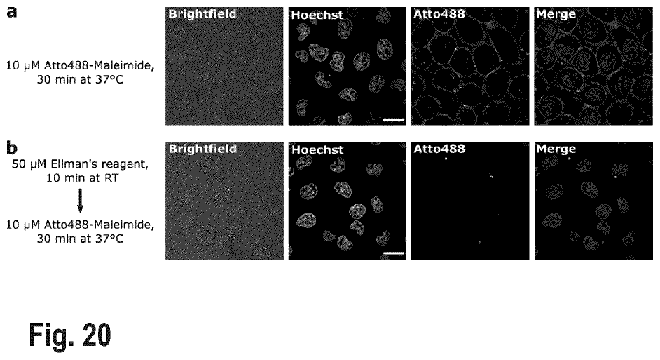

Next, the inventors wanted to explore other cysteine-selective reactions in this context. Maleimides are also thiol-selective and form more stable bonds (under biological conditions) than disulfides, which makes characterization easier. The inventors first wanted to confirm that there are addressable, surface-exposed thiols on cells. To that end, the inventors labelled cells with a cell-impermeable, maleimide-functionalized fluorophore (FIG. 20). The fluorophore showed effective membrane staining, which could be strongly reduced by first blocking thiols on the cells with Ellman's reagent (FIG. 20).

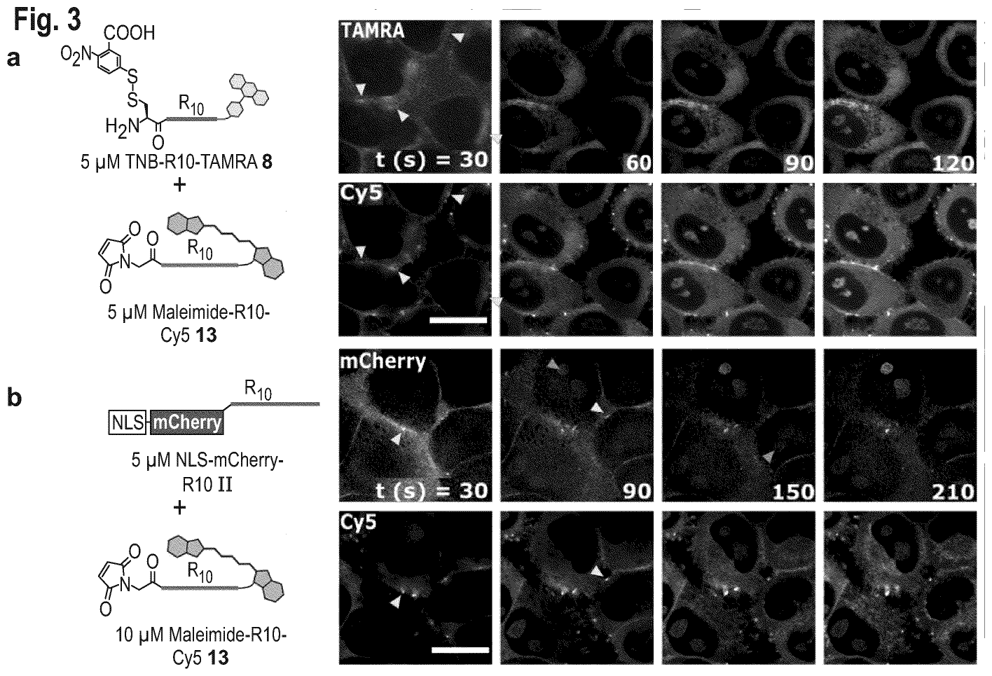

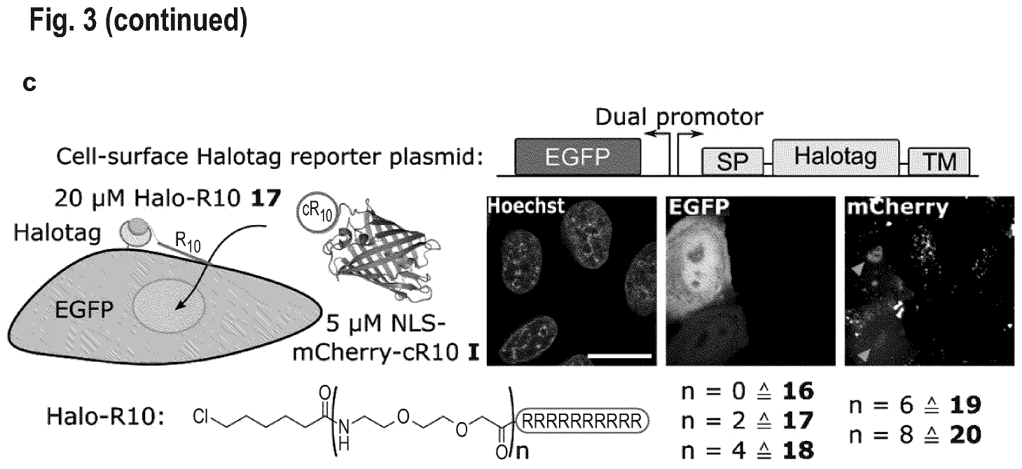

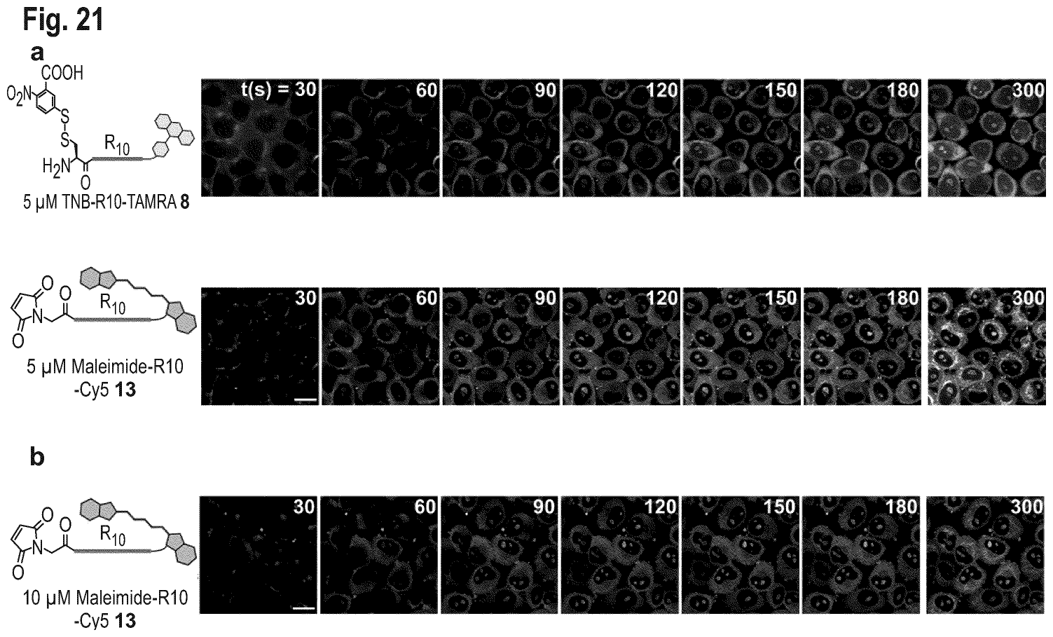

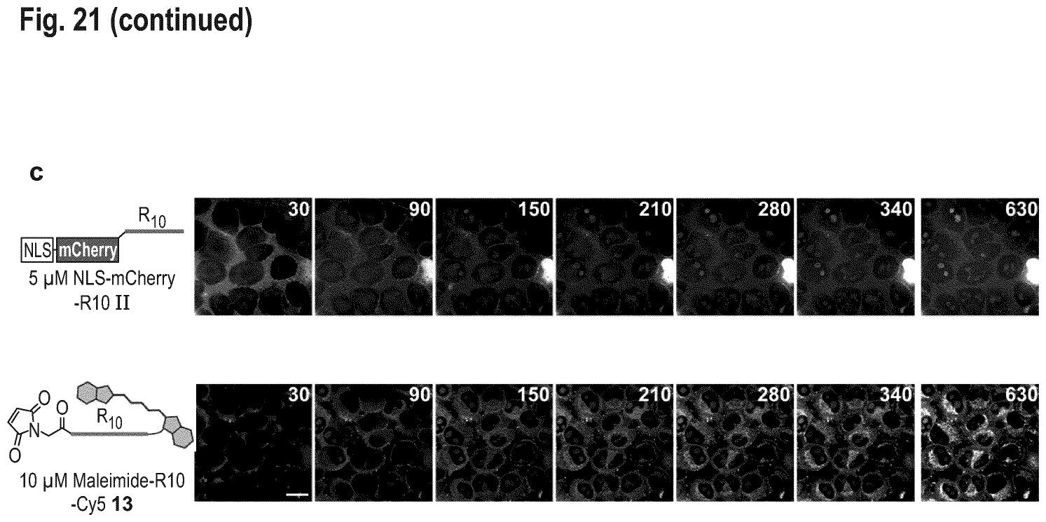

The inventors then synthesized a fluorescent, maleimide-functionalized linear R10 (Maleimide-R10-Cy5) 13, which can be traced separately by fluorescent microscopy. First, the inventors wanted to confirm that this peptide shows similar uptake behavior as the fluorescent TAMRA-labeled TNB-activated R10 peptide 8. Indeed, when the two fluorescent peptides are incubated with cells simultaneously, they stain the same nucleation zones and are taken up at similar rates (FIG. 3a and FIG. 21a), and peptide 13 also shows staining of nucleation zones alone (FIG. 21b).

The inventors then co-delivered R10-modified mCherry II together with the newly synthesized Maleimide-R10-Cy5 peptide 13 (FIG. 3b and FIG. 21c). The inventors observed that the protein was localized at the same nucleation zones and is subsequently taken up into cells, although the protein requires more time to reach the nucleolus (note the longer steps in the time-lapse experiment). This observation supports the assumption that the protein crosses nucleation zones, which are “pre-labelled” by the reactive peptide additives.

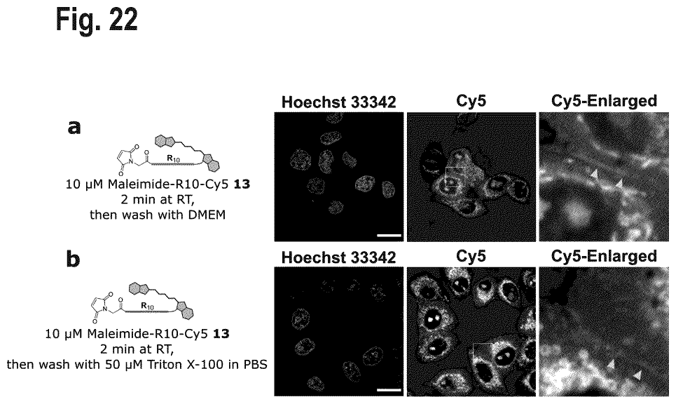

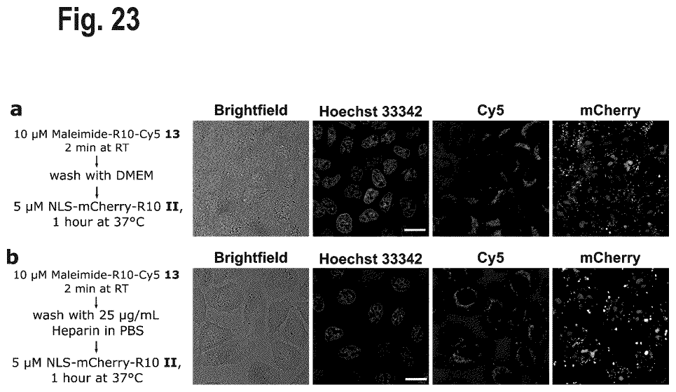

As additional evidence for the covalent modification of a membrane component with 13, the inventors treated cells with the peptide and subsequently washed the cells with either medium or 50 μM Triton X-100 to remove unbound peptide. The peptide stained membranes even after washing with the detergent (FIG. 22). The inventors also treated cells with 13 and delivered NLS-mCherry-R10 II into these cells after washing with 25 μg/mL heparin (FIG. 23). Washing with heparin should remove cell-penetrating peptides that are non-covalently bound to the cell membrane (Wadia, J. S., Stan, R. V. & Dowdy, S. F. Transducible TAT-HA fusogenic peptide enhances escape of TAT-fusion proteins after lipid raft macropinocytosis. Nat Med 10, 310-315, doi:10.1038/nm996 (2004)). The successful delivery of mCherry II suggests that the covalently bound peptide can be sufficient for protein delivery.

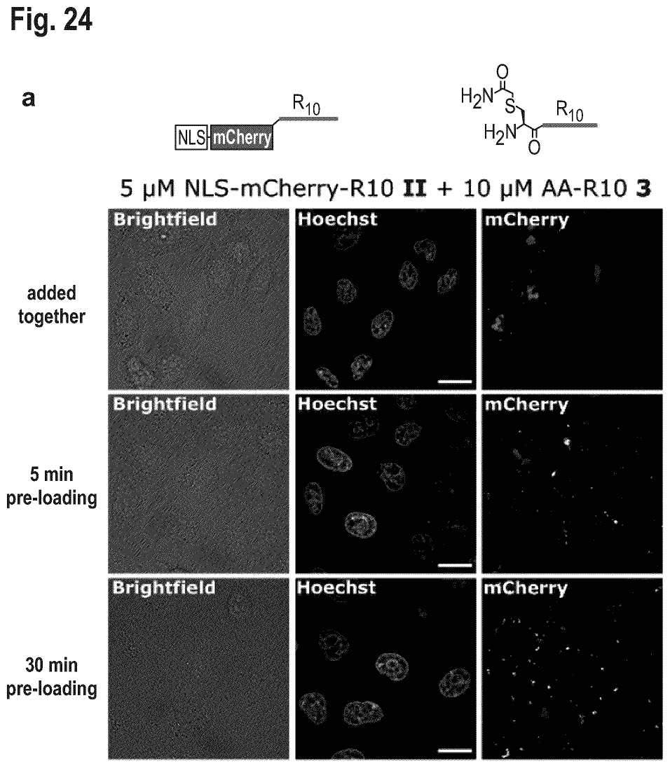

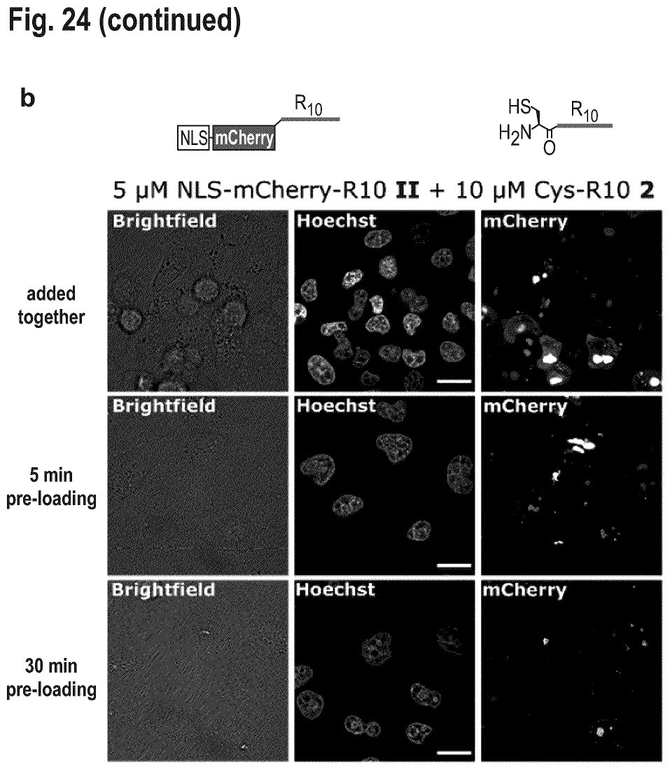

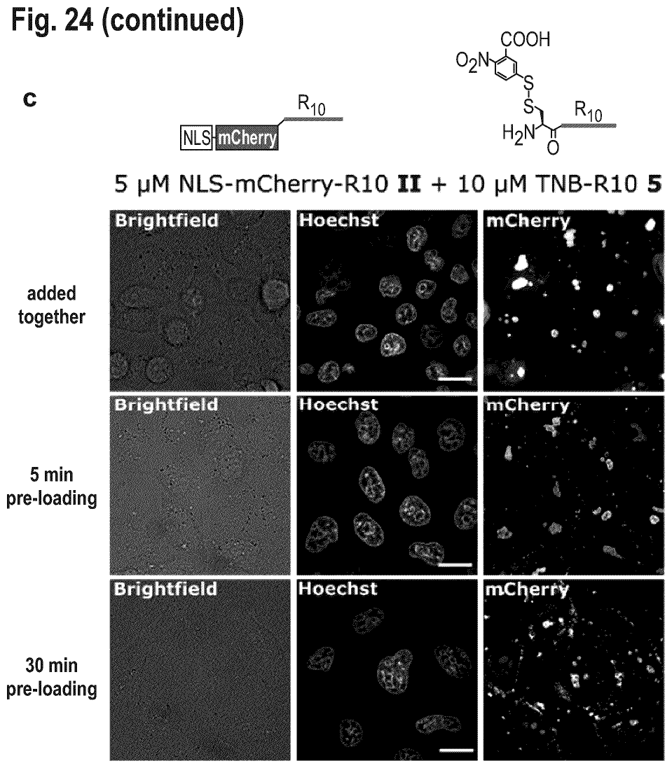

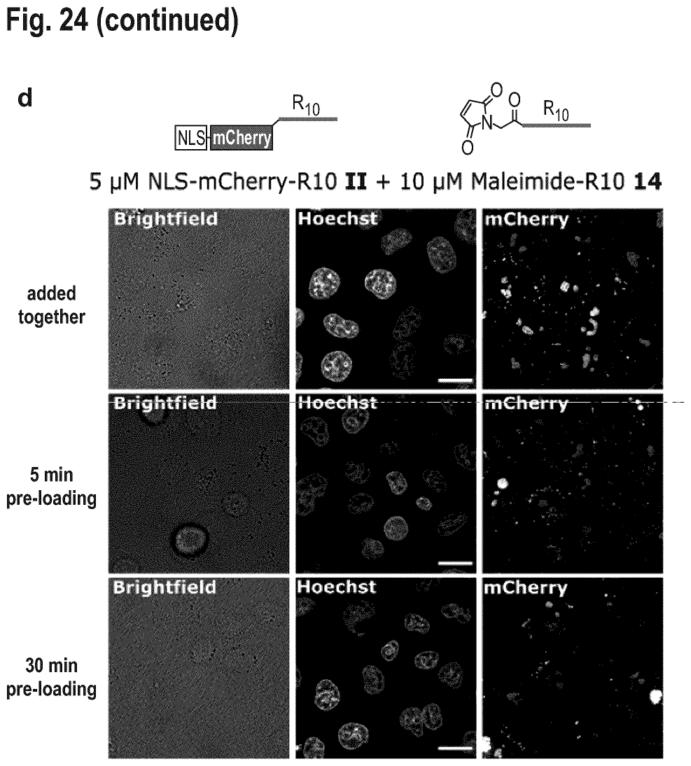

To explore this concept further, the inventors treated cells with peptides 2, 3, 5 or with a non-fluorescent maleimide-R10 peptide 14 in a first step. After certain time points, the inventors removed the peptide solution and added the R10-conjugated mCherry II (FIG. 24). For the maleimide- and TNB-R10 peptides 5 and 12, the inventors could still observe nuclear delivery after 5 minutes of “pre-labelling” with the peptide, and successful, albeit reduced, delivery of mCherry II after 30 minutes.

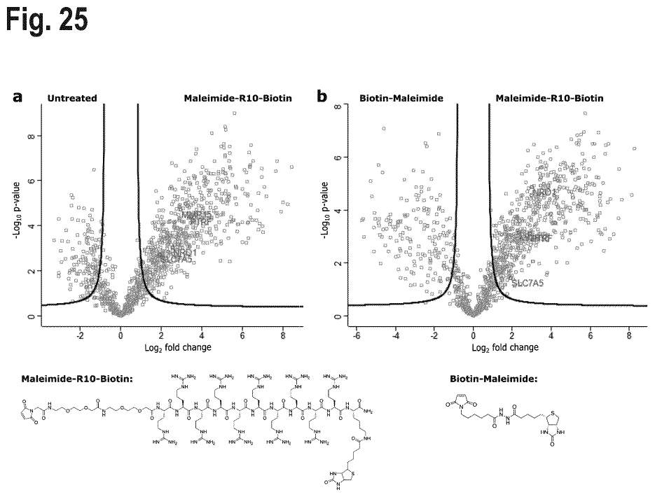

To identify potential reaction partners of the cysteine-reactive peptides, the inventors synthesized a biotinylated version of the maleimide-R10 peptide (15) and applied it to cells followed by a streptavidin pulldown, tryptic digestion, and protein identification by mass spectrometry. Label-free-quantification of identified proteins revealed several membrane proteins enriched by the R10-peptide over untreated cells and a biotin-maleimide control (FIG. 25). This suggests that there is no single target but rather several proteins that the peptides can react with.

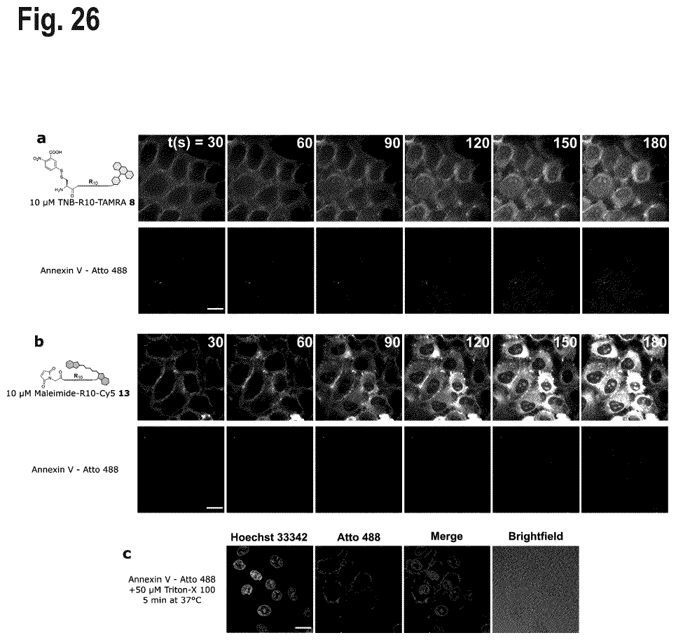

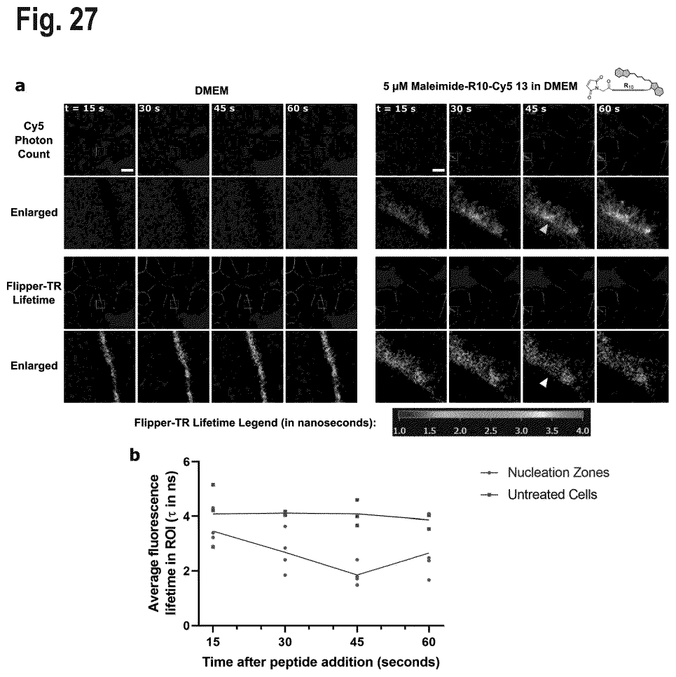

To investigate changes in the membrane at nucleation zones, the inventors performed uptake of CPPs in the presence of the phosphatidylserine-binding protein annexin V. It had previously been suggested that the accumulation of CPPs at nucleation zones leads to a local membrane inversion, facilitating cargo uptake (Hirose, H. et al. Transient focal membrane deformation induced by arginine-rich peptides leads to their direct penetration into cells. Mol Ther 20, 984-993, doi:10.1038/mt.2011.313 (2012)). We could not detect any enrichment of phosphatidylserine (FIG. 26); however, the inventors employed Flipper-TR, a fluorescent membrane tension probe (Colom, A. et al. A fluorescent membrane tension probe. Nat Chem 10, 1118-1125, doi:10.1038/s41557-018-0127-3 (2018)) and could observe a reduction of membrane tension at nucleation zones, which points to a local deformation of the membrane (FIG. 27).

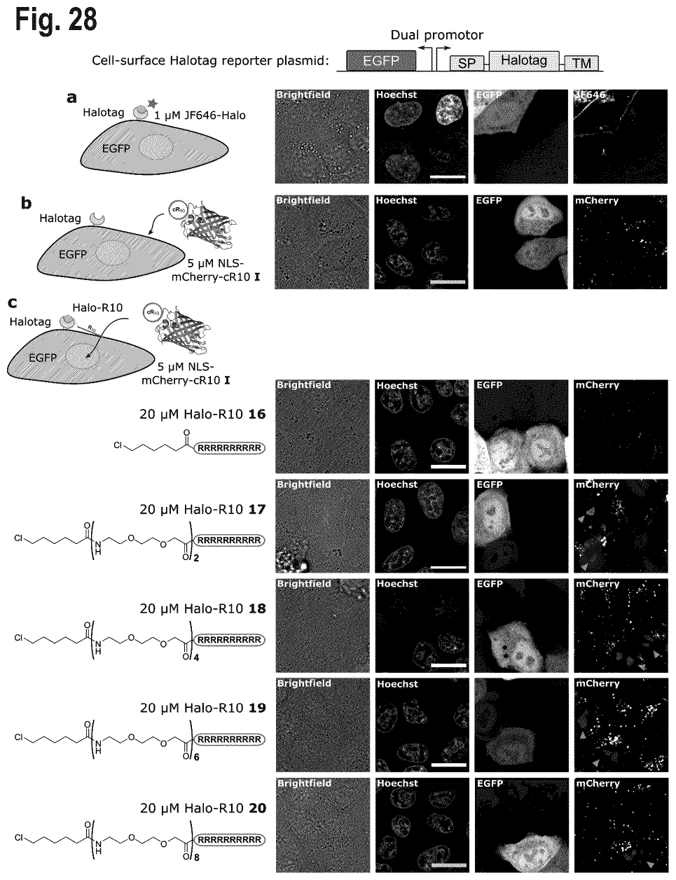

Taken together, the results support that thiol-reactive CPPs increase cellular delivery of cargoes through the covalent linking of peptides to the cell membrane. To elaborate this further, the inventors generated a plasmid that would lead to expression of an EGFP reporter inside transfected cells along with a Halotag (England, C. G., Luo, H. & Cai, W. HaloTag technology: a versatile platform for biomedical applications. Bioconjug Chem 26, 975-986, doi:10.1021/acs.bioconjchem.5b00191 (2015)) on the cell surface (simplified plasmid map in FIG. 3c, validation of the plasmid and additional controls in FIG. 28). The inventors then synthesized a series of chloroalkane-modified R10 peptides 16-20 with varying polyethylene glycol linker lengths for covalent labeling of the expressed halotag. The inventors then added the “Halo-R10” peptides 16-20 to the cells together with NLS-mCherry-cR10 I. The inventors observed no nucleolar staining for the peptide with no ethylene glycol between the chloroalkane and the R10 peptide (FIG. 28c). For all peptides containing a linker, the inventors saw nucleolar mCherry staining in transfected cells (FIG. 3c, brighter arrows, cells showing EGFP signal and FIG. 28c peptides 18-20), but not in untransfected cells. However, all cells showed significant endosomal uptake and 20 μM of the Halo-R10 were needed to achieve nucleolar staining. This lower efficiency may be due to the limited amount and reactivity of the halotag protein on the cell surface.

### Example 4: Cargo Delivery Using TNB-R10 is Robust in Various Cell Lines and Accepts Recombinant CPPs and Cysteine-Containing Proteins

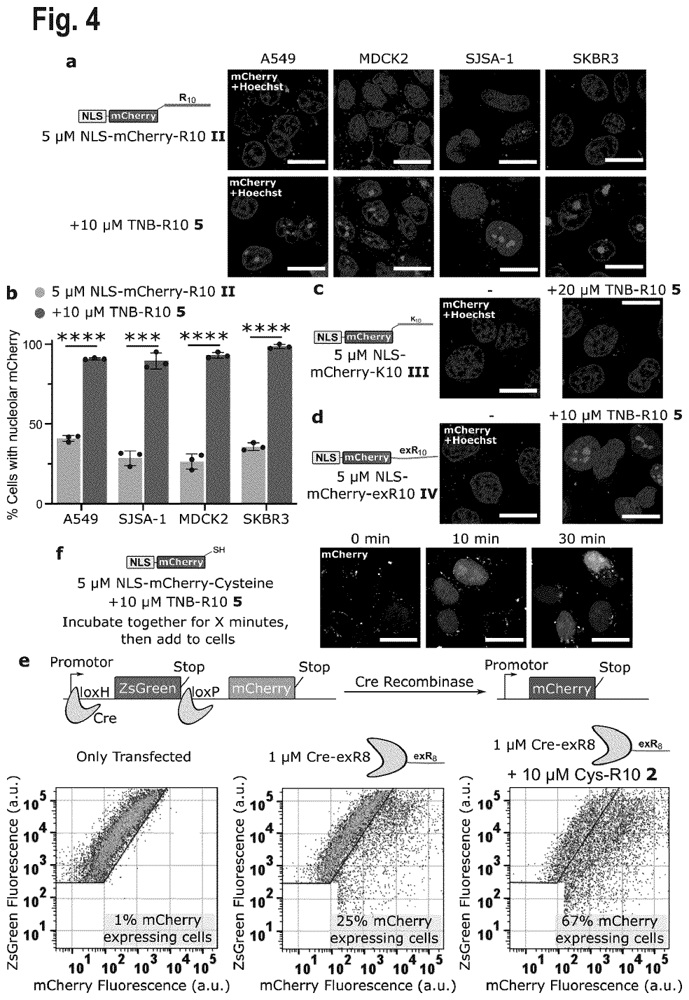

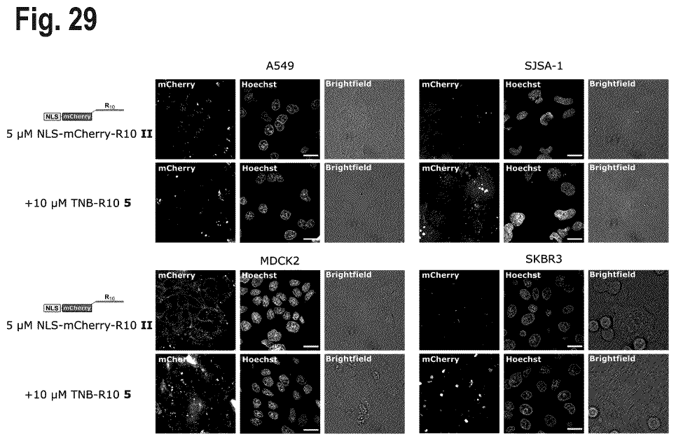

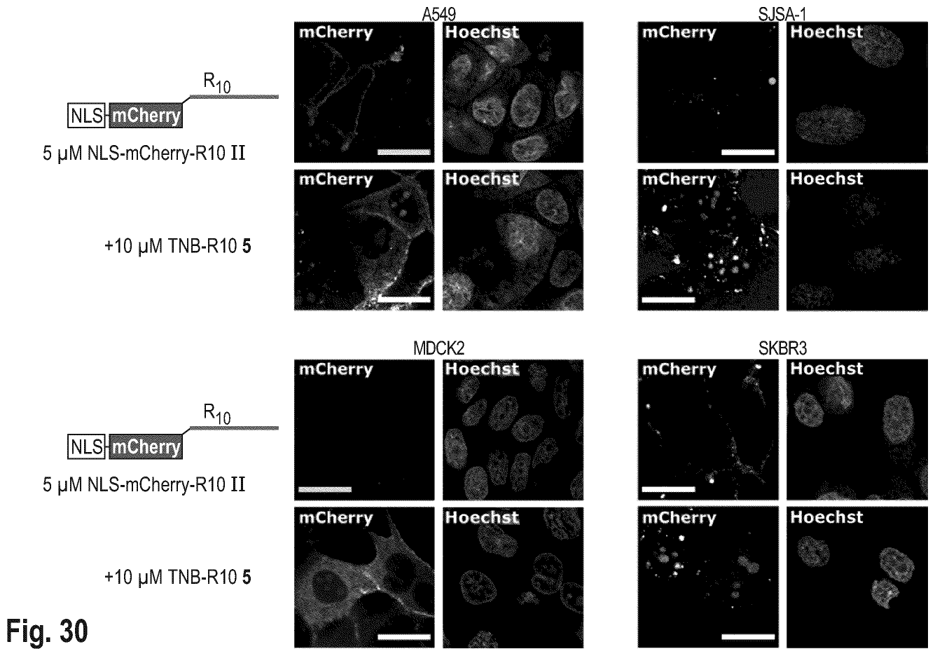

To test if membrane transduction of proteins can be achieved in different cell lines, the inventors tested our protocol in four additional cancer cell lines from different tissue origin (A549, MDCK2, SJSA-1 and SKBR3). We recorded uptake and nucleolar staining of 5 μM mCherry-R10 II in presence of 10 μM TNB-R10 5 in all tested cell lines at 37° C. (FIG. 4a-b and FIG. 29) and 4° C. (FIG. 30).

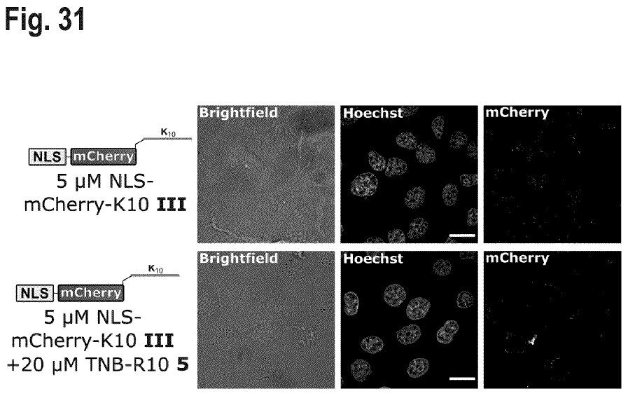

Most of our findings point to an energy-independent mode of uptake, but to probe whether TNB-R10 5 can also lead to endosomal leakage of an entrapped cargo, the inventors used peptide 5 on cells in combination with an mCherry variant (NLS-mCherry-K10 Ilil) that had been modified with a K10 peptide (via maleimide chemistry, see FIG. 8). The K10 peptide should be sufficient to bring the protein in contact to the cell membrane and deliver it into endosomes through active transport, but should not transduce, as lysine-rich peptides do not share the crucial characteristics of arginine-rich peptides in membrane interaction (Robison, A. D. et al. Polyarginine Interacts More Strongly and Cooperatively than Polylysine with Phospholipid Bilayers. J Phys Chem B 120, 9287-9296, doi:10.1021/acs.jpcb.6b05604 (2016). Tesei, G. et al. Self-association of a highly charged arginine-rich cell-penetrating peptide. Proc Natl Acad Sci USA 114, 11428-11433, doi:10.1073/pnas.1712078114 (2017)). Indeed, incubation of 5 μM mCherry-K10 alone or in combination with 20 μM peptide 5 did not lead to nuclear localization but only punctate fluorescence indicative of endosomal entrapment (FIG. 4c and FIG. 31).

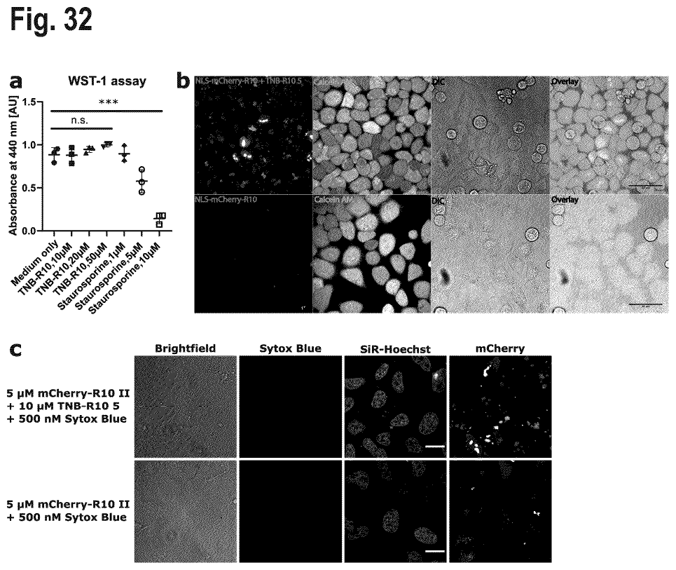

Additionally, peptide 5 showed no signs of cytotoxicity or decreased cell viability up to 50 μM peptide (FIG. 32a). Cells that took up mCherry with or without 5 showed staining with Calcein AM, a cell-permeable caged fluorophore that shows intracellular fluorescence in cells with active metabolism (FIG. 32b). Performing the uptake in presence of the dead cell stain Sytox blue did also not lead to nuclear staining with the dye (FIG. 32c). Taken together, these experiments suggest that TNB-R10 peptide 5 does not lead to disruption of the endosomal or cellular membrane.

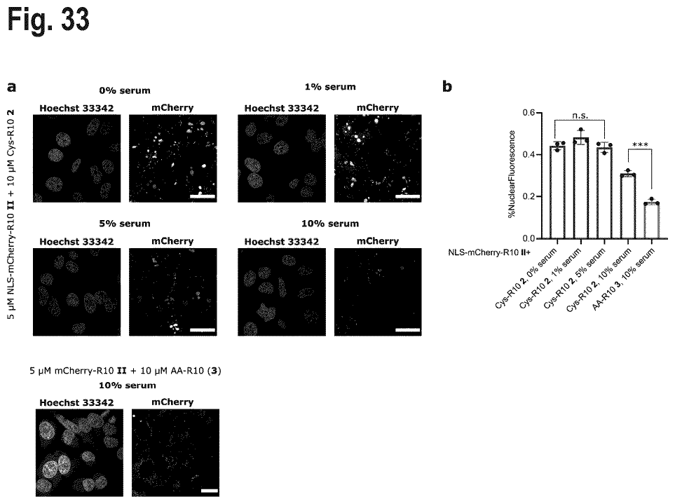

Methods of cargo delivery that rely on endosomal escape are often susceptible to the presence of serum, as they require effective endocytosis of both the cargo and the endosomolytic agent (Erazo-Oliveras, A. et al. Protein delivery into live cells by incubation with an endosomolytic agent. Nat Methods 11, 861-867, doi:10.1038/nmeth.2998 (2014)). The inventors hypothesized that peptide 5 would likely react with thiols in the serum, but the co-delivery with 2 (Cys-R10) should still function. While the presence of serum did lead to reduced efficiency at 10% serum, 5% serum or lower had a negligible effect on uptake (FIGS. 33a, b). Interestingly, even in presence of serum the thiol-containing peptide 2 performed significantly better than the alkylated variant 3 (FIG. 33a, b). Reduction in efficiency in presence of large amounts of serum may be due to the thiols in serum or the unspecific binding of CPPs to serum proteins (Lattig-Tunnemann, G. et al. Backbone rigidity and static presentation of guanidinium groups increases cellular uptake of arginine-rich cell-penetrating peptides. Nat Commun 2, 453, doi:10.1038/ncomms1459 (2011)).

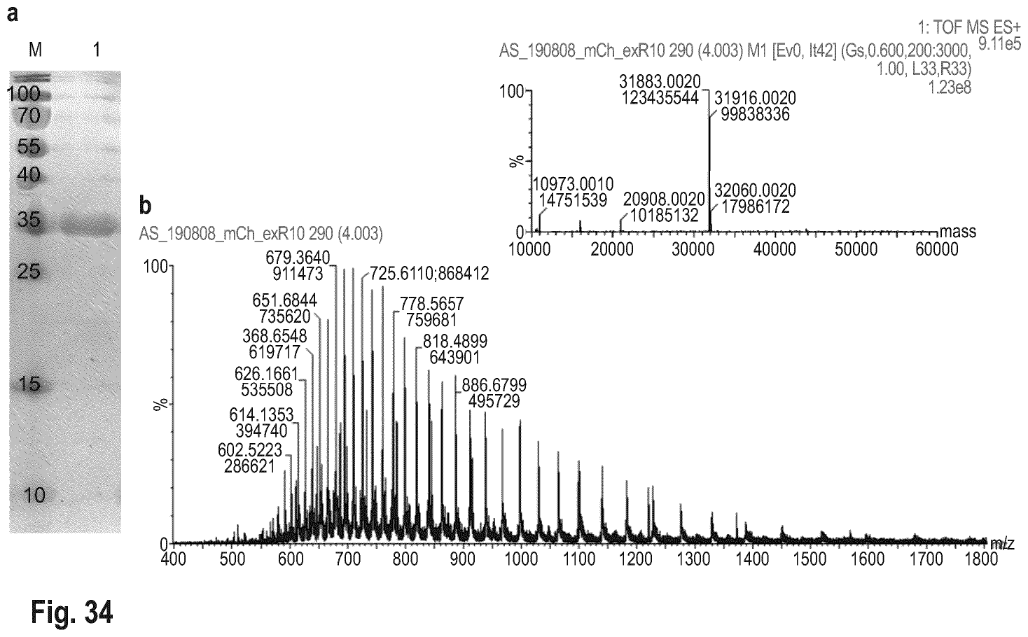

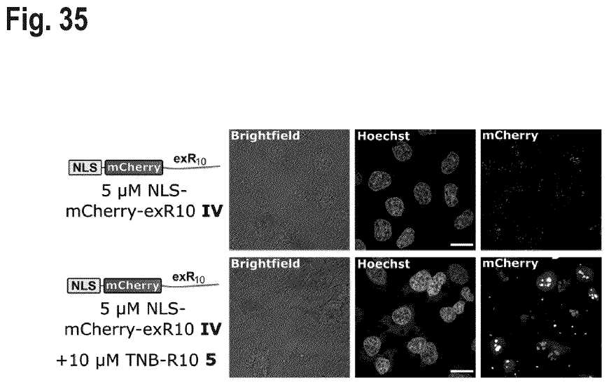

The inventors also tested if recombinantly expressed CPP-fusion proteins can be delivered, as these require much less equipment and effort to produce. To test this, the inventors expressed and purified mCherry with a C-terminal R10 peptide (NLS-mCherry-exR10 IV, characterization in FIG. 34). mCherry-exR10 showed similar behavior to the semi-synthetic variant, showing predominantly endosomal uptake alone at a low, 5 μM concentration, which can be efficiently rescued by addition of peptide 5 (FIG. 4d and FIG. 35). This mCherry variant does not contain any cysteines, meaning it cannot form a disulfide with 5, thus demonstrating that the recombinant polyarginine is enough for co-transport.

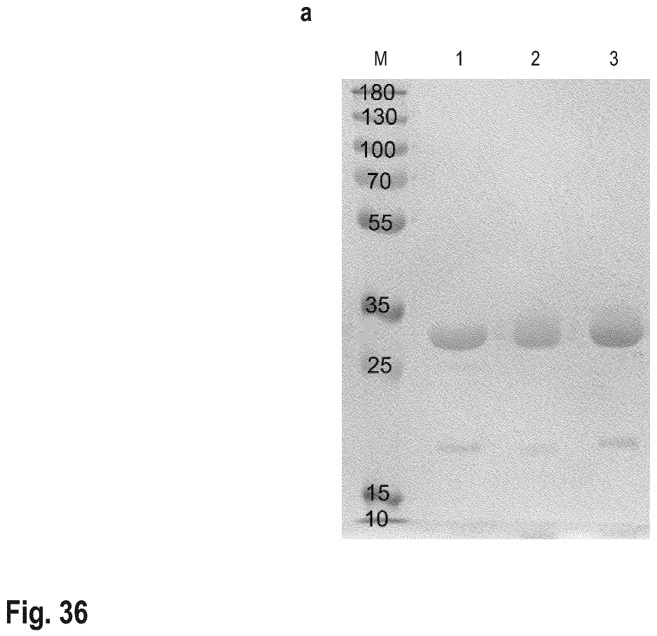

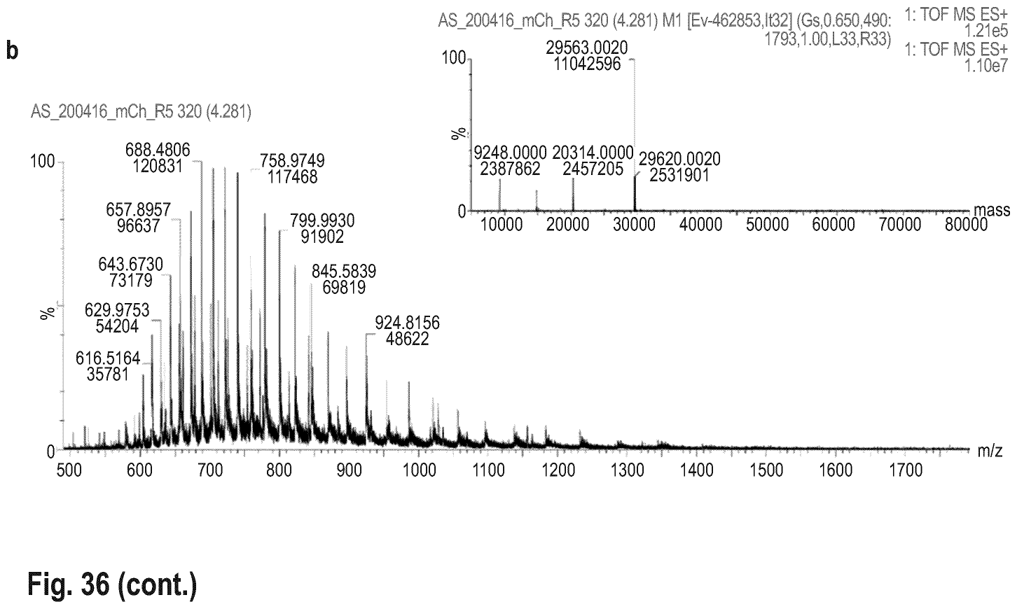

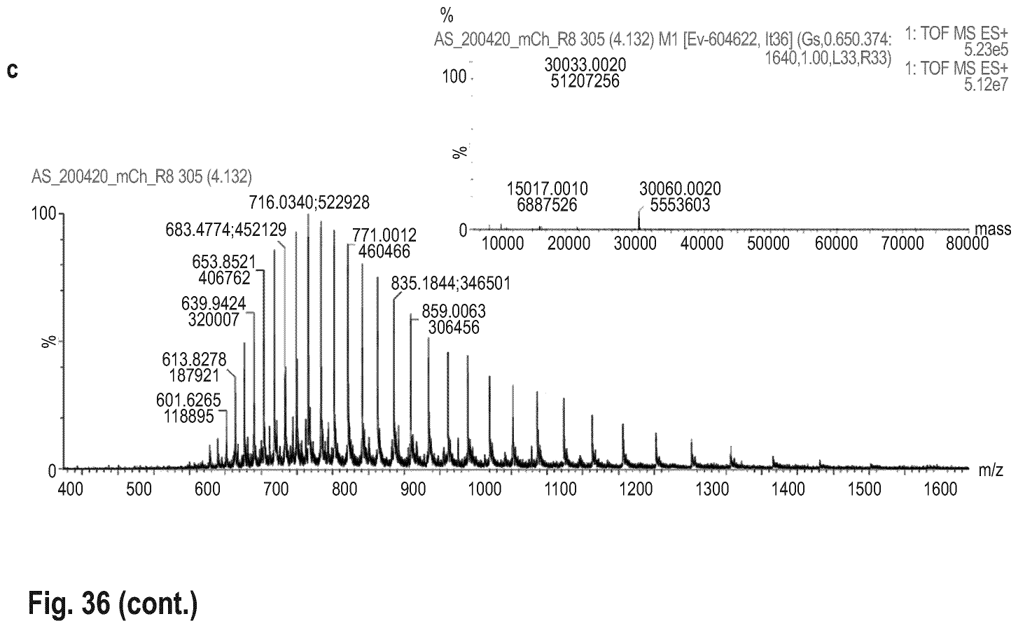

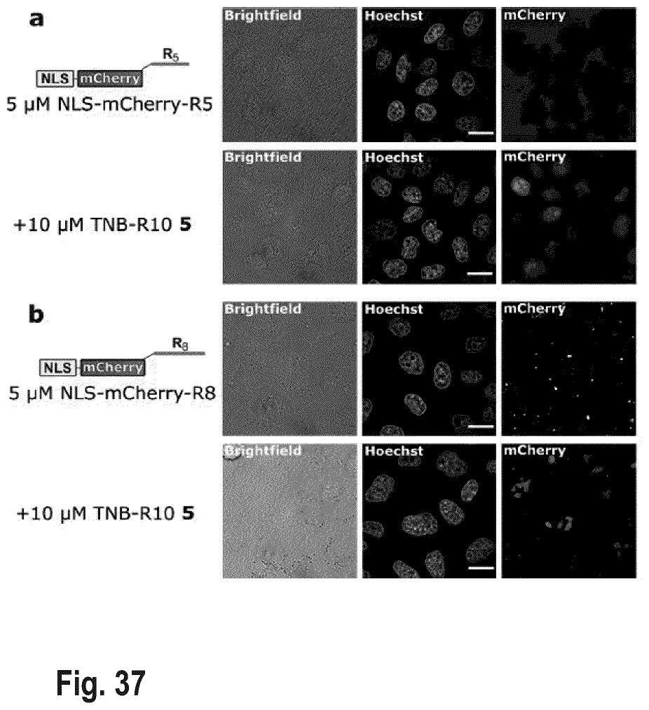

In the same vein, the inventors also made mCherry variants modified with R5 and R8 peptides and co-delivered them into cells with TNB-R10 5 (FIGS. 36-37). The R8 peptide showed comparable results to the R10 peptide, while the inventors saw a clear drop in efficiency with the R5 peptide.

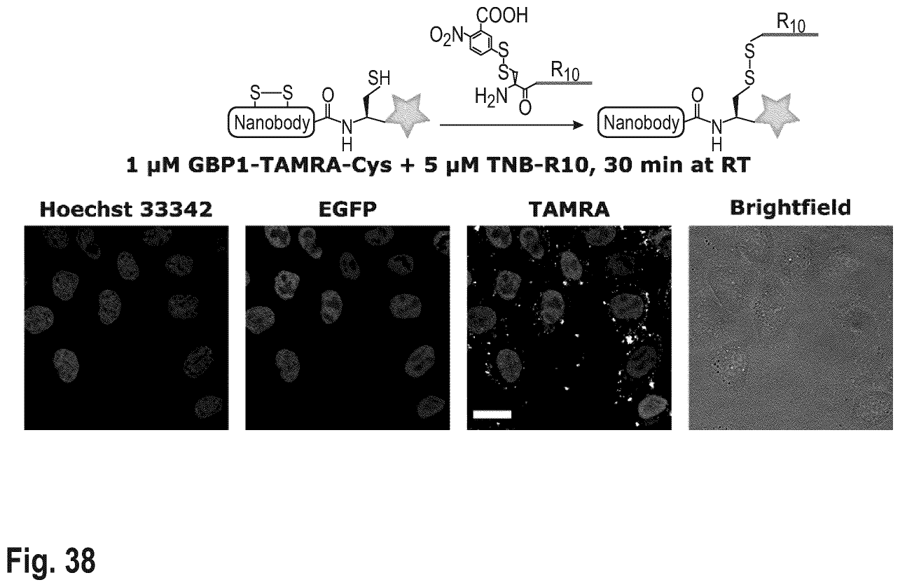

Since peptide 5 can readily react with thiols, the inventors tested if applying a labeling mixture of a protein containing a free thiol and CPP-additive 5 without intermediate work-up would facilitate the uptake protocol. Upon mixing 5 μM mCherry with a free cysteine were mixed with 15 μM peptide 5, cell-permeability of the mCherry was already visible after ten minutes, which further improved after a 30-minute incubation (FIG. 4e). This same protocol also worked well for a cysteine containing fluorescent nanobody (FIG. 38). It should be noted that here, the CPP is linked to both protein cargoes via an intracellularly cleavable disulfide, which results in broad nuclear staining (because of a nuclear antigen for the nanobody) as opposed to the nucleolar localization observed before (Schneider, A. F. L., Wallabregue, A. L. D., Franz, L. & Hackenberger, C. P. R. Targeted Subcellular Protein Delivery Using Cleavable Cyclic Cell-Penetrating Peptides. Bioconjug Chem 30, 400-404, doi:10.1021/acs.bioconjchem.8b00855 (2019). Herce, H. D. et al. Cell-permeable nanobodies for targeted immunolabelling and antigen manipulation in living cells. Nat Chem 9, 762-771, doi:10.1038/nchem.2811 (2017)).

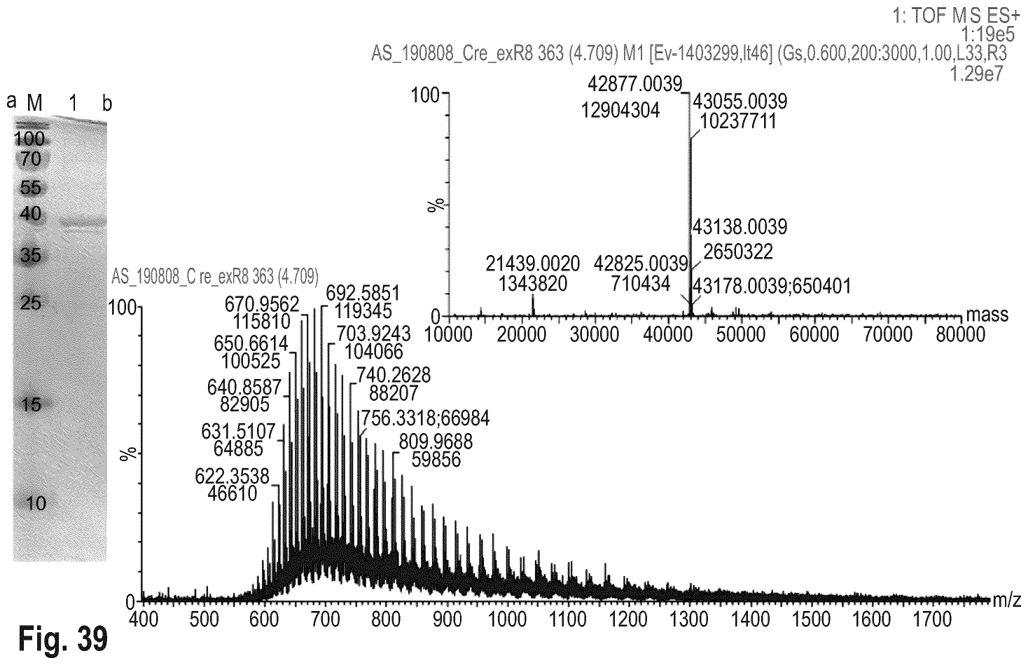

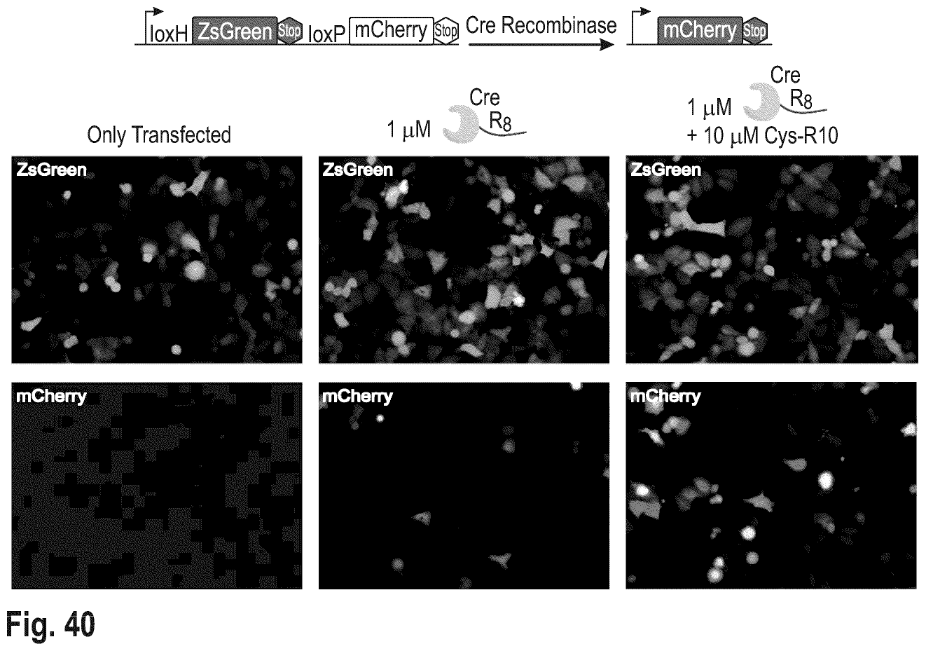

To challenge the delivery protocol, the inventors expressed and purified Cre recombinase fused to a C-terminal R8 peptide (Cre-exR8, characterization in FIG. 39). Fusions of Cre recombinase with the arginine-rich HIV TAT peptide have been previously reported to aid in cell uptake (Peitz, M., Pfannkuche, K., Rajewsky, K. & Edenhofer, F. Ability of the hydrophobic FGF and basic TAT peptides to promote cellular uptake of recombinant Cre recombinase: a tool for efficient genetic engineering of mammalian genomes. Proc Natl Acad Sci USA 99, 4489-4494, doi:10.1073/pnas.032068699 (2002)). The inventors transfected HeLa cells with a Cre activity reporter plasmid (Cre Stoplight 2.4 (Yang, Y. S. & Hughes, T. E. Cre stoplight: a red/green fluorescent reporter of Cre recombinase expression in living cells. Biotechniques 31, 1036, 1038, 1040-1031, doi:10.2144/01315st03 (2001)) that leads to a change in fluorescence from green to red when the enzyme is present within cells (FIG. 4f). The inventors then treated cells with 1 μM Cre-exR8 alone or with added 10 μM Cys-R10 2 in presence of 5% serum and then monitored expression of the reporter gene by flow cytometry and microscopy. As expected, the addition of peptide led to a strong increase in expression of the Cre reporter, indicating successful delivery of active Cre into the nucleus (FIG. 4f and microscopy in FIG. 40).

### Example 5: The TNB-R10 CPP-Additive Allows Cytosolic Delivery of Functional IgG-Antibodies

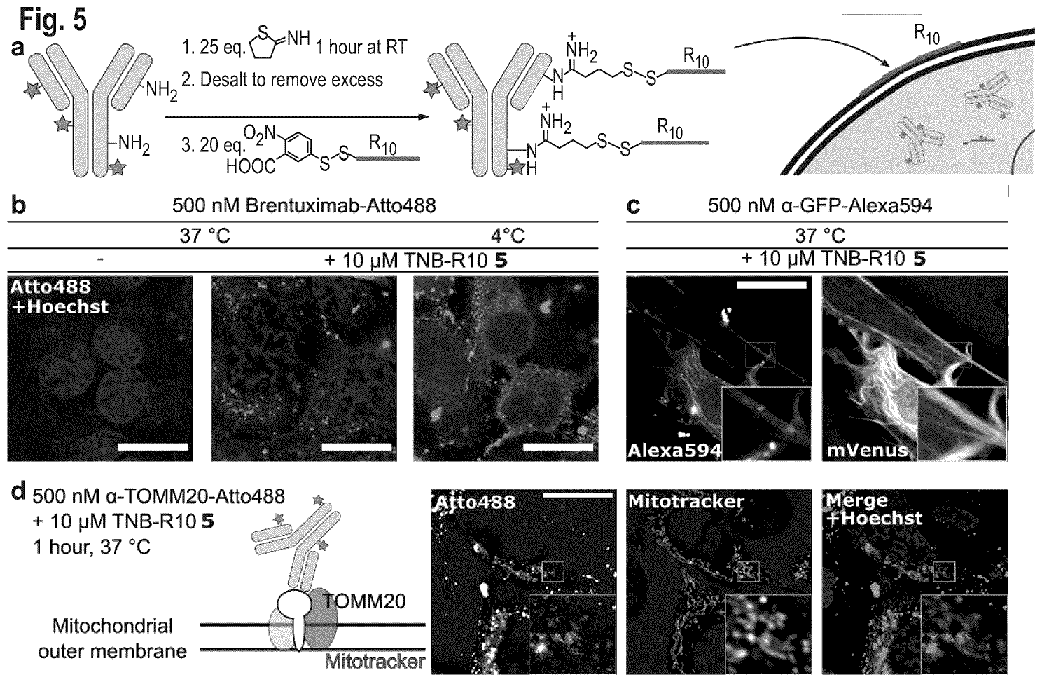

Antibodies are exceptionally useful proteins in molecular biology and pharmacology, targeting most of the human proteome. Nevertheless, the cellular delivery of full-length antibodies is particularly challenging due to the complex and quite large architecture with a molecular weight of 150 kDa and a length of 15 nanometers, approximately. Some methods to deliver full length antibodies into cells already exist, although they mostly rely on endosomal escape (Erazo-Oliveras, A. et al. Protein delivery into live cells by incubation with an endosomolytic agent. Nat Methods 11, 861-867, doi:10.1038/nmeth.2998 (2014). Akishiba, M. et al. Cytosolic antibody delivery by lipid-sensitive endosomolytic peptide. Nature Chemistry, doi:10.1038/nchem.2779 (2017)). To test whether it is possible to deliver a full-length IgG antibody into cells at 4° C., the inventors first used the fluorescently labeled therapeutic antibody Brentuximab. Thiolation was performed with 2-iminothiolane (Jue, R., Lambert, J. M., Pierce, L. R. & Traut, R. R. Addition of sulfhydryl groups to Escherichia coli ribosomes by protein modification with 2-iminothiolane (methyl 4-mercaptobutyrimidate). Biochemistry 17, 5399-5406, doi:10.1021/bi00618a013 (1978)). As before, by addition of peptide 5, the antibody can be modified with the cell-penetrating peptide via a disulfide bond, while the excess cell-penetrating peptide should simultaneously aid in the cellular uptake (FIG. 5a). Indeed, treatment of cells with the antibody led to cellular delivery at 37° C., but not in the absence of CPP-additive 5 (FIG. 5b). A fluorescent signal could not be observed in the nucleus (counterstained with Hoechst), which is likely due to the size of the antibody excluding it from permeation through nuclear pores. Even at 4° C., antibody uptake could also be observed in most cells demonstrating energy-independent membrane transduction of an antibody (FIG. 5b).

To validate that the delivered antibodies are still intact and functional after cellular uptake, the inventors subsequently tested two additional commercial antibodies in our protocols. Here, the inventors first transfected a plasmid encoding a GFP mutant (Lifeact-mVenus) into HeLa cells and functionalized a fluorescently labelled (Alexa 594) anti-GFP antibody as before. Incubation of cells with the antibody showed uptake of the antibody into cells and colocalization of the antibody and mVenus signals within the cell (FIG. 5c, pearson correlation coefficient (PCC) in shown inset=0.80).

Finally, the inventors also tested an antibody against the endogenous mitochondrial receptor TOMM20. As with Brentuximab, the inventors first fluorescently labelled the antibody, followed by thiolation and mixing with CPP-additive 5. Incubation of HeLa cells with the mixture and a mitochondrial marker (MitoTracker Red CMXRos) led to noticeable endosomal entrapment, but also visible colocalization of the two components (FIG. 5d, PCC in shown inset=0.58).

### Example 6: Addition of a Hydrophobic Amino Acid Moiety to CPP Additive Increases Delivery of Cargoes Through the Membrane

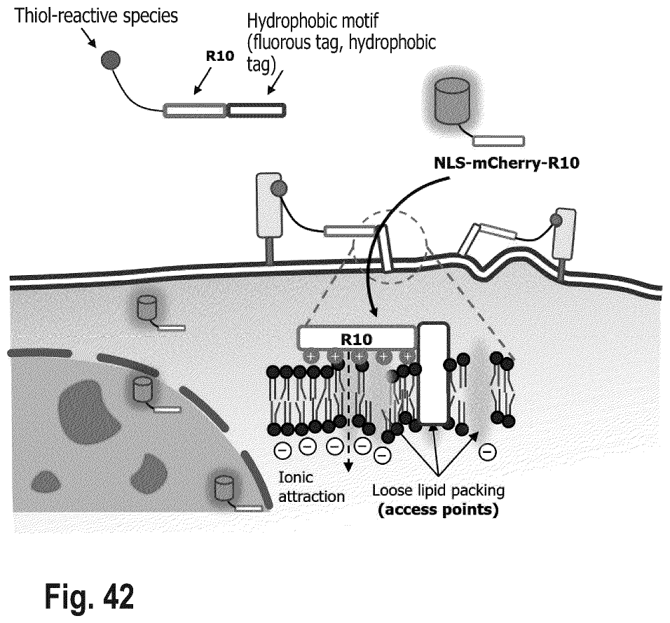

The inventors tested whether integrating a hydrophobic moiety onto CPP-additives may influence the delivery of a cargo through the membrane. It was hypothesized that integrating a hydrophobic moiety may cause membrane lipid packing loosening, which could enhance cellular uptake efficiency, thus resulting in a decrease of the needed application concentrations for both the CPP-additive and the CPP-cargo (see FIG. 42 for a schematic drawing of this concept; and Pujals, S.; Miyamae, H.; Afonin, S.; Murayama, T.; Hirose, H.; Nakase, I.; Taniuchi, K.; Umeda, M.; Sakamoto, K.; S. Ulrich, A.; Futaki, S. Curvature Engineering: Positive Membrane Curvature Induced by Epsin N-Terminal Peptide Boosts Internalization of Octaarginine. ACS Chem. Biol. 2013, 8, 1894-1899; and Murayama, T.; Masuda, T.; Afonin, S.; Kawano, K.; Takatani-Nakase, T.; Ida, H.; Takahashi, Y.; Fukuma, T.; Ulrich, A. S.; Futaki, S. Loosening of Lipid Packing Promotes Oligoarginine Entry into Cells. Angew. Chemie int. Ed. 2017, 56, 7644-7647). In this regard, CPP additives accumulate on the cell membrane and may loosen local membrane lipid packing (Schneider, A. F. L.; Kithil, M.; Cardoso, M. C.; Lehmann, M.; Hackenberger, C. P. R. Cellular Uptake of Large Biomolecules Enabled by Cell-Surface-Reactive Cell-Penetrating Peptide Additives. Nat. Chem. 2021 136 2021, 13, 530-539). At first, a CPP additive comprising a hydrophobic amino acid moiety was tested.

The experiment was carried out with CPP additive Mal-PEG₂-R10-ILFF, and with Mal-PEG₂-R10 as reference. The sequences are shown in the following:

Mal-PEG₂-R10:
Maleimide-PEG-PEG-R-R-R-R-R-R-R-R-R-R-amide
Mal-PEG₂-R10-ILFF:
Maleimide-PEG-PEG-R-R-R-R-R-R-R-R-R-R-G-G-I-L-
F-F-amide.

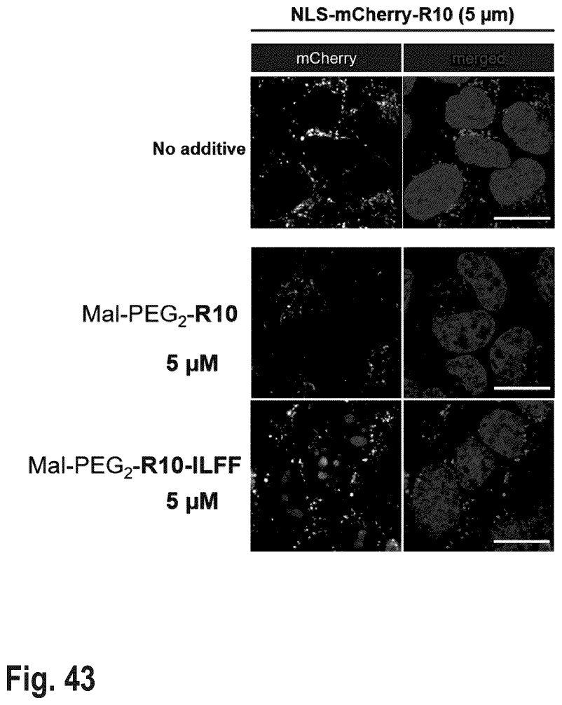

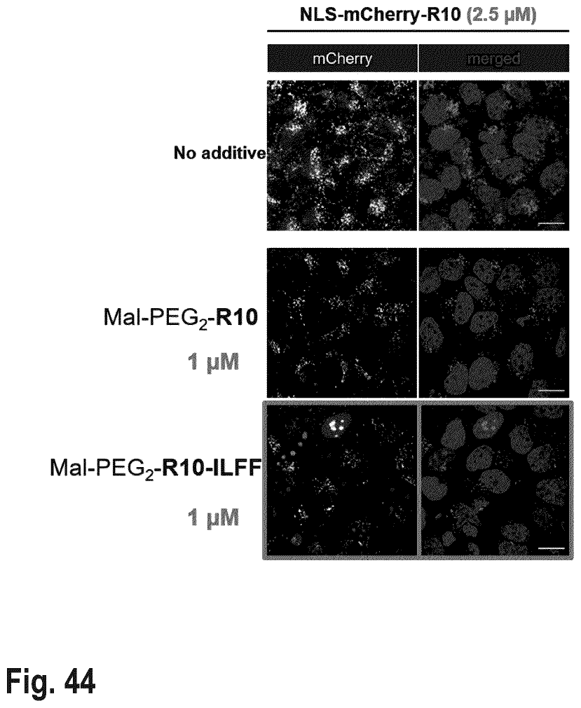

Experimental Procedure: HeLa cells seeded onto 96-well glass-bottom plates were treated with 5 μM or 1 μM Mal-PEG₂-R10 or Mal-PEG₂-R10-ILFF, followed by the addition of NLS-mCherry-R10 (final concentration=5 μM or 2.5 μM). The treated cells were incubated for 1 h at 37° C. with 5% CO₂. After incubation, the cells were washed with phosphate buffered saline (PBS). The nucleus was counterstained using Hoechst 33342. Imaging was performed using Nikon-CSU spinning disk microscopy. Scale bars=20 μm. FIG. 43 shows the spinning disk microscopy images of cells treated with 5 μM of CPP additive, followed by 5 μM NLS-mCherry-R10. FIG. 44 shows the spinning disk microscopy images of cells treated with 1 μM of CPP additive, followed by 2.5 μM NLS-mCherry-R10.
Results: The CPP additives with hydrophobic amino acid moiety showed better nucleolar staining indicating improved cellular uptake of the cargo (FIG. 43). Decreasing the treatment concentrations of both the CPP additive and cargo protein is possible when using the hydrophobic CPP additives (red outlines, FIG. 44). This may result from interaction of hydrophobic amino acid (such as, e.g., Leu, Ile, Phe, and Trp) moieties on the CPP additive with the membrane. Insertion of the side chains between membrane lipids may influence the local packing state of the membrane resulting in loose packing areas where CPP-cargoes could enter more efficiently.

### Example 7: Addition of a Fluorous Tag to CPP Additive Increases Delivery of Cargoes Through the Membrane

The inventors also tested whether a CPP additive comprising a perfluoroalkyl moiety may influence the delivery of a cargo through the membrane. It was hypothesized that fluoroalkyl groups might give rise to fluorophilic self-assembly on the cell membrane, influence hydrophobic interactions with the membrane, and may influence interaction with CPP-cargoes (Chuard, N.; Fujisawa, K.; Morelli, P.; Saarbach, J.; Winssinger, N.; Metrangolo, P.; Resnati, G.; Sakai, N.; Matile, S. Activation of Cell-Penetrating Peptides with lonpair-πInteractions and Fluorophiles. J. Am. Chem. Soc. 2016, 138, 11264-11271).

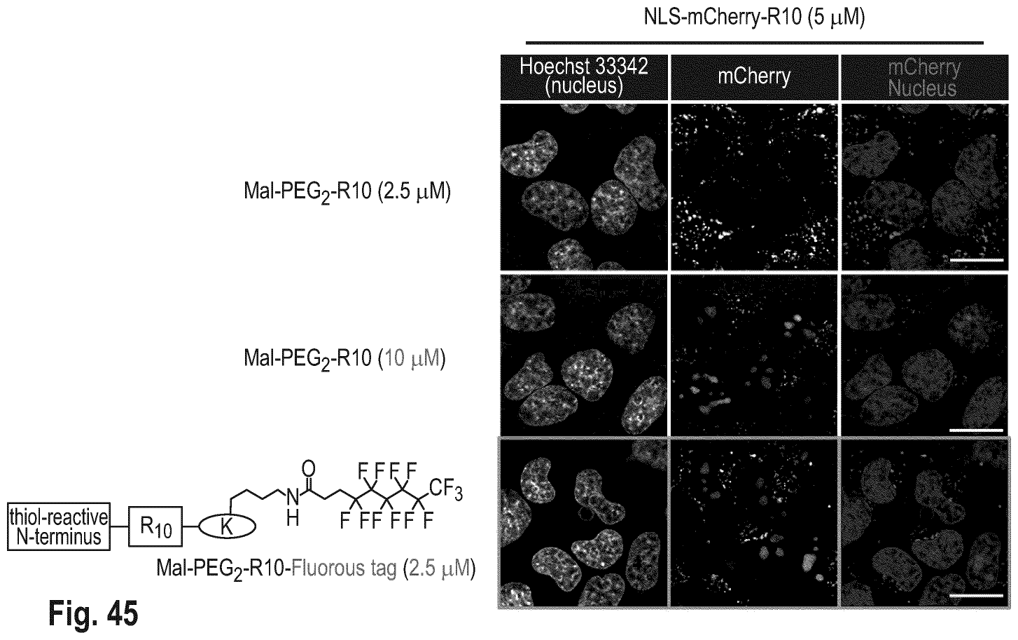

Experimental Procedure: The experiments were carried out with Mal-PEG₂-R10-fluorous tag, and Mal-PEG₂-R10 as reference. The sequence of Mal-PEG₂-R10 is described in Example 6. The structure of Mal-PEG₂-R10-fluorous tag is shown in FIG. 45. HeLa cells seeded onto 96-well glass-bottom plates were treated with 2.5 or 5 μM of Mal-PEG₂-R10 or 2.5 μM or Mal-PEG₂-R10-fluorous tag, followed by the addition of NLS-mCherry-R10 (final concentration=5 μM). The treated cells were incubated for 1 h at 37° C. with 5% CO₂. After incubation, the cells were washed with phosphate buffered saline (PBS). The nucleus was counterstained using Hoechst 33342. Imaging was performed using Nikon-CSU spinning disk microscopy. Scale bars=20 μm. FIG. 45 shows the spinning disk microscopy images of cells treated with Mal-PEG₂-R10 or Mal-PEG₂-R10-fluorous tag.

Results: The results for the Mal-PEG₂-R10 additive are replicable across different experiments. Lowered concentrations (2.5 μM) showed unfavorable uptake of NLS-mCherry. Mal-PEG₂-R10-fluorous tag showed good NLS-mCherry-R10 uptake at low concentration (2.5 μM, green outlines), which is visually similar to treating cells with 10 μM of Mal-PEG₂-R10.

The following examples represent general material and methods in accordance with the present invention.

### Example 8

### Supporting Methods

### General Materials and Methods

### Solvents and Chemicals

Solvents (DMF, DCM) were purchased from Thermo Fisher Scientific (USA). Amino acids, rink amide resin and coupling reagents were purchased from Iris Biotech (Germany). 5(6)-Carboxytetramethylrhodamine (TAMRA) was purchased from Merck (Germany). HATU was purchased from Bachem (Switzerland). DIEA and TFA were purchased from Carl Roth (Germany).

Salts, LB medium, antibiotics and other buffer components were purchased from Carl Roth (Germany).

Mammalian cell culture media and fetal bovine serum were purchased from VWR (USA).

### Analytical UPLC-MS

UPLC-UV traces were obtained on a Waters H-class instrument equipped with a Quaternary Solvent Manager, a Waters autosampler and a Waters TUV detector with an Acquity UPLC-BEH C18 1.7 μm, 2.1×50 mm RP column. The following gradient was used: A=H₂O+0.1% TFA, B=MeCN+0.1% TFA 5-95% B 0-5 min, flow rate 0.6 mL/min. UPLC-UV chromatograms were recorded at 220 nm.

### Preparative HPLC

Preparative HPLC of peptides was done on a Gilson PLC 2020 system using a Nucleodur C18 Htec Spum column (Macherey-Nagel, 100 A, 5 m, 250 mm×32 mm, 30 mL/min). The following gradient was used in all purifications: A=H₂O+0.1% TFA, B=MeCN+0.1% TFA 5% B 0-10 min, 5-50% B 10-60 min, 50-99% 60-80 min.

### High Resolution Mass Spectrometry (HRMS)

High resolution mass spectra were measured on a Xevo G2-XS QTof (Waters) mass spectrometer coupled to an acquity UPLC system running on water and acetonitrile, both with 0.01% formic acid. Protein spectra were devonvoluted using the MaxEnt 1 tool.

### Size Exclusion Chromatography of Proteins

Size exclusion chromatography was done on an AKTA Purifier system (GE Healthcare) on a Superdex S75 increase 16/600 column (GE Healthcare) for all proteins except antibodies, which were purified after fluorescent labelling on a Superose 6 16/600 column (GE Healthcare).

### SDS-PAGE

Proteins were mixed with 4× reducing Laemmli buffer (Bio-Rad) and boiled at 95° C. for 5 minutes before separation on 15% SDS-PAGE gels. In-gel fluorescence was imaged first, followed by Coomassie staining and imaging. Gels were imaged on a ChemiDoc XRS+ system (Bio-Rad).

### Software

Microscopy pictures were processed with ImageJ including the FIJI package. Graphing and statistics were done using Graphpad Prism 8. Flow cytometry data was processed and analyzed using FlowJo.

### Peptide Synthesis

All peptides were synthesized by standard Fluorenylmethoxycarbonyl (Fmoc)-solid-phase peptide synthesis (SPPS) on Rink amide resin (0.05 mmol scale, 0.22 mmol/g).

Amino acid couplings were done using five equivalents of amino acid with five equivalents of HCTU (O-(1H-6-Chlorobenzotriazole-1-yl)-1,1,3,3-tetramethyluronium hexafluorophosphate) and four equivalents of Oxyma (Ethyl cyanohydroxyiminoacetate) with ten equivalents of DIEA (N,N-Diisopropylethylamine) in DMF (Dimethylformamide). Fmoc removal was accomplished by incubating the resin three times for five minutes with a 20% solution of piperidine in DMF.

Cyclization of the cyclic R10 peptides was done by incorporation of a lysine and glutamic acid residue flanking the CPP sequence, orthogonally protected by N-Allyloxycarbonyl (Alloc) and allyl, respectively. The orthogonal protecting groups were removed using palladium tetrakis (Pd(PPh₃)₄(0.1 equivalents) with phenylsilane (25 equivalents) in dry dichloromethane (DCM) for 30 min at ambient temperature under argon atmosphere. To remove the Pd catalyst afterwards, the resin was washed additionally with 0.2 M DIEA in DMF. Cyclization followed with one equivalent of 1-[Bis(dimethylamino)methylene]-1H-1,2,3-triazolo[4,5-b]pyridinium 3-oxid hexafluorophosphate (HATU) and two equivalents of DIEA in DMF for two hours at room temperature.

Arginine was incorporated with Pbf protection, cysteine was incorporated on the N-termini with Boc and Trityl protection. In the K10 peptide, Lysine with Boc protection on the side chain was used.

In the synthesis of the fluorescent peptides bearing a TAMRA- or Cy5-fluorophore on the side-chain of a lysine, or in the synthesis of the peptide bearing a fluorous tag, the linear synthesis was completed first with a lysine that was orthogonally protected with N-methyltrityl (Mtt). After completion of the linear synthesis, Mtt was removed using 2% TFA with 2% TIS in DCM, five times for two minutes and the fluorophore was subsequently coupled with one equivalent of fluorophore and one equivalent of HATU and four equivalents of DIEA. The procedure was used in an analogous manner for coupling with the fluorous tag.

For the synthesis of the peptides with an N-terminal TAMRA fluorophore, the N-terminal Fmoc protection was removed as above and the fluorophore was coupled using one equivalent of the fluorophore with one equivalent of HATU and four equivalents of DIEA.

The linear sequences of all peptides used in this study is found in supplementary table 1, the final structure and analytical data in supplementary FIG. 1.

TABLE 1
Linear sequences of peptides used in this study. PEG* corresponds to two
consecutively coupled units of 8-amino-3,6-dioxaoctanoic acid. Uppercase letters
are L-amino acids while lower case letters are D-amino acids.

| Peptide | Sequence |
|---|---|
| TAMRA-CR10 | TAMRA-K(Alloc)RrRrRrRrRrE(Allyl)-Amide |
| Cys-TAMRA | C-PEG*-K(Mtt)-G-Amide |
| Maleimide-cR10 | Maleimidoacetic acid-PEG*-K(Alloc)RrRrRrRrRrE(Allyl)-Amide |
| Cys-cR10 | C-PEG*-K(Alloc)RrRrRrRrRrE(Allyl)-Amide |
| Maleimide-R10 | Maleimidoacetic acid-PEG*-RRRRRRRRRR-Amide |
| Cys-R10 | C-PEG*-RRRRRRRRRR-Amide |
| Maleimide-K10 | Maleimidoacetic acid-PEG*-KKKKKKKKKK-Amide |
| Cys-R10-TAMRA | C-PEG*-RRRRRRRRRR-K(Mtt)-Amide |
| TAMRA-R5-Cys-R5 | RRRRRCRRRRR-Amide |
| TAMRA-R10-Cys | RRRRRRRRRRC-Amide |
| Maleimide-R10- | Maleimidoacetic acid-PEG*-RRRRRRRRRRK(Mtt)-Amide |
| Cy5/Biotin | |
| Halo-R10 | 6-chlorohexanoic acid-PEG*-RRRRRRRRRR-Amide |
| Maleimide-R5 | Maleimidoacetic acid-PEG*-RRRRR-Amide |
| Maleimide-R8 | Maleimidoacetic acid-PEG*-RRRRRRRR-Amide |
| Mal-PEG₂-R10 | Maleimidoacetic acid-PEG*-RRRRRRRRRR-Amide |
| Mal-PEG₂-R10-ILFF | Maleimidoacetic acid-PEG*-RRRRRRRRRRGGILFF-Amide |
| Mal-PEG₂-R10- | Maleimidoacetic acid-PEG*-RRRRRRRRRRK(Mtt)-Amide |
| fluorous tag | |

For the synthesis of the alkylated peptides in which the cysteine is modified with iodoacetamide, the cysteine equivalent was taken up in water at a 5 mM concentration and 5 equivalents of iodoacetamide were added for 1 hour at RT. The resulting peptide was immediately purified using reverse phase HPLC to prevent overalkylation. The di-R10 dimer was generated by incubating the Cys-R10 peptide in oxygenated 5 mM HEPES buffer at pH 7.5 for 3 days at room temperature. The Ellman's reagent (thionitrobenzoic acid, TNB) peptides were generated by reacting the cysteine variants at a 5 mM concentration with 10 equivalents of Ellman's reagent (5,5′-dithiobis-(2-nitrobenzoic acid)) and the resulting peptides were purified by reverse phase HPLC.

### Functionalized Decaarginine Synthesis

Peptides were synthesized by standard Fluorenylmethoxycarbonyl (Fmoc)-solid-phase peptide synthesis (SPPS) on Rink amide resin (0.05 mmol scale, 0.22 mmol/g). Fmoc-L-Arginine(Pbf)-OH was coupled using five equivalents of the amino acid with five equivalents of HCTU (O-(1H-6-Chlorobenzotriazole-1-yl)-1,1,3,3-tetramethyluronium hexafluorophosphate) and four equivalents of Oxyma (Ethyl cyanohydroxyiminoacetate) with ten equivalents of DIEA (N,N-Diisopropylethylamine) in DMF (Dimethylformamide) for 1 hour at room temperature under agitation.

Fmoc removal was accomplished by incubating the resin three times for five minutes with a 20% solution of piperidine in DMF. Immediately N-terminally to the 10 arginine residues a linker was coupled (8-(9-Fluorenylmethyloxycarbonyl-amino)-3,6-dioxaoctanoic acid) sequentially two times with the same conditions as the arginine amino acid.

For the maleimide- and halotag-modified peptides, the corresponding building block (2-maleimidoacetic acid or 6-chlorohexanoic acid, respectively) were coupled onto the N-terminus of the linker with five equivalents of the acid, four equivalents of HCTU and ten equivalents of DIEA. Cleavage from the solid support was accomplished using a cocktail consisting of 95% Trifluoroacetic acid (TFA), 2.5% Triisopropylsilane (TIS) and 2.5% water. The peptides were then purified by reverse-phase high pressure liquid chromatography (HPLC).

For the TNB-modified cysteine peptide (“TNB” means thionitrobenzoic acid), Boc-L-Cysteine(Trityl)-OH was coupled onto the N-terminus of the linker as with the arginine above. Cleavage from the solid support was accomplished using a cocktail consisting of 95% TFA, 2.5% TIS and 2.5% water. The peptide was then purified via HPLC. The purified lyophilizate was dissolved in water and treated with 10 equivalents of Ellman's reagent (5,5-dithio-bis-(2-nitrobenzoic acid)) dissolved in acetonitrile. The resulting solution was incubated for 10 minutes at room temperature and the target peptide was purified using HPLC.

### Protein-CPP Conjugation

To conjugate the maleimide-functionalized R10, cR10 and K10 peptides to the thiol containing proteins, the proteins were diluted to 50 μM concentration in 5 mM HEPES at pH 7.5, 140 mM NaCl, 2.5 mM KCL, 5 mM Glycin. 5 equivalents of the maleimide-peptide were added, and the solution was incubated overnight at room temperature. Excess cell-penetrating peptide was removed by desalting in a spin column.

For the in situ CPP conjugation and cell uptake, proteins were diluted to 5 or 25 μM in HEPES buffer (5 mM HEPES at pH 7.5, 140 mM NaCl, 2.5 mM KCL, 5 mM Glycin) and 25 or 75 μM TNB-R10 (for the nanobody and mCherry, respectively) were added for the indicated times. The proteins were then diluted to 1 or 5 μM with DMEM and immediately used in cell experiments.

### Cellular Uptake Experiments

Cell culturing is described in the supplementary methods, along with a list of cell lines used in this study. For microscopy experiments, 20′000 cells (10′000 in the case of the GFP-PCNA HeLa Kyoto cell line⁷) were seeded into the wells of a 96-well glass bottom plate. The cells were left to adhere and grow for 24 hours at 37° C. with 5% CO₂. For 37° C. experiments, the cells were washed once with DMEM before addition of the protein samples in DMEM. The cells were incubated for 1 hour at 37° C. The cells were then washed three times with DMEM with 10% fetal bovine serum (FBS). Cells were generally imaged live with incubation at 37° C. and 5% CO₂. For the quantitative microscopy experiments, the cells were fixed using 4% PFA in PBS for 30 minutes at room temperature after washing.

For 4° C. experiments, the cells were pre-chilled at 4° C. for 1 hour. The cells were then washed with cold DMEM and the proteins were added in cold DMEM to the cells. The cells were incubated at 4° C. for 1 hour. Afterwards, the cells were washed thrice with cold DMEM with 10% FBS, before fixation with 4% PFA in PBS for 30 minutes at room temperature.

For uptake experiments with live data acquisition, cells on a 96-well plate (seeded as above) were placed into the microscope covered with 100 μl DMEM containing Hoechst stain. The nuclear stain was used to find the center of the nuclei. The autofocus was then turned on and 100 μl of the peptide solution at twice the final concentration were added to the well (to give the final concentration on the cells). Recording of images was started 30 seconds after addition of the peptide solution.

For the experiment with the anti-GFP antibody, cells were seeded as above and after 24 hours the cells were transfected with the GFP-mutant-plasmid (Lifeact-mVenus), using Lipofectamine 2000. The cells were then incubated for another 24 hours before treatment with the CPP-conjugate. For the TOMM20 antibody uptake, 200 nM MitoTracker were added to the antibody-CPP mixture before addition to the cells.

### Microscopy

Confocal microscopy images were acquired on a Nikon-CSU spinning disc microscope with an CSU—X1 (Andor) and a live cell incubation chamber (OKOlab). All images shown in this work were acquired using a PlanApo 60×NA 1.4 oil objective (Nikon) and an EMCCD (AU888, Andor). Brightfield images were acquired along with fluorescence images. Standard laser, a quad Dicroic (400-410,486-491, 560-570, 633-647, AHF) and Emission filters were used in the acquisition of confocal fluorescence images (BFP (Hoechst 33342), ex.: 405 nm em.:450/50: GFP (Atto488, mVenus), ex.: 488 em.:525/50, RFP (TAMRA, mCherry, Alexa 594, MitoTracker Red CMXRos), ex.: 561 em.:600/50 nm and iRFP (Cy5, SiR-Hoechst), ex.: 640 em.:685/50 nm. The microscopy images of cells treated with Cre recombinase were acquired on a Nikon Eclipse Ti2 epifluorescence microscope using the GFP and RFP filter sets. The microscopy pictures of the anti-TOMM20 antibody uptake were taken using an additional 1.5× optical magnification.

Quantification of cellular uptake was done using a script for FIJI, see section “Quantification Script”. Briefly, the Hoechst stain was used as a mask for the nuclei. The red fluorescence channel was background subtracted and the red fluorescence within the nuclear mask and outside of it was quantified. Nuclear fluorescence was either normalized to the nuclear area (absolute fluorescence graph in FIG. 2) or to the sum of nuclear and outside fluorescence (relative fluorescence graph in SI FIG. 6). Pearson's correlation coefficient was calculated using the Coloc2 tool in Fiji.

### Antibody Modification and Uptake

A list of antibodies can be found in table 2. Antibodies were used at a 0.5 mg/mL concentration (˜6.7 μM). The anti-GFP antibody was purchased as a fluorophore conjugate. The Brentuximab and anti-TOMM20 antibody were first labelled fluorescently using 8 equivalents of NHS-Atto488 (Atto-Tec GmbH) for 1 hour at room temperature before purification via gel filtration on a superpose 6 column. All antibodies were then modified with 25 equivalents of Traut's reagent (2-Iminothiolane) for 1 hour at room temperature. Excess reagent was removed using a desalting column. Then, 20 equivalents of TNB-R10 were added immediately and the antibodies were incubated in the fridge until use. The antibodies were diluted to 500 nM in DMEM before cell experiments.

TABLE 2
Antibodies used in this study.

| Antibody | Source |
|---|---|
| Brentuximab | Ludwig-Maximilians-Universität |
| | (LMU) München, Germany |
| Alexa Fluor 594 anti-GFP | BioLegend (USA) |
| Antibody, Clone 1GFP63 | |
| Anti-Tom20/Tomm20, clone 2F8.1 | Merck (Germany) |

### Cloning, Protein Expression and Purification

### GBP1 Nanobody:

The GBP1 nanobody was expressed and labelled through expressed protein ligation (EPL), similarly to a previously published protocol (Herce, H. D. et al. Cell-permeable nanobodies for targeted immunolabelling and antigen manipulation in living cells. Nat Chem 9, 762-771, doi:10.1038/nchem.2811 (2017)). Briefly, the nanobody was expressed in BL21 DE3 cells as a fusion protein with the DnaE intein and a chitin binding domain (pTXB1 vector system).

Protein sequence, which is included in the
sequence listing as SEQ ID NO: 1 (Nanobody
sequence after intein cleavage underlined):
MADVQLVESGGALVQPGGSLRLSCAASGFPVNRYSMRWYRQAPGKEREW
VAGMSSAGDRSSYEDSVKGRFTISRDDARNTVYLQMNSLKPEDTAVYYC
NVNVGFEYWGQGTQVTVSSAAACITGDALVALPEGESVRIADIVPGARP
NSDNAIDLKVLDRHGNPVLADRLFHSGEHPVYTVRTVEGLRVTGTANHP
LLCLVDVAGVPTLLWKLIDEIKPGDYAVIQRSAFSVDCAGFARGKPEFA
PTTYTVGVPGLVRFLEAHHRDPDAQAIADELTDGRFYYAKVASVTDAGV
QPVYSLRVDTADHAFITNGFVSHATGLTGLNSGLTTNPGVSAWQVNTAY
TAGQLVTYNGKTYKCLQPHTSLAGWEPSNVPALWQLQ*
The ″*″ symbolizes the stop codon.

For the expression, T7 express cells (New England Biolabs) were transformed with the plasmid and grown overnight at 37° C. in 5 mL of LB medium with 100 μg/mL ampicillin. The next day, the expression culture in 250 mL LB medium with ampicillin was inoculated with 1 mL of the starter culture. The culture was incubated at 37° C. until it reached an OD600 of 0.6. Protein expression was then induced using 1 mM IPTG and the culture was incubated for 16 hours at 18° C. Cells were collected by centrifugation at 4000×g for 15 minutes. The cells were washed once in PBS, then resuspended in lysis buffer (20 mM Tris-HCl, pH 8.5, 500 mM NaCl, 1 mM EDTA, 0.1% Triton X-100, 100 μg/mL lysozyme and 25 μg/mL DNAse 1), lysed using sonication (3×2 min, 30% Amplitude), followed by debris centrifugation at 25′000×g for 30 min.

For the purification, the clear lysate was loaded on 2 mL of chitin-agarose, equilibrated in EPL buffer (20 mM Tris-HCl pH 8.5, 500 mM NaCl). The agarose beads were washed with 20 column volumes of EPL buffer. Then, a TAMRA- and cysteine-functionalized peptide (see SI FIG. 1b) was coupled to the C-terminus of the protein using EPL. For this, the protein was reacted on the chitin column with 1 mM peptide in 20 mM Tris-HCl pH 8.5, 500 mM NaCl and 100 mM sodium 2-mercaptoethanesulfonate for 16 hours while shaking at room temperature. The next day, the protein was washed off the column using 5 mL of EPL buffer. The protein was further purified from the reaction mixture using size exclusion chromatography on a Superdex 75 16/60 column in 5 mM HEPES at pH 7.5, 140 mM NaCl, 2.5 mM KCL, 5 mM Glycin. Peak fractions were pooled, and protein aliquots were shock-frozen and stored at −80° C.

### NLS-mCherry-Cysteine:

The protein was expressed as published previously (Sarabipour, S., King, C. & Hristova, K. Uninduced high-yield bacterial expression of fluorescent proteins. Anal Biochem 449, 155-157, doi:10.1016/j.ab.2013.12.027 (2014)).

Protein sequence, which is included in the
sequence listing as SEQ ID NO: 2 (Sequence after
thrombin cleavage underlined, chromophore in
bold, cysteine in bold and italic):
MGSSHHHHHHSSGLVPRGSHMPAAKRVKLDMVSKGEEDNMAIIKEFMRF
KVHMEGSVNGHEFEIEGEGEGRPYEGTQTAKLKVTKGGPLPFAWDILSP
QFMYGSKAYVKHPADIPDYLKLSFPEGFKWERVMNFEDGGVVTVTQDSS
LQDGEFIYKVKLRGTNFPSDGPVMQKKTMGWEASSERMYPEDGALKGEI
KQRLKLKDGGHYDAEVKTTYKAKKPVQLPGAYNVNIKLDITSHNEDYTI
VEQYERAEGRHSTGGMDELYKACA*
The ″*″ symbolizes the stop codon.

For the expression, BL21 DE3 cells were transformed with the plasmid. A single colony from an agar plate was picked and grown for 24 hours at 37° C. in 250 mL of LB medium with 40 μg/mL Kanamycin. Induction was not necessary. Cells were collected by centrifugation at 4000×g for 15 minutes. The cells were washed once in PBS, then resuspended in lysis buffer and lysed using sonication (3×2 min, 30% Amplitude), followed by debris centrifugation at 25′000×g for 30 min.

For the purification, the clear lysate was loaded on 2 mL of Ni-NTA agarose. The beads were washed with 20 column volumes of PBS with 20 mM imidazole. The protein was then eluted using 2 mL of PBS containing 500 mM imidazole. The purification tag was removed by the addition of thrombin (1:1000 v/v), overnight at 37° C. for 18 hours. The protein was further purified by size exclusion chromatography using a Superdex 75 16/60 column in 5 mM HEPES at pH 7.5, 140 mM NaCl, 2.5 mM KCL, 5 mM Glycine. Peak fractions were pooled, and protein aliquots were shock-frozen and stored at −80° C.

### NLS-mCherry-exR10 IV:

The NLS-mCherry-exR10 construct was cloned from the NLS-mCherry plasmid using Gibson assembly (Gibson, D. G. Enzymatic assembly of overlapping DNA fragments. Methods Enzymol 498, 349-361, doi:10.1016/B978-O-12-385120-8.00015-2 (2011)). A 7 amino acid long linker and 10 arginines were introduced at the C-terminus using overlap extension PCR, and the thrombin cleavage site was exchanged for a TEV protease cleavage site in the same PCR reaction. The construct was cloned back into the pET28a(+) bacterial expression plasmid in the assembly reaction.

Protein sequence, which is included in the
sequence listing as SEQ ID NO: 3 (NLS in bold,
Chromophore underlined, R10 sequence in italic
and bold):
MGSSHHHHHHSSGENLYFQGPAAKRVKLDMVSKGEEDNMAIIKEFMRFK
VHMEGSVNGHEFEIEGEGEGRPYEGTQTAKLKVTKGGPLPFAWDILSPQ
FMYGSKAYVKHPADIPDYLKLSFPEGFKWERVMNFEDGGVVTVTQDSSL
QDGEFIYKVKLRGTNFPSDGPVMQKKTMGWEASSERMYPEDGALKGEIK
QRLKLKDGGHYDAEVKTTYKAKKPVQLPGAYNVNIKLDITSHNEDYTIV
EQYERAEGRHSTGGMDELYKASGSGSGRRRRRRRRRR*
The ″*″ symbolizes the stop codon.

NLS-mCherry-exR10 was expressed in BL21 DE3 cells transformed with the plasmid. The cells were grown overnight at 37° C. in 5 mL of LB medium with 40 μg/mL kanamycin. The next day, the expression culture in 250 mL LB medium with kanamycin was inoculated with 1 mL of the starter culture. After incubation at 37° C., when the culture reached an OD600 of 0.6, expression was induced with 0.5 mM IPTG, and the culture was incubated for 16 hours at 18° C. The cells were first harvested by centrifugation at 4000×g for 15 minutes, washed once with PBS, then resuspended in lysis buffer and lysed using sonication (3×2 min, 30% Amplitude), followed by debris centrifugation at 25′000×g for 30 min.

For the purification, the clear lysate was loaded on 2 mL of Ni-NTA agarose. The beads were washed with 20 column volumes of PBS with 20 mM imidazole. The protein was then eluted with 2 mL of PBS containing 500 mM imidazole. The purification tag was not removed as it led to unexpected degradation, possibly of the C-terminal R10 peptide. The protein was further purified by size exclusion chromatography using a Superdex 75 16/60 column in 5 mM HEPES at pH 7.5, 140 mM NaCl, 2.5 mM KCL, 5 mM Glycine. Peak fractions were pooled, and protein aliquots were shock-frozen and stored at −80° C.

### NLS-Cre-exR8:

A plasmid encoding NLS-Cre recombinase was obtained from addgene (Plasmid #62730). The Cre-exR8 construct was cloned using overlap extension PCR from the original plasmid by appending 8 arginines to the C-terminus of the protein and by appending a TEV protease cleavage site on the N-terminus of the protein. The PCR product was inserted into the pET28a vector using Gibson assembly.

Protein sequence, which is included in the
sequence listing as SEQ ID NO: 4 (NLS in bold,
R8 peptide in italic and bold):
MGSSHHHHHHSSGENLYFQGPKKKRKVSNLLTVHQNLPALPVDATSDEV
RKNLMDMFRDRQAFSEHTWKMLLSVCRSWAAWCKLNNRKWFPAEPEDVR
DYLLYLQARGLAVKTIQQHLGQLNMLHRRSGLPRPSDSNAVSLVMRRIR
KENVDAGERAKQALAFERTDFDQVRSLMENSDRCQDIRNLAFLGIAYNT
LLRIAEIARIRVKDISRTDGGRMLIHIGRTKTLVSTAGVEKALSLGVTK
LVERWISVSGVADDPNNYLFCRVRKNGVAAPSATSQLSTRALEGIFEAT
HRLIYGAKDDSGQRYLAWSGHSARVGAARDMARAGVSIPEIMQAGGWTN
VNIVMNYIRNLDSETGAMVRLLEDGDASGRRRRRRRR*
The ″*″ symbolizes the stop codon.

NLS-Cre-exR8 was expressed by transforming the corresponding plasmid into BL21 DE3 cells, which were grown overnight at 37° C. in 5 mL of LB medium with 40 μg/mL kanamycin. The next day, a culture in 250 mL LB medium containing kanamycin was inoculated with 1 mL of the starter culture and grown at 37° C. until the OD600 reached 0.6. Expression was induced with 0.5 mM IPTG and the cells were incubated for another 16 hours at 18° C. The cells were harvested using centrifugation at 4000×g for 15 minutes, washed with PBS once, then taken up in 100 mM NaH₂PO₄ with 10 mM Tris pH 8.0, 300 mM NaCl, 10 mM imidazole and lysed using sonication (3×2 min, 30% Amplitude), followed by debris centrifugation at 25′000×g for 30 min.

For the purification, the clear lysate was loaded on 2 mL of Ni-NTA agarose equilibrated in phosphate buffer (100 mM NaH₂PO₄ with 10 mM Tris pH 8.0, 300 mM NaCl, 10 mM imidazole). The protein was washed with 20 column volumes of the same buffer and subsequently eluted with the same buffer containing 250 mM imidazole. The protein was further purified on a Superdex 75 16/60 column in 100 mM NaH₂PO₄ with 10 mM Tris pH 8.0, 300 mM NaCl. Peak fractions were combined, frozen in liquid nitrogen and stored at −80° C. until use.

### Cloning of Plasmids for Transfection:

The Cre Stoplight 2.4 plasmid (Yang, Y. S. & Hughes, T. E. Cre stoplight: a red/green fluorescent reporter of Cre recombinase expression in living cells. Biotechniques 31, 1036, 1038, 1040-1031, doi:10.2144/01315st03 (2001)) was obtained from addgene (Plasmid #37402).

For the cell-surface halotag-reporter plasmid, a dual cytomegalovirus (CMV)-reporter plasmid that led to expression of EGFP within the cell along with a peroxidase on the cell surface (addgene plasmid #31156) was used as a starting point. A sequence encoding the halotag was generated by PCR from the pHTN vector (Promega). The peroxidase sequence was then replaced with the halotag sequence using Gibson cloning.

### Mammalian Cell Culture

Cell lines were grown at 37° C. in a humidified atmosphere with 5% CO₂. A list of cell lines with their corresponding media can be found in supplementary table 1.

TABLE 3
Cell lines used in this study.

| Cell line | Medium |
|---|---|
| HeLa CCL-2 | DMEM 4.5 g/L Glucose + 10% fetal bovine |
| | serum (FBS), 1% Penicillin-Streptomycin |
| | (PS) |
| HeLa Kyoto | DMEM 4.5 g/L Glucose + 10% FBS, 1% PS |
| HeLa Kyoto GFP-PCNA | DMEM 4.5 g/L Glucose + 10% FBS, 1% PS |
| (Chagin, V. O. et al. 4D | |
| Visualization of replication | |
| foci in mammalian cells | |
| corresponding to individual | |
| replicons. Nat Commun 7, | |
| 11231, | |
| doi: 10.1038/ncomms11231 | |
| (2016). Leonhardt, H. et al. | |
| Dynamics of DNA | |
| replication factories in | |
| living cells. J Cell Biol 149, | |
| 271-280, | |
| doi: 10.1083/jcb.149.2.271 | |
| (2000)) | |
| SKBR-3 | DMEM/Ham's F-12 + 10% FBS, 1% PS |
| A549 | DMEM/Ham's F-12 + 10% FBS, 1% PS |
| MDCK-2 | DMEM 1 g/L Glucose + 10% FBS, 1% PS |
| SJSA-1 | RPMI 1640 + 10% FBS, 1% PS |

For the Calcein AM cell viability assay, 20′000 HeLa Kyoto cells were seeded on ibidi slides. The cells were left to adhere and grow for 24 hours at 37° C. and 5% C02. The cells were washed once with DMEM before addition of either 5 μM NLS-mCherry-R10 in DMEM alone or 5 μM NLS-mCherry-R10 with 10 μM TNB-R10 in DMEM. After one hour incubation at 37° C. and 5% C02 cells were washed three times with DMEM before treatment with 5 μM Calcein AM (Sigma; diluted from a 500 mM stock solution of Calcein AM in dimethyl sulfoxide (DMSO)) in DMEM. Microscopy images were collected three and six hours after addition of Calcein AM. Confocal microscopic images were acquired on a Leica SP511 equipped with an oil immersion ACS APO 63x/1.3 NA objective with laser line at 561 nm and 488 nm using similar settings for the same fluorophores.

### On-Cell Biotinylation, Pulldown and Proteomics

Each treatment and pulldown was performed in quadruplicate to obtain reliable label-free quantification (LFQ) data. 500′000 HeLa Kyoto cells were seeded in each well of a 6-well plate. The cells were incubated for 48 hours at 37° C. and 5% C02 to adhere and grow to near-confluency. The cells were then washed three times with PBS, then incubated for 30 minutes at room temperature with either PBS only, or 20 μM of commercially available Biotin-Maleimide (Sigma-Aldrich) or the Maleimide-R10-biotin peptide. Cells were then washed twice with PBS.

For cell lysis 500 μl of lysis buffer (2% NP-40, 1% Triton X-100, 10% glycerol, EDTA free protease inhibitor tablet in PBS) was added to the cells. Cells were scraped off the plates and transferred to a reaction tube followed by incubation on ice on a shaker for 30 min. The cell extracts were centrifuged for 20 min (20800×g, at 4° C.) to pellet the insoluble material.

The supernatants were mixed with 100 μl of Streptavidin-agarose beads and the beads were incubated for 1 hour at 4° C. Beads were washed twice with lysis buffer, then another two times times with 300 mM NaCl in lysis buffer.

Then, 50 μl of 4×Laemmli buffer containing beta-mercaptoethanol were added to the beads and they were boiled at 75° C. for 15 minutes to denature proteins and disturb the streptavidin-biotin interaction.

20 μl for each condition were loaded on a 10% SDS-PAGE gel. The samples were only run until approximately 1 cm into the separating gel. This part was cut out and used for the in-gel tryptic digest. Samples in the gel were reduced, alkylated and digested with trypsin followed by extraction from the gel as described previously (Shevchenko, A., Tomas, H., Havlis, J., Olsen, J. V. & Mann, M. In-gel digestion for mass spectrometric characterization of proteins and proteomes. Nat Protoc 1, 2856-2860, doi:10.1038/nprot.2006.468 (2006)).

MS-spectra were input into MaxQuant to identify proteins from the Uniprot Homo Sapiens database. All cysteines were specified with a static modification for carbamidomethylation (+57.02146). Perseus was used to process the data (two-tailed t-tests, false discovery rate=0.1) and generate the sample matrix and volcano plots.

### Flow Cytometry

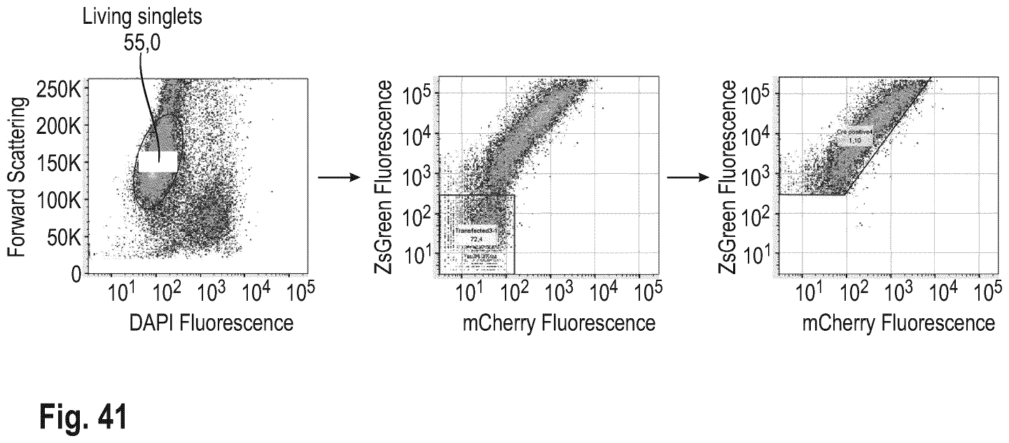

For the Cre recombinase experiments, 200′000 cells were seeded in each well of a 12-well plate. The cells were incubated for 24 hours at 37° C. to settle, then transfected with the reporter plasmid (Cre Stoplight 2.4) using Lipofectamine 2000. The cells were incubated for another 24 hours, then treated with Cre recombinase (or medium) in DMEM with 5% FCS and incubated for 24 more hours. Microscopy pictures were then taken, and the cells were detached with accutase, dead cells stained with DAPI and all cells measured on a LSRFortessa (BD Biosciences, USA) flow cytometer. Dead cells and multiplets were removed in the analysis through gating, followed by untransfected cells that showed no fluorescence in either the green or red channel. At least 10′000 cells were counted for each condition. The gating strategy is illustrated in FIG. 41.

1. A compound of formula (1): 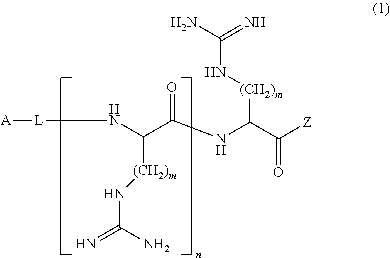
wherein:
A is a moiety capable of binding to a cell surface;
L is a linker or a bond;
m is each independently an integer ranging from 0 to 10;
n is an integer ranging from 1 to 20;
Z is selected from the group consisting of NR¹R², OR³, an amino acid, a peptide comprising 2 to 10 amino acids, and a hydrophobic moiety;
R¹ and R² are each independently selected from hydrogen and (C₁-C₆)alkyl; wherein optionally, when R¹ and R² are (C₁-C₆)alkyl, R¹ and R² together with the nitrogen atom to which they are attached form a four- to seven-membered ring;
R³ is hydrogen or (C₁-C₆)alkyl;
or a pharmaceutically acceptable salt thereof.

2. The compound according to claim 1, wherein
A is a thiol-reactive moiety.

3. The compound according to claim 1, wherein Z is selected from the group consisting of NR¹R², OR³, an amino acid, and a peptide comprising 2 to 10 amino acids.

4. The compound according to claim 1, wherein Z is a hydrophobic moiety.

5. The compound according to claim 1, having formula (2): 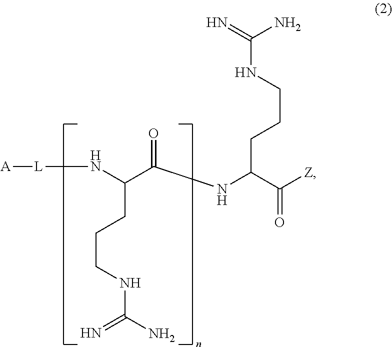
wherein A, L, n and Z are as defined in claim 1.

6. The compound according to claim 1, wherein L is
—[B¹—(CH₂CH₂B²)ₕ—(CH₂)ⱼ—C(O)]ₖ—,
wherein B¹ and B² are independently selected from the group consisting of CH₂, NH and O; wherein h is an integer ranging from 1 to 4; j is 1 or 2; and k is an integer ranging from 1 to 10.

7.-9. (canceled)

10. A method for delivering a cargo into a cell, the method comprising incubating a compound comprising a moiety capable of binding to the cell surface and a guanidine moiety together with a cargo and a cell, wherein the cargo is connected with a group comprising a guanidine moiety, thereby allowing delivering of the cargo into the cell, and wherein the compound is a compound according to claim 1.

11. The method according to claim 10, wherein
(i) the cargo is connected such that the group is conjugated with or fused to a group comprising a guanidine moiety;
(ii) the cargo is selected from peptide, protein, enzyme, nanobody, oligonucleotide, nanoparticle and antibody;
(iii) the delivered cargoes are antibodies;
(iv) the moiety of the compound capable of binding to the cell surface is a thiol-reactive moiety, or wherein the moiety is capable of binding to the cell surface via an enzymatic reaction; and/or
(v) the compound comprising a moiety capable of binding to the cell surface and a guanidine moiety further comprises a hydrophobic moiety.

12. The method according to claim 10, the method comprising:
(a) incubating the compound comprising a moiety capable of binding to the cell surface and a guanidine moiety together with the cargo connected with the group comprising a guanidine moiety to obtain a solution comprising the compound and the cargo connected with the group,
(b) incubating the solution of step (a) with the cell, thereby allowing delivering of the cargo into the cell.

13. The method according to claim 12, wherein the moiety of the compound is capable of binding to a tag or target structure on the cell surface, and wherein the method comprises:
(a) transfecting a cell with a tag such that the tag is expressed on the cell surface, or modifying the cell surface with a target structure,
(b) incubating the compound comprising the moiety capable of binding to the tag or target structure on the cell surface and a guanidine moiety together with the cargo connected with the group comprising a guanidine moiety to obtain a solution comprising the compound and the cargo connected with the group,
(c) incubating the solution of step (b) with the cell, thereby allowing delivering of the cargo into the cell; preferably wherein in (c) the incubating the solution of (b) with the cell is carried out for a time of 1 minute to 24 hours.

14. The method according to claim 13, wherein the tag is a Halotag, or wherein the structure is a bioorthogonal reporter on the cell surface.

15.-17. (canceled)

18. A kit comprising a compound, the compound comprising a moiety capable of binding to a cell surface and a guanidine moiety, wherein the compound is as defined in claim 1.

19. The compound according to claim 1, wherein A is 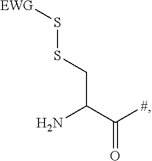
wherein #indicates the attachment point to the L; and
EWG is an electron-withdrawing group.

20. The compound according to claim 19, wherein EWG is selected from the group consisting of 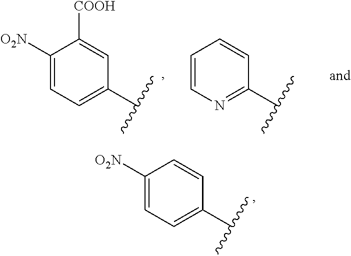 [SMILES: *c1ccc([N+](=O)[O-])c(C(=O)O)c1 ; *c1ccccn1 ; *c1ccc([N+](=O)[O-])cc1]
wherein [symbol] indicates the attachment point to the S.

21. The compound according to claim 1, wherein A is 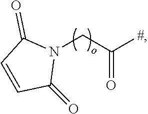
wherein o is an integer ranging from 0 to 10, and #indicates the attachment point to the L.

22. The compound according to claim 1, wherein A is capable of binding to the cell surface via an enzymatic reaction.

23. The compound according to claim 22, wherein A is capable of binding to a Halotag.

24. The compound according to claim 4, wherein Z is a peptide comprising 2 to 10 amino acids independently selected from the group consisting of glycine, alanine, valine, leucine, isoleucine, proline, phenylalanine, methionine, tryptophan and one or more hydrophobic unnatural amino acid(s).

25. The compound according to claim 4, wherein Z comprises or is (C₁-C₂₀)perfluoroalkyl.

26. The compound according to claim 1, wherein the compound has formula (2b): 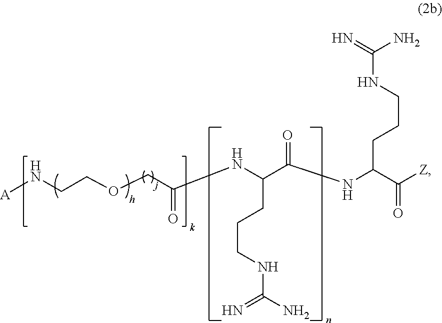
wherein A, h, j, k, n and Z are as defined in claim 1.


SEQUENCE LISTING (17 sequences)

SEQ ID NO: 1 - protein, 380 aa, Artificial
  GBP1 Nanobody
MADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSAAACITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQ

SEQ ID NO: 2 - protein, 269 aa, Artificial
  NLS-mCherry-Cysteine
MGSSHHHHHHSSGLVPRGSHMPAAKRVKLDMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKACA

SEQ ID NO: 3 - protein, 282 aa, Artificial
  NLS-mCherry-exR10 IV
MGSSHHHHHHSSGENLYFQGPAAKRVKLDMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKASGSGSGRRRRRRRRRR

SEQ ID NO: 4 - protein, 380 aa, Artificial
  NLS-Cre-exR8
MGSSHHHHHHSSGENLYFQGPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDASGRRRRRRRR

SEQ ID NO: 5 - protein, 10 aa, Artificial
  Maleimide-R10
  BINDING (1)..(1): Maleimidoacetic acid-PEG*, PEG* corresponds to two consecutively coupled units of 8-amino-3,6-dioxaoctanoic acid
  BINDING-Amide (10)..(10)
RRRRRRRRRR

SEQ ID NO: 6 - protein, 11 aa, Artificial
  Cys-R10
  BINDING-PEG* (1)..(2): PEG* corresponds to two consecutively coupled units of 8-amino-3,6-dioxaoctanoic acid
  BINDING-Amide (11)..(11)
CRRRRRRRRRR

SEQ ID NO: 7 - protein, 10 aa, Artificial
  Maleimide-K10
  BINDING (1)..(1): Maleimidoacetic acid-PEG*, PEG* corresponds to two consecutively coupled units of 8-amino-3,6-dioxaoctanoic acid
  BINDING-Amide (10)..(10)
KKKKKKKKKK

SEQ ID NO: 8 - protein, 12 aa, Artificial
  Cys-R10-TAMRA
  BINDING-PEG* (1)..(2): PEG* corresponds to two consecutively coupled units of 8-amino-3,6-dioxaoctanoic acid
  BINDING-Mtt-Amide (12)..(12): Mtt corresponds to N-methyltrityl
CRRRRRRRRRRK

SEQ ID NO: 9 - protein, 11 aa, Artificial
  TAMRA-R5-Cys-R5
  BINDING-Amide (11)..(11)
RRRRRCRRRRR

SEQ ID NO: 10 - protein, 11 aa, Artificial
  TAMRA-R10-Cys
  BINDING-Amide (11)..(11)
RRRRRRRRRRC

SEQ ID NO: 11 - protein, 11 aa, Artificial
  Maleimide-R10-Cy5/Biotin
  BINDING (1)..(1): Maleimidoacetic acid-PEG*, PEG* corresponds to two consecutively coupled units of 8-amino-3,6-dioxaoctanoic acid
  BINDING-Mtt-Amide (11)..(11): Mtt corresponds to N-methyltrityl
RRRRRRRRRRK

SEQ ID NO: 12 - protein, 10 aa, Artificial
  Halo-R10
  BINDING (1)..(1): 6-chlorohexanoic acid-PEG*, PEG* corresponds to two consecutively coupled units of 8-amino-3,6-dioxaoctanoic acid
  BINDING-Amide (10)..(10)
RRRRRRRRRR

SEQ ID NO: 13 - protein, 5 aa, Artificial
  Maleimide-R5
  BINDING (1)..(1): Maleimidoacetic acid-PEG*, PEG* corresponds to two consecutively coupled units of 8-amino-3,6-dioxaoctanoic acid
  BINDING-Amide (5)..(5)
RRRRR

SEQ ID NO: 14 - protein, 8 aa, Artificial
  Maleimide-R8
  BINDING (1)..(1): Maleimidoacetic acid-PEG*, PEG* corresponds to two consecutively coupled units of 8-amino-3,6-dioxaoctanoic acid
  BINDING-Amide (8)..(8)
RRRRRRRR

SEQ ID NO: 15 - protein, 10 aa, Artificial
  Mal-PEG2-R10
  BINDING (1)..(1): Maleimidoacetic acid-PEG*, PEG* corresponds to two consecutively coupled units of 8-amino-3,6-dioxaoctanoic acid
  BINDING-Amide (10)..(10)
RRRRRRRRRR

SEQ ID NO: 16 - protein, 16 aa, Artificial
  Mal-PEG2-R10-ILFF
  BINDING (1)..(1): Maleimidoacetic acid-PEG*, PEG* corresponds to two consecutively coupled units of 8-amino-3,6-dioxaoctanoic acid
  BINDING-Amide (16)..(16)
RRRRRRRRRRGGILFF

SEQ ID NO: 17 - protein, 11 aa, Artificial
  Mal-PEG2-R10-fluorous tag
  BINDING (1)..(1): Maleimidoacetic acid-PEG*, PEG* corresponds to two consecutively coupled units of 8-amino-3,6-dioxaoctanoic acid
  BINDING-Mtt-Amide (11)..(11): Mtt corresponds to N-methyltrityl
RRRRRRRRRRK